SEQUENCE LISTING (111 sequences)

SEQ ID NO: 1 - DNA, 1522 bp, Clostridium difficile
taaagagtttgatcctggctcaggacgaacgctgtcggcgcgcctaacacatgcaagtcgaacgaagttgctttgaatgaattcttcggaaggaatttgattcaacttagtggcggacgggtgagtaacgcgtgagtaacctgcctttcagagggggataacgtctggaaacggacgctaataccgcataacatattggtttcgcatggagctgatatcaaaggagcaatccgctgaaagatggactcgcgtccaattagctagttggtgaggtaacggcccaccaaggcgacgattggtagccggactgagaggttgaacggccacattgggactgagacacggcccagactcctacgggaggcagcagtggggaatattgcacaatgggggaaaccctgatgcagcgacgccgagtgagggaagaaggttttcggattgtaaacctctgtccttggtgaagataatgacggtaaccaaggaggaagctacggctaactacgtgccagcagccgcggtaatacgtaggtagcgagcgttgtccggaattactgggtgtaaagggagcgtaggcgggattgcaagttgaatgttaaatctatgggctcaacccatagccgcgttcaaaactgcagttcttgagtgaagtagaggcaggcggaattcctagtgtagcggtgaaatgcgtaaatattaggaggaacaccagtggcgaaggcggcctgctgggctttaactgacgctgaggctcgaaagcgtgggtagcaaacaggattagataccctggtagtccacgctgtaaacgatgattactaggtgtggggggactgaccccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccggtgcatagcctagagataggtgaagcccttcggggcaccgagacaggtggtgcatggttgtcgtcagctcgtgtcgtgagatgttgggttaagtcccgcaacgagcgcaacccttacgtttagttgctacgcaagagcactctagacggactgccgttgacaaaacggaggaaggtggggatgacgtcaaatcatcatgccccttatgacctgggctacacacgtactacaatggctattaacagagggaagcaagatggtgacatggagcaaacccctaaaaatagtctcagttcggattgcaggctgcaacccgcctgcatgaagccggaattgctagtaatcgcggatcagcatgccgcggtgaatacgttcccgggccttgtacacaccgcccgtcacaccatgagagttggcaacacccgaagccgatagtctaaccgcaagggggacgtcgtcgaaggtggggttgatgattggggtgaagtcgtaacaaggtagccgtatcggaaggtgcggctggatcacctccttt

SEQ ID NO: 2 - DNA, 1528 bp, Artificial Sequence
  Blautia_SC102
tcagagagtttgatcctggctcaggatgaacgctggcggcgtgcttaacacatgcaagtcgaacggggattatttcattgaagcttcggcagatttggtttaatcctagtggcggacgggtgagtaacgcgtgggtaacctgccttatacagggggataacagtcagaaatggctgctaataccgcataagcgcacagggccgcatggcccggtgtgaaaaactgaggtggtataagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcataacaagtctgatgtgaaaggctggggcttaaccccgggactgcattggaaactgttaagcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcacagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatctgcctgaccggtgagtaacgtcacctttccttcgggacaggcaagacaggtggtgcatggttgtcgtcagctcgtgtcgtgagatgttgggttaagtcccgcaacgagcgcaacccctatccccagtagccagcatgtaaaggtgggcactctgaggagactgccagggataacctggaggaaggtggggatgacgtcaaatcatcatgccccttatgatttgggctacacacgtgctacaatggcgtaaacagagggaagcgaaagggtgacctggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgaaagggaggagctgccgaaggcgggacggatgactggggtgaagtcgtaacaaggtagccgtatcggaaggtgcggctggatcacctccttt

SEQ ID NO: 3 - DNA, 1526 bp, Artificial Sequence
  Blautia_SC109
atgagagtttgatcctggctcaggatgaacgctggcggcgtgcttaacacatgcaagtcgagcgaagcaatttaagtgaagttttcggatggatcttagattgactgagcggcggacgggtgagtaacgcgtggataacctgcctcacacagggggataacagttagaaatgactgctaataccgcataagcgcacggtaccgcatggtacagtgtgaaaaactccggtggtgtgagatggatccgcgtctgattaggtagttggtgaggtaacggcccaccaagccgacgatcagtagccgacctgagagggtgaccggccacattgggactgagacacggcccaaactcctacgggaggcagcagtggggaatattgcacaatgggcgaaagcctgatgcagcaacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgcagacggcactgcaagtctgaagtgaaagcccggggctcaaccccgggactgctttggaaactgtagagctagagtgctggagaggcaagcggaattcctagtgtagcggtgaaatgcgtagatattaggaagaacaccagtggcgaaggcggcttgctggacagtaactgacgttcaggctcgaaagcgtggggagcaaacaggattagataccctggtagtccacgccgtaaacgatgaatactaggtgttggtgggcaaagcccatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcgtgatgaccggaacttaaccgttccttcccttcggggcatcatagacaggtggtgcatggttgtcgtcagctcgtgtcgtgagatgttgggttaagtcccgcaacgagcgcaacccttatcctcagtagccagcagtaagatgggcactctggggagactgccagggataacctggaggaaggtggggatgacgtcaaatcatcatgccccttatgatttgggctacacacgtgctacaatggcgtaaacaaagggaagcaagagggtgacctggagcaaatcccaaaaataacgtcccagttcggactgtagtctgcaacccgactacacgaagctggaatcgctagtaatcgcagatcagaatgctgcggtgaatacgttcccgggtcttgtacacaccgcccgtcacaccatgggagtcagcaacgcccgaagtcagtgactcaaccgcaaggagagagctgccgaaggcggggcaggtaactggggtgaagtcgtaacaaggtagccgtatcggaaggtgcggctggatcacctccttt

SEQ ID NO: 4 - DNA, 1526 bp, Artificial Sequence
  Blautia_SC109
atgagagtttgatcctggctcaggatgaacgctggcggcgtgcttaacacatgcaagtcgagcgaagcaatttaagtgaagttttcggatggatcttagattgactgagcggcggacgggtgagtaacgcgtggataacctgcctcacacagggggataacagttagaaatgactgctaataccgcataagcgcacggtaccgcatggtacagtgtgaaaaactccggtggtgtgagatggatccgcgtctgattaggtagttggtgaggtaacggcccaccaagccgacgatcagtagccgacctgagagggtgaccggccacattgggactgagacacggcccaaactcctacgggaggcagcagtggggaatattgcacaatgggcgaaagcctgatgcagcaacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgcagacggcactgcaagtctgaagtgaaagcccggggctcaaccccgggactgctttggaaactgtagagctagagtgctggagaggcaagcggaattcctagtgtagcggtgaaatgcgtagatattaggaagaacaccagtggcgaaggcggcttgctggacagtaactgacgttcaggctcgaaagcgtggggagcaaacaggattagataccctggtagtccacgccgtaaacgatgaatactaggtgttggtgggcaaagcccatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcgtgatgaccggaacttaaccgttccttcccttcggggcatcatagacaggtggtgcatggttgtcgtcagctcgtgtcgtgagatgttgggttaagtcccgcaacgagcgcaacccttatcctcagtagccagcagtaagatgggcactctgtggagactgccagggataacctggaggaaggtggggatgacgtcaaatcatcatgccccttatgatttgggctacacacgtgctacaatggcgtaaacaaagggaagcaagagggtgacctggagcaaatcccaaaaataacgtcccagttcggactgtagtctgcaacccgactacacgaagctggaatcgctagtaatcgcagatcagaatgctgcggtgaatacgttcccgggtcttgtacacaccgcccgtcacaccatgggagtcagcaacgcccgaagtcagtgactcaaccgcaaggagagagctgccgaaggcggggcaggtaactggggtgaagtcgtaacaaggtagccgtatcggaaggtgcggctggatcacctccttt

SEQ ID NO: 5 - DNA, 1525 bp, Clostridium methylpentosum
attaagagtttgatcctggctcaggacgaacgctggcggcgcgcctaacacatgcaagtcgaacggagttgttttggagaagcccttcggggtggaactgattcaacttagtggcggacgggtgagtaacacgtgagcaacctgccttacagaggggaataacgtttggaaacgaacgctaataccgcataacataacggaatcgcatggttttgttatcaaagattatatcgctgtaagatgggctcgcgtctgattagatagttggtgaggtaatggctcaccaagtcgacgatcagtagccggactgagaggttgaacggccacattgggactgagacacggcccagactcctacgggaggcagcagtggggaatattgcacaatgggggaaaccctgatgcagcgacgccgcgtgaaggaagaaggccttcgggttgtaaacttctgtcttcagggacgataatgacggtacctgaggaggaagctccggctaactacgtgccagcagccgcggtaatacgtagggagcgagcgttgtccggaattactgggtgtaaagggagcgtaggcgggattgcaagttgaatgtgaaatctatgggcttaacccataaactgcgttcaaaactgcagttcttgagtgaagtagaggcaggcggaattcctagtgtagcggtgaaatgcgtagatattaggaggaacaccagtggcgaaggcggcctgctgggctttaactgacgctgaggctcgaaagcgtgggtagcaaacaggattagataccctggtagtccacgccgtaaacgatgattactaggtgtaggggggtcaaccttctgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttacatttagttgctacgcaagagcactctagatggactgccgttgacaaaacggaggaaggtggggatgacgtcaaatcatcatgccccttatgacctgggctacacacgtactacaatggctattaacagagggaagcaaaacagtgatgtggagcaaacccctaaaaatagtctcagttcggattgtaggctgcaactcgcctacatgaagccggaattgctagtaatcgcggatcagcatgccgcggtgaatacgttcccgggccttgtacacaccgcccgtcacaccatgagagttggcaacacccgaagtcagtagtctaaccgcaaggaggacgctgccgaaggtggggttgatgattagggtgaagtcgtaacaaggtagccgtatcggaaggtgcggctggatcacctccttt

SEQ ID NO: 6 - DNA, 1530 bp, Pseudoflavonifractor capillosus
tattgagagtttgatcctggctcaggatgaacgctggcggcgtgcttaacacatgcaagtcgaacggagagctcatgacagaggattcgtccaatggattgggtttcttagtggcggacgggtgagtaacgcgtgaggaacctgcctcggagtggggaataacagtccgaaaggactgctaataccgcataatgcagctgagtcgcatgacactggctgccaaagatttatcgctctgagatggcctcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggatgaaggctttcgggttgtaaacttcttttatcagggacgaaataaatgacggtacctgatgaataagccacggctaactacgtgccagcagccgcggtaatacgtaggtggcaagcgttatccggatttactgggtgtaaagggcgtgtaggcgggactgcaagtcaggtgtgaaaaccacgggctcaacctgtggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatccgactaacgaagcagagatgcattaggtgcccttcggggaaagtcgagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaagcaatgccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 7 - DNA, 1501 bp, Ethanoligenens harbinense
ttggagagtttgatcctggctcaggacgaacgctggcggcgcgcctaacacatgcaagtcgagcggagtccttcgggacttagcggcggacgggtgagtaacgcgtgagcaacctggccttcagagggggataacgtctggaaacggacgctaataccgcatgacatggcggagtcgcatggctctgccatcaaaggagtaatccgctgagggatgggctcgcgtccgattaggtagttggtgaggtaacggctcaccaagcccgcgatcggtagccggactgagaggttggccggccacattgggactgagacacggcccagactcctacgggaggcagcagtgggggatattgcacaatggaggaaactctgatgcagcgacgccgcgtgagggaagaaggtcttcggattgtaaacctctgtctttggggacgaatcaatgacggtacccaaggaggaagccacggctaactacgtgccagcagccgcggtaatacgtaggtggcaagcgttgtccggaattactgggtgtaaagggtgcgcaggcggggcggcaagttggatgtgaaaactccgggctcaacccggagcctgcattcaaaactgtcgctcttgagtgaagtagaggcaggcggaattcccggtgtagcggtgaaatgcgtagatatcgggaggaacaccagtggcgaaggcggcctgctgggcttttactgacgctgaggcacgaaagcatgggtagcaaacaggattagataccctggtagtccatgccgtaaacgatgattgctaggtgtggggggtctgaccccttccgtgccggagttaacacaataagcaatccacctggggagtacggccgcaaggttgaaactcaaaggaattgacgggggcccgcacaagcagtggagtatgtggtttaattcgaagcaacgcgaagaaccttaccaggtcttgacatccaccgaatcccccagagatgggggagtgcccttcggggagcggtgagacaggtggtgcatggttgtcgtcagctcgtgtcgtgagatgttgggttaagtcccgcaacgagcgcaacccttgtgaatagttgctacgaaagagcactctattcagaccgccgttgacaaaacggaggaaggtggggatgacgtcaaatcatcatgccccttatgacctgggctacacacgtactacaatggccatcaacagagggaagcaaggccgcgaggtggagcgaacccctaaaaatggtctcagttcagattgcaggctgaaacccgcctgcatgaagatggaattgctagtaatcgcggatcagcatgccgcggtgaatacgttcccgggccttgtacacaccgcccgtcacaccatgagagccggggacacccgaagtcggttgggtaaccgtaaggagcccgccgccgaaggtggaatcggtaattggggtgaagtcgtaacaaggtagccgtatcggaaggtgcggctggatcacctccttt

SEQ ID NO: 8 - DNA, 1510 bp, Ruminococcus albus
tattaagagtttgatcctggctcaggacgaacgctggcggcacgcttaacacatgcaagtcgaacgagcgaaagagtgcttgcactctctagctagtggcggacgggtgagtaacacgtgagcaatctgcctttcggagagggataccaattggaaacgattgttaatacctcataacataacgaagccgcatgactttgttatcaaatgaatttcgccgaaagatgagctcgcgtctgattaggtagttggtgaggtaacggcccaccaagccgacgatcagtagccggactgagaggttgaacggccacattgggactgagacacggcccagactcctacgggaggcagcagtggggaatattgcacaatgggcgaaagcctgatgcagcgatgccgcgtgagggaagaaggttttaggattgtaaacctctgtctttggggacgataatgacggtacccaaggaggaagctccggctaactacgtgccagcagccgcggtaatacgtagggagcgagcgttgtccggaattactgggtgtaaagggagcgtaggcgggattgcaagtcaggtgtgaaatttaggggcttaacccctgaactgcacttgaaactgtagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggctgaaactcaaaggaattgacggggacccgcacaagcagtggagtatgtggtttaattcgaagcaacgcgaagaaccttaccaggtcttgacatcgtacgcatagcatagagatatgtgaaatcccttcggggacgtatagacaggtggtgcatggttgtcgtcagctcgtgtcgtgagatgttggggttaagtcccgcaacgagcgcaacccttactgttagttgctacgcaagagcactctagcaggactgccgttgacaaaacggaggaaggtggggatgacgtcaaatcatcatgccccttatgacctgggctacacacgtactacaatggctgttaacagagggaagcaaaacagtgatgtggagcaaaaccctaaaagcagtcttagttcggattgtaggctgcaacccgcctacatgaagtcggaattgctagtaatcgcggatcagcatgccgcggtgaatacgttcccgggccttgtacacaccgcccgtcacgccatgggagtcggtaacacccgaagcctgtgttctaaccgcaaggaggaagcagtcgaaggtgggattgatgactggggtgaagtcgtaacaaggtagccgtatcggaaggtgcggctggatcacctccttt

SEQ ID NO: 9 - DNA, 1504 bp, Ruminococcus champanellensis
tatgaagagtttgatcctggctcaggacgaacgctggcggcacgcctaacacatgcaagtcgaacggagataaagacttcggtttttatcttagtggcggacgggtgagtaacacgtgagcaacctgcctctgagagagggatagcttctggaaacggatggtaatacctcataacatagcggtaccgcatgatactgctatcaaagatttatcgctcagagatgggctcgcgtctgattagctagatggtgaggtaacggctcaccatggcgacgatcagtagccggactgagaggttgaacggccacattgggactgagacacggcccagactcctacgggaggcagcagtggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatcgcagatcagcatgctgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacctccttt

SEQ ID NO: 10 - DNA, 1530 bp, Flavonifractor plautii
tattgagagtttgatcctggctcaggatgaacgctggcggcgtgcttaacacatgcaagtcgaacggggtgctcatgacggaggattcgtccaatggattgagttacctagtggcggacgggtgagtaacgcgtgaggaacctgccttggagaggggaataacactccgaaaggagtgctaataccgcatgaagcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaggcagagatgcgttaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 11 - DNA, 1528 bp, Oscillibacter valericigenes
tatagagagtttgatcctggctcaggacgaacgctggcggcgtgcttaacacatgcaagtcgaacggagcacccttgattgaggtttcggccaaatgagaggaatgcttagtggcggactggtgagtaacgcgtgaggaacctgcctttcagagggggacaacagttggaaacgactgctaataccgcatgatacatttgggcgacatcgcttgaatgtcaaagatttatcgctgaaagatggcctcgcgtctgattagatagttggtgaggtaacggcccaccaagtcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagtagaagacggtaccccttgaataagccacggctaactacgtgccagcagccgcggtaatacgtaggtggcaagcgttgtccggatttactgggtgtaaagggcgtgtagccgggaaggtaagtcagatgtgaaatctgggggctcaacctccaaactgcatttgaaactacttttcttgagtatcggagaggtaatcggaattccttgtgtagcggtgaaatgcgtagatataaggaagaacaccagtggcgaaggcggattactggacgacaactgacggtgaggcgcgaaagcgtggggagcaaacaggattagataccctggtagtccacgctgtaaacgatcaatactaggtgtgcggggactgaccccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccaggacttgacatcctactaacgaggtagagatacgtcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaagccgtgaggtggagcgaacccctaaaagccgtctcagttcggatcgcaggctgcaactcgcctgcgtgaagtcggaatcgctagtaatcgcggatcagaatgccgcggtgaatacgttcccgggccttgtacacaccgcccgtcacaccatgagagtcgggaacacccgaagtccgtagcctaacagcaatgagggcgcggccgaaggtgggtttgataattggggtgaagtcgtaacaaggtagccgtatcggaaggtgcggctggatcacctccttt

SEQ ID NO: 12 - DNA, 1528 bp, Oscillibacter ruminantium
tatagagagtttgatcctggctcaggacgaacgctggcggcgtgcttaacacatgcaagtcgaacggaacacccttgacagaggtttcggccaatgaagaggaatgtttagtggcggactggtgagtaacgcgtgaggaacctgcctttcagagggggacaacagttggaaacgactgctaataccgcatgaagcagcgaggggacatccccttgctgtcaaagatttatcgctgaaagatggcctcgcgtctgattagctagttggtggggtaacggcccaccaaggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttaacagggaagagaagaagacggtacctgttgaataagccacggctaactacgtgccagcagccgcggtaatacgtaggtggcaagcgttgtccggatttactgggtgtaaagggcgtgtagccgggaaggcaagtcagatgtgaaatctggaggctcaacctccaaactgcatttgaaactgcttttcttgagtatcggagaggtaatcggaattccttgtgtagcggtgaaatgcgtagatataaggaagaacaccagtggcgaaggcggattactggacgacaactgacggtgaggcgcgaaagcgtggggagcaaacaggattagataccctggtagtccacgctgtaaacgatcaatactaggtgtgcggggactgaccccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccaggacttgacatcctactaacgaggtagagatacgtcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaagccgtgaggcagagcgaacccctaaaagccgtctcagttcggatcgtaggctgcaactcgcctacgtgaagtcggaatcgctagtaatcgcggatcagaatgccgcggtgaatacgttcccgggccttgtacacaccgcccgtcacaccatgagagtcgggaacacccgaagcccgtagcctaactgcaaagagggcgcggtcgaaggtgggttcgataattggggtgaagtcgtaacaaggtagccgtatcggaaggtgcggctggatcacctccttt

SEQ ID NO: 13 - DNA, 1029 bp, Clostridium sporosphaeroides
caaaggagcaatccgctgaaagatggactcgcgtccgattagccagttggcggggtaaaggcccaccaaagcgacgatcggtagccgggttgagagactgaacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctctttaagtcgggcgtgaaagctgtgggcttaacccacaaattgcgttcgaaactggagggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaggcagagatgcattaggtgcccttcggggaaagttgagacaggtggtgcatggttgtcgtcagctcgtgtcgtgagatgttgggttaagtcccgcaacgagcgcaacccttgtgattagttgctacgcaagagcactctaatcagactgccgttgacaaaacggaggaaggtggggacgacgtcaaatcatcatgccccttatgacctgggctacacacgtactacaatggtcgccaacagagggaagcca

SEQ ID NO: 14 - DNA, 1504 bp, Ruminococcus callidus
taaagagtttgatcctggctcaggacgaacgctggcggcacgcttaacacatgcaagtcgaacggagaacattgagcttgcttaatgttcttagtggcggacgggtgagtaacacgtgagtaacctgcctctgagagtgggatagcttctggaaacggatggtaataccgcataacatcatggattcgcatgtttctgtgatcaaagatttatcgcttagagatggactcgcgtctgattagctagttggtaaggtaacggcttaccaaggcgacgatcagtagccggactgagaggttgatcggccacattgggactgagacacggcccagactcctacgggaggcagcagtggggaatattgcacaatgggggaaaccctgatgcagcgatgccgcgtggaggaagaaggttttcggattgtaaactcctgttgaagaggacgataatgacggtactcttttagaaagctccggctaactacgtgccagcagccgcggtaatacgtagggagcgagcgttgtccggaattactgggtgtaaagggagcgtaggcgggatggcaagtcagatgtgaaaactatgggctcaacccatagactgcatttgaaactgttgttcttgagtgaggtagaggtaagcggaattcctggtgtagcggtgaaatgcgtagagatcaggaggaacatcggtggcgaaggcggcttactgggcctttactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgagtgacgtacctagagataggtattttcttcggaacacaaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggctacacacgtactacaatggcaatataacagagggaagcaatacagcgatgtggagcaaatccccaaaaattgtcccagttcagattgcaggctgcaactcgcctgcatgaagtcggaattgctagtaatcgcagatcagcatgctgcggtgaatacgttcccgggccttgtacacaccgcccgtcacaccatgggagtcggtaacacccaaagccggtcgtctaaccttcgggaggatgccgtctaaggtgggattgatgactggggtgaagtcgtaacaaggtagccgtatcggaaggtgcggctggatcacctcctt

SEQ ID NO: 15 - DNA, 1510 bp, Ruminococcus flavefaciens
ataaagagtttgatcctggctcaggacgaacgctggcggcacgcttaacacatgcaagtcgaacggagataatttgagtttacttaggttatcttagtggcggacgggtgagtaacacgtgagcaacctaccttagagagagggatagcttctggaaacggatggtaatacctcataacataactgaaccgcatgatttagttatcaaagatttatcactctgagatgggctcgcgtctgattagatagttggtgaggtaacggctcaccaagtcgacgatcagtagccggactgagaggttgaacggccacattgggactgagacacggcccagactcctacgggaggcagcagtggggaatattgcacaatgggggaaaccctgatgcagcgatgccgcgtggaggaagaaggttttcggattgtaaactcctgtcttaaaggacgataatgacggtactttaggaggaagctccggctaactacgtgccagcagccgcggtaatacgtagggagcgagcgttgtccggaattactgggtgtaaagggagcgtaggcgggagcgcaagtcagatgtgaaatacatgggctcaacccatgggctgcatttgaaactgtgtttcttgagtgaagtagaggtaagcggaattcctggtgtagcggtgaaatgcgtagatatcaggaggaacaccggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgtatgcatagtctagagatagatgaaattccttcggggacatatagacaggtggtgcatggttgtcgtcagctcgtgtcgtgagatgttgggttaagtcccgcaacgagcgcaacccttacctttagttgctacgcaagagcactctaaagggactgccgttgacaaaacggaggaaggtggggatgacgtcaaatcatcatgccccttatgacctgggctacacacgtactacaatggcaattaacaaagagaagcaagacggtgacgtggagcgaatctcaaaaaattgtcccagttcagattgcaggctgcaactcgcctgcatgaagtcggaattgctagtaatcgcggatcagcatgccgcggtgaatacgttcccgggccttgtacacaccgcccgtcacaccatgggagtcggtaacacccgaagtcggtagtctaacagcaatgaggacgccgccgaaggtgggattgatgactggggtgaagtcgtaacaaggtagccgtatcggaaggtgcggctggatcacctccttt

SEQ ID NO: 16 - DNA, 1309 bp, Clostridium jeddahense
caaaggagcaatccgctgaaagatggactcgcgtccgattagccagttggcggggtaaaggcccaccaaagcgacgatcggtagccgggttgagagactgaacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctttttaagtcgggcgtgaaagctgtgggcttaacccacaaattgcgttcgaaactggaaggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaggcagagatgcattaggtgcccttcggggaaagttgagacaggtggtgcatggttgtcgtcagctcgtgtcgtgagatgttgggttaagtcccgcaacgagcgcaacccttgtgattagttgctacgcaagagcactctaatcagactgccgttgacaaaacggaggaaggtggggacgacgtcaaatcatcatgccccttatgacctgggctacacacgtactacaatggtcgctaacagagggaagccaagccgcgaggtggagcaaacccccaaaagcgatctcagttcggattgtaggctgcaacccgcctacatgaagttggaattgctagtaatcgcggatcagcatgccgcggtgaatacgttcccgggccttgtacacaccgcccgtcacaccatgggagccggtaatacccgaagccaatagtctaaccgcaagggggacgttgtcgaaggtaggattggcgactggggtgaagtcgtaacaaggtagccgtatcggaaggtgcggctggatcacctccttt

SEQ ID NO: 17 - DNA, 1421 bp, Clostridium viride
gcttagtggcggacgggtgagtaacgcgtgagtaacctgccttggagtggggaataacacatcgaaaggtgtgctaataccgcatgatgcaacgggatcgcatggttctgttgccaaagatttatcgctctgagatggactcgcgtctgattagctagttggtgaggtaatggctcaccaaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggccctcgggttgtaaacttcttttattcgagacgaaacaaatgacggtaccgaatgaataagccacggctaactacgtgccagcagccgcggtaatacgtaggtggcaagcgttatccggatttactgggtgtaaagggcgtgtaggcgggactgcaagtcagatgtgaaattccagggctcaactctggacctgcatttgaaactgtagttcttgagtgatggagaggcaggcggaattccgagtgtagcggtgaaatgcgtagatattcggaggaacaccagtggcgaaggcggcctgctggacattaactgacgctgaggcgcgaaagcgtggggagcaaacaggattagataccctggtagtccacgctgtaaacgatggatactaggtgtggggggactgaccccttccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatccctctgaccggtctagagataggccctcccttcggggcagaggtgacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtactacaatggcgcttaacagagggaggcaataccgcgaggtggagcaaacccctaaaaggcgtcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaacagcaatgagggcgcggccgaaggtgggttcgataattggggtgaagtcgtaacaaggtagccgtatcggaaggtgcggctggatcacctccttt

SEQ ID NO: 18 - DNA, 1509 bp, Ruminococcus albus
tattaagagtttgatcctggctcaggacgaacgctggcggcacgcttaacacatgcaagtcgaacgagcgaaagagtgcttgcactctctagctagtggcggacgggtgagtaacacgtgagcaatctgcctttcggagagggataccaattggaaacgattgttaatacctcataacataacgaagccgcatgactttgttatcaaatgaatttcgccgaaagatgagctcgcgtctgattaggtagttggtgaggtaacggcccaccaagccgacgatcagtagccggactgagaggttgaacggccacattgggactgagacacggcccagactcctacgggaggcagcagtggggaatattgcacaatgggcgaaagcctgatgcagcgatgccgcgtgagggaagaaggttttaggattgtaaacctctgtctttggggacgataatgacggtacccaaggaggaagctccggctaactacgtgccagcagccgcggtaatacgtagggagcgagcgttgtccggaattactgggtgtaaagggagcgtaggcgggattgcaagtcaggtgtgaaatttaggggcttaacccctgaactgcacttgaaactgtagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggctgaaactcaaaggaattgacgggggcccgcacaagcagtggagtatgtggtttaattcgaagcaacgcgaagaaccttaccaggtcttgacatcgtacgcatagcatagagatatgtgaaatcccttcggggacgtatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggctacacacgtactacaatggctgttaacagagggaagcaaaacagtgatgtggagcaaaaccctaaaagcagtcttagttcggattgtaggctgcaacccgcctacatgaagtcggaattgctagtaatcgcggatcagcatgccgcggtgaatacgttcccgggccttgtacacaccgcccgtcacgccatgggagtcggtaacacccgaagcctgtgttctaaccgcaaggaggaagcagtcgaaggtgggattgatgactggggtgaagtcgtaacaaggtagccgtatcggaaggtgcggctggatcacctccttt

SEQ ID NO: 19 - DNA, 945 bp, Agathobaculum desmolans
caagttgggagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatcccggtgaccgtcctagagataggacttcccttcggggcaacggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaaaatcgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaaggggggcgcggccgaaggtaggggtggtaattagggtgaagtcgtaacaaggtagccgtatcggaaggtgcggctggatcacctccttt

SEQ ID NO: 20 - DNA, 1509 bp, Ruminococcus bicirculans
attaagagtttgatcctggctcaggacgaacgctggcggcacgcttaacacatgcaagtcgaacgagagaagaggagcttgcttttctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgtcaacagagggaagcaaagccgcgaggtggagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtctaaccgcaaggaggacgcagtcgaaggtgggattgatgactggggtgaagtcgtaacaaggtagccgtatcggaaggtgcggctggatcacctccttt

SEQ ID NO: 21 - DNA, 1526 bp, Ruthenibacterium lactatiformans
aatgaagagtttgatcctggctcaggacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgtaaggaggacgccgccgaaggtaaaactggtgattggggtgaagtcgtaacaaggtagccgtatcggaaggtgcggctggatcacctccttt

SEQ ID NO: 22 - DNA, 1529 bp, Clostridium phoceensis
tattgagagtttgatcctggctcaggatgaacgctggcggcgtgcttaacacatgcaagtcgaacggagtgccttagaaagaggattcgtccaattgataaggttacttagtggcggacgggtgagtaacgcgtgaggaacctgcctcggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggctacacacgtactacaatggtggtaaacagagggaagcaagaccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagtctaaccgcaagggggacgcggccgaaggtgggttcgataattggggtgaagtcgtaacaaggtagccgtatcggaaggtgcggctggatcacctccttt

SEQ ID NO: 23 - DNA, 1529 bp, Intestinimonas massiliensis
tattgagagtttgatcctggctcaggatgaacgctggcggcgtgcttaacacatgcaagtcgaacggaacgccaaggaaagagttttcggacaatggaattggttgtttagtggcggacgggtgagtaacgcgtgagtaacctgccttggagtggggaataacacagtgaaaattgtgctaataccgcatgatatattggtgtcgcatggcactgatatcaaagatttatcgctctgagatggactcgcgtctgattagatagttggcggggtaacggcccaccaagtcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaacagggacgaagcaagtgacggtacctgttgaataagccacggctaactacgtgccagcagccgcggtaatacgtaggtggcaagcgttatccggatttactgggtgtaaagggcgtgtaggcgggactgcaagtcagatgtgaaaactatgggctcaacccatagcctgcatttgaaactgtagttcttgagtgtcggagaggcaatcggaattccgtgtgtagcggtgaaatgcgtagatatacggaggaacaccagtggcgaaggcggattgctggacgataactgacgctgaggcgcgaaagcgtggggagcaaacaggattagataccctggtagtccacgccgtaaacgatggatactaggtgtggggggtctgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaaccagagatggattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggttaacagagggaggcaaagccgcgaggcagagcaaacccctaaaagccgtcccagttcggattgcaggctgaaacccgcctgtatgaagtcggaatcgctagtaatcgcggatcagcatgccgcggtgaatacgttcccgggccttgtacacaccgcccgtcacaccatgagagtcgggaacacccgaagtccgtagcctaactgcaaagggggcgcggccgaaggtgggttcgataattggggtgaagtcgtaacaaggtagccgtatcggaaggtgcggctggatcacctccttt

SEQ ID NO: 24 - DNA, 1513 bp, Anaeromassilibacillus senegalensis
tttagagagtttgatcctggctcaggacgaacgctggcggcgtgcctaacacatgcaagtcgaacggagttagaagagcttgctcttctaacttagtggcggacgggtgagtaacgcgtgagtaacctgcctttcagagggggataacgttctgaaaagaacgctaataccgcatgacgtcatagtaccgcatggtacagtgatcaaaggagcaatccgctgaaagatggactcgcgtccgattagctagttggtggggtaaaggctcaccaaggcgacgatcggtagccggactgagaggttgaacggccacattgggactgagacacggcccagactcctacgggaggcagcagtgggggatattgcacaatgggggaaaccctgatgcagcaacgccgcgtgaaggaagaaggtcttcggattgtaaacttctgtcctatgggaagataatgacggtaccataggaggaagctccggctaactacgtgccagcagccgcggtaatacgtagggagcaagcgttgtccggatttactgggtgtaaagggtgcgtaggcggatctgcaagtcagtagtgaaatcccggggcttaaccccggaactgctattgaaactgtgggtcttgagtgaggtagaggcaggcggaattcccggtgtagcggtgaaatgcgtagagatcgggaggaacaccagtggcgaaggcggcctgctgggccttaactgacgctgaggcacgaaagcatgggtagcaaacaggattagataccctggtagtccatgccgtaaacgatgattactaggtgtgggtggtctgaccccatccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctactaacgaagcagagatgcattaggtgcctttcggggaaagtagagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggtcgttaacagagagaagcaatactgcgaagtggagcaaaactctaaaaacggtctcagttcggattgtaggctgcaacccgcctacatgaagttggaattgctagtaatcgcggatcagcatgccgcggtgaatacgttcccgggccttgtacacaccgcccgtcacaccatgggagccggtaatacccgaagtcagtagtctaaccgcaaggaggacgctgccgaaggtaggattggcgactggggtgaagtcgtaacaaggtagccgtatcggaaggtgcggctggatcacctccttt

SEQ ID NO: 25 - DNA, 1503 bp, Ruminococcus champanellensis
tatgaagagtttgatcctggctcaggacgaacgctggcggcacgcctaacacatgcaagtcgaacggagataaagacttcggtttttatcttagtggcggacgggtgagtaacacgtgagcaacctgcctctgagagagggatagcttctggaaacggatggtaatacctcataacatagcggtaccgcatgatactgctatcaaagatttatcgctcagagatgggctcgcgtctgattagctagatggtgaggtaacggctcaccatggcgacgatcagtagccggactgagaggttgaacggccacattgggactgagacacggcccagactcctacgggaggcagcagtggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatcgcagatcagcatgctgcggtgaatacgttcccgggccttgtacacaccgcccgtcacaccatggagtcggtaacacccgaagccagtagcctaaccgcaaggagggcgctgtcgaaggtgggattgatgactggggtgaagtcgtaacaaggtagccgtatcggaaggtgcggctggatcacctccttt

SEQ ID NO: 26 - DNA, 1526 bp, Bittarella massiliensis
ataaagagtttgatcctggctcaggacgaacgctggcggcgcgcctaacacatgcaagtcgaacggacacatccgacggaatagcttgctaggaagatggatgttgttagtggcggacgggtgagtaacacgtgagcaacctgcctcggagtgggggacaacagttggaaacgactgctaataccgcatacggtggtcgggggacatcccctggctaagaaaggatctatgatccgctctgagatgggctcgcgtctgattagctagttggcggggtaacggcccaccaaggcaacgatcagtagccggactgagaggttgaacggccacattgggactgagacacggcccagactcctacgggaggcagcagtggggaatattgcacaatggggggaaccctgatgcagcgacgccgcgtgagggaagaaggttttcggattgtaaacctctgtcttgtgggacgataatgacggtaccacaggaggaagccatggctaactacgtgccagcagccgcggtaatacgtagatggcgagcgttgtccggaattactgggtgtaaagggagtgtaggcgggatcataagttgcgtgtgaaatgcaggggctcaacccctgaactgcgcgcaaaactgtggttcttgagtgaagtagaggcaggcggaattcccggtgtagcggtggaatgcgtagatatcgggaggaacaccagtggcgaaggcggcctgctgggcttttactgacgctgaggctcgaaagcatggggagcaaacaggattagataccctggtagtccatgccgtaaacgatgattactaggtgtggggggataaccccctccgtgccggagttaacacaataagtaatccacctggggagtacgaccgcaaggttgaaactcaaaggaattgacgggggcccgcacaagcagtggagtatgtggtttaattcgaagcaacgcgaaaaaccttaccaggtcttgacatctatcgctatcccaagagattgggagttcccttcggggacggtaagacaggtggtgcatggttgtcgtcagctcgtgtcgtgagatgttgggttaagtcccgcaacgagcgcaacccttactgttagttgctacgcaagagcactctaacgggactgccgttgacaaaacggaggaaggtggggatgacgtcaaatcatcatgccctttatgacctgggctacacacgtactacaatggccgcaaacaacgagcagcgaaaccgcgaggtggagcgaatctataaaagcggtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcagtagtctaaccgcaagggggacgctgccgaaggtggggctggtgattggggtgaagtcgtaacaaggtagccgtatcggaaggtgcggctggatcacctccttt

SEQ ID NO: 27 - DNA, 1521 bp, Butyricicoccus porcorum
tttagagagtttgatcctggctcaggatgaacgctggcggcgtgcctaacacatgcaagtcgaacggagcactgagacttcggtttttgtgcttagtggcggacgggtgagtaacgcgtgagcaatctgcctttcagagggggataacgactggaaacggtcgctaataccgcataacgtattttgcaggcatctgcgagataccaaaggagcaatccgctgaaagatgagctcgcgtctgattagatagttggtgaggtaacggcccaccaagtcgacgatcagtagccggactgagaggttgaacggccacattgggactgagacacggcccagactcctacgggaggcagcagtggggaatattgcgcaatgggggaaaccctgacgcagcaacgccgcgtgatcgaagaaggtcttcggattgtaaagatcttttatcagggacgaagaaagtgacggtacctgatgaataagctccggctaactacgtgccagcagccgcggtaatacgtagggagcgagcgttatccggatttactgggtgtaaagggcgagtaggcgggctggtaagttggaagtgaaatgtcggggcttaaccccggaactgctttcaaaactgctggtcttgagtgatggagaggcaggcggaattcctagtgtagcggtgaaatgcgtagatattaggaggaacaccagtggcgaaggcggcctgctggacattaactgacgctgaggagcgaaagcgtggggagcaaacaggattagataccctggtagtccacgccgtaaacgatggatactaggtgtgggaggtattgaccccttccgtgccgcagttaacacaataagtatcccacctggggagtacgaccgcaaggttgaaactcaaaggaattgacgggggcccgcacaagcagtggagtatgtggtttaattcgaagcaacgcgaagaaccttacctggtcttgacatcccggtgaccggcatagagatatgcctttcccttcggggacagcggtgacaggtggtgcatggttgtcgtcagctcgtgtcgtgagatgttgggttaagtcccgcaacgagcgcaacccttattgttagttgatacatttagttgatcactctagcgagactgccgttgacaaaacggaggaaggtggggacgacgtcaaatcatcatgccccttatgaccagggctacacacgtactacaatggcagacatacagagggaagcaaagctgtgaggcagagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccggtaatgcccgaagtccgtagtctaaccgcaaggaggacgcggccgaaggcaggactggtaattagggtgaagtcgtaacaaggtagccgtatcggaaggtgcggctggatcacctccttt

SEQ ID NO: 28 - DNA, 1532 bp, Acutalibacter muris
tttagagagtttgatcctggctcaggacgaacgctggcggcgtgcctaatacatgcaagtcgaacggagatattcgctgatgaagtacttcggtaatgaatcttggatatcttagtggcggacgggtgagtaacgcgtgagcaacctgcctttcagagggggataacgtttggaaacgaacgctaataccgcatgacattatcttatcgcatggtaggataatcaaaggagcaatccgctgaaagatgggctcgcgtccgattaggtagttggtggggtaacggcccaccaagccgacgatcggtagccggactgagaggttggacggccacattgggactgagacacggcccagactcctacgggaggcagcagtaagggatattggtcaatgggggaaaccctgaaccagcaacgccgcgtgagggaagacggttttcggattgtaaacctctgtcctctgtgaagatgatgacggtagcagaggaggaagctccggctaactacgtgccagcagccgcggtaatacgtagggagcgagcgttgtccggatttactgggtgtaaagggtgcgtaggcggcttggcaagtcagtagtgaaatccatgggcttaacccatgaactgctattgaaactgtcgagcttgagtgaagtagaggtaggcggaattcccggtgtagcggtgaaatgcgtagagatcgggaggaacaccagtggcgaaggcggcctactgggctttaactgacgctgaggcacgaaagcgtgggtagcaaacaggattagataccctggtagtccacgctgtaaacgatgattactaggtgtgggtggactgaccccatccgtgccggagttaacacaataagtaatccacctggggagtacggccgcaaggctgaaactcaaaggaattgacgggggcccgcacaagcagtggagtatgtggattaattcgatgcaacgcgaagaaccttaccaggtcttgacatcccgctaacgaggtagagatacgttaggtgcccttcggggaaagcggagacaggtggtgcatggttgtcgtcagctcgtgtcgtgagatgttgggttaagtcccgcaacgagcgcaacccttactgttagttgctacgcaagagcactctagcaggaccgccgttgacaaaacggaggaaggtggggatgatgtcaaatcatcatgccccttatgacctgggcctcacacgtactacaatggccattaacagagggaggcaaagccgcgaggcagagcaaaaccctaaaaatggtcccagttcggatcgcaggctgcaacccgcctgcgtgaagttggaattgctagtaatcgcggatcagcatgccgcggtgaatacgttcccgggccttgtacacaccgcccgtcacaccatggaagtcggtaatgcccgaagtcagtagcctaaccgcaaggggggcgctgccgaaggcaggattgatgactggggtgaagtcgtaacaaggtagccgtatcggaaggtgcggctggatcacctccttt

SEQ ID NO: 29 - DNA, 1548 bp, Clostridium leptum
tttagagagtttgatcctggctcaggacgaacgctggcggcgtgcctaacacatgcaagtcgaacggagttaaattcgacacccgagtatccggccgggaggcggggtgctgggggttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatggactcgcgtccgattagccagttggcggggtaacggcccaccaaagcgacgatcggtagccggactgagaggttgaacggccacattgggactgagacacggcccagactcctacgggaggcagcagtgggggatattgcacaatgggggaaaccctgatgcagcaacgccgcgtgagggaagaaggttttcggattgtaaacctctgttcttagtgacgataatgacggtagctaaggagaaagctccggctaactacgtgccagcagccgcggtaatacgtagggagcgagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccggtgtagcggtgaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtggggggtctgaccccctccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgtctaacgaagcagagatgcattaggtgcccttcggggaaaggcgagacaggtggtgcatggttgtcgtcagctcgtgtcgtgagatgttgggttaagtcccgcaacgagcgcaacccttgtttctagttgctacgcaagagcactctagagagactgccgttgacaaaacggaggaaggtggggacgacgtcaaatcatcatgccccttatgacctgggccacacacgtactacaatggctgtaaacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggccttgtacacaccgcccgtcacaccatgggagccggtaatacccgaagccagtagttcaaccgcaaggagagcgctgtcgaaggtaggattggcgactggggtgaagtcgtaacaaggtagccgtatcggaaggtgcggctggatcacctccttt

SEQ ID NO: 30 - DNA, 1527 bp, Ruminococcus bromii
ttagagagtttgatcctggctcaggacgaacgctggcggcgtgcctaacacatgcaagtcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttctgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaaaatgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggatgttaacagagggaagcaagacagtgatgtggagcaaacccctaaaaacattctcagttcagattgcaggctgcaacccgcctgcatgaagatggaattgctagtaatcgcggatcagaatgccgcggtgaatacgttcccgggccttgtacacaccgcccgtcacaccatgggagccggtaatacccgaagtcagtagtccaacctcgtgaggacgctgccgaaggtaggattggcgactggggtgaagtcgtaacaaggtagccgtatcggaaggtgcggctggatcacctccttt

SEQ ID NO: 31 - DNA, 1528 bp, Monoglobus pectinilyticus
atcgagagtttgatcctggctcaggacgaacgctggcggcgtgcctaacacatgcaagtcgagcgagaaatttttaacggatcccttcggggagaagataaggatggaaagcggcggacgggtgagtaacgcgtgagtaacctgcctttaggagggggacaacattccgaaagggatgctaataccgcataaaattattgtatcgcatggtataataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgttaacaaagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgtagcttaaccttagggagagcgcagtcgaaggtgggattgataattagggtgaagtcgtaacaaggtagccgtatcggaaggtgcggctggatcacctccttt

SEQ ID NO: 32 - DNA, 1501 bp, Ethanoligenens harbinense; identical to SEQ ID NO: 7

SEQ ID NO: 33 - DNA, 1538 bp, Neglecta timonensis
tttagagagtttgatcctggctcaggacgaacgctggcggcgtgcctaacacatgcaagtcgaacggagatagacgctgaaagggagacagcttgctgtaagaatttcttgtttatcttagtggcggacgggtgagtaacgcgtgagtaacctgcctttcagagggggataacgtctggaaacggacgctaataccgcatgagaccacagcttcacatggagcggcggtcaaaggagcaatccgctgaaagatggactcgcgtccgattagatagttggcggggtaacggcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagatagtgacggtagcagaggaggaagctccggctaactacgtgccagcagccgcggtaatacgtagggagcgagcgttgtccggatttactgggtgtaaagggtgcgtaggcggctctgcaagtcagaagtgaaatccatgggcttaacccatgaactgcttttgaaactgtagagcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccctccgtgccggagttaacacaataagtaatccacctggggagtacgaccgcaaggttgaaactcaaaggaattgacgggggcccgcacaagcagtggagtatgtggattaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggcctcacacgtactacaatggccattaacagagggaagcaagcccgcgaggtggagcaaaaccctaaaaatggtctcagttcggatcgtaggctgaaacccgcctgcgtgaagttggaattgctagtaatcgcggatcagcatgccgcggtgaatacgttcccgggccttgtacacaccgcccgtcacaccatgggagccggtaatacccgaagtcagtagtctaaccgcaagggggacgctgccgaaggtaggattggcgactggggtgaagtcgtaacaaggtagccgtatcggaaggtgcggctggatcacctccttt

SEQ ID NO: 34 - DNA, 1522 bp, Anaerotruncus rubiinfantis
aaagagtttgatcctggctcaggacgaacgctggcggcgcgcctaacacatgcaagtcgaacggagtttatccgactgaagttttcggatggaagatggataaacttagtggcggacgggtgagtaacacgtgagcaacctgcctttcagagggggataacgattggaaacgatcgctaataccgcataacattatgaggagacatcttcttataatcaaaggagcaatccgctgaaagatgggctcgcggccgattagctagatggtggggtaacggcccaccatggcgacgatcggtagccggactgagaggttgaacggccacattgggactgagacacggcccagactcctacgggaggcagcagtgggggatattgcacaatggaggaaactctgatgcagcgacgccgcgtgagggaagacggtcttcggattgtaaacctctgtcttaggggaagaaaatgacggtaccctaagaggaagctccggctaactacgtgccagcagccgcggtaatacgtagggagcgagcgttgtccggaattactgggtgtaaagggagcgtaggcgggatggcaagttggatgtttaaactaacggctcaactgttaggtgcatccaaaactgctgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcataccatagagatatgggaagtccttcgggacatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcacttaaacaaagggcagcaacgtcgcgaggcgaagcgaatcccgaaaaagtgtctcagttcggatcgcaggctgcaacccgcctgcgtgaagtcggaattgctagtaatcgcggatcagcatgccgcggtgaatacgttcccgggccttgtacacaccgcccgtcacaccatgggagtcggtaacacccgaagccagtagtctaactgcaaagaggacgctgtcgaaggtgggattgatgactggggtgaagtcgtaacaaggtagccgtatcggaaggtgcggctggatcacctccttt

SEQ ID NO: 35 - DNA, 1522 bp, Massilioclostridium coli
  modified_base (819)..(825): a, c, t, g, unknown or other
attaagagtttgatcctggctcaggacgaacgctggcggcgcgcctaacacatgcaagtcgaacggagatacctgttagatcccttcggggtgacgatggactatcttagtggcggacgggtgagtaacacgtgagcaacctgccttacagagtgggataacgtttggaaacgaacgctaataccgcataacattaacttatcgcatggtaagataatcaaagaaattcgctgtaagatgggctcgcgtctgattagatagttggtgaggtaacggctcaccaagtcgacgatcagtagccggactgagaggttgaacggccacattgggactgagacacggcccagactcctacgggaggcagcagtggggaatattgcacaatgggggaaaccctgatgcagcgacgccgcgtgagggaagaaggttttcggattgtaaacctctgtcttcagggacgatagtgacggtacctgaggaggaagctccggctaactacgtgccagcagccgcggtaatacgtagggagcgagcgttgtccggaattactgggtgtaaagggagcgtaggcgggacagcaagttgaatgtgaaatctatgggctcaacccataaactgcgttcaaaactgttgttcttgagtgaagtagaggtaggcggaattcctagtgtagcggtgaaatgcgtagatattaggaggaacaccagtggcgaaggcggcctactgggctttaactgacgctgaggctcgaaagcgtgggtagcaaacaggattagataccctggtagtccacgctgtaaacgatgattactaggtgtnnnnnnntcaaccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccaactaacgagatagagatatgttaggtgcccttcggggaaagttgagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggccacacacgtactacaatggctattaacagagggaagcaataccgcgaggaggagcaaacccctaaaaatagtctcagttcggattgcaggctgcaacccgcctgcatgaagccggaattgctagtaatcgcggatcagcatgccgcggtgaatacgttcccgggccttgtacacaccgcccgtcacaccatgagagttggcaacacccgaagccagtagcctaaccgcaaggagggcgctgtcgaaggtggggttgatgattagggtgaagtcgtaacaaggtagccgtatcggaaggtgcggctggatcacctccttt

SEQ ID NO: 36 - DNA, 1508 bp, Angelakisella massiliensis
aatgaagagtttgatcctggctcaggacgaacgctggcggcgcgcctaacacatgcaagtcgaacggagtaagatgagcttgcttatcttacttagtggcggacgggtgagtaacacgtgagcaacctgccttcgagtggggaataacagtcggaaacgactgctaataccgcataacacattgggatggcatcatcctgatgtcaaagatttatcgctcgaagatgggctcgcgtccgattagctagttggcggggtaacggcccaccaaggcgacgatcggtagccggactgagaggttgatcggccacattgggactgagacacggcccagactcctacgggaggcagcagtgggggatattgcacaatgggggaaaccctgatgcagcgacgccgcgtgtaggaagacggtcctctggattgtaaactactgtcttcagggacgataatgacggtacctgaggaggaagctccggctaactacgtgccagcagccgcggtaatacgtagggagcgagcgttgtccggaattactgggtgtaaagggagcgtaggcggggaggcaagttggatgtgaaaactatcggctcaactgatagactgcattcaaaactgtttctcttgagtgaagtagaggcaggcgggattcctagtgtagcggtgaaatgcgtagatattaggaggaacaccagtggcgaaggcggcctgctgggcttttactgacgctgaggctcgaaagtgtggggagcaaacaggattagataccctggtagtccacaccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatctcctgcataacctagagataggtgaagtccttcgggacaggaagacaggtggtgcatggttgtcgtcagctcgtgtcgtgagatgttgggttaagtcccgcaacgagcgcaacccttgtttttagttgctacgcaagagcactctaaagagactgccgttgacaaaacggaggaaggtggggatgacgtcaaatcatcatgccccttatgacctgggctacacacgtactacaatggcaattaacagagggaagcgacaccgcgaggtggagcaaaaccctaaaaattgtcccagttcagattgcaggctgcaactcgcctgcatgaagtcggaattgctagtaatcgcggatcagcatgccgcggtgaatacgttcccgggccttgtacacaccgcccgtcacaccatgggagtcggtaacacccgaagtcagtagcctaaccgcaaggagggcgctgccgaaggtgggattgatgactggggtgaagtcgtaacaaggtagccgtatcggaaggtgcggctggatcacctccttt

SEQ ID NO: 37 - DNA, 1423 bp, Sporobacter termitidis
tattgagagtttgatcctggctcaggacgaacgctggcggcgtgcctaacacatgcaagtcgaacggagacaattggttcgctgattgtcttagtggcggacgggtgagtaacgcgtgagcaatctgcccttcggagggggacaacagctggaaacggctgctaataccgcataatgtatattcaaggcatcttggatataccaaagatttatcgccgaaggatgagctcgcgtctgattagctagttggtgaggtaaaggctcaccaaggctgcgatcagtagccggactgagaggttgaacggccacattgggactgagatacggcccagactcctacgggaggcagcagtggggaatattgggcaatgggggcaaccctgacccagcaacgccgcgtgaaggaagaaggccttcgggttgtaaacttctttgaccagggacgaaacaaatgacggtacctggaaaacaagccacggctaactacgtgccagcagccgcggtaatacgtaggtggcaagcgttgtccggatttactgggtgtaaagggcgcgtaggcgggagtacaagtcagatgtgaaatctgggggcttaaccctcaaactgcatttgaaactgtatttcttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacaggattagataccctggtagtccacgctgtaaacgatgaatactaggtgtggggggactgaccccctccgtgccggagttaacacaataagtattccacctggggagtacggccgcaaggttgaaactcaaaggaattgacgggggcccgcacaagcagtggattatgtggtttaattcgaagcaacgcgaagaaccttaccagggcttgacatcgtactaacgaagcagagatgcattaggtgcccttcggggaaagtatagacaggtggtgcatggttgtcgtcagctcgtgtcgtgagatgttgggttaagtcccgcaacgagcgcaacccctattgttagttgctacgcgagagcactctagcgagactgccgttgacaaaacggaggaaggtggggacgacgtcaaatcatcatgccccttatgtcctgggctacacacgtaatacaatggcgctcaacagagggaagcaagaccgcgaggtggagcaaatccctaaaaggcgtctcagttcagattgcaggctgcaactcgcctgcatgaagtcggaattgctagtaatcgcggatcagcatgccgcggtgaatacgttcccgggccttgtacacaccgcccgtcacaccatgagagccgggaacacccgaagtccgtagtctaaccgcaa

SEQ ID NO: 38 - DNA, 1513 bp, Negativibacillus massiliensis
acaaagagtttgatcctggctcaggacgaacgctggcggcgcgcctaacacatgcaagtcgaacggagttgtgttgaaagcttgctggatatacaacttagtggcggacgggtgagtaacacgtgagtaacctgcctctcagagtggaataacgtttggaaacgaacgctaataccgcataacgtgagaagagggcatcctctttttaccaaagatttatcgctgagagatgggctcgcggccgattaggtagttggtgagataacagcccaccaagccgacgatcggtagccggactgagaggttgatcggccacattgggactgagacacggcccagactcctacgggaggcagcagtgggggatattgcacaatgggggaaaccctgatgcagcgacgccgcgtgagggaagacggttttcggattgtaaacctctgtctttagggacgaaaaaatgacggtacctaaggaggaagccacggctaactacgtgccagcagccgcggtaatacgtaggtggcaagcgttgtccggaattactgggtgtaaagggagcgtaggcggggagacaagttgaatgtctaaactatcggcttaactgatagtcgcgttcaaaactatcactcttgagtgcagtagaggtaggcggaattcctagtgtagcggtgaaatgcgtagatattaggaggaacaccagtggcgaaggcggcctactgggctgtaactgacgctgaggctcgaaagcgtgggtagcaaacaggattagataccctggtagtccacgccgtaaacgatgattactaggtgtggggggactgaccccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcgagcgacgaaccaagagattggttcttccttcgggacgcgaagacaggtggtgcatggttgtcgtcagctcgtgtcgtgagatgttgggttaagtcccgcaacgagcgcaacccttatcattagttgctacgcaagagcactctaatgagactgccgttgataaaacggaggaaggtggggatgacgtcaaatcatcatgccccttatgacctgggctacacacgtactacaatggtgatcaaacagagggaagcaacacagcgatgtgaagcaaatcccgaaaaatcatctcagttcagattgcaggctgcaactcgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacctccttt

SEQ ID NO: 39 - DNA, 1522 bp, Massilimaliae massiliensis
  modified_base (820)..(826): a, c, t, g, unknown or other
aaagagtttgatcctggctcaggacgaacgctgtcggcgcgcctaacacatgcaagtcgaacgaagctgcatcgaacgaattcttcggaaagagattggtacagcttagtggcggacgggtgagtaacgcgtgagtaacctgcctttcagagggggataacgtttggaaacgaacgctaataccgcataacatattaaattcgcatggatttgatatcaaaggagcaatccgctgaaagatggactcgcgtccaattagctagttggtgaggtaacggcccaccaaggcgacgattggtagccggactgagaggttgaacggccacattgggactgagacacggcccagactcctacgggaggcagcagtggggaatattgcacaatgggggaaaccctgatgcagcgacgccgagtgagggaagaaggttttcggattgtaaacctctgtccttggtgaagataatgacggtagccaaggaggaagctacggctaactacgtgccagcagccgcggtaatacgtaggtagcgagcgttgtccggaattactgggtgtaaagggagcgtaggcgggattgcaagttgaatgtcaaatctacgggcttaacccgtagccgcgttcaaaactgcagttcttgagtgaagtagaggcaggcggaattcctagtgtagcggtgaaatgcgtaaatattaggaggaacaccagtggcgaaggcggcctgctgggctttaactgacgctgaggctcgaaagcgtgggtagcaaacaggattagataccctggtagtccacgctgtaaacgatgattactaggtgtnnnnnnnactgaccccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcgtgcgcatagcctagagataggtgaagcccttcggggcgcatagacaggtggtgcatggttgtcgtcagctcgtgtcgtgagatgttgggttaagtcccgcaacgagcgcaacccttacgtttagttgctacgcaagagcactctagacggactgccgttgacaaaacggaggaaggtggggatgacgtcaaatcatcatgccccttatgacctgggctacacacgtactacaatggctattaacagagggaagcaagatggtgacatggagcaaacccctaaaaatagtctcagttcggattgcaggctgcaacccgcctgcatgaagccggaattgctagtaatcgcggatcagcatgccgcggtgaatacgttcccgggccttgtacacaccgcccgtcacaccatgagagttggcaacacccgaagccgatagtctaaccgcaagggggacgtcgtcgaaggtggggttgatgattggggtgaagtcgtaacaaggtagccgtatcggaaggtgcggctggatcacctccttt

SEQ ID NO: 40 - DNA, 1200 bp, Intestinibacillus massiliensis
tagtggcggacgggtgagtaacgcgtgagcaatctgcctttaggagggggataacgaccggaaacggtcgctaataccgcatgaagtgccgggtgggcatccacctggcaccaaaggagcaatccgcctttagatgagctcgcgtcccattagctagttggtgaggtaacggcccaccaaggcgacgatgggtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatgagggacgaaaaatgacggtacctcaagaataagctccggctaactacgtgccagcagccgcggtaatacgtagggagcaagcgttatccggatttactgggtgtaaagggcgagtaggcgggctggcaagttgggagtgaaatccgggggcttaacccccgaactgctttcaaaactgctggccttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccggagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatccctctgaccggtacagagatgtaccttcccttcggggcaggggtgacaggtggtgcatggttgtcgtcagctcgtgtcgtgagatgttgggttaagtcccgcaacgagcgcaacccttattgttagttgatacattcagttgatcactctagcgagactgccgttgacaaaacggaggaaggtggggacgacgtcaaatcatcatgccccttatgacctgggctacacacgtactacaatggcagtcatacagagggaagcaaagccgcgaggtggagcaaatccctaaaagctgtcccagttcagattgcaggctgca

SEQ ID NO: 41 - DNA, 1314 bp, Eubacterium coprostanoligenes
tgtaccaaagctattgcgctgaaggatgggctcgcgtctgattagatagttggtggggtaacggcctaccaagtcgacgatcagtagccggactgagaggttgaacggccacattgggactgagacacggcccagactcctacgggaggcagcagtggggaatattgcacaatgggcgcaagcctgatgcagcaacgccgcgtggaggaagacggttttcggattgtaaactcctgttcttagtgaagaaaaatgacggtagctaaggagcaagccacggctaactacgtgccagcagccgcggtaatacgtaggtggcaagcgttgtccggaattactgggtgtaaagggagcgcaggcgggggagcaagtcagctgtgaaatctatgggcttaacccataaactgcagttgaaactgttcttcttgagtgaagtagaggttggcggaattccgagtgtagcggtgaaatgcgtagatattcggaggaacaccggtggcgaaggcggccaactgggcttttactgacgctgaggctcgaaagtgtggggagcaaacaggattagataccctggtagtccacactgtaaacgatgataactaggtgtagggggtctgaccccttctgtgccgcagctaacgcaataagttatccacctggggagtacgaccgcaaggttgaaactcaaaggaattgacggggacccgcacaagcagtggattatgtggtttaattcgatgcaacgcgaagaaccttaccagcacttgacatccaactaacgaaatagagatatattaggtgcccctcggggaaagttgagacaggtggtgcatggttgtcgtcagctcgtgtcgtgagatgttgggttaagtcccgcaacgagcgcaacccctgccattagttgctacgcaagagcactctaatgggaccgctaccgacaaggtggaggaaggtggggatgacgtcaaatcatcatgccccttatgtgctgggctacacacgtaatacaatggtcgttaacaaagagaagcaataccgcgaggtggagcaaaacttcaaaaacgatctcagttcggactgtaggctgaaactcgcctgcacgaagttggaattgctagtaatcgtggatcagcatgccacggtgaatacgttcccgggtcttgtacacaccgcccgtcacaccatgggagccggtaatacccgaagtcagtagtctaaccttaatggaggacgctgccgaaggtaggattggcgactggggtgaagtcgtaacaaggtagccgtaggagaacctgcggctggatcacctccttt

SEQ ID NO: 42 - DNA, 1511 bp, Provencibacterium massiliense
ctaaagagtttgatcctggctcaggacgaacgctggcggcgcgcctaacacatgcaagtcgaacggagaaatgctgagcttgctttgcattttttagtggcggacgggtgagtaacacgtgagcaacctgcctttgtgaggggaataacgtctggaaacggacgctaataccgcataacgtcaaggaaccgcatggttttttgaccaaagattttatcgcaaaaagatgggctcgcggctgattagctagttggcggggtaacggcccaccaaggcgacgatcagtagccggactgagaggttgatcggccacattgggactgagacacggcccagactcctacgggaggcagcagtgggggatattgcacaatgggggaaaccctgatgcagcgacgccgcgtgagggaagacggttttcggattgtaaacctctgtcttcagggacgaaatcaatgacggtacctgaggaggaagccacggctaactacgtgccagcagccgcggtaatacgtaggtggcaagcgttgtccggaattactgggtgtaaagggagcgtaggcgggaatgcaagttgaatgtttaaactatcggctcaactgataatcgcgttcaaaactgcatttcttgagtggagtagaggcaggcggaattcctagtgtagcggtgaaatgcgtagatattaggaggaacaccagtggcgaaggcggcctgctgggctctaactgacgctgaggctcgaaagcgtgggtagcaaacaggattagataccctggtagtccacgccgtaaacgatgattactaggtgtggggggactgaccccttccgtgccggagttaacacaataagtaatccacctggggagtacggtcgcaagactgaaactcaaaggaattgacgggggcccgcacaagcagtggagtatgtggtttaattcgaagcaacgcgaagaaccttaccaggtcttgacatcgtgcgcataccgtagagatacgggaagtccttcgggacgcatagacaggtggtgcatggttgtcgtcagctcgtgtcgtgagatgttgggttaagtcccgcaacgagcgcaacccttattattagttgctacgcaagagcactctaatgagactgccgttgacaaaacggcggaaggtggggatgacgtcaaatcatcatgccccttatgacctgggctacacacgtactacaatggcacttaacagagggaagcaagaccgcgaggtggagcaaacccccaaaaagtgtctcagttcggattgcaggctgcaacccgcctgtatgaagtcggaattgctagtaatcgcggatcagcatgccgcggtgaatacgttcccgggccttgtacacaccgcccgtcacaccatgagagccggtaacacccgaagtcagtagcctaaccgcaaggagggcgctgccgaaggtgggattggtgattagggtgaagtcgtaacaaggtagccgtatcggaaggtgcggctggatcacctccttt

SEQ ID NO: 43 - DNA, 1519 bp, Papillibacter cinnamivorans
tattgagagtttgatcctggctcaggacgaacgctggcggcgtgcctaacacatgcaagtcgaacgaaaataccaaagcagcaatgcgggggtattttagtggcggacgggtgagtaacgcgtgagcaatctgccttttggagggggataccgactggaaacggtcgttaataccgcataacgtatatggacgacatcgtccgtataccaaaggagcaatccgccgaaagatgagctcgcgtctgattagctagttggcggggtaaaggcccaccaaggcgacgatcagtagccggactgagaggttgaacggccacattgggactgagatacggcccagactcctacgggaggcagcagtggggaatattgggcaatgggcgaaagcctgacccagcaacgccgcgtgaaggaagaaggccttcgggttgtaaacttctttgaccagggaagaagaagtgacggtacctggaaaacaagccacggctaactacgtgccagcagccgcggtaatacgtaggtggcaagcgttgtccggatttactgggtgtaaagggcgtgtaggcgggattgcaagtcagatgtgaaatgccggggcttaaccccggagctgcatttgaaactgtagttcttgagtgatggagaggcaggcggaattcctagtgtagcggtgaaatgcgtagatattaggaggaacaccagtggcgaaggcggcctgctggacattaactgacgctgaggcgcgaaagcgtggggagcaaacaggattagataccctggtagtccacgctgtaaacgatggatactaggtgtgggaggtctgaccccttccgtgccggagttaacacaataagtatcccacctggggagtacgatcgcaaggttgaaactcaaaggaattgacgggggcccgcacaagcagtggagtatgtggtttaattcgaagcaacgcgaagaaccttaccaggatttgacatcctactaacgaggtagagatacgtcaggtgcccttcggggaaagtagagacaggtggtgcatggttgtcgtcagctcgtgtcgtgagatgttgggttaagtcccgcaacgagcgcaacccttattgctagttgctacgcaagagcactctagcgagactgccgttgacaaaacggaggaaggcggggacgacgtcaaatcatcatgccccttatgtcctgggctacacacgtactacaatggcggttaacagagggaagcaagacagtgatgtggagcaaatccctaaaaaccgtctcagttcggatcgcaggctgcaacccgcctgcgtgaagtcggaattgctagtaatcgcggatcagcatgccgcggtgaatacgttcccgggccttgtacacaccgcccgtcacaccatgagagtcgggaatacccgaagtccgtagtctaaccgcaagggggacgcggccgaaggtaggttcgataattggggtgaagtcgtaacaaggtagccgtatcggaaggtgcggctggatcacctccttt

SEQ ID NO: 44 - DNA, 1517 bp, Clostridium merdae
tttagagagtttgatcctggctcaggacgaacgctggcggcatgcctaacacatgcaagtcgaacggagtaagagagaagcttgcttagctcttacttagtggcggacgggtgagtaacgcgtgagtaacctgcctttcagagggggataacgttctgaaaagaacgctaataccgcataacatattggtgtcgcatggcactggtatcaaaggagcaatccgctgaaagatggactcgcgtccgattagctagttggtggggtaaaggcctaccaaggcgacgatcggtagccgggttgagagactgaacggccacattgggactgagacacggcccagactcctacgggaggcagcagtgggggatattgcacaatgggcgaaagcctgatgcagcaatgccgcgtgagggaagacggttttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggctctttaagtcgggcgtgaaagctgtgggctcaacccacaaattgcgttcgaaactggagagcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccctgtgattagttgctacgcaagagcactctaatcagactgccgttgacaaaacggaggaaggtggggacgacgtcaaatcatcatgccccttatgacctgggctacacacgtactacaatggtcgctaacagagggaagccaagccgcgaggtggagcaaacccccaaaagcggtctcagttcggattgtaggctgcaacccgcctacatgaagttggaattgctagtaatcgcggatcagcatgccgcggtgaatacgttcccgggccttgtacacaccgcccgtcacaccatgggagccggtaatacccgaagccaatagtctaaccgcaaggaggacgttgtcgaaggtaggattggcgactggggtgaagtcgtaacaaggtagccgtatcggaaggtgcggctggatcacctccttt

SEQ ID NO: 45 - DNA, 1508 bp, Marasmitruncus massiliensis
aaagagtttgatcctggctcaggacgaacgctggcggcgcgcctaacacatgcaagtcgaacggacagaagagaagcttgcttagcttctgttagtggcggacgggtgagtaacacgtgagtaacctgcctttcagagggggataacgattggaaacgatcgctaataccgcatgatgttgcgatgggacatcctattgcaaccaaaggagtaatccgctgaaagatgggctcgcggccgattagatagttggtgaggtaacggcccaccaagtcagcgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtcttaggggaagaaaatgacggtaccctaagaggaagctccggctaactacgtgccagcagccgcggtaatacgtagggagcgagcgttgtccggaattactgggtgtaaagggagcgtaggcggggcagcaagttggatgtttaaactaccggcttaaccggtaactgcatccaaaactgcagttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgtgcgcataccatagagatatgggaagcccttcggggcgcatagacaggtggtgcatggttgtcgtcagctcgtgtcgtgagatgttgggttaagtcccgcaacgagcgcaacccttattactagttgctacgcaagagcactctagtgagactgccgttgacaaaacggaggaaggtggggatgacgtcaaatcatcatgccccttatgacctgggctacacacgtactacaatggcacttaaacagagggctgctacatcgcgagatgaagcgaatcccgaaaaagtgtctcagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagccagtagtctaaccgcaagggggacgctgtcgaaggtgggattgatgactggggtgaagtcgtaacaaggtagccgtatcggaaggtgcggctggatcacctccttt

SEQ ID NO: 46 - DNA, 1522 bp, Massilimaliae timonensis; identical to SEQ ID NO: 1

SEQ ID NO: 47 - DNA, 1524 bp, Pygmaiobacter massiliensis
attaagagtttgatcctggctcaggacgaacgctggcggcgcgcctaacacatgcaagtcgaacggagcttgcacttctgaagttttcggatggacgaggtacaagcttagtggcgaacgggtgagtaacacgtgaagaacctgcccttcagtgggggacaacagttggaaacgactgctaataccgcataagaccacagtaccgcatggtacagtgatcaaaggatttattcgctgaaggatggcttcgcgtccgattaggtagttggtgaggtaacggcccaccaagcctacgatcggtagccggactgagaggttgatcggccacattgggactgagacacggcccagactcctacgggaggcagcagtggggaatattgcacaatggaggaaactctgatgcagcgacgccgcgtgagggaagaaggtcttcggattgtaaacctctgtcttcagggacgataatgacggtacctgaggaggaagcaccggctaactacgtgccagcagccgcggtaaaacgtagggtgcaagcgttgtccggaattactgggtgtaaagggagcgcaggcgggaagataagttggatgtttaatctacgggctcaacccgtatcagcattcaaaactatttttcttgagtagtgcagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcccgtgcatagtgtagagatacatgaagtccttcgggacacggtgacaggtggtgcatggttgtcgtcagctcgtgtcgtgagatgttgggttaagtcccgcaacgagcgcaacccttattgctagttactacgaaagaggactctagcaagactgccgttgacaaaacggaggaaggtggggatgacgtcaaatcatcatgccctttatgacctgggccacacacgtactacaatggctattaacaaagagatgctaagccgcgaggtggagcgaacctcataaaaatagtctcagttcggattgcaggctgcaactcgcctgcatgaagccggaattgctagtaatcgcggatcagcatgccgcggtgaatacgttcccgggccttgtacacaccgcccgtcacaccatgagagccggggggacccgaagtcagtagtctaaccgcaaggaggacgctgccgaaggtaaaactggtgattggggtgaagtcgtaacaaggtagccgtatcggaaggtgcggctggatcacctccttt

SEQ ID NO: 48 - DNA, 1517 bp, Clostridium minihomine
tttagagagtttgatcctggctcaggacgaacgctggcggcatgcctaacacatgcaagtcgaacggagtaagagataagcttgcttaactcttacttagtggcggacgggtgagtaacgcgtgagtaacctgcctttcagagggggataacgttctgaaaagaacgctaataccgcatgatatatcggtgtcgcatggcactgatatcaaaggagcaatccgctgaaagatggactcgcgtccgattagccagttggcggggtaatggcccaccaaagcgacgatcggtagccgggttgagagactggacggccacattgggactgagacacggcccagactcctacgggaggcagcagtgggggatattgcacaatggaggaaactctgatgcagcaatgccgcgtgagggaagacggtcttcggattgtaaacctctgtccttggtgaagataatgacggtagccaaggaggaagctccggctaactacgtgccagcagccgcggtaatacgtagggagcaagcgttgtccggatttactgggtgtaaagggtgcgtaggcggcttttcaagtcgggcgtgaaagctgtgggcttaacccacaaattgcgttcgaaactggagagcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggctgaaactcaaaggaattgacgggggcccgcacaagcagtggagtatgtggtttaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccctgtgattagttgctacgcaagagcactctaatcagactgccgttgacaaaacggaggaaggtggggacgacgtcaaatcatcatgccccttatgacctgggctacacacgtactacaatggtcgttaacaacgggaagctaagccgcgaggtggcgcaaatccccaaaaacggtctcagttcggattgtaggctgcaacccgcctacatgaagttggaattgctagtaatcgcggatcagcatgccgcggtgaatacgttcccgggccttgtacacaccgcccgtcacaccacgggagccggtaatacccgaagccgatagtctaaccgcaaggaggacgtcgtcgaaggtaggattggcgactggggtgaagtcgtaacaaggtagccgtatcggaaggtgcggctggatcacctccttt

SEQ ID NO: 49 - DNA, 1523 bp, Neobitarella massiliensis
taaagagtttgatcctggctcaggacgaacgctggcggcgcgcttaacacatgcaagtcgaacggacacatccgacggaatagcttgctaggaagatggatgttgttagtggcggacgggtgagtaacacgtgagcaacctacctcagagtgggggacaacagttggaaacgactgctaataccgcataagatggcagggtcgcatggcctggtcataaaaggagcaattcgctctgagatgggctcgcgtctgattagctagttggtgaggtaacggctcaccaaggcaacgatcagtagccggactgagaggttgaacggccacattgggactgagacacggcccagactcctacgggaggcagcagtggggaatattgcacaatgggggaaaccctgatgcagcgacgccgcgtgagggaagacggttttcggattgtaaacctctgtcttgtgggacgatagtgacggtaccacaggaggaagccatggctaactacgtgccagcagccgcggtaatacgtagatggcgagcgttgtccggaattactgggtgtaaagggagtgtaggcgggctggtaagttgaatgtgaaaccttcgggctcaacccggagcgtgcgttcaaaactgctggtcttgagtgaagtagaggcaggcggaattcccggtgtagcggtggaatgcgtagatatcgggaggaacaccagtggcgaaggcggcctgctgggcttttactgacgctgaggctcgaaagcatgggtagcaaacaggattagataccctggtagtccatgccgtaaacgatgattactaggtgtggggggattgaccccctccgtgccggagttaacacaataagtaatccacctggggagtacgaccgcaaggttgaaactcaaaggaattgacgggggcccgcacaagcagtggagtatgtggtttaattcgaagcaacgcgaaaaaccttaccaggtcttgacatccatcgccaggctaagagattagctgttcccttcggggacgatgagacaggtggtgcatggttgtcgtcagctcgtgtcgtgagatgttgggttaagtcccgcaacgagcgcaacccttactattagttgctacgcaagagcactctaatgggactgccgttgacaaaacggaggaaggtggggatgacgtcaaatcatcatgccccttatgacctgggctacacacgtactacaatggccgttaacagagagcagcgataccgcgaggtggagcgaatctagaaaaacggtctcagttcggattgcaggctgaaactcgcctgcatgaagtcggaattgctagtaatcgcggatcagcatgccgcggtgaatacgttcccgggccttgtacacaccgcccgtcacaccatgagagccggtaacacccgaagtcagtagcctaaccgcaaggagggcgctgccgaaggtggggctggtaattggggtgaagtcgtaacaaggtagccgtatcggaaggtgcggctggatcacctccttt

SEQ ID NO: 50 - DNA, 1509 bp, Faecalibacterium prausnitzii
tataaagagtttgatcctggctcaggacgaacgctggcggcgcgcctaacacatgcaagtcgaacgagcgagagagagcttgctttcttgagcgagtggcgaacgggtgagtaacgcgtgaggaacctgcctcaaagagggggacaacagttggaaacgactgctaataccgcataagcccacggctcggcatcgagcagagggaaaaggagcaatccgctttgagatggcctcgcgtccgattagctggttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacgatgctggaaacagtatttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtaggagaacctgcggctggatcacctccttt

SEQ ID NO: 51 - DNA, 1509 bp, Ruminococcus flavefaciens
taaagagtttgatcctggctcaggacgaacgctggcggcacgcttaacacatgcaagtcgaacggagataatttgagtttacttggattatcttagtggcggacgggtgagtaacacgtgagcaacctgcctttgagagagggatagcttctggaaacggatggtaatacctcataacataattgaagggcatcctttaattatcaaagatttatcactcaaagatgggctcgcatctgattagatagttggtgaggtaacggctcaccaagtcgacgatcagtagccggactgagaggttgaacggccacattgggactgagacacggcccagactcctacgggaggcagcagtggggaatattgcacaatgggggaaaccctgatgcagcgatgccgcgtggaggaagaaggttttcggattgtaaactcctgtcttaaaggacgataatgacggtactttaggaggaagctccggctaactacgtgccagcagccgcggtaatacgtagggagcgagcgttgtccggaattactgggtgtaaagggagcgtaggcgggattgcaagtcagatgtgaaatacatgggctcaacccatgggctgcatttgaaactgtagttcttgagtgaagtagaggtaagcggaattcctggtgtagcggtgaaatgcgtagatatcaggaggaacaccggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgtatgcataacttagagataagtgaaatcccttcggggacatatagacaggtggtgcatggttgtcgtcagctcgtgtcgtgagatgttgggttaagtcccgcaacgagcgcaacccttacctttagttgctacgcaagagcactctaaagggactgccgttgacaaaacggaggaaggtggggatgacgtcaaatcatcatgccccttatgacctgggctacacacgtactacaatggcaattaacaaagagaagcaagacagcgatgtggagcaaatctcgaaaaattgtcccagttcagattgcaggctgcaactcgcctgcatgaagtcggaattgctagtaatcgtggatcagcatgccacggtgaatacgttcccgggccttgtacacaccgcccgtcacaccatgggagtcggtaacacccgaagtcggtagtctaacagcaatgaggacgccgccgaaggtgggattgatgactggggtgaagtcgtaacaaggtagccgtatcggaaggtgcggctggatcacctccttt

SEQ ID NO: 52 - DNA, 1529 bp, Ruminococcaceae bacterium
tattgagagtttgatcctggctcaggatgaacgctggcggcgtgcttaacacatgcaagtcgaacggagtgcctttgaaagaggattcgtccaattgataaggttacttagtggcggacgggtgagtaacgcgtgaggaacctgccttggagtggggaataacacagtgaaaattgtgctaataccgcataatgcagttgggccgcatggctctgactgccaaagatttatcgctctgagatggcctcgcgtctgattagctagttggtggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttcttagggacgaagcaagtgacggtacctaaggaataagccacggctaactacgtgccagcagccgcggtaatacgtaggtggcaagcgttatccggatttactgggtgtaaagggcgtgtaggcgggattgcaagtcagatgtgaaaaccacgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccttccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccgaggcccggtctagagatagacctttctcttcggagacctcggtgacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaacccctaaaagccatcccagttcggattgcaggctgcaacccgcctgcatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 53 - DNA, 1248 bp, Ruminococcus albus
ggcccaccaagccgacgatcagtagccggactgagaggttgaacggccacattgggactgagacacggcccagactcctacgggaggcagcagtggggaatattgcacaatgggcgaaagcctgatgcagcgatgccgcgtgagggaagaaggttttaggattgtaaacctctgtcttcggggacgataatgacggtacccgaggaggaagctccggctaactacgtgccagcagccgcggtaatacgtagggagcgagcgttgtccggaattactgggtgtaaagggagcgtaggcgggactgcaagtcaggtgtgaaatgtaggggcttaacccctaccctgcacttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcgtgagcatagcttagagataagtgaaatcccttcggggactcatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggctacacacgtactacaatggctgttaacagagggaagcaaagcagtgatgcagagcaaaaccctaaaagcagtcttagttcggattgtaggctgcaacccgcctacatgaagtcggaattgctagtaatcgcggatcagcatgccgcggtgaatacgttcccgggccttgtacacaccgcccgtcacgccatgggagtcggtaacacccgaagcctgtgttctaaccgcaaggaggaagcagtcgaaggtgggattgatgactggggtgaagtcgtaacaaggtagccgtatcggaaggtgcggctggatcacctccttt

SEQ ID NO: 54 - DNA, 1285 bp, Anaerotruncus sp.
agatgggctcgcggccgattagctagttggtggggcaacggcccaccaaggcgacgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtctttggggaagaaaatgacggtacccaaagaggaagctccggctaactacgtgccagcagccgcggtaatacgtagggagcgagcgttgtccggaattactgggtgtaaagggagcgtaggcgggcgagaaagttgaatgttaaatctaccggcttaactggtagctgcgttcaaaacttcttgtcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgtgcgcatagcctagagataggtgaagcccttcggggcgcacagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactgaaacagagggaagcgacatcgcgaggtgaagcgaatcccaaaaaagtgtcccagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacctccttt

SEQ ID NO: 55 - DNA, 1527 bp, Oscillibacter sp.
tatagagagtttgatcctggctcaggacgaacgctggcggcgtgcttaacacatgcaagtcgaacggagcacccctgaaggagttttcggacaacggaagggaatgcttagtggcggactggtgagtaacgcgtgaggaacctgccttccagagggggacaacagttggaaacgactgctaataccgcatgaaacatttgaaccgcatggtttgaatgtcaaagatttatcgctggaagatggcctcgcgtctgattagctagtaggcggggtaacggcccacctaggcgacgatcagtagccggactgagaggttgaccggccacattgggactgagatacggcccagactcctacgggaggcagcagtggggaatattgggcaatgggcgcaagcctgacccagcaacgccgcgtgaaggaagaaggctttcgggttgtaaacttcttttaagagggaagagaagaagacggtacctcttgaataagccacggctaactacgtgccagcagccgcggtaatacgtaggtggcaagcgttgtccggatttactgggtgtaaagggcgtgcagccgggaagacaagtcagatgtgaaatcccgcggctcaaccgcggaactgcatttgaaactgtttttcttgagtaccggagaggtcatcggaattccttgtgtagcggtgaaatgcgtagatataaggaagaacaccagtggcgaaggcggatgactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatggagaggaccgctctagagatagggttttcccttcggggacctctcacacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaatccgcgaggaggagcgaacccccaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaacagcaatgagggcgcggccgaaggtgggttcgataattggggtgaagtcgtaacaaggtagccgttcgagaacgagcggctggatcacctccttt

SEQ ID NO: 56 - DNA, 1245 bp, Clostridiales bacterium
attagctagttggtgaggtaacggcccaccaaggcgacgatcagtagccggactgagaggttgaccggccacattgggactgagacacggcccagactcctacgggaggcagcagtggggaatattgcacaatgggggaaaccctgatgcagcaacgccgcgtgagtgatgacggccttcgggttgtaaagctctgtcttcagggacgataatgacggtacctgaggaggaagccacggctaactacgtgccagcagccgcggtaatacgtaggtggcgagcgttatccggatttactgggcgtaaaggatgcgtaggtggaattttaagtgggatgtgaaatacccgggctcaacctgggaactgcattccaaactggaattctagagtgcaggagaggaaagcggaattcctagtgtagcggtgaaatgcgtagagattaggaagaacaccagtggcgaaggcggcttgctggacagtaactgacgctaaggcgcgaaagcgtggggagcaaacaggattagataccctggtagtccacgccgtaaacgatgggtactaggtgtaggggtttcgatacctctgtgccgccgtaaacacaataagtaccccgcctggggagtacggtcgcaagattaaaactcaaaggaattgacgggggcccgcacaagtagcggagcatgtggtttaattcgaagcaacgcgaagaaccttaccaggtcttgacatcccggcgaccggtgtagagatacaccttcttcttcggaagcgccggtgacaggtggtgcatggttgtcgtcagctcgtgtcgtgagatgttgggttaagtcccgcaacgagcgcaacccctatagttagttgctaacagtaagatgagcactctagctagactgccgtggttaacgcggaggaaggtggggatgacgtcaaatcatcatgccccttatgtctagggctacacacgtgctacaatggcgagaacaaagagaagcaagaccgcgaggtggagcaaaactcataaaactcgtcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 57 - DNA, 1007 bp, Oscillibacter sp.
tatagagagtttgatcctggctcaggacgaacgctggcggcgtgcttaacacatgcaagtcgaacggagcacccttgactgaggtttcggccaaatgataggaatgcttagtggcggactggtgagtaacgcgtgaggaacctaccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatcctactaacgaagtagagat

SEQ ID NO: 58 - DNA, 1529 bp, Firmicutes bacterium
tattgagagtttgatcctggctcaggatgaacgctggcggcgtgcttaacacatgcaagtcgaacggaggacccctgaaggagttttcggacaactgaagggaatccttagtggcggacgggtgagtaacgcgtgagtaacctgccttggagtggggaataacagctggaaacagctgctaataccgcatgatatgtctgtgtcgcatggcactggacatcaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcaagcgttatccggatttactgggtgtaaagggcgtgtaggcgggactgcaagtcagatgtgaaaaccacgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggcgaccggtgtagagatacactttcttcttcggaagcgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaaaaccgcgaggtggagcaaatccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaacagcaatgggggcgcggccgaaggtgggttcgataattggggtgaagtcgtaacaaggtagccgtatcggaaggtgcggctggatcacctccttt

SEQ ID NO: 59 - DNA, 1515 bp, Ruminococcus sp.
aattaagagtttgatcctggctcaggacgaacgctggcggcacgcttaacacatgcaagtcgaacggggttacaagataagcttgcttaatttgtaacctagtggcggacgggtgagtaacacgtgagcaatctgcccttaagagggggataccagttagaaatgactgttaataccgcataagatagtagtaccgcatggtacagctataaaagatttatcgcttaaggatgagctcgcgtctgattagctagttggtgaggtaacggcccaccaaggcaacgatcagtagccggactgagaggttggacggccacattgggactgagacacggcccagactcctacgggaggcagcagtggggaatattgcacaatggaggaaactctgatgcagcgatgccgcgtgagggaagaaggttttaggattgtaaacctctgttgacagggacgataatgacggtacctgttgaggaagctccggctaactacgtgccagcagccgcggtaatacgtagggagcgagcgttgtccggaattactgggtgtaaagggagcgtaggcgggatcgcaagtcaggtgtgaaatgcgggggctcaacccccgaactgcacttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagctaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtacgcatagcatagagatatgtgaaatcccttcggggacgtatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgccaacagagggaagcaaagcagtgatgcagagcaaagccccaaaagcagtcttagttcggattgcaggctgaaacccgcctgcatgaagtcggaattgctagtaatcgcagatcagcatgctgcggtgaatacgttcccgggccttgtacacaccgcccgtcacgccatgggagtcggtaacacccgaagcctgtagcccaaccgcaaggaggacgcagtcgaaggtgggattgatgactggggtgaagtcgtaacaaggtagccgtatcggaaggtgcggctggatcacctccttt

SEQ ID NO: 60 - DNA, 1510 bp, Ruminococcus sp.
ttaaagagtttgatcctggctcaggacgaacgctggcggcacgcttaacacatgcaagtcgaacggagttttagagagcttgctttttaaaacttagtggcggacgggtgagtaacacgtgagcaatctgcctttcagagggggatagcagttggaaacgactgataataccgcataatatagtaggatcgcatggttcaactatcaaagatttatcgctgaaagatgagctcgcgtctgattagatagttggtgaggtaacggctcaccaagtcgacgatcagtagccggactgagaggttgaacggccacattgggactgagacacggcccagactcctacgggaggcagcagtggggaatattgcacaatgggcgcaagcctgatgcagcgatgccgcgtgagggaagaaggttttaggattgtaaacctctgtcttcagggacgataatgacggtacctgaggaggaagctccggctaactacgtgccagcagccgcggtaatacgtagggagcgagcgttgtccggaattactgggtgtaaagggagtgcaggcgggactgcaagtcagatgtgaaatgtaggggcttaacccctgaactgcatttgaaactgtagttcttgagtgaagtagaggtaagcggaattcctagtgtagcggtgaaatgcgtagatattaggaggaacatcagtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggctgaaactcaaaggaattgacgggggcccgcacaagcagtggagtatgtggattaattcgaagcaacgcgaagaaccttaccaggtcttgacatcgtacgcatagcatagagatatgtgaaatcccttcggggacggacagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgttaacagagagaagcgacatagtgatatgaagcaaaaccctaaaagcagtctcagttcggattgcaggctgaaacccgcctgcatgaagtcggaattgctagtaatcgcggatcagcatgccgcggtgaatacgttcccgggccttgtacacaccgcccgtcacaccatgggagtcggtaacacccgaagtcagtagcctaaccgtaaggagggcgctgccgaaggtgggattgatgactggggtgaagtcgtaacaaggtagccgtatcggaaggtgcggctggatcacctccttt

SEQ ID NO: 61 - DNA, 1305 bp, Ruminococcus flavefaciens
caaagatttatcactcagagatgggctcgcgtctgattagatagttggtgaggtaacggctcaccaagtcgacgatcagtagccggactgagaggttgaacggccacattgggactgagacacggcccagactcctacgggaggcagcagtggggaatattgcacaatggggggaaccctgatgcagcgatgccgcgtggaggaagaaggttttcggattgtaaactcctgtcttaaaggacgataatgacggtactttaggaggaagctccggctaactacgtgccagcagccgcggtaatacgtagggagcgagcgttgtccggaattactgggtgtaaagggagcgtaggcgggactgcaagtcagatgtgaaatgccggggcttaaccccggagctgcatttgaaactgtggttcttgagtgaagtagaggcaagcggaattcctggtgtagcggtgaaatgcgtagatatcaggaggaacaccggtggcgaaggcggcttgctgggcttttactgacgctgaggctcgaaagcgtgggtagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgtatgcatagcatagagatatgtgaaatctcttcggagacatatagacaggtggtgcatggttgtcgtcagctcgtgtcgtgagatgttgggttaagtcccgcaacgagcgcaacccttacctttagttgctacgcaagagcactctaaagggactgccgttgacaaaacggaggaaggtggggatgacgtcaaatcatcatgccccttatgacctgggctacacacgtactacaatggcaatcaacaaagagaagcaagacagtgatgtggagcgaatctcaaaaaattgtcccagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagtcagtagtctaacagcaatgaggacgctgccgaaggtgggattgatgactggggtgaagtcgtaacaaggtagccgtatcggaaggtgcggctggatcacctccttt

SEQ ID NO: 62 - DNA, 1507 bp, Ruminococcus sp.
ataaagagtttgatcctggctcaggatgaacgctggcggcacgcctaacacatgcaagtcgaacggagtttaagagagcttgctcttttaaacttagtggcggacgggtgagtaacacgtgagcaacctgcctttcagagagggatagcttctggaaacggatggtaatacctcataacatattgatacggcatcgtattgatatcaaagatttatcgctgaaagatgggctcgcgtctgattagctggttggtgaggtaacggcccaccaaggcaacgatcagtagccggactgagaggttgaacggccacattgggactgagacacggcccagactcctacgggaggcagcagtggggaatattgcacaatgggcgcaagcctgatgcagcgatgccgcgtgagggaagaaggttttcggattgtaaacctctgtcatcggggacgaaaatgacggtacccgagaaggaagctccggctaactacgtgccagcagccgcggtaatacgtagggagcaagcgttatccggaattactgggtgtaaagggagtgtaggcgggactgcaagtcagatgtgaaatatgccggctcaactggcagactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggctgaaactcaaaggaattgacgggggcccgcacaagcagtggagtatgtggtttaattcgaagcaacgcgaagaaccttaccaggtcttgacatcgagtgaagtatcaagagattgatatgtcttcggacacaaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggctacacacgtactacaatggcaatcgaacagagggaagcaatacagcgatgtaaagcaaaacccgaaaaaattgtctcagttcggattgcaggctgcaacccgcctgcatgaagtcggaattgctagtaatcgcagatcagcatgctgcggtgaatacgttcccgggccttgtacacaccgcccgtcacaccatgggagtcggtaacacccgaagccagtagtccaaccgcaaggaggacgctgtcgaaggtgggattgatgactggggtgaagtcgtaacaaggtagccgtatcggaaggtgcggctggatcacctccttt

SEQ ID NO: 63 - DNA, 1560 bp, Unknown
  source: /note=“Description of Unknown bacterium MS4 sequence”
tttagagagtttgatcctggctcaggacgaacgctggcggcgtgcctaacacatgcaagtcgaacggaattaagtttaacaccgaacactttgtttggtggggacacctgaccgagtggtgggtgttgagcttaatttagtggcggacgggtgagtaacgcgtgagtaacctgcctttcagagggggataacgtctggaaacggacgctaataccgcatgacatatttgggctgcatggtctgaatatcaaaggagcaatccgctgaaagatggactcgcgtccgattagctagttggtgagataaaggcccaccaaggcgacgatcggtagccggactgagaggttgaacggccacattgggactgagacacggcccagactcctacgggaggcagcagtgggggatattgcacaatggaggaaactctgatgcagcaacgccgcgtgagggaagacggttttcggattgtaaacctctgtccttggtgacgaaacaaatgacggtagccaaggaggaagctccggctaactacgtgccagcagccgcggtaatacgtagggagcaagcgttgtccggatttactgggtgtaaagggtgcgtaggcggctctgcaagtcaggcgtgaaatatatgggcttaacccatagactgcgtttgaaactgtggagcttgagtgaagtagaggtaggcggaattcccggtgtagcggtgaaatgcgtagagatcgggaggaacaccagtggcgaaggcggcttactgggctttaactgacgctgaggcacgaaagcatgggtagcaaacaggattagataccctggtagtccatgccgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagcagagatgcatcaggtgcccttcggggaaagttgagacaggtggtgcatggttgtcgtcagctcgtgtcgtgagatgttgggttaagtcccgcaacgagcgcaacccctgtgattagttgctacgcaagagcactctaatcagactgccgttgacaaaacggaggaaggtggggacgacgtcaaatcatcatgccctttatgacctgggctacacacgtactacaatggctgttaacaaagggaagcaagaccgcgaggtggagcaaaacctaaaaaacagtctcagttcggatcgcaggctgcaacccgcctgcgtgaagttggaattgctagtaatcgcggatcatcatgccgcggtgaatacgttcccgggccttgtacacaccgcccgtcacaccatgggagccggtaatacccgaagtcagtagcctaaccgcaagggaggcgctgccgaaggtaggattggcgactggggtgaagtcgtaacaaggtagccgtatcggaaggtgcggctggatcacctccttt

SEQ ID NO: 64 - DNA, 1530 bp, Intestinimonas butyriciproducens
tattgagagtttgatcctggctcaggatgaacgctggcggcgtgcttaacacatgcaagtcgaacggagcacccctgacggagttttcggacaacgaaagggaatgcttagtggcggacgggtgagtaacgcgtgagtaacctgccttggagtggggaataacagccggaaacggctgctaataccgcatgatgtatctggatcgcatggttctggataccaaagatttatcgctctgagatggactcgcgtctgattagctagttggtgaggtaatggctcaccaaggcgacgatcagtagccggactgagaggttggccggccacattgggactgagacacggcccagactcctacgggaggcagcagtggggaatattgggcaatgggcgaaagcctgacccagcaacgccgcgtgaaggaagaaggccctcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggagtgcaagtcagatgtgaaaactatgggctcaacccatagcctgcatttgaaactgtacttcttgagtgatggagaggcaggcggaattccctgtgtagcggtgaaatgcgtagatatagggaggaacaccagtggcgaaggcggcctgctggacattaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccaggacttgacatcctactaacgaagcagagatgcataaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggtcaacagagggaagcaaagccgcgaggtggagcaaatccctaaaagccgtcccagttcggattgcaggctgaaactcgcctgtatgaagtcggaatcgctagtaatcgcggatcagcatgccgcggtgaatacgttcccgggccttgtacacaccgcccgtcacaccatgagagtcgggaacacccgaagtccgtagcctaacagcaatgggggcgcggccgaaggtgggttcgataattggggtgaagtcgtaacaaggtagccgtatcggaaggtgcggctggatcacctccttt

SEQ ID NO: 65 - DNA, 1529 bp, Oscillibacter sp.
tattgagagtttgatcctggctcaggacgaacgctggcggcgtgcttaacacatgcaagtcgaacgagaatctactgaaagagttttcggacaatggatgtagaggaaagtggcggacgggtgagtaacgcgtgaggaacctgccttgaagagggggacaacagttggaaacgactgctaataccgcatgatgcataggggtcgcatgatctttatgccaaagatttatcgcttcaagatggcctcgcgtctgattagctagttggcggggtaacggcccaccaaggcgacgatcagtagccggactgagaggttgaacggccacattgggactgagatacggcccagactcctacgggaggcagcagtggggaatattgggcaatgggcgcaagcctgacccagcaacgccgcgtgaaggaagaaggctttcgggttgtaaacttcttttaagagggaagagcagaagacggtacctctagaataagccacggctaactacgtgccagcagccgcggtaatacgtaggtggcaagcgttgtccggatttactgggtgtaaagggcgtgcagccgggtctgcaagtcagatgtgaaatccatgggctcaacccatgaactgcatttgaaactgtagatcttgagtgtcggaggggcaatcggaattcctagtgtagcggtgaaatgcgtagatattaggaggaacaccagtggcgaaggcggattgctggacgataactgacggtgaggcgcgaaagtgtggggagcaaacaggattagataccctggtagtccacactgtaaacgatgaatactaggtgtgcggggactgaccccctgcgtgccgcagtaaacacaataagtattccacctggggagtacgatcgcaaggttgaaactcaaaggaattgacgggggcccgcacaagcggtggattatgtggtttaattcgaagcaacgcgaagaaccttaccagggtttgacatcctgctaacgaagtagagatacattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatatcctgggctacacacgtaatacaatggcggtcaacagagggaagcaaagccgcgaggcagagcaaacccccaaaagccgtcccagttcggattgtaggctgcaactcgcctgcatgaagtcggaatcgctagtaatcgcggatcagcatgccgcggtgaatacgttcccgggccttgtacacaccgcccgtcacaccatgagagtcgggaacacccgaagtccgtagcctaacctgaaaaggagggcgcggccgaaggtgggttcgataattggggtgaagtcgtaacaaggtagccgttcgagaacgagcggctggatcacctccttt

SEQ ID NO: 66 - DNA, 1545 bp, Candidatus Soleaferrea massiliensis
attaagagtttgatcctggctcaggacgaacgctggcggcgcgcttaacacatgcaagtcgaacggggttgtttctgacactcagtgggtaatcggtagattgctgattgagtgttgggaataacctagtggcggacgggtgagtaacacgtgagcaacctacctttcagagggggataacgtttggaaacgaacgctaataccgcatgatataattggatggcatcatctgattatcaaaggagcaatccgctgaaagatgggctcgcggccgattaggtagttggagtggtaacggcacaccaagccgacgatcggtagccggactgagaggttgaacggccacattgggactgagacacggcccagactcctacgggaggcagcagtggggaatattgcacaatgggcgaaagcctgatgcagcgacgccgcgtgagggaagacggttttcggattgtaaacctctgtcttatgtgacgataatgacggtagcataggaggaagccacggctaactacgtgccagcagccgcggtaatacgtaggtggcaagcgttgtccggaattactgggtgtaaagggagcgtaggcgggaatgcaagttgaatgttaaatctaccggctcaaccggtagctgcgttcaaaactgtatttcttgagtgaagtagaggcaggcggaattcctagtgtagcggtgaaatgcgtagatattaggaggaacaccagtggcgaaggcggcctgctgggcttttactgacgctgaggctcgaaagcgtggggagcaaacaggattagataccctggtagtccacgctgtaaacgatgattactaggtgtggggggtctgaccccttccgtgccggagttaacacaataagtaatccacctggggagtacgaccgcaaggttgaaactcaaaggaattgacggggacccgcacaagcagtggagtatgtggtttaattcgaagcaacgcgaagaaccttaccaggtcttgacatccaactaacgaggcagagatgcgttaggtgcccttcggggaaagttgagacaggtggtgcatggttgtcgtcagctcgtgtcgtgagatgttgggttaagtcccgcaacgagcgcaacccttactattagttgctacgcaagagcactctaatgggactgccgttgacaaaacggaggaaggtggggatgacgtcaaatcatcatgccccttatgacctgggccacacacgtactacaatggtgttcaacagagggaagcaaaactgtgaagtggagcaaacccctaaaagacatcccagttcggatcgtaggctgcaacccgcctacgtgaagttggaattgctagtaatcgcggatcagcatgccgcggtgaatacgttcccgggtcttgtacacaccgcccgtcacaccatgagagtcggtaacacccgaagtcagtagcctaaccgcaaggagggcgctgccgaaggtgggattgatgattagggtgaagtcgtaacaaggtagccgtatcggaaggtgcggctggatcacctccttt

SEQ ID NO: 67 - DNA, 1510 bp, Clostridium cellulosi
ttagagagtttgatcctggctcaggacgaacgctggcggcgcgcctaacacatgcaagtcgagcggagatagtacttcggttctatcttagcggcggacgggtgagtaacgcgtgagcaacctgcccttgagcgggggatagcgtctggaaacggacggtaataccgcataatgtacgttggaggcatctccgatgtaccaaaggagaaatccactcaaggatgggctcgcgtccgattaggtagttggtgaggtaatggcccaccaagcctgcgatcggtagccggactgagaggttgtacggccacattgggactgagacacggcccagactcctacgggaggcagcagtgggggatattgcacaatggaggaaactctgatgcagcgacgccgcgtgagggaagaaggtcttcggattgtaaacctctgtctttcgggacgaaggaagtgacggtaccgaaagaggaagccacggctaactacgtgccagcagccgcggtaatacgtaggtggcgagcgttgtccggaattactgggtgtaaagggtgcgtaggcgggttgtcaagttggatgtgaaatctctgggcttaactcagaggttgcattcaaaactggcgatcttgagtgaggtagaggcaggcggaattcccggtgtagcggtgaaatgcgtagatatcgggaggaacaccagtggcgaaggcggcctgctgggccttaactgacgctgaggcacgaaagcatggggagcaaacaggattagataccctggtagtccatgctgtaaacgatgattgctaggtgtgggtggactgaccccatccgtgccggagttaacacaataagcaatccacctggggagtacggccgcaaggttgaaactcaaaggaattgacgggggcccgcacaagcagtggagtatgtggtttaattcgaagcaacgcgaagaaccttaccaggtcttgacatccaccgaatccggaagagattctggagtgcccttcggggagcggtgagacaggtggtgcatggttgtcgtcagctcgtgtcgtgagatgttgggttaagtcccgcaacgagcgcaacccttgttaatagttgctacgcaagagcactctattaagactgccgttgataaaacggaggaaggtggggatgacgtcaaatcatcatgccccttatgacctgggctacacacgtactacaatggccgccaacaaagggaagcaataccgcgaggtggagcgaatccccaaaagcggtcccagttcagattgcaggctgcaacccgcctgcatgaagacggaattgctagtaatcgcggatcagcatgccgcggtgaatacgttcccgggccttgtacacaccgcccgtcacaccatgagagccggaaacacccgaagtcgtttgcgtaaccgaaaggagcgcggcgccgaaggtgggatcggtgattggggtgaagtcgtaacaaggtagccgtatcggaaggtgcggctggatcacctccttt

SEQ ID NO: 68 - DNA, 1509 bp, Clostridia bacterium
tctaagagtttgatcctggctcaggacgaacgctggcggcgcgcctaacacatgcaagtcgaacgagccgaggggagcttgctccccagagctagtggcggacgggtgagtaacacgtgagcaacctgcctttcagagggggataacgtttggaaacgaacgctaataccgcataacataccgggaccgcatgattctggtatcaaaggagcaatccgctgaaagatgggctcgcgtccgattagctagttggcggggtaacggcccaccaaggcgacgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtctttggggacgataatgacggtacccaaggaggaagctccggctaactacgtgccagcagccgcggtaatacgtagggagcgagcgttgtccggaattactgggtgtaaagggagcgtaggcggggtctcaagtcgaatgttaaatctaccggctcaactggtagctgcgttcgaaactggggctcttgagtgaagtagaggcaggcggaattcctagtgtagcggtgaaatgcgtagatattaggaggaacaccagtggcgaaggcggcctgctgggcttttactgacgctgaggctcgaaagcgtggggagcaaacaggattagataccctggtagtccacgccgtaaacgatgattactaggtgtgggggactgaccccttccgtgccggagttaacacaataagtaatccacctggggagtacgaccgcaaggttgaaactcaaaggaattgacgggggcccgcacaagcagtggattatgtggtttaattcgaagcaacgcgaagaaccttaccaggtcttgacatcgagtgacggctctagagatagagctttccttcgggacacaaagacaggtggtgcatggttgtcgtcagctcgtgtcgtgagatgttgggttaagtcccgcaacgagcgcaacccttattattagttgctacattcagttgagcactctaatgagactgccgttgacaaaacggaggaaggtggggatgacgtcaaatcatcatgccccttatgacctgggctacacacgtaatacaatggcgatcaacagagggaagcaagaccgcgaggtggagcaaacccctaaaagtcgtctcagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagtcagtagcctaaccgcaaagagggcgctgccgaaggtgggattgatgactggggtgaagtcgtaacaaggtagccgtatcggaaggtgcggctggatcacctccttt

SEQ ID NO: 69 - DNA, 1513 bp, Clostridia bacterium
tttagagagtttgatcctggctcaggacgaacgctggcggcgtgcctaacacatgcaagtcgaacggagttaagagagcttgctcttttaacttagtggcggacgggtgagtaacgcgtgagtaacctgcctttcagaggggaataacattctgaaaagaatgctaataccgcatgagatcgtagtatcgcatggtacagcgaccaaaggagcaatccgctgaaagatggactcgcgtccgattagctagttggtgagataaaggcccaccaaggcgacgatcggtagccggactgagaggttgaacggccacattgggactgagacacggcccagactcctacgggaggcagcagtgggggatattgcacaatgggggaaaccctgatgcagcaacgccgcgtgaaggaagaaggtcttcggattgtaaacttctgtcctcagggaagataatgacggtacctgaggaggaagctccggctaactacgtgccagcagccgcggtaatacgtagggagcaagcgttgtccggatttactgggtgtaaagggtgcgtaggcggatctgcaagtcagtagtgaaatcccagggcttaaccctggaactgctattgaaactgtgggtcttgagtgaggtagaggcaggcggaattcccggtgtagcggtgaaatgcgtagagatcgggaggaacaccagtggcgaaggcggcctgctgggccttaactgacgctgaggcacgaaagcatgggtagcaaacaggattagataccctggtagtccatgccgtaaacgatgattactaggtgtgggtggtctgaccccatccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctgctaacgaggtagagatacgttaggtgcccttcggggaaagcagagacaggtggtgcatggttgtcgtcagctcgtgtcgtgagatgttgggttaagtcccgcaacgagcgcaacccctgctattagttgctacgcaagagcactctaataggactgccgttgacaaaacggaggaaggtggggacgacgtcaaatcatcatgccccttatgacctgggctacacacgtactacaatggccgtcaacagagagaagcaaagccgcgaggtggagcaaaactctaaaaacggtcccagttcggatcgtaggctgcaacccgcctacgtgaagttggaattgctagtaatcgcggatcatcatgccgcggtgaatacgttcccgggccttgtacacaccgcccgtcacaccatgggagccggtaatacccgaagtcagtagtctaaccgcaagggggacgctgccgaaggtaggattggcgactggggtgaagtcgtaacaaggtagccgtatcggaaggtgcggctggatcacctccttt

SEQ ID NO: 70 - DNA, 1525 bp, Clostridia bacterium
tttagagagtttgatcctggctcaggatgaacgctggcggcgtgcctaacacatgcaagtcgaacggggttattttggaaatctcttcggagatggaattcttaacctagtggcggacgggtgagtaacgcgtgagcaatctgcctttaggagggggataacagtcggaaacggctgctaataccgcataatacgtttgggaggcatctcttgaacgtcaaagattttatcgcctttagatgagctcgcgtctgattagctggttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggcctcgggttgtaaagatctttaatcagggacgaaaaatgacggtacctgaagaataagctccggctaactacgtgccagcagccgcggtaatacgtagggagcaagcgttatccggatttactgggtgtaaagggcgcgcaggcgggccggcaagttgggagtgaaatcccggggcttaaccccggaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatcccgatgaccggcgtagagatacgccctctcttcggagcatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaataccgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaaggggggcgcggccgaaggtaggggtggtaattagggtgaagtcgtaacaaggtagccgtatcggaaggtgcggctggatcacctccttt

SEQ ID NO: 71 - DNA, 1526 bp, Fournierella massiliensis
tatgaagagtttgatcctggctcaggacgaacgctggcggcgcgcctaacacatgcaagtcgaacggagcttgcttgtcagatcctttcggggtgacgacttgtaagcttagtggcgaacgggtgagtaacacgtgagtaacctgccccagagtgggggacaacagttggaaacgactgctaataccgcataagcccacggaaccgcatggttcagagggaaaaggagcaattcgctttgggatggactcgcgtccgattagctagatggtgaggtaacggcccaccatggcgacgatcggtagccggactgagaggttgatcggccacattgggactgagacacggcccagactcctacgggaggcagcagtggggaatattgcacaatgggggaaaccctgatgcagcgacgccgcgtggaggaagaaggccttcgggttgtaaactcctgtcgtaagggacgatagtgacggtaccttacaagaaagccacggctaactacgtgccagcagccgcggtaaaacgtaggtggcaagcgttgtccggaattactgggtgtaaagggagcgcaggcgggtctgcaagttggaagtgaaacccatgggctcaacccatgaactgctttcaaaactgcggatcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcccgtgcatagcatagagatatgtgaagtccttcgggacacggagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggcaattaacaaagagaagcaaagccgcgaggtggagcaaacctcataaaaattgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtccgtagcctaaccgcaaggagggcgcggccgaaggtaaaactggtgattggggtgaagtcgtaacaaggtagccgtatcggaaggtgcggctggatcacctccttt

SEQ ID NO: 72 - DNA, 1518 bp, Clostridium sp.
tttagagagtttgatcctggctcaggacgaacgctggcggcgtgcctaacacatgcaagtcgaacggaaacagattgaagcttgctttgaactgttttagtggcggacgggtgagtaacgcgtgaggaacctgcctttcagagggggataacgtctggaaacggacgctaataccgcatgacattttgttgccgcatggtgataaaatcaaaggagcaatccgctgagagatggactcgcgtccgattagccggttggcggggtaacggcccaccaaagcaacgatcggtagccgggctgagaggctgaacggccacattgggactgagacacggcccagactcctacgggaggcagcagtgggggatattgcacaatggaggaaactctgatgcagcaacgccgcgtgagggaagaaggttttcggattgtaaacctctgtcctcagggacgataatgacggtacctgaggaggaagctccggctaactacgtgccagcagccgcggtaatacgtagggagcaagcgttgtccggatttactgggtgtaaagggtgcgtaggcggcactgcaagtcaggtgtgaaaaccatgggcttaacttatggattgcacttgaaactgtggtgcttgagtgaagtagaggcaggcggaattcccggtgtagcggtgaaatgcgtagagatcgggaggaacaccagtggcgaaggcggcctgctgggctttaactgacgctgaggcacgaaagcatgggtagcaaacaggattagataccctggtagtccatgccgtaaacgatgattactaggtgtggggggtctgaccccttccgtgccggagttaacacaataagtaatccacctgggaagtacgaccgcaaggttgaaactcaaaggaattgacgggggcccgcacaagcagtggagtatgtggtttaattcgaagcaacgcgaagaaccttaccaggtcttgacatccaactaacgaagcagagatgcatcaggtgcccttcggggaaagttgagacaggtggtgcatggttgtcgtcagctcgtgtcgtgagatgttgggttaagtcccgcaacgagcgcaacccttgtgattagttgctacgctaagagcactctaatcagactgccgttgacaaaacggaggaaggtggggacgacgtcaaatcatcatgccccttatgacctgggctacacacgtactacaatggccgttaacaacgggaagcgaagccgcgaggcggagcaaaaccccaaaaacggtctcagttcggatcgcaggctgcaacccgcctgcgtgaagctggaattgctagtaatcgcggatcatcatgccgcggtgaatacgttcccgggccttgtacacaccgcccgtcacaccatgggagccggtaatacccgaagtcggtagcctaaccgcaaggaaggcgccgccgaaggtaggattggcgactggggtgaagtcgtaacaaggtagccgtatcggaaggtgcggctggatcacctccttt

SEQ ID NO: 73 - DNA, 1511 bp, Ruminococcaceae bacterium
tttagagagtttgatcctggctcaggacgaacgctggcggcgtgcctaacacatgcaagtcgaacgaaactttttgcttcggtagaaagtttagtggcggacgggtgagtaacgcgtgaggaacctgcctttcagagggggataatgtctggaaacggacactaataccgcatgacattttctgttcacatggacagaaaatcaaaggagcaatctgctgaaagatggactcgcgtccgattagctagatggtgagataatagcccaccatggcgacgatcggtagccggactgagaggttgaacggccacattgggactgagacacggcccagactcctacgggaggcagcagtgggggatattgcacaatggaggaaactctgatgcagcaacgccgcgtgaaggaagacggtcttcggattgtaaacttttgtacctagggacgataatgacggtacctaggcagcaagctccggctaactacgtgccagcagccgcggtaatacgtagggagcgagcgttgtccggatttactgggtgtaaagggtgcgtaggcggccaagcaagtcagctgtgaaaactatgggcttaacccatagcctgcaattgaaactgtttggcttgagtgaagtagaggtaggtggaattcccggtgtagcggtgaaatgcgtagagatcgggaggaacaccagtggcgaaggcgacctactgggctttaactgacgctgaagcacgaaagcatgggtagcaaacaggattagataccctggtagtccatgctgtaaacgatgattactaggtgtggggggtctgaccccttccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggctacacacgtactacaatggccgttaacagagagaagcgataccgcgaggtggagcgaacctcaaaaagcggtctcagttcggattgcaggctgaaacccgcctgcatgaagttggaattgctagtaatcgcggatcataatgccgcggtgaatacgttcccgggccttgtacacaccgcccgtcacaccatgggagccggtaatacccgaagtcagtagtctaaccgcaaggaggacgctgccgaaggtaggattggcgactggggtgaagtcgtaacaaggtagccgtatcagaaggtgcggctggatcacctccttt

SEQ ID NO: 74 - DNA, 1530 bp, Flavonifractor sp.
tattgagagtttgatcctggctcaggatgaacgctggcggcgtgcttaacacatgcaagtcgaacggagtgctcatgacggagttttcggacaacggattgagttacttagtggcggacgggtgagtaacgcgtgaggaacctgccttggagtggggaataacagttggaaacagctgctaataccgcataatgcagttgggtcgcatggccctgactgccaaagatttatcgctctgagatggcctcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggatgaaggctttcgggttgtaaacttcttttgtcagggacgaaacaaatgacggtacctgacgaataagccacggctaactacgtgccagcagccgcggtaatacgtaggtggcaagcgttatccggatttactgggtgtaaagggcgtgtaggcgggattgcaagtcagatgtgaaaaccaggggctcaacctctggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcataaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggtggttaacagagggaggcaaaaccgcgaggtggagcaaatccctaaaagccatcccagttcggattgcaggctgcaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 75 - DNA, 991 bp, Flavonifractor sp.
tattgagagtttgatcctggctcaggatgaacgctggcggcgtgcttaacacatgcaagtcgaacggagtgctcatgacggaggattcgtccaacggattgagttacttagtggcggacgggtgagtaacgcgtgaggaacctgcctcggagtggggaataacagcccgaaagggttgctaataccgcatgatgcagttgggccgcatggctctgactgccaaagatttatcgctctgagatggcctcgcgtctgattagctggttggcggggtaacggcccaccaaggcgacgatcagtagccggactgagaggttgaccggccacattgggactgagacacggcccagactcctacgggaggcagcagtggggaatattgggcaatgggcgcaagcctgacccagcaacgccgcgtgaaggatgaaggctttcgggttgtaaacttcttttattcgggacgaagaaaatgacggtaccgaatgaataagccacggctaactacgtgccagcagccgcggtaatacgtaggtggcaagcgttatccggatttactgggtgtaaagggcgtgtaggcgggactgcaagtcagatgtgaaaactatgggctcaacccatagcctgcatttgaaactgtagttcttgagtgctggagaggcaatcggaattccgtgtgtagcggtgaaatgcatagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatccc

SEQ ID NO: 76 - DNA, 1530 bp, Flavonifractor sp.
tattgagagtttgatcctggctcaggatgaacgctggcggcgtgcttaacacatgcaagtcgaacggagtgctcatgacagaggattcgtccaatggattgagttacttagtggcggacgggtgagtaacgcgtgaggaacctgcctcggagtggggaataacagaccgaaaggcctgctaataccgcatgatacagttgggtcgcatggctctgactgtcaaagatttatcgctctgagatggcctcgcgtctgattagctagttggcggggtaacggcccaccaaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttctcggggacgaaacaaatgacggtacctgaggaataagccacggctaactacgtgccagcagccgcggtaatacgtaggtggcgagcgttatccggatttactgggtgtaaagggcgtgtaggcgggattgcaagtcagacgtgaaaactatgggctcaacccatagcctgcgtttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaagcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaacagcaatgggggcgcggccgaaggtgggttcgataattggggtgaagtcgtaacaaggtagccgtatcggaaggtgcggctggatcacctccttt

SEQ ID NO: 77 - DNA, 1525 bp, Anaerofilum sp.
tataaagagtttgatcctggctcaggacgaacgctggcggcgcgcctaacacatgcaagtcgaacggagctatttcgatagatcccttcggggtgacattggcttagcttagtggcgaacgggtgagtaacacgtgaggaacctgcccttcagagggggacaacagttggaaacgactgctaataccgcataagaccacagagccgcatggctcaggggtcaaaggagaaatccgctgaaggatggcctcgcgtccgattaggtagttggcggggtaacggcccaccaagccgacgatcggtagccggactgagaggttgaacggccacattgggactgagacacggcccagactcctacgggaggcagcagtggggaatattgcacaatgggggaaaccctgatgcagcgacgccgcgtgagggaagaagattttcggattgtaaacctctgtcttcggggacgataatgacggtacccgaggaggaagccacggctaactacgtgccagcagccgcggtaatacgtaggtggcaagcgttgtccggaattactgggtgtaaagggagcgcaggcgggtttgcaagttggatgtttaatcgaggggctcaacccctttccgcattcaaaactgcagatcttgagtggtgcagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtggggggattgaccccctccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcccgtgcatagcatagagatatgtgaagtccttcgggacacggagacaggtggtgcatggttgtcgtcagctcgtgtcgtgagatgttgggttaagtcccgcaacgagcgcaacccttactgatagttactacgcaagaggactctatcgggactgccgttgacaaaacggaggaaggtggggatgacgtcaaatcatcatgccctatatgacctgggctacacacgtactacaatggctatgaacaaagagaagcgaagccgcgaggcagagcaaacctcataaaaatagtctcagttcggactgcaggctgcaactcgcctgcacgaagccggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctccttt

SEQ ID NO: 78 - DNA, 895 bp, Anaeromassilibacillus sp.
tcttgttgcttagtggcggacgggtgagtaacacgtgagtaacctgcctctcagagggggataacgtcttgaaaaggacgctaataccgcatgatatctcttgaccgcatggtcgggagatcaaaggagcaatccgctgagagatggactcgcgtccgattagccagttggcggggtaacggcccaccaaagcaacgatcggtagccggactgagaggttgaacggccacattgggactgagacacggcccagactcctacgggaggcagcagtgggggatattgcacaatgggggaaaccctgatgcagcaacgccgcgtgaaggatgaaggtcttcggattgtaaacttttgtcctatgggaagaagaaagtgacggtaccataggaggaagctccggctaactacgtgccagcagccgcggtaatacgtagggagcaagcgttgtccggatttactgggtgtaaagggtgcgtaggcggaagagcaagtcagtagtgaaatctgggggcttaacccccaaactgctattgaaactgtttttcttgagtggagtagaggtaggcggaattcccggtgtagcggtgaaatgcgtagagatcgggaggaacaccagtggcgaaggcggcctactgggctctaactgacgctgaggcacgaaagtgtgggtagcaaacaggattagataccctggtagtccacaccgtaaacgatgattactaggtgtggggggtctgaccccctccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaa

SEQ ID NO: 79 - DNA, 1420 bp, Pseudoflavonifractor sp.
aagtggcggacgggtgagtaacgcgtgaggaacctgcctcggagtggggaataacagttggaaacagctgctaataccgcataatgcaacggaatcgcatgactctgttgccaaagatttatcgctctgagatggcctcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaacaaatgacggtacctgacgaataagccacggctaactacgtgccagcagccgcggtaatacgtaggtggcaagcgttatccggatttattgggtgtaaagggcgtgtaggcgggactgcaagtcagatgtgaaaaccacgggctcaacctgtggcctgcatttgaaactgtagttcttgagtgtcggagaggcaatcggaattccgtgtgtagcggtgaaatgcgtagatatacggaggaacaccagtggcgaaggcggattgctggacgataactgacgctgaggcgcgaaagcgtggggagcaaacaggattagataccctggtagtccacgccgtaaacgatggatactaggtgtggggggactgaccccctccgtgccgcagttaacacagtaagtatcccacctggggagtacgatcgcaaggttgaaactcaaaggaattgacgggggcccgcacaagcggtggagtatgtggtttaattcgaagcaacgcgaagaaccttaccaggacttgacatcctactaacgaagcagagatgcattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggcggttaacaaagagaggcaataccgcgaggtggagcaaatctcaaaaagccgtcccagttcggattgcaggctgcaacccgcctgcatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 80 - DNA, 1529 bp, Pseudoflavonifractor sp.
tattgagagtttgatcctggctcaggatgaacgctggcggcgtgcttaacacatgcaagtcgaacggagagcgtatgacagaggattcgtccaatggattgcgtttcttagtggcggacgggtgagtaacgcgtgaggaacctgcctcggagtggggaataacacaacgaaagctgtgctaataccgcatgatgcagctgggtcgcatgactctggctgccaaagatttatcgctctgagatggcctcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggccctcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggattgcaagtcagatgtgaaaaccacgggctcaacctgtggcctgcatttgaaactgcagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccgacgaccggtgtagagatacacttttctcttcggagacgtcggtgacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggtggtcaacagagggaggcaaaaccgcgaggtggagcaaacccctaaaagccatcccagttcggattgcaggctgcaacccgcctgcatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 81 - DNA, 1423 bp, Anaeromassilibacillus sp.
tttagtggcggacgggtgagtaacgcgtgagtaacctgccttcaagaggggaataacgttctgaaaagaacgctaataccgcataacatacggatgtcgcatggcaaccgtatcaaagattttatcgcttgaagatggactcgcgtccgattagccagttggcggggtaacggcccaccaaagcgacgatcggtagccggactgagaggttgaacggccacattgggactgagacacggcccagactcctacgggaggcagcagtgggggatattgcgcaatgggggcaaccctgacgcagcaacgccgcgtgaacgatgaaggtcttcggattgtaaagttcttttattaaggacgaagaagtgacggtacttaatgaataagctccggctaactacgtgccagcagccgcggtaatacgtagggagcaagcgttgtccggatttactgggtgtaaagggtgcgtaggcggcagagcaagtcagatgtgaaatccgtgggcttaacccacgaactgcatttgaaactgttttgcttgagtgaagtagaggcaggcggaattccctgtgtagcggtgaaatgcgtagagatagggaggaacaccagtggcgaaggcggcctgctgggctttaactgacgctgaggcacgaaagcgtgggtagcaaacaggattagataccctggtagtccacgccgtaaacgatgattactaggtgtggggggtctgaccccctccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaggtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggccatcaacagagggaagcaaagcagcgatgcagagcaaacccctaaaaatggtcccagttcagattgcaggctgcaactcgcctgtatgaagtcggaattgctagtaatcgcggatcagcatgccgcggtgaatacgttcccgggccttgtacacaccgcccgtcacaccatgggagccggtaatacccgaagtcagtagtctaaccgcaaggaggacgctgccgaaggtaggattggcgactggggtgaagtcgtaacaaggtagccgtatcggaaggtgcggctggatcacctccttt

SEQ ID NO: 82 - DNA, 1524 bp, Gemmiger sp.
taaagagtttgatcctggctcaggacgaacgctggcggcgcgcctaacacatgcaagtcgaacggagttattttggctgaagttttcggatggacgccgggataacttagtggcgaacgggtgagtaacacgtgaggaacctgcccttgagtgggggacaacagttggaaacgactgctaataccgcataagcccacagagccgcatggctcagggggaaaaggagcaattcgcttaaggatggactcgcgtccaattaggtagatggtgaggtaacggcccaccatgccgacgattggtagccggactgagaggttgaacggccacattgggactgagacacggcccagactcctacgggaggcagcagtggggaatattgcacaatgggggaaaccctgatgcagcgacgccgcgtgaaggaagaaggccttcgggttgtaaacttctgtcgtaagggacgataatgacggtaccttacaagaaagccacggctaactacgtgccagcagccgcggtaaaacgtaggtggcaagcgttgtccggaattactgggtgtaaagggagcgcaggcggggaggcaagttggaagtgaaaagcgtgggctcaacccacgacctgctttcaaaactgtctctcttgagtagtgcagaggtaagcggaattcccggtgtagcggtggaatgcgtagatatcgggaggaacaccagtggcgaaggcggcttactgggcaccaactgacgctgaggctcgaaagcatgggtagcaaacaggattagataccctggtagtccatgccgtaaacgatgattactaggtgtggggagattgaccctctccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttatcgtcagttactacgcaagaggactctggcgagactgccgttgacaaaacggaggaaggtggggatgacgtcaaatcatcatgccctttatgacctgggctacacacgtactacaatggcgatcaacaaagagaagcgaagccgcgaggcggagcaaacctcataaacatcgtcccagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtccgtagcctaaccgcaaggagggcgcggccgaaggtaaaactggtgattggggtgaagtcgtaacaaggtagccgtatcggaaggtgcggctggatcacctccttt

SEQ ID NO: 83 - DNA, 1529 bp, Flavonifractor sp.
tattgagagtttgatcctggctcaggatgaacgctggcggcgtgcttaacacatgcaagtcgaacggagtgctcatgacagaggattcgtccaatggaatgagttacttagtggcggacgggtgagtaacgcgtgagtaacctgccttggagtggggaataacacaacgaaagctgtgctaataccgcataatgcagctgagtcgcatggctctggctgccaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcaagcgttatccggatttactgggtgtaaagggcgtgtaggcgggattgcaagtcagatgtgaaaaccatgggctcaactcatggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggcttagagataggcttttcccttcggggacaccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggccacacacgtactacaatggtggttaacagagggaagcaatgccgcgaggcggagcaaacccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcttaaccgcaaggagggcgcggccgaaggtgggttcgataattggggtgaagtcgtaacaaggtagccgtatcggaaggtgcggctggatcacctccttt

SEQ ID NO: 84 - DNA, 1530 bp, Flavonifractor sp.
tattgagagtttgatcctggctcaggatgaacgctggcggcgtgcttaacacatgcaagtcgaacggagaacccctgatagaggattcgtccaattgaagggaattcttagtggcggacgggtgagtaacgcgtgaggaacctgccttggagtggggaataacagtccgaaaggactgctaataccgcataatgcagttgggccgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggccctcgggttgtaaacttcttttgacagggacgaagaaaatgacggtacctgtcgaataagccacggctaactacgtgccagcagccgcggtaatacgtaggtggcaagcgttatccggatttactgggtgtaaagggcgtgtaggcgggctggcaagtcagatgtgaaaaccatgggctcaacccatggcctgcatttgaaactgttggtcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctgctaacgaagtagagatacattaggtgcccttcggggaaagcagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggttaacagagggaagcaaaaccgcgaggtggagcaaatccctaaaagccgtcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 85 - DNA, 1513 bp, Eubacteriaceae bacterium
tttagagagtttgatcctggctcaggacgaacgctggcggcgtgcctaacacatgcaagtcgaacggacgagaaggtgcttgcaccttcaagttagtggcggacgggtgagtaacgcgtgagcaacctgcctcaaagagggggataacgtctggaaacggacgctaataccgcatgacgtattcgataggcatctattgaataccaaaggagcaatccgctttgagatgggctcgcgtctgattagctagttggtggggtaaaggcctaccaaggcgacgatcagtagccggactgagaggttgaacggccacattgggactgagacacggcccagactcctacgggaggcagcagtgggggatattgcacaatgggggaaaccctgatgcagcaacgccgcgtgaaggaagacggttttcggattgtaaacttctgttcttagtgacgataatgacggtagctaaggagaaagctccggctaactacgtgccagcagccgcggtaatacgtagggagcgagcgttgtccggaattactgggtgtaaagggagcgtaggcgggagatcaagtcagatgtgaaaactatgggctcaacccataacctgcatttgaaactggttttcttgagtgaagtagaggcaggcggaattccgagtgtagcggtgaaatgcgtagatattcggaggaacaccagtggcgaaggcggcctgctgggcttttactgacgctgaggctcgaaagcatggggagcaaacaggattagataccctggtagtccatgccgtaaacgatgattactaggtgtggggtggctgacccattccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgactaacgaagtagagatacattaggtgcccttcggggaaagtcgagacaggtggtgcatggttgtcgtcagctcgtgtcgtgagatgttgggttaagtcccgcaacgagcgcaacccttgtcattagttgctacgcaagagcactctaatgagactgccgttgacaaaacggaggaaggtggggacgacgtcaaatcatcatgccccttatgacctgggctacacacgtactacaatggccgttaacagagggaagcaatactgtgaagtggagcaaacccctaaaaacggtcccagttcagattgcaggctgcaacccgcctgcatgaagtcggaattgctagtaatcgcggatcagcatgccgcggtgaatacgttcccgggccttgtacacaccgcccgtcacaccatgggagccggtaatacccgaagtcggtagtctaaccgcaaggagggcgccgccgaaggtaggattggcgactggggtgaagtcgtaacaaggtagccgtatcggaaggtgcggctggatcacctccttt

SEQ ID NO: 86 - DNA, 1419 bp, Ruminococcaceae bacterium
tttagagagtttgatcctggctcaggacgaacgctggcggcgtgcctaacacatgcaagtcgaacggagttgaggagcttgctccttaacttagtggcggacgggtgagtaacgcgtgagtaacctgcctctgagaggggaataacgttctgaaaagaacgctaataccgcatgacacatatttgccgcatgacagatatgtcaaagattttatcgctcagagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttctcagggacgaaatttgacggtacctgaggaataagctccggctaactacgtgccagcagccgcggtaatacgtagggagcaagcgttgtccggatttactgggtgtaaagggtgcgtaggcggctttgtaagtcagatgtgaaatctatgggctcaacccataaactgcatttgaaactacagagcttgagtgaagtagaggcaggcggaattccctgtgtagcggtgaaatgcgtagagatagggaggaacaccagtggcgaaggcggcctgctgggctttaactgacgctgaggcacgaaagcgtgggtagcaaacaggattagataccctggtagtccacgctgtaaacgatgattactaggtgtggggggactgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgactaacgaagtagagatacatcaggtgcccttcggggaaagtcgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggccatcaacagagggaagcaaaacagcgatgtggagcaaacccctaaaaatggtctcagttcagattgcaggctgcaacccgcctgcatgaagtcggaattgctagtaatcgcggatcagcatgccgcggtgaatacgttcccgggccttgtacacaccgcccgtcacaccatgggagccggtaatacccgaagtcagtagcttaacct

SEQ ID NO: 87 - DNA, 1527 bp, Ruminococcus bromii
ttagagagtttgatcctggctcaggacgaacgctggcggcgtgcctaacacatgcaagtcgaacggaactgcttcgaaggatttcttcggaatgacattgattcagtttagtggcggacgggtgagtaacgcgtgagtaacctgccttcaagagggggataacattctgaaaagaatgctaataccgcatgacatatgattgtcgcatggcagacatatcaaagatttatcgcttgaagatggactcgcgtccgattagttagttggtgaggtaacggcccaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaataatgacggtacttaatgaataagctccggctaactacgtgccagcagccgcggtaatacgtagggagcaagcgttgtccggatttactgggtgtaaagggtgcgtaggcggctaagcaagtcagatgtgaaatctatgggctcaacccataaactgcatttgaaactgcatagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgagatagagatatgttaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatgggcgttaacagagggaagcaaaatagcgatatggagcaaacccctaaaaacgttctcagttcagattgcaggctgcaacccgcctgcatgaagtcggaattgctagtaatcgcggatcagcatgccgcggtgaatacgttcccgggccttgtacacaccgcccgtcacaccatgggagccggtaatacccgaagtcagtagttcaaccgcaaggagagcgctgccgaaggtaggattggcgactggggtgaagtcgtaacaaggtagccgtatcggaaggtgcggctggatcacctccttt

SEQ ID NO: 88 - DNA, 1415 bp, Ruminococcus sp.
tcagtggcggacgggtgagtaacacgtgagcaatctgcctttaagaggggaataacgactggaaacggtcggtaataccgcataacatatcgaagccgcatgactttgatatcaaagatttatcgcttaaagatgagctcgcgtctgattagctagttggtgaggtaacggcccaccaaggcgacgatcagtagccggactgagaggttgaacggccacattgggactgagacacggcccagactcctacgggaggcagcagtggggaatattgcacaatgggcgcaagcctgatgcagcgatgccgcgtgagggaagaaggttttcggattgtaaacctctgttgacagggacgataatgacggtacctgttcagaaagctccggctaactacgtgccagcagccgcggtaatacgtagggagcgagcgttgtccggaattactgggtgtaaagggagtgtaggcgggactgcaagtcagatgtgaaatgtaggggctcaacccctgacctgcatttgaaactgtagttcttgagtgaagtagaggtaagcggaattcccagtgtagcggtgaaatgcgtagatattgggaggaacatcagtggcgaaggcggcttactgggctttaactgacgctgaggctcgaaagcgtggggagcaaacaggattagataccctggtagtccacgccgtaaacgatgattactaggtgtggggggattgaccccttccgtgccgcagttaacacaataagtaatccacctggggagtacggtcgcaagactgaaactcaaaggaattgacgggggcccgcacaagcagtggagtatgtggattaattcgaagcaacgcgaagaaccttaccaggtcttgacatcgtacgcatagtgtagagatacatgaagtccttcgggacgtatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgttaacagagggaagcgaagccgcgaggtggagcaaatccccaaaagcagtcttagttcggattgtaggctgcaacccgcctacatgaagtcggaattgctagtaatcgcagatcagcatgctgcggtgaatacgttcccgggccttgtacacaccgcccgtcacaccatgggagttggtaacacccgaagtcagtagcctaaccgcaaggagggcgctgccgaaggtgggatcgatgactggggtgaagtcgtaacaaggtagccgtatcggaaggtgcggctggatcacctccttt

SEQ ID NO: 89 - DNA, 822 bp, Ruminococcaceae bacterium
attaagagtttgatcctggctcaggacgaacgctggcggcacgcttaacacatgcaagtcgaacggagttatttgagcttgcttaaataacttagtggcggacgggtgagtaacacgtgagcaatctgcctttcagagggggatagcagttggaaacgactgataataccgcataatataacgaaaccgcatgaccctgctatcaaagatttatcgctgaaagatgagctcgcgtctgattaggtagttggtgaggtaacggctcaccaagccgacgatcagtagccggactgagaggttgaacggccacattgggactgagacacggcccagactcctacgggaggcagcagtggggaatattgcacaatgggcgcaagcctgatgcagcgatgccgcgtgagggaagaaggttttaggattgtaaacctctgtcctatggaaagataatgacggtaccataggaggaagctccggctaactacgtgccagcagccgcggtaatacgtagggagcgagcgttgtccggaattactgggtgtaaagggagtgtaggcgggactgcaagtcagatgtgaaaactatgggcttaacccatagactgcatttgaaactgcagttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttc

SEQ ID NO: 90 - DNA, 1523 bp, Ruminococcaceae bacterium
ctaagagtttgatcctggctcaggacgaacgctggcggcacgcctaacacatgcaagtcgaacggagctattttagcggaagccttcgggcagaagctggcttagcttagtggcggacgggtgagtaacacgtgagcaacctgcctttgcgagggggataacgtttggaaacgaacgctaataccgcataatgtcagaaggtcgcatgattttctgaccaaagatttatcgcgcaaagatgggctcgcgtccgattagatagttggtgaggtaacggcccaccaagtctgcgatcggtagccggactgagaggttgaacggccacattgggactgagacacggcccagactcctacgggaggcagcagtgggggatattgcacaatggagggaactctgatgcagcgatgccgcgtgagggaagacggtcttcggattgtaaacctctgtcttcagggacgaacacaatgacggtacctgaggaggaagctccggctaactacgtgccagcagccgcggtaatacgtagggagcaagcgttgtccggaattactgggtgtaaagggagtgtaggcgggtctccaagtccgttgtcaaatctatcggctcaaccgatagccgcggcggaaactggaggtcttgagtgaagtagaggcaggcggaattcctagtgtagcggtgaaatgcgtagatattaggaggaacaccagtggcgaaggcggcctgctgggctttaactgacgctgaggctcgaaagtgtggggagcaaacaggattagataccctggtagtccacactgtaaacgatgattactaggtgtggggggactgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcataccatagagatatgggaagcccttcggggcatccagacaggtggtgcatggttgtcgtcagctcgtgtcgtgagatgttgggttaagtcccgcaacgagcgcaacccttatccttagttgctacgcaagagcactctaaagagactgccgttgacaaaacggaggaaggtggggatgacgtcaaatcatcatgccccttatgacctgggctacacacgtactacaatggcgattaacaaagggatgcaacacggcgacgtgaagcggaacccaaaaaatcgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagtcagtagcctaaccgcaaggagggcgctgccgaaggtgggattgatgactggggtgaagtcgtaacaaggtagccgtatcggaaggtgcggctggatcacctccttt

SEQ ID NO: 91 - DNA, 1415 bp, Hydrogenoanaerobacterium saccharovorans
agtttagtggcggacgggtgagtaacacgtgagcaacctgcctttcagaggggaataacattcggaaacgaatgctaataccgcataatgcaacgagatggcatcatcttgctgccaaagatttatcgctgaaagatgggctcgcgcccgattagctagttggtgaggtaatggcccaccaaggcaacgatcggtagccggactgagaggttgatcggccacattgggactgagacacggcccagactcctacgggaggcagcagtgggggatattgcacaatgggcgaaagcctgatgcagcgacgccgcgtgagggaagacggttttcggattgtaaacctctgtcttcagggacgataatgacggtacctgaggaggaagcaccggctaactacgtgccagcagccgcggtaatacgtagggtgcaagcgttgtccggaattactgggtgtaaagggagcgtaggcgggattgtaagttggatgtgtaatgtaccggctcaaccggtaacttgcattcaaaactgcagttcttgagtgaagtagaggcaggcggaattcctagtgtagcggtgaaatgcgtagatattaggaggaacaccagtggcgaaggcggcctgctgggcttttactgacgctgaggctcgaaagcatgggtagcaaacaggattagataccctggtagtccatgccgtaaacgatgattactaggtgtgggtgtgcaagcatccgtgccgcagctaacgcaataagtaatccacctggggagtacggccgcaaggctgaaactcaaaggaattgacgggggcccgcacaagcagtggattatgtggtttaattcgaagcaacgcgaagaaccttaccaggtcttgacatcccttgcataccatagagatatgggaagcccttcggggcaaggagacaggtggtgcatggttgtcgtcagctcgtgtcgtgagatgttgggttaagtcccgcaacgagcgcaacccttactattagttgctacgcaagagcactctaataggactgccgttgacaaaacggaggaaggtggggatgacgtcaaatcatcatgccccttatgacctgggctacacacgtaatacaatgacgataaacagagggtagcgaagccgcgaggtggagccaatccccaaaagtcgtctcagttcggattgcaggctgcaactcgcctgcatgaagtcggaattgctagtaatcgcaggtcagcatactgcggtgaatacgttcccgggccttgtacacaccgcccgtcacaccatgggagtcggtaacacccgaagccagtagtctaaccgcaaggaggacgctgtcgaaggtgggattgatgactggggtgaagtcgtaacaaggtagccgtatcggaaggtgcggctggatcacctccttt

SEQ ID NO: 92 - DNA, 1302 bp, Ruminococcaceae bacterium
caaagatttatcgctgtgagatggattcgcgtccgattagatagttggtgaggtaacggcccaccaagtcgacgatcggtagccggactgagaggttgaacggccacattgggactgagacacggcccagactcctacgggaggcagcagtgggggatattgcacaatgggcgcaagcctgatgcagcgacgccgcgtgtgggaagacggccctcgggttgtaaaccactggctttggggacgataatgacggtacccaaggaggaagctccggctaactacgtgccagcagccgcggtaatacgtagggagcgagcgttgtccggaattactgggtgtaaagggagcgtaggcgggagtgcaagttgaatgtttaatctatgggctcaacccatatcagcgttcaaaactgcatttcttgagtgaagtagaggttggcggaattcctagtgtagcggtgaaatgcgtagatattaggaggaacaccagtggcgaaggcggccaactgggcttttactgacgctgaggctcgaaagcgtggggagcaaacaggattagataccctggtagtccacgccgtaaacgatgaatactaggtgtggggggactgaccccttccgtgccgcagttaacacaataagtattccacctggggagtacggccgcaaggctgaaactcaaaggaattgacgggggcccgcacaagcagtggattatgtggtttaattcgaagcaacgcgaagaaccttaccaggccttgacatctcctgagtagcctagagataggtgatgcccttcggggcaggaagacaggtggtgcatggttgtcgtcagctcgtgtcgtgagatgttgggttaagtcccgcaacgagcgcaacccttacggatagttgctacgcaagagcactctatcaggactgccgttgacaaaacggaggaaggtggggatgacgtcaaatcatcatgccccttatggcctgggctacacacgtaatacaatggcgtttaacagagggaagcaagaccgcgaggtggagcgaatcctcaaaaggcgtctcagttcagattgcaggctgcaacccgcctgcatgaagtcggaattgctagtaatcgcggatcagcatgccgcggtgaatacgttctcgggccttgtacacaccgcccgtcacaccatggaagtcggtaacacccgaagtcagtagcctaaccgcaaggggggcgctgccgaaggtgggattggtaactggggtgaagtcgtaacaaggtagccgtatcggaaggtgcggctggatcacctccttt

SEQ ID NO: 93 - DNA, 1312 bp, Oscillibacter sp.
tgccaaagatttatcgctgaaagatggcctcgcgtctgattagctagttggtggggtaacggcccaccaaggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttaagtgggaagagcagaagacggtaccacttgaataagccacggctaactacgtgccagcagccgcggtaatacgtaggtggcaagcgttgtccggatttactgggtgtaaagggcgtgtagccgggtgtgcaagtcagatgtgaaatctggaggctcaacctccaaactgcatttgaaactgtgcatcttgagtatcggagaggtaatcggaattccttgtgtagcggtgaaatgcgtagatataaggaagaacaccagtggcgaaggcggattactggacgacaactgacggtgaggcgcgaaagcgtggggagcaaacaggattagataccctggtagtccacgctgtaaacgatcaatactaggtgtgcggggactgatcccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccagggcttgacatcctactaatgaagcagagatgcattaagtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggttaacagagggatgcaaatccgcgaggaggagcgaaccccgaaaagccgtctcagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 94 - DNA, 1529 bp, Pseudoflavonifractor sp.
tattgagagtttgatcctggctcaggatgaacgctggcggcgtgcttaacacatgcaagtcgaacggagagccaatgacggagttttcggacaacggatttggtttcttagtggcggacgggtgagtaacgcgtgagcaacctgccttggagtggggaataacagctggaaacagttgctaataccgcataatgcagcgaggggacatcctcttgctgccaaagatttatcgctctgagatggactcgcgtctgattagctggttggcggggtaacggcccaccaaggcgacgatcagtagccggactgagaggttgaccggccacattgggactgagatacggcccagactcctacgggaggcagcagtggggaatattgggcaatgggcgaaagcctgacccagcaacgccgcgtgaaggaagaaggccctcgggttgtaaacttcttttatcagggacgaaacaaatgacggtacctgatgaataagccacggctaactacgtgccagcagccgcggtaatacgtaggtggcaagcgttatccggatttactgggtgtaaagggcgtgtaggcgggtctgcaagtcaggtgtgaaattccagggctcaaccctggaactgcacttgaaactgtgggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgctagttgctacgcaagagcactctagcgagactgccgttgacaaaacggaggaaggtggggacgacgtcaaatcatcatgccccttatgtcctgggccacacacgtactacaatggcggtcaacagagggaagcaataccgcgaggtggagcgaatccctaaaagccgtcccagttcggattgcaggctgaaacccgcctgcatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgtatcggaaggtgcggctggatcacctccttt

SEQ ID NO: 95 - DNA, 1511 bp, Neglecta sp.
ttagagagtttgatcctggctcaggacgaacgctggcggcgtgcctaacacatgcaagtcgaacggagttaagagaagcttgcttttattaacttagtggcggacgggtgagtaacgcgtgagcaatctgcctttcagtggggaataacgttctgaaaagaacgctaataccgcataatattgttgagccgcatggtttgataatcaaaggatttattcgctgaaagatgagctcgcgtccgattagatagttggtgaggtaacggctcaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagataatgacggtagcagaggaggaagctccggctaactacgtgccagcagccgcggtaatacgtagggagcaagcgttgtccggatttactgggtgtaaagggtgcgtaggcggctatgcaagtcaggagtgaaatctatgggcttaacccataaactgctcttgaaactgtatagcttgagtgaagtagaggtaggcggaattcccggtgtagcggtggaatgcgtagagatcgggaggaacaccagtggcgaaggcggcctactgggctttaactgacgctgaagcacgaaagcgtgggtagcaaacaggattagataccctggtagtccacgccgtaaacgatgattactaggtgtggggggtctgaccccctccgtgccggagttaacacaataagtaatccacctggggagtacggtcgcaagactgaaactcaaaggaattgacgggggcccgcacaagcagtggagtatgtggattaattcgaagcaacgcgaagaaccttaccaggtcttgacatccctctgaccgctctagagatagagcttctcttcggagcagaggtgacaggtggtgcatggttgtcgtcagctcgtgtcgtgagatgttgggttaagtcccgcaacgagcgcaacccctatgattagttgctacgcaagagcactctaatcagactgccgttgacaaaacggaggaaggtggggatgacgtcaaatcatcatgccccttatgacctgggcctcacacgtactacaatggccgttaacaacgggatgcaatatagcgatatggagcaaaaccccaaaaacggtctcagttcggattgtaggctgaaactcgcctgcatgaagctggaattgctagtaatcgcagatcagaatgctgcggtgaatacgttcccgggccttgtacacaccgcccgtcacaccatgggagccggtaatacccgaagtcagtagcctaaccgtaaggagggcgctgccgaaggtagggttggcgactggggtgaagtcgtaacaaggtagccgtatcggaaggtgcggctggatcacctccttt

SEQ ID NO: 96 - DNA, 1530 bp, Clostridium sp.
tattgagagtttgatcctggctcaggatgaacgctggcggcgtgcttaacacatgcaagtcgaacggagtgctcatgacggagttttcggacaacggattgggttacttagtggcggacgggtgagtaacgcgtgaggaacctgcctcggagtggggaataacataccgaaaggtgtgctaataccgcataatgcagttgggtcgcatgactctgactgccaaagatttatcgctctgagatggcctcgcgtctgattagctagttggcggggtaacggcccaccaaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaaacaaatgacggtacctgacgaataagccacggctaactacgtgccagcagccgcggtaatacgtaggtggcaagcgttatccggatttactgggtgtaaagggcgtgtaggcgggactgcaagtcaggtgtgaaaaccaggggctcaacctctggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggtggttaacagagggaagcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 97 - DNA, 1521 bp, Anaerotruncus sp.
caaagagtttgatcctggctcaggacgaacgctggcggcgcgcctaacacatgcaagtcgaacggagtgttttcacggaagttttcggatggaagtggttacacttagtggcggacgggtgagtaacacgtgagcaacctgcctttcagagggggataacagttggaaacgactgctaataccgcatgatattaccgggtcacatggcctggcaatcaaaggagcaatccgctgaaagatgggctcgcgtccgattagccagttggcggggtaatggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtcttaggggaagaaaatgacggtaccctaagaggaagctccggctaactacgtgccagcagccgcggtaatacgtagggagcgagcgttgtccggaattactgggtgtaaagggagcgtaggcgggatgccaagtagaatgttaaatccatcggctcaactggtggcagcgttctaaactggcgttcttgagtgaggtagaggcaggcggaattcctagtgtagcggtgaaatgcgtagatattaggaggaacaccagtggcgaaggcggcctgctgggccttaactgacgctgaggctcgaaagcgtggggagcaaacaggattagataccctggtagtccacgccgtaaacgatgaatcctaggtgtggggggactgacaccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcataccatagagatatgggaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactcaaacagagggaagcgacaccgcgaggtgaagcggatcccaaaaaagtgtctcagttcggatcgcaggctgcaacccgcctgcgtgaagtcggaattgctagtaatcgcggatcagcatgccgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacctccttt

SEQ ID NO: 98 - DNA, 1515 bp, Anaeromassilibacillus sp.
ttttgagagtttgatcctggctcaggacgaacgctggcggcgtgcctaacacatgcaagtcgaacgaagctttgaggagcttgctttttaaagcttagtggcggacgggtgagtaacgcgtgagcaacctgcctctcagagggggataacgttttgaaaagaacgctaataccgcataacatatcggaaccgcatgattctgatatcaaaggagcaatccgctgagagatgggctcgcgtccgattagttagttggtgaggtaacggctcaccaagactacgatcggtagccggactgagaggttgatcggccacattgggactgagacacggcccagactcctacgggaggcagcagtgggggatattgcgcaatgggggaaaccctgacgcagcaacgccgcgtgaaggaagaaggtcttcggattgtaaacttcttttgtcagggacgaagaaagtgacggtacctgacgaataagctccggctaactacgtgccagcagccgcggtaatacgtagggagcgagcgttgtccggatttactgggtgtaaagggtgcgtaggcggccgagcaagtcagttgtgaaaactatgggcttaacccataacgtgcaattgaaactgtccggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctgagaatccttaagagattagggagtgccttcgggaactcagagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggccattaacagagggaagcaaaaccgcgaggcagagcaaacccctaaaaatggtcccagttcggattgtaggctgcaacccgcctacatgaagttggaattgctagtaatcgcggatcagcatgccgcggtgaatacgttcccgggccttgtacacaccgcccgtcacaccatgggagccggtaatacccgaagtcagtagtctaacagcaatgaggacgctgccgaaggtaggattggcgactggggtgaagtcgtaacaaggtagccgtatcggaaggtgcggctggatcacctccttt

SEQ ID NO: 99 - DNA, 1512 bp, Gemmiger formicilis
tataaagagtttgatcctggctcaggacgaacgctggcggcgcgcctaacacatgcaagtcgaacggaacttgagagagcttgctttttcaagtttagtggcgaacgggtgagtaacgcgtgagtaacctgccctggagtgggggacaacagttggaaacgactgctaataccgcataagcccacggcaccgcatggtactgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgtacgggacgataatgacggtaccgtacaagaaagccacggctaactacgtgccagcagccgcggtaaaacgtaggtggcaagcgttgtccggaattactgggtgtaaagggagcgcaggcggaccggcaagttggaagtgaaatctatgggctcaacccataaattgctttcaaaactgctggccttgagtagtgcagaggtaggcggaattcccggtgtagcggtggaatgcgtagatatcgggaggaacaccagtggcgaaggcggcctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaataccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtccgtagtctaaccgcaaggaggacgcggccgaaggtaaaactggtgattggggtgaagtcgtaacaaggtagccgtatcggaaggtgcggctggatcacctccttt

SEQ ID NO: 100 - DNA, 1529 bp, Unknown
  source: /note=“Description of Unknown Ruminococcaceae unnamed sp 1 sequence”
ttagagagtttgatcctggctcaggacgaacgctggcggcgtgcctaacacatgcaagtcgaacggaacttctttaaaggatttcttcggaatgaatttgattaagtttagtggcggacgggtgagtaacgcgtgagtaacctgcctctaagaggggaataacattctgaaaagaatgctaataccgcataatatatatttatcgcatggtagatatatcaaagatttatcgcttagagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggaaaccctgacgcagcaacgccgcgtgaaggatgaaggtcttcggattgtaaacttcttttattaaggacgaagaaagtgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccatagcctgcatttgaaactgcagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctactaacgagatagagatatgttaggtgcccttcggggaaagtagagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggacattaacagagggaagcaatacagtgatgtggagcaaacccctaaaaatgttctcagttcagattgcaggctgcaacccgcctgtatgaagatggaattgctagtaatcgcagatcagcatgctgcggtgaatacgttcccgggccttgtacacaccgcccgtcacaccatgggagccggtaatacccgaagtcagtagtctaaccgcaaggaggacgctgccgaaggtaggattggcgactggggtgaagtcgtaacaaggtagccgtatcggaaggtgcggctggatcacctccttt

SEQ ID NO: 101 - DNA, 1210 bp, Unknown
  source: /note=“Description of Unknown Ruminococcaceae unnamed sp 2 sequence”
gaacggccacattgggactgagacacggcccagactcctacgggaggcagcagtggggaatattgcgcaatgggggaaaccctgacgcagcaacgccgcgtgattgaagaaggccttcgggttgtaaagatctttaattggggacgaaaaatgacggtacccaaagaataagctccggctaactacgtgccagcagccgcggtaatacgtagggagcaagcgttatccggatttactgggtgtaaagggcgagtaggcgggctggcaagttgggagtgaaatcccggggcttaaccccggaactgctttcaaaactgctggtcttgagtgatggagaggcaggcggaattccgtgtgtagcggtgaaatgcgtagatatacggaggaacaccagtggcgaaggcggcctgctggacattaactgacgctgaggagcgaaagcgtggggagcaaacaggattagataccctggtagtccacgccgtaaacgatggatactaggtgtgggaggtattgaccccttccgtgccggagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatccctctgaccgccctagagatagggtttcccttcggggcagaggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaaaacagtgatgtggagcaaatccctaaaagctgtcccagttcagattgcaggctgcaactcgcctgcatgaagtcggaattgctagtaatcgcggatcagcatgccgcggtgaatacgttcccgggccttgtacacaccgcccgtcacaccatgagagccggtaatacccgaagtccgtagcctaaccgcaaggagggcgcggccgaaggtaggactggtaattagggtgaagtcgtaacaaggtagccgtatcggaaggtgcggctggatcacctccttt

SEQ ID NO: 102 - DNA, 1307 bp, Gemmiger formicilis
aaaaggatttattcgctttaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtagatggaattcccggtgtagcggtggaatgcgtagatatcgggaggaacaccagtggcgaaggcggtctactgggcaccaactgacgctgaggctcgaaagcatgggtagcaaacaggattagataccctggtagtccatgccgtaaacgatgattactaggtgttgggggattgaccccctcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagcacagagatgtgtgaaatccttcgggacatcgagacaggtggtgcatggttgtcgtcagctcgtgtcgtgagatgttgggttaagtcccgcaacgagcgcaacccttattgccagttactacgttaagaggactctggcgagactgccgttgacaaaacggaggaaggtggggatgacgtcaaatcatcatgccctttatgacctgggctacacacgtactacaatggcgttaaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcgatagtctaaccgcaaggaggacgtcgccgaaggtaaaactggtgattggggtgaagtcgtaacaaggtagccgtatcggaaggtgcggctggatcacctccttt

SEQ ID NO: 103 - DNA, 1423 bp, Unknown
  source: /note=“Description of Unknown Ruminococcaceae unnamed sp 3 sequence”
gcttagtggcggactggtgagtaacgcgtgaggaacctgcctttcagagggggacaacagttggaaacgactgctaataccgcatgatgcatattgaccgcatggtcggtatgtcaaagatttatcgctgaaagatggcctcgcgtctgattagcttgttggtgaggtaacggcccaccaaggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttctttgacaggggaagagtagaagacggtaccctgaaaacaagccacggctaactacgtgccagcagccgcggtaatacgtaggtggcaagcgttgtccggatttactgggtgtaaagggcgtgtagccgggaaggcaagtcagatgtgaaatctggaggctcaacctccaaactgcatttgaaactgtctttcttgagtatcggagaggtaatcggaattccttgtgtagcggtgaaatgcgtagatataaggaggaacaccagtggcgaaggcggattactggacgacaactgacggtgaggcgcgaaagcgtggggagcaaacaggattagataccctggtagtccacgctgtaaacgatcaatactaggtgtgcggggactgaccccctgcgtgccggagttaacacaataagtattgcacctggggagtacgatcgcaaggttgaaactcaaaggaattgacgggggcccgcacaagcggtggattatgtggtttaattcgaagcaacgcgaagaaccttaccagggcttgacatcctactaatgaagcagagatgcattaagtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtaatacaatggcggtaaacagagggatgcaaagccgtgaggtggagcgaacccctaaaagccgtcccagttcggattgcaggctgcaacccgcctgcatgaagtcggaatcgctagtaatcgcggatcagcatgccgcggtgaatacgttcccgggccttgtacacaccgcccgtcacaccatgagagtcgggaacacccgaagcccgtagcctaacagcaatgagggcgcggtcgaaggtgggttcgataattggggtgaagtcgtaacaaggtagccgtatcggaaggtgcggctggatcacctccttt

SEQ ID NO: 104 - DNA, 1062 bp, Unknown
  source: /note=“Description of Unknown Ruminococcaceae unnamed sp 4 sequence”
tatagagagtttgatcctggctcaggacgaacgctggcggcgtgcttaacacatgcaagtcgaacggagcacccctgaatgaggtttcggccaaaggaagggaatgcttagtggcggactggtgagtaacgcgtgaggaacctgcctttcagagggggacaacagttggaaacgactgctaataccgcatgacacatgaatggggcatcccattgatgtcaaagatttatcgctgaaagatggcctcgcgtcccattagctagtaggcggggtaacggcccacctaggcgacgatgggtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaacagtagaagagggtacctgacgaataagccacggctaactacgtgccagcagccgcggtaatacgtaggtggcaagcgttgtccggatttactgggtgtaaagggcgtgcagccgggctggcaagtcaggcgtgaaatcccagggctcaaccctggaactgcgtttgaaactgctggtcttgagtaccggagaggtcatcggaattccttgtgtagcggtgaaatgcgtagatataaggaagaacaccagtggcgaaggcggatgactggacggcaactgacggtgaggcgcgaaagcgtggggagcaaacaggattagataccctggtagtccacgctgtaaacgatcaatactaggtgtgcggggactgaccccctgcgtgccgcagttaacacaataagtattgcacctggggagtacgatcgcaaggttgaaactcaaaggaattgacgggggcccgcacaagcggtggattatgtggtttaattcgaagcaacgcgaagaaccttaccagggcttgacatcctactaacgaagtagagatacattaggtgcccttcggggaaagtagagacaggtggtgcatggttgtcgtcagct

SEQ ID NO: 105 - DNA, 1531 bp, Unknown
  source: /note=“Description of Unknown Ruminococcaceae unnamed sp 5 sequence”
tttagagagtttgatcctggctcaggacgaacgctggcggcgtgcctaacacatgcaagtcgaacggagttatttaaatagaacccttcggggtgacgttttaataacttagtggcggacgggtgagtaacgcgtgagtaacctgcctttcagagggggataacgtcctgaaaaggacgctaataccgcatgatatatttgtgccgcatggtatggatatcaaaggagcaatccgctggaagatggactcgcgtccgattagctagttggaggggtaacggcccaccaaggcgacgatcggtagccggactgagaggttgaacggccacattgggactgagacacggcccagactcctacgggaggcagcagtgggggatattgcgcaatgggggaaaccctgacgcagcaacgccgcgtgaaggaagaaggttttcggattgtaaacttcttttctaagggacgaagaagtgacggtaccttaggaataagctccggctaactacgtgccagcagccgcggtaatacgtagggagcaagcgttgtccggatttactgggtgtaaagggtgcgtaggcggcaatgcaagtcagatgtgaaatgcacgggctcaacccgtgagctgcatttgaaactgtgttgcttgagtgaggtagaggcaggcggaattcccggtgtagcggtgaaatgcgtagagatcgggaggaacaccagtggcgaaggcggcctgctgggccttaactgacgctgatgcacgaaagcgtgggtagcaaacaggattagataccctggtagtccacgctgtaaacgatgattactaggtgtggggggtctgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccagctaacgaagtagagatacattaggtgcccttcggggaaagctgagacaggtggtgcatggttgtcgtcagctcgtgtcgtgagatgttgggttaagtcccgcaacgagcgcaacccttgctgttagttgctacgcaagagcactctaacaggactgccgttgacaaaacggaggaaggtggggacgacgtcaaatcatcatgccccttatgacctgggctacacacgtactacaatggccgtcaacagagggaagcaagaccgcgaggtggagcaaacccccaaaaacggccccagttcggattgtaggctgcaacccgcctacatgaagtcggaattgctagtaatcgcggatcagcatgccgcggtgaatacgttcccgggccttgtacacaccgcccgtcacaccatgggagccggtaatacccgaagtcagtagcctaaccgcaaggagggcgctgccgaaggtaggattggcgactggggtgaagtcgtaacaaggtagccgtatcggaaggtgcggctggatcacctccttt

SEQ ID NO: 106 - DNA, 1531 bp, Unknown
  source: /note=“Description of Unknown Ruminococcaceae unnamed sp 6 sequence”
acgagagtttgatcctggctcaggacgaacgctggcggcgtgcctaacacatgcaagtcgaacgagaatctttgaacagatcttttcggagtgacgttcaaagaggaaagtggcggacgggcgagtaacgcgtgagtaacctgcccataagagggggataatccatggaaacgtggactaataccgcatattgtagttaagttgcatgacttgattatgaaagatttatcgcttatggatggactcgcgtcagattagatagttggtgaggtaacggctcaccaagtcaacgatctgtagccgaactgagaggttgatcggccgcattgggactgagacacggcccagactcctacgggaggcagcagtggggaatattgcgcaatgggggcaaccctgacgcagcaacgccgcgtgcaggaagaaggtcttcggattgtaaactgttgtcgcaagggaagaagacagtgacggtaccttgtgagaaagtcacggctaactacgtgccagcagccgcggtaatacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggctttctggacgacaactgacgctgaggcgcgaaagtgtggggagcaaacaggattagataccctggtagtccacaccgtaaacgatggatactaggtgtaggaggtatcgaccccttctgtgccgcagttaacacaataagtatcccacctggggagtacgaccgcaaggttgaaactcaaaggaattgacgggggcccgcacaagcagtggagtatgtggtttaattcgaagcaacgcgaagaaccttacctgggcttgacatccctggaatcgagtagagatacttgagtgccttcgggaatcaggtgacaggtggtgcatggttgtcgtcagctcgtgtcgtgagatgttgggttaagtcccgcaacgagcgcaacccctattgtcagttgccatcattaagttgggcactctggcgagactgccggtgacaaatcggaggaaggtggggacgacgtcaaatcatcatgccccttatgcccagggctacacacgtactacaatggccgataacaaagtgcagcgaaaccgtgaggtggagcgaatcacaaaactcggtctcagttcagattgcaggctgcaactcgcctgcatgaagttggaattgctagtaatcgcggatcagaatgccgcggtgaatacgttcccgggccttgtacacaccgcccgtcacaccatgagagtcgataacacccgaagcctgtgagctaacctttaggaggcagcagtcgaaggtggggttgatgattggggtgaagtcgtaacaaggtagccgtatcggaaggtgcggctggatcacctccttt

SEQ ID NO: 107 - DNA, 1523 bp, Unknown
  source: /note=“Description of Unknown Ruminococcaceae unnamed sp 7 sequence”
attaagagtttgatcctggctcaggacgaacgctggcggcgcgcctaacacatgcaagtcgaacgaagtttcataacggaagttttcggatggaagatatgaaacttagtggcggacgggtgagtaacacgtgagcaacctgccttttagagggggataacgtttggaaacgaacgctaataccgcataacgtagtcgatcggcatcgattgactaccaaaggagcaatccgctgaaagatgggctcgcgtccgattagatagttggcggggtaacggcccaccaagtcgacgatcggtagccggactgagaggttgatcggccacattgggactgagacacggcccagactcctacgggaggcagcagtggggaatattgcacaatgggggaaaccctgatgcagcgacgccgcgtgagggaagaaggttttcggattgtaaacctctgtccttggtgacgataatgacggtagccaaggaggaagccacggctaactacgtgccagcagccgcggtaatacgtaggtggcaagcgttgtccggaattactgggtgtaaagggagcgtaggcgggaaagcaagttgaatgtttaaactatcggctcaaccgataatcgcgttcaaaactgtttttcttgagtgaagtagaggtaggcggaattcctagtgtagcggtgaaatgcgtagatattaggaggaacaccagtggcgaaggcggcctactgggctttaactgacgctgaggctcgaaagcgtgggtagcaaacaggattagataccctggtagtccacgccgtaaacgatgattactaggtgtggggggatcaacccttccgtgccgcagcaaacgcaataagtaatccacctggggagtacgaccgcaaggttgaaactcaaaggaattgacggggacccgcacaagcagtggagtatgtggtttaattcgaagcaacgcgaagaaccttaccaggtcttgacatccaacgaactcgctagagatagcaaggtgcccttcggggagcgttgagacaggtggtgcatggttgtcgtcagctcgtgtcgtgagatgttgggttaagtcccgcaacgagcgcaacccttactgatagttgctacgcaagagcactctatcgggactgccgttgacaaaacggaggaaggtggggatgacgtcaaatcatcatgccccttatgacctgggctacacacgtactacaatggctattaacaacgggaagcgaagaggtgactcggagccaatccaaaaaaatagtctcagttcggattgcaggctgcaactcgcctgcatgaagccggaattgctagtaatcgcggatcagcatgccgcggtgaatacgttcccgggtcttgtacacaccgcccgtcacaccatgagagttggcaacacccgaagtcagtagtctaaccgcaaggaggacgctgccgaaggtggggtcgatgattggggtgaagtcgtaacaaggtagccgtatcggaaggtgcggctggatcacctccttt

SEQ ID NO: 108 - DNA, 1505 bp, Eubacterium siraeum
  modified_base (442)..(448): a, c, t, g, unknown or other
caaagagtttgatcctggctcaggacgaacgctggcggcgcgcctaacacatgcaagtcgaacggtgaagaggagcttgctcctcggatcagtggcggacgggtgagtaacacgtgagcaacctggctctaagagggggacaacagttggaaacgactgctaataccgcataacgtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacgggaggcagcagtgggggatattggacaatgggggcaaccctgatccagcgacgccgcgtgagggaagaaggttttcggattgtaaacctctgttgacggagnnnnnnntgatggtatccgtttagaaagccacggctaactacgtgccagcagccgcggtaatacgtaggtggcaagcgttgtccggaattactgggtgtaaagggagtgtaggcgggatatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctgggcttttactgacgctgaggctcgaaagcgtggggagcaaacaggattagataccctggtagtccacgccgtaaacgatgattactaggtgtggggggattgaccccttccgtgccggagtaaacacaataagtaatccacctggggagtacgaccgcaaggttgaaactcaaaggaattgacgggggcccgcacaagcagtggagtatgtggtttaattcgacgcaacgcgaagaaccttaccaggtcttgacatcgagtgaccgcctaagagattaggctttcccttcggggacacaaagacaggtggtgcatggttgtcgtcagctcgtgtcgtgagatgttgggttaagtcccgcaacgagcgcaacccttatcattagttgctacgcaagagcactctaatgagactgccgttgacaaaacggaggaaggtggggatgacgtcaaatcatcatgccctttatgacctgggctacacacgtactacaatggcgtttaacaaagagaagcaaagccgcgaggcagagcaaatctccaaaaaacgtctcagttcggattgtaggctgcaactcgcctacatgaagtcggaattgctagtaatcgtaggtcagcatactacggtgaatacgttcccgggccttgtacacaccgcccgtcaaaccatgagagttggcaacacccgaagtcggtagtctaaccgcaaggaggacgccgccgaaggtggggttgatgattagggttaagtcgtaacaaggtagccgtatcggaaggtgcggctggatcacctccttt

SEQ ID NO: 109 - DNA, 1548 bp, Clostridium leptum; identical to SEQ ID NO: 29

SEQ ID NO: 110 - DNA, 1521 bp, Anaerotruncus colihominis
caaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 111 - DNA, 1103 bp, Subdoligranulum variabile
  modified_base (1)..(1): a, c, t, g, unknown or other
naagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagatgcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctccttt

(19) United States
(12) Patent Application Publication
(10) Pub. No.: US 2021/0361721 A1
(43) Pub. Date: Nov. 25, 2021

(54) TREATMENT OF A CANCER BY MICROBIOME MODULATION

(71) Applicants: SERES THERAPEUTICS, INC., Cambridge, MA (US); Board of Regents, The University of Texas System, Austin, TX (US)

(72) Inventors: Jennifer WORTMAN, Arlington, MA (US); Diao LIYANG, Belmont, MA (US); Christopher DESJARDINS, Somerville, MA (US); Georgios MARNELLOS, Cambridge, MA (US); Matthew HENN, Belmont, MA (US); Jennifer WARGO, Houston, TX (US); Vancheswaran GOPALAKRISHNAN, Houston, TX (US)

(21) Appl. No.: 15/733,682

(22) PCT Filed: Mar. 28, 2019
(86) PCT No.: PCT/US2019/024519

§ 371 Date: Sep. 28, 2020

Related U.S. Application Data
(60) Provisional application No. 62/818,601, filed on Mar. 14, 2019.
(60) Provisional application No. 62/649,453, filed on Mar. 28, 2018.

(51) Int. Cl. A61K 35/741; A61K 35/742; A61K 35/745; A61K 39/395; A61K 31/675; C12Q 1/689; C12Q 1/04

(52) U.S. Cl. CPC A61K 35/741; A61K 35/742; A61K 35/745; C12Q 1/04; A61K 31/675; C12Q 1/689; A61K 39/3955

(57) ABSTRACT

Methods are provided for identifying donors of fecal matter that can improve a subject's response to a checkpoint inhibitor in the treatment of cancer. Methods and compositions are also provided for using donated fecal matter in the treatment of cancer.

### CROSS-REFERENCE TO RELATED APPLICATIONS

This application is a national phase application under 35 U.S.C. § 371 of International Application No. PCT/US 2019/024519, filed Mar. 28, 2019, which claims priority to U.S. Patent Application No. 62/649,453, filed Mar. 28, 2018, and 62/818,601, filed Mar. 14, 2019, each of which are incorporated herein by reference in their entirety.

### INTRODUCTION

Mammals are colonized by microbes in the gastrointestinal (GI) tract, on the skin, and in other epithelial and tissue niches such as the oral cavity, eye surface and vagina. The gastrointestinal tract harbors an abundant and diverse microbial community. Hundreds of different species may form a commensal community in the GI tract of a healthy person. Interactions between microbial strains in these populations and between microbes and the host, e.g., the host immune system, shape the community structure, with availability of and competition for resources affecting the distribution of microbes. Such resources may be food, location and the availability of space to grow or a physical structure to which the microbe may attach. For example, host diet is involved in shaping the GI tract flora.

Harnessing the host immune system by microbiome modulation constitutes a promising approach for the treatment of cancer because of its potential to specifically target tumor cells while limiting harm to normal tissue, with durability of benefit associated with immunologic memory. Enthusiasm for this approach has been fueled by recent clinical success, particularly with antibodies that block immune inhibitory pathways, for example the CTLA-4 and the PD-1/PD-L1 pathways (Hodi et al. New Engl J Med 363:711-723 (2010); Hamid et al. New Engl J Med 369:134-144 (2013); herein incorporated by reference in their entireties). Early data have indicated that clinical responses to these immunotherapies are more frequent in patients who show evidence of an endogenous T cell response ongoing in the tumor microenvironment at baseline (Tumeh et al. Nature 51:568-571 (2014); Spranger et al. Sci Transl Med 5:200ra116 (2013); Ji et al. Cancer Immunol Immunother: CII 61, 1019-1031 (2012); Gajewski et al. Cancer J 16:399-403 (2010); herein incorporated by reference in their entireties). However, many cancer therapeutics have limited efficacy and there is a need to extend the range of patients who can benefit from these treatments. A number of factors can influence the efficacy of a cancer treatment, for example, smoking history, diabetes, obesity, and tumor size. It has been suggested that the microbiome of an individual can be a factor influencing efficacy.

Fecal transplantation and some individual species have been proposed as treatments for patients suffering from certain cancers either as sole treatments or as adjunctive therapy with other cancer treatments. Fecal transplantation, however, is generally a procedure of last resort because of, for example, the difficulty in producing a consistent product, the potential to transmit infectious or allergenic agents between hosts, and variability between fecal donors. There is a need for improved methods of selecting fecal donors and/or defined microbiome compositions that can be used to effect anti-tumor activity, alone or in combination with other cancer treatment methods, e.g., checkpoint inhibitors.

### SUMMARY

In one aspect, methods are provided for identifying donors of fecal matter that can improve a subject's response to an immune checkpoint inhibitor comprising determining whether the microbiome of the potential donor comprises bacteria belonging to one or more species that are phylogenetic descendants of the most recent common ancestor (MRCA) of Faecalibacterium prausnitzii and Flavonifractor plautii, i.e., they belong to the family Ruminococcaceae as defined herein.

In another aspect, methods are provided for identifying donors of fecal matter that can improve a subject's response to a checkpoint inhibitor comprising determining whether the microbiome of the potential donor comprises bacteria belonging to one or more species that have 16S rDNA sequence identity of at least 94.5% to 16S rDNA sequences of species belonging to the family Ruminococcaceae. In some embodiments, the one or more species may have 16S rDNA sequence identity of at least 98.7% to 16S rDNA sequences of species belonging to the family Ruminococcaceae.

In another aspect, methods are provided for identifying donors of fecal matter that can improve a subject's response to a checkpoint inhibitor comprising determining whether the microbiome of the potential donor comprises bacteria belonging to one or more species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In another aspect, methods are provided for identifying donors of fecal matter that can improve a subject's response to a checkpoint inhibitor comprising determining whether the microbiome of the potential donor comprises one or more strain of bacteria belonging to one or more of clade 101, clade 14, clade 126, clade 61, clade 125 or clade 135 as defined herein.

In some aspects, fecal material from identified donors can be used, e.g., in fecal microbiome transplantation or in a processed form derived from such material, for example a preparation enriched in Firmicutes (e.g., Clostridia, Clostridiales, or spore formers), that are in vegetative and/or spore form.

In another aspect, therapeutic compositions are provided that are derived from fecal matter obtained from a donor identified using a method described herein.

In another aspect, methods of treating a cancer in a mammalian subject are provided comprising administering to the subject a therapeutic composition derived from fecal matter obtained from a donor identified using a method described herein.

In another aspect, methods are provided for identifying donated fecal matter that can improve a subject's response to a checkpoint inhibitor comprising determining whether the donated fecal matter comprises bacteria belonging to one or more species that are phylogenetic descendants of the MRCA of Faecalibacterium prausnitzii and Flavonifractor plautii.

In another aspect, methods are provided for identifying donated fecal matter that can improve a subject's response to a checkpoint inhibitor comprising determining whether the microbiome of the potential donor comprises bacteria belonging to one or more species that have 16S rDNA sequence identity of at least 94.5% to 16S rDNA sequences of species belonging to the family Ruminococcaceae. In some embodiments, the one or more species may have 16S rDNA sequence identity of at least 98.7% to 16S rDNA sequences of species belonging to the family Ruminococcaceae.

In another aspect, methods are provided for identifying donated fecal matter that can improve a subject's response to a checkpoint inhibitor comprising determining whether the donated fecal matter comprises bacteria belonging to one or more species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In another aspect, methods are provided for identifying donated fecal matter that can improve a subject's response to a checkpoint inhibitor comprising determining whether the donated fecal matter comprises one or more strain of bacteria belonging to one or more of clade 101, clade 14, clade 126, clade 61, clade 125 or clade 135 as defined herein.

In some aspects, fecal material from identified donated fecal matter can be used, e.g., in fecal microbiome transplantation or in a processed form derived from such material, for example a preparation enriched in Firmicutes (e.g., Clostridia, Clostridiales, or spore formers), that are in vegetative and/or spore form.

In another aspect, therapeutic compositions are provided that are derived from donated fecal matter identified using a method described herein.

In another aspect, methods of treating a cancer in a mammalian subject are provided comprising administering to the subject a therapeutic composition derived from donated fecal matter identified using a method described herein.

In one aspect, provided are therapeutic compositions comprising an effective amount of an isolated population of bacteria belonging to one or more of the family Ruminococcaceae, e.g., the genera Ruminococcus, Gemmiger, Faecalibacterium, Subdoligranulum or combinations thereof. In some embodiments, the therapeutic composition may comprise bacteria belonging to at least two, three or four of the genera listed.

In another aspect, provided are therapeutic compositions comprising an effective amount of an isolated population of bacteria that are phylogenetic descendants of the most recent common ancestor (MRCA) of Faecalibacterium prausnitzii and Flavonifractor plautii. In another aspect, provided are therapeutic compositions comprising an effective amount of an isolated population of bacteria that have 16S rDNA sequence identity of at least 94.5% to 16S rDNA sequences of species belonging to the family Ruminococcaceae. In some embodiments, the bacteria have 16S rDNA sequence identity of at least 98.7% to 16S rDNA sequences of species belonging to the family Ruminococcaceae. In some embodiments, the therapeutic compositions may comprise one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In some embodiments, the therapeutic composition may comprise at least two, three, four, five or more of the species listed. In another aspect, provided are therapeutic compositions comprising an effective amount of an isolated population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In another aspect, provided are therapeutic compositions comprising an effective amount of an isolated population of bacteria belonging to one or more of the genera Alistipes, Bacteroides, Blautia, Clostridium, Eubacterium, Parabacteroides or combinations thereof. In another aspect, provided are therapeutic compositions comprising an effective amount of an isolated population of bacteria belonging to one or more of the genera Barnesiella, Bifidobacterium, Blautia, Erysipelotrichaceae, Odoribacter, Parabacteroides or combinations thereof. In some embodiments, the therapeutic composition may comprise bacteria belonging to at least two, three, four, five or more of the genera listed.

In another aspect, provided are therapeutic compositions comprising an effective amount of an isolated population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, provided are therapeutic compositions comprising an effective amount of an isolated population of bacteria species selected from Alistipes senegalensis, Bacteroides dorei, Blautia_SC109, Clostridium_SC64, Eubacterium_biforme, Parabacteroides distasonis or combinations thereof. In another aspect, provided are therapeutic compositions comprising an effective amount of an isolated population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In some embodiments, the therapeutic composition may comprise at least two, three, four, five or more of the species listed.

In one aspect, provided are therapeutic compositions comprising an effective amount of a purified population of bacteria belonging to one or more of the genera Ruminococcus, Gemmiger, Faecalibacterium, Subdoligranulum or combinations thereof. In some embodiments, the therapeutic composition may comprise bacteria belonging to at least two, three or four the genera listed.

In another aspect, provided are therapeutic compositions comprising an effective amount of a purified population of bacteria belonging to one or more of the genera Alistipes, Bacteroides, Bamesiella, Bifidobacterium, Blautia, Clostridium, Eubacterium, Erysipelotrichaceae, Odoribacter, Parabacteroides or combinations thereof. In another aspect, provided are therapeutic compositions comprising an effective amount of a purified population of bacteria belonging to one or more of the genera Alistipes, Bacteroides, Blautia, Clostridium, Eubacterium, Parabacteroides or combinations thereof. In another aspect, provided are therapeutic compositions comprising an effective amount of a purified population of bacteria belonging to one or more of the genera Bamesiella, Bifidobacterium, Blautia, Erysipelotrichaceae, Odoribacter, Parabacteroides or combinations thereof. In some embodiments, the therapeutic composition may comprise bacteria belonging to at least two, three, four, five or more of the genera listed.

In another aspect, provided are therapeutic compositions comprising an effective amount of a purified population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, provided are therapeutic compositions comprising an effective amount of a purified population of bacteria species selected from Alistipes senegalensis, Bacteroides dorei, Blautia_SC109, Clostridium_SC64, Eubacterium_biforme, Parabacteroides distasonis or combinations thereof. In another aspect, provided are therapeutic compositions comprising an effective amount of a purified population of bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In some embodiments, the therapeutic composition may comprise at least two, three, four, five or more of the species listed.

In some embodiments, the therapeutic compositions further comprise an anticancer agent. In some embodiments, the anticancer agent is a checkpoint inhibitor. In some embodiments, the checkpoint inhibitor is selected from an anti-PD-1 antibody, an anti-CTLA-4 antibody, an anti-PD-L1 antibody or combinations thereof. In some embodiments, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors, STI-A1010 or combinations thereof. In some embodiments, the anticancer agent is cyclophosphamide.

In some embodiments, each isolated population of bacteria in the therapeutic composition is present in the composition at a concentration of at least about 1×10² viable colony forming units. In some embodiments, each isolated population of bacteria in the therapeutic composition is present in the composition at a concentration of about 1×10² to 1×10⁹ viable colony forming units.

In some embodiments, a fraction of the isolated population of bacteria in the therapeutic composition comprises a spore-forming bacteria. In some embodiments, a fraction of the isolated population of bacteria in the therapeutic composition is in spore form.

In some embodiments, the therapeutic compositions further comprise a pharmaceutically acceptable excipient. In some embodiments, the therapeutic compositions are formulated for delivery to the intestine. In some embodiments, the therapeutic compositions are enterically coated. In some embodiments, the therapeutic compositions are formulated for oral administration. In some embodiments, the therapeutic compositions are formulated into a food or beverage.

In some embodiments the therapeutic compositions can reduce the rate of tumor growth in an animal model.

In one aspect, methods of treating a cancer in a mammalian subject are provided comprising administering to the subject a therapeutic composition comprising an effective amount of an isolated population of bacteria belonging to one or more of the genera Ruminococcus, Gemmiger, Faecalibacterium, Subdoligranulum or combinations thereof. In some embodiments of the methods, the therapeutic composition may comprise bacteria belonging to at least two, three or four the genera listed.

In another aspect, methods of treating a cancer in a mammalian subject are provided comprising administering to the subject a therapeutic composition comprising an effective amount of an isolated population of bacteria that are phylogenetic descendants of the MRCA of Faecalibacterium prausnitzii and Flavonifractor plautii. In another aspect, methods of treating a cancer in a mammalian subject are provided comprising administering to the subject a therapeutic composition comprising an effective amount of an isolated population of bacteria that have 16S rDNA sequence identity of at least 94.5% to 16S rDNA sequences of species belonging to the family Ruminococcaceae. In some embodiments, the bacteria have 16S rDNA sequence identity of at least 98.7% to 16S rDNA sequences of species belonging to the family Ruminococcaceae. In some embodiments, the therapeutic compositions may comprise one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In some embodiments, the therapeutic composition may comprise at least two, three, four, five or more of the species listed.

In some embodiments, the composition is formulated for multiple administrations. In some embodiments, the composition is formulated for at least 1, 2, 3, 4, 5, 6, 7, or 8 administrations.

In some embodiments, the purified population of bacteria comprises bacteria from at least two genera or species, and wherein the ratio of the two bacteria is 1:1. In some embodiments, the purified population of bacteria comprises bacteria from at least, at most, or exactly 1, 2, 3, 4, 5, 6, 7, 8, 9, 10, 11, 12, 14, 16, 20, 30, 40, or 50 (or any derivable range therein) different families, genera, or species of bacteria. In some embodiments, the ratio of one family, genera, or species of bacteria to another family, genera, or species of bacteria present in the composition is at least, at most, or exactly 1:1, 1:2, 1:3, 1:4, 1:5, 1:6, 1:7, 1:8, 1:9, 1:10, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:250, 1:300, 1:350, 1:400, 1:450, 1:500, 1:600, 1:700, 1:800, 1:900, 1:1000, 1:1500, 1:2000, 1:2500, 1:3000, 1:3500, 1:4000, 1:4500, 1:5000, 1:1550, 1:6000, 1:6500, 1:7000, 1:7500, 1:8000, 1:8500, 1:9000, 1:9500, 1:10000, 1:1200, 1:14000, 1:16000, 1:18000, 1:20000, 1:30000, 1:40000, 1:50000, 1:60000, 1:70000, 1:80000, 1:90000, or 1:100000 (or any derivable range therein).

The compositions of the disclosure may exclude one or more bacteria genera or species described herein or may include less than 1×10⁶, 1×10⁵, 1×10⁴, 1×10³, or 1×10² cells or viable CFU (or any derivable range therein) of one or more of the bacteria described herein.

In another aspect, methods of treating a cancer in a mammalian subject are provided comprising administering to the subject a therapeutic composition comprising an effective amount of an isolated population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In another aspect, methods of treating a cancer in a mammalian subject are provided comprising administering to the subject a therapeutic composition comprising an effective amount of an isolated population of bacteria belonging to one or more of the genera Alistipes, Bacteroides, Blautia, Clostridium, Eubacterium, Parabacteroides or combinations thereof. In another aspect, methods of treating a cancer in a mammalian subject are provided comprising administering to the subject a therapeutic composition comprising an effective amount of an isolated population of bacteria belonging to one or more of the genera Bamesiella, Bifidobacterium, Blautia, Erysipelotrichaceae, Odoribacter, Parabacteroides or combinations thereof. In some embodiments of the methods, the therapeutic composition may comprise bacteria belonging to at least two, three, four, five or more of the genera listed.

In another aspect, methods of treating a cancer in a mammalian subject are provided comprising administering to the subject a therapeutic composition comprising an effective amount of an isolated population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, methods of treating a cancer in a mammalian subject are provided comprising administering to the subject a therapeutic composition comprising an effective amount of an isolated population of bacteria species selected from Alistipes senegalensis, Bacteroides dorei, Blautia_SC109, Clostridium_SC64, Eubacterium_biforme, Parabacteroides distasonis or combinations thereof. In another aspect, methods of treating a cancer in a mammalian subject are provided comprising administering to the subject a therapeutic composition comprising an effective amount of an isolated population of bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In some embodiments of the methods, the therapeutic composition may comprise at least two, three, four, five or more of the species listed.

In one aspect, methods of treating a cancer in a mammalian subject are provided comprising administering to the subject a therapeutic composition comprising an effective amount of a purified population of bacteria belonging to one or more of the genera Ruminococcus, Gemmiger, Faecalibacterium, Subdoligranulum or combinations thereof. In some embodiments of the methods, the therapeutic composition may comprise bacteria belonging to at least two, three or four the genera listed.

In another aspect, methods of treating a cancer in a mammalian subject are provided comprising administering to the subject a therapeutic composition comprising an effective amount of a purified population of bacteria belonging to one or more of the genera Alistipes, Bacteroides, Bamesiella, Bifidobacterium, Blautia, Clostridium, Eubacterium, Erysipelotrichaceae, Odoribacter, Parabacteroides or combinations thereof. In another aspect, methods of treating a cancer in a mammalian subject are provided comprising administering to the subject a therapeutic composition comprising an effective amount of a purified population of bacteria belonging to one or more of the genera Alistipes, Bacteroides, Blautia, Clostridium, Eubacterium, Parabacteroides or combinations thereof. In another aspect, methods of treating a cancer in a mammalian subject are provided comprising administering to the subject a therapeutic composition comprising an effective amount of a purified population of bacteria belonging to one or more of the genera Bamesiella, Bifidobacterium, Blautia, Erysipelotrichaceae, Odoribacter, Parabacteroides or combinations thereof. In some embodiments of the methods, the therapeutic composition may comprise bacteria belonging to at least two, three, four, five or more of the genera listed.

In another aspect, methods of treating a cancer in a mammalian subject are provided comprising administering to the subject a therapeutic composition comprising an effective amount of a purified population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, methods of treating a cancer in a mammalian subject are provided comprising administering to the subject a therapeutic composition comprising an effective amount of a purified population of bacteria species selected from Alistipes senegalensis, Bacteroides dorei, Blautia_SC109, Clostridium_SC64, Eubacterium_biforme, Parabacteroides distasonis or combinations thereof. In another aspect, methods of treating a cancer in a mammalian subject are provided comprising administering to the subject a therapeutic composition comprising an effective amount of a purified population of bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In some embodiments of the methods, the therapeutic composition may comprise at least two, three, four, five or more of the species listed.

In some embodiments, the therapeutic compositions used in the methods of treating cancer further comprise an anticancer agent. In some embodiments, the anticancer agent is a checkpoint inhibitor. In some embodiments, the checkpoint inhibitor is selected from an anti-PD-1 antibody, an anti-CTLA-4 antibody, an anti-PD-L1 antibody or combinations thereof. In some embodiments, the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors, STI-A1010 or combinations thereof. In some embodiments, the anticancer agent is cyclophosphamide.

In some embodiments of the methods, each isolated population of bacteria in the therapeutic composition is present in the composition at a concentration of at least about 1×10² viable colony forming units. In some embodiments of the methods, each isolated population of bacteria in the therapeutic composition is present in the composition at a concentration of about 1×10² to 1×10⁹ viable colony forming units.

In some embodiments of the methods, a fraction of the isolated population of bacteria in the therapeutic composition comprises a spore-forming bacteria. In some embodiments of the methods, a fraction of the isolated population of bacteria in the therapeutic composition is in spore form.

In some embodiments of the methods, the therapeutic compositions further comprise a pharmaceutically acceptable excipient. In some embodiments of the methods, the therapeutic compositions are formulated for delivery to the intestine. In some embodiments of the methods, the therapeutic compositions are enterically coated. In some embodiments, the therapeutic compositions are formulated for oral administration. In some embodiments of the methods, the therapeutic compositions are formulated into a food or beverage.

In some embodiments of the methods, the mammalian subject is a human.

In some embodiments of the methods, the cancer is selected from metastatic melanoma, melanoma of the skin, non-small cell lung cancer, kidney cancer, bladder cancer, head and neck cancers, Merkel cell skin cancer (Merkel cell carcinoma), or Hodgkin lymphoma.

In some embodiments of the methods, prior to administration of the isolated population of bacteria, the subject is subjected to antibiotic treatment and/or a bowel cleanse.

In one aspect, methods of identifying if a mammalian subject is a candidate for anticancer treatment are provided, the method comprising: a) obtaining a microbiome sample from the subject, b) determining the prevalence of the genera of bacteria in the microbiome sample, and c) determining that the subject is a candidate for anticancer treatment if the microbiome sample comprises bacteria belonging to one or more of the genera Ruminococcus, Gemmiger, Faecalibacterium, Subdoligranulum or combinations thereof.

In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria belonging to one or more species that are phylogenetic descendants of the MRCA of Faecalibacterium prausnitzii and Flavonifractor plautii. In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria belonging to one or more species that have 16S rDNA sequence identity of at least 94.5% to 16S rDNA sequences of species belonging to the family Ruminococcaceae. In some embodiments, the one or more species may have 16S rDNA sequence identity of at least 98.7% to 16S rDNA sequences of species belonging to the family Ruminococcaceae.

In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria belonging to one or more species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In another aspect, methods of identifying a mammalian subject as a candidate for anticancer treatment are provided, the method comprising: a) obtaining a microbiome sample from the subject, b) determining the prevalence and/or abundance of the genera of bacteria in the microbiome sample, and c) determining that the subject is a candidate for anticancer treatment if the microbiome sample comprises bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In another aspect, methods of identifying a mammalian subject as a candidate for anticancer treatment are provided, the method comprising: a) obtaining a microbiome sample from the subject, b) determining the prevalence and/or abundance of the genera of bacteria in the microbiome sample, and c) determining that the subject is a candidate for anticancer treatment if the microbiome sample comprises bacteria belonging to one or more of the genera Alistipes, Bacteroides, Blautia, Clostridium, Eubacterium, Parabacteroides or combinations thereof. In methods in which a microbiome sample is obtained, in some cases the microbiome sample is obtained from a fecal sample. In some cases the microbiome sample is obtained by mucosal biopsy.

In another aspect, methods of identifying a mammalian subject as a candidate for anticancer treatment are provided, the method comprising: a) obtaining a microbiome sample from the subject, b) determining the prevalence and/or abundance of the genera of bacteria in the microbiome sample, and c) determining that the subject is a candidate for anticancer treatment if the microbiome sample comprises one or more of the genera Bamesiella, Bifidobacterium, Blautia, Erysipelotrichaceae, Odoribacter, Parabacteroides or combinations thereof. In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Alistipes senegalensis, Bamesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Alistipes senegalensis, Bacteroides dorei, Blautia_SC109, Clostridium_SC64, Eubacterium_biforme, Parabacteroides distasonis or combinations thereof. In another aspect, methods of identifying a mammalian subject as a candidate for anticancer treatment are provided, the method comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In methods in which a microbiome sample is obtained, in some cases the microbiome sample is obtained from a fecal sample. In some cases, the microbiome sample is obtained by mucosal biopsy.

In another aspect, provided herein are methods of treating cancer comprising administering an anticancer treatment to a subject determined to have a microbiome sample comprising bacteria that are phylogenetic descendants of the most recent common ancestor (MRCA) of Faecalibacterium prausnitzii and Flavonifractor plautii. In another aspect, provided herein are methods of treating cancer comprising administering an anticancer treatment to a subject determined to have a microbiome sample comprising bacteria that have 16S rDNA sequence identity of at least 94.5% to 16S rDNA sequences of species belonging to the family Ruminococcaceae. In some embodiments, the bacteria have 16S rDNA sequence identity of at least 98.7% to 16S rDNA sequences of species belonging to the family Ruminococcaceae. In another aspect, provided herein are methods of treating cancer comprising administering an anticancer treatment to a subject determined to have a microbiome sample comprising bacteria belonging to one or more of the genera Ruminococcus, Gemmiger, Faecalibacterium, Subdoligranulum or combinations thereof. In another aspect, provided herein are methods of treating cancer comprising administering an anticancer treatment to a subject determined to have a microbiome sample comprising bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In another aspect, provided herein are methods of treating cancer comprising administering an anticancer treatment to a subject determined to have a microbiome sample comprising bacteria belonging to one or more of the genera Alistipes, Bacteroides, Blautia, Clostridium, Eubacterium, Parabacteroides or combinations thereof. In another aspect, provided herein are methods of treating cancer comprising administering an anticancer treatment to a subject determined to have a microbiome sample comprising one or more of the genera Bamesiella, Bifidobacterium, Blautia, Erysipelotrichaceae, Odoribacter, Parabacteroides or combinations thereof. In another aspect, provided herein are methods of treating cancer comprising administering an anticancer treatment to a subject determined to have a microbiome sample comprising bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof. In another aspect, provided herein are methods of treating cancer comprising administering an anticancer treatment to a subject determined to have a microbiome sample comprising bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In another aspect, provided herein are methods of treating cancer comprising administering an anticancer treatment to a subject determined to have a microbiome sample comprising bacteria species selected from Alistipes senegalensis, Bacteroides dorei, Blautia_SC109, Clostridium_SC64, Eubacterium_biforme, Parabacteroides distasonis or combinations thereof. In another aspect, provided herein are methods of treating cancer comprising administering an anticancer treatment to a subject determined to have a microbiome sample comprising bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

In another aspect, provided herein are methods comprising evaluating a microbiome profile for bacteria that are phylogenetic descendants of the most recent common ancestor (MRCA) of Faecalibacterium prausnitzii and Flavonifractor plautii in a sample from a subject. In another aspect, provided herein are methods comprising evaluating a microbiome profile for bacteria that have 16S rDNA sequence identity of at least 94.5% to 16S rDNA sequences of species belonging to the family Ruminococcaceae in a sample from a subject. In some embodiments, the bacteria have 16S rDNA sequence identity of at least 98.7% to 16S rDNA sequences of species belonging to the family Ruminococcaceae. In another aspect, provided herein are methods comprising evaluating a microbiome profile for bacteria belonging to one or more of the genera Ruminococcus, Gemmiger, Faecalibacterium, Subdoligranulum or combinations thereof in a sample from the subject. In another aspect, provided herein are methods comprising evaluating a microbiome profile for bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof in a sample from a subject. In another aspect, provided herein are methods comprising evaluating a microbiome profile for bacteria belonging to one or more of the genera Alistipes, Bacteroides, Blautia, Clostridium, Eubacterium, Parabacteroides or combinations thereof in a sample from a subject. In another aspect, provided herein are methods comprising evaluating a microbiome profile for one or more of the genera Barnesiella, Bifidobacterium, Blautia, Erysipelotrichaceae, Odoribacter, Parabacteroides or combinations thereof in a sample from a subject. In another aspect, provided herein are methods comprising evaluating a microbiome profile for bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof in a sample from a subject. In another aspect is a method comprising evaluating a microbiome profile for bacteria species selected from Alistipes senegalensis, Bamesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof in a sample from a subject. In another aspect is a method comprising evaluating a microbiome profile for bacteria species selected from Alistipes senegalensis, Bacteroides dorei, Blautia_SC109, Clostridium_SC64, Eubacterium_biforme, Parabacteroides distasonis or combinations thereof in a sample from a subject. In another aspect, provided herein are methods comprising evaluating a microbiome profile for bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof in a sample from a subject.

In some embodiments, the method further comprises comparing the microbiome profile to a control microbiome. In some embodiments, the control microbiome comprises a microbiome sample from a subject determined to be a responder to an anticancer treatment. In some embodiments, the control microbiome comprises a microbiome sample from a subject determined to be a non-responder to an anticancer treatment.

In some embodiments of the methods of identifying a mammalian subject as a candidate for anticancer treatment, the subject is determined to be a candidate for checkpoint inhibitor anticancer treatment. In some embodiments of the methods of identifying a mammalian subject as a candidate for anticancer treatment, the subject is determined to be a candidate for cyclophosphamide anticancer treatment.

In some embodiments of the methods of identifying a mammalian subject as a candidate for anticancer treatment, the mammalian subject is a human.

In some embodiments of the methods of identifying a mammalian subject as a candidate for anticancer treatment, the cancer is selected from metastatic melanoma, melanoma of the skin, non-small cell lung cancer, kidney cancer, bladder cancer, head and neck cancers, Merkel cell skin cancer (Merkel cell carcinoma), or Hodgkin lymphoma.

In some embodiments, the subject has previously been treated for the cancer. In some embodiments, the subject has been determined to be a non-responder to the previous treatment. In some embodiments, the subject has been determined to have a have a toxic response to the previous treatment. In some embodiments, the previous treatment comprises immune checkpoint blockade monotherapy or combination therapy. In some embodiments, the cancer is recurrent cancer. In some embodiments, the subject has not received a prior anticancer therapy.

In one aspect, therapeutic compositions are provided comprising an effective amount of an isolated population of bacteria belonging to one or more of the genera Ruminococcus, Gemmiger, Faecalibacterium and Subdoligranulum.

In another aspect, therapeutic compositions are provided comprising an effective amount of an isolated population of bacteria belonging to one or more species that are phylogenetic descendants of the MRCA of Faecalibacterium prausnitzii and Flavonifractor plautii. In another aspect, therapeutic compositions are provided comprising an effective amount of an isolated population of bacteria belonging to one or more species that have 16S rDNA sequence identity of at least 94.5% to 16S rDNA sequences of species belonging to the family Ruminococcaceae. In some embodiments, the one or more species may have 16S rDNA sequence identity of at least 98.7% to 16S rDNA sequences of species belonging to the family Ruminococcaceae. In another aspect, therapeutic compositions are provided comprising an effective amount of an isolated population of bacteria belonging to one or more species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In another aspect, therapeutic compositions are provided comprising an effective amount of an isolated population of bacteria belonging to one or more of the genera Alistipes, Bacteroides, Bamesiella, Bifidobacterium, Blautia, Clostridium, Eubacterium, Erysipelotrichaceae, Odoribacter and Parabacteroides. In another aspect, therapeutic compositions are provided comprising an effective amount of an isolated population of bacteria belonging one or more of to the genera Bamesiella, Bifidobacterium, Blautia, Erysipelotrichaceae, Odoribacter and Parabacteroides.

In another aspect, therapeutic compositions are provided comprising an effective amount of an isolated population of bacteria species Alistipes senegalensis, Bacteroides dorei, Blautia_SC109, Clostridium_SC64, Eubacterium_biforme and Parabacteroides distasonis. In another aspect, therapeutic compositions are provided comprising an effective amount of an isolated population of bacteria species Bamesiella intestinihominis, Bifidobacterium bifidum, Bifidobacterium longum, Blautia_SC102, Blautia_SC109, Clostridium innocuum, Odoribacter splanchnicus and Parabacteroides distasonis.

In one aspect, therapeutic compositions are provided comprising an effective amount of a purified population of bacteria belonging to one or more of the species listed in Tables 1A, 1B, 2A, 2B, 3A, 3B, 4A, 4B, 5A, 5B, 6A, 6B, 7A, 7B, 8A, 8B, 10 or 11. In another aspect, therapeutic compositions are provided comprising an effective amount of a purified population of bacteria belonging to two or more of the species listed in Tables 1A, 1B, 2A, 2B, 3A, 3B, 4A, 4B, 5A, 5B, 6A, 6B, 7A, 7B, 8A, 8B, 10 or 11. In another aspect, therapeutic compositions are provided comprising an effective amount of a purified population of bacteria belonging to three or more of the species listed in Tables 1A, 1B, 2A, 2B, 3A, 3B, 4A, 4B, 5A, 5B, 6A, 6B, 7A, 7B, 8A, 8B, 10 or 11. In another aspect, therapeutic compositions are provided comprising an effective amount of a purified population of bacteria belonging to four or more of the species listed in Tables 1A, 1B, 2A, 2B, 3A, 3B, 4A, 4B, 5A, 5B, 6A, 6B, 7A, 7B, 8A, 8B, 10 or 11.

In one aspect, therapeutic compositions are provided comprising an effective amount of a purified population of bacteria belonging to one or more of the species listed in Table 1A. In another aspect, therapeutic compositions are provided comprising an effective amount of a purified population of bacteria belonging to one or more of the species listed in Table 1B. In another aspect, therapeutic compositions are provided comprising an effective amount of a purified population of bacteria belonging to one or more of the species listed in Table 10. In another aspect, therapeutic compositions are provided comprising an effective amount of a purified population of bacteria belonging to one or more of the species listed in Table 11.

In another aspect, therapeutic compositions are provided comprising an effective amount of a purified population of bacteria belonging to two or more of the species listed in Table 1A. In another aspect, therapeutic compositions are provided comprising an effective amount of a purified population of bacteria belonging to two or more of the species listed in Table 1B. In another aspect, therapeutic compositions are provided comprising an effective amount of a purified population of bacteria belonging to two or more of the species listed in Table 10. In another aspect, therapeutic compositions are provided comprising an effective amount of a purified population of bacteria belonging to two or more of the species listed in Table 11.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. 16S Alpha Diversity. The figure is a plot showing Observed, Shannon, and Inverse Simpson 16S alpha diversity scores of the microbiome in responder and non-responder patients. Error bars represent the distribution of scores. Responders (left bar within each panel); non-responders (1 bar within each panel). Where outliers are present, they are shown as individual points—otherwise, boxes extend from the first to third quartiles of the data, with whiskers extending the length of the data. Outliers are defined as points which lie outside of the first quartile minus 1.5*IQR (“interquartile range”, e.g. the distance between the first to third quartiles), or the third quartile plus 1.5*IQR.

FIG. 2. Prevalence Analysis. The figure is a volcano plot of differential 16S rDNA prevalence results. Significantly differentially prevalent OTUs/genera are marked with a rectangular label (p-value <=0.10, Fisher's exact test).

FIG. 3 is a plot showing Bray-Curtis Beta Diversity. Approximately 200 samples from healthy donors collected by the Human Microbiome Project (HMP) were used to generate a set of background samples to compare to the collected WMS data. Bray-Curtis dissimilarity across the WMS and HMP data was represented in a multidimensional scaling (MDS) format, and Linear Discriminant Analysis (LDA) was used to generate a classification line to separate responder and non-responder samples.

FIG. 4 is a plot showing the Species Data overlaid on Bray-Curtis Beta Diversity. Individual species data from the samples were mapped onto the MDS plot of FIG. 3. Circled species are all members of the family Ruminococcaceae and these data demonstrate that Ruminococcaceae are associated with responders.

FIG. 5 is a graph showing how the relative abundance of Bacteroidia are associated with response to checkpoint therapy. Samples are ordered by decreasing relative abundance. Data from responder samples are shown in gray while non-responders are shown in black. The cut-off (dashed line) maximizes sensitivity while maintaining 100% specificity.

FIG. 6 is a phylogenetic tree of Ruminococcaceae derived from 16S rDNA sequences demonstrating that a clade-based definition of Ruminococcaceae more accurately represents phylogenetic relationships. Taxa classified as Ruminococcaceae in NCBI are in black; taxa in other families are underlined. NCBI-based classification is clearly not consistent with phylogeny. Here, a definition of Ruminococcaceae based on an internal clade system (clades 14, 61, 101, 125, and 131) is consistent with phylogeny. Clade 13 was excluded as it is highly divergent from the remaining Ruminococcaceae.

FIG. 7 is a graph showing that clade-based relative abundance of Ruminococcaceae is associated with response to checkpoint therapy. Samples are ordered by decreasing relative abundance. Responders are shown in gray while non-responders are shown in black. The threshold was increased from 9.5% with the NCBI-based definition of Ruminococcaceae to 12% with the clade-based definition, as a greater number of Ruminococcaceae species were detected by the latter, resulting in higher per sample abundances. The threshold was chosen to maximize sensitivity while maintaining 100% specificity.

FIG. 8 is a plot showing the distribution of Ruminococcaceae clade-based abundance with Bacteroidia clade-based abundance. Eighty percent of responders fall outside of lower left quadrant.

FIG. 9 is a plot of a receiver operating characteristic (ROC) curve for Ruminococcaceae clade-based relative abundance in combined dataset (n=112) as a predictor of response to checkpoint therapy.

FIG. 10 is a plot of a distribution of Ruminococcaceae clade-based abundance in the combined dataset (n=112). Seventy-two percent of total non-responders lie to the left of the dotted line (<12% Ruminococcaceae), while 68% of total responders lie to right of the line (>=12% Ruminococcaceae). Bacteroidia relative abundance is plotted to allow visual separation of samples.

FIG. 11 is a plot of a ROC curve for Ruminococcaceae clade-based relative abundance in combined dataset excluding stable disease patients (n=85) as a predictor of response to checkpoint therapy.

### DETAILED DESCRIPTION

### I. Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in to context of the term “consisting of” or “consisting essentially of.” “Microbiome” refers to the communities of microbes that live in or on an individual's body, both sustainably and transiently, including eukaryotes, archaea, bacteria, and viruses (including bacterial viruses (i.e., phage)).

“Dysbiosis” refers to a state of the microbiota or microbiome of the GI tract or other body area, including mucosal or skin surfaces in which the normal diversity and/or function of the ecological network is disrupted. Any disruption from the preferred (e.g., ideal) state of the microbiota can be considered a dysbiosis, even if such dysbiosis does not result in a detectable decrease in health. This state of dysbiosis may be unhealthy, it may be unhealthy under only certain conditions, or it may prevent a subject from becoming healthier. Dysbiosis may be due to a decrease in diversity, the overgrowth of one or more pathogens or pathobionts, symbiotic organisms able to cause disease only when certain genetic and/or environmental conditions are present in a patient, or the shift to an ecological network that no longer provides a beneficial function to the host and therefore no longer promotes health.

A “spore” or a population of “spores” includes bacteria (or other single-celled organisms) that are generally viable, more resistant to environmental influences such as heat and bacteriocidal agents than vegetative forms of the same bacteria, and typically are capable of germination and out-growth. “Spore-formers” or bacteria “capable of forming spores” are those bacteria containing the genes and other necessary features to produce spores under suitable environmental conditions.

The terms “pathogen”, “pathobiont” and “pathogenic” in reference to a bacterium or any other organism or entity includes any such organism or entity that is capable of causing or affecting a disease, disorder or condition of a host organism containing the organism or entity.

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

“Inhibition” of a pathogen encompasses the inhibition of any desired function or activity of the bacterial compositions of the present invention. Demonstrations of pathogen inhibition, such as decrease in the growth of a pathogenic bacterium or reduction in the level of colonization of a pathogenic bacterium are provided herein and otherwise recognized by one of ordinary skill in the art. Inhibition of a pathogenic bacterium's “growth” may include inhibiting the increase in size of the pathogenic bacterium and/or inhibiting the proliferation (or multiplication) of the pathogenic bacterium. Inhibition of colonization of a pathogenic bacterium may be demonstrated by measuring the amount or burden of a pathogen before and after a treatment. An “inhibition” or the act of “inhibiting” includes the total cessation and partial reduction of one or more activities of a pathogen, such as growth, proliferation, colonization, and function.

The “colonization” of a host organism includes the transitory (e.g., for 1 day, 2 days, 3 days, 4 days, 5 days, 6 days, or 1 week) or non-transitory (e.g., greater than one week, at least two weeks, at least three weeks, at least 4 weeks, at least 6 weeks, at least 8 weeks, at least 3 month, at least 4 months, at least 6 months) residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the lumen of the gastrointestinal tract or adhered to the mucosal surface of the gastrointestinal tract. Measuring reductions of adherent pathogens may be demonstrated, e.g., by a biopsy sample, or luminal reductions may be measured indirectly, e.g., indirectly by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

A “cytotoxic” activity or bacterium includes the ability to kill another bacterial cell, such as a pathogenic bacterial cell or a closely related species of strain. A “cytostatic” activity or bacterium includes the ability to inhibit, partially or fully, growth, metabolism, and/or proliferation of a bacterial cell, such as a pathogenic bacterial cell.

To be free of “non-comestible products” means that a bacterial composition or other material provided herein does not have a substantial amount of a non-comestible product, e.g., a product or material that is inedible, harmful or otherwise undesired in a product suitable for administration, e.g., oral administration, to a human subject.

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as micro RNA and ribosomal RNA, the epigenome, plasmids, and all other types of genetic information.

“Augmentation” of a type of bacterium, e.g., a species, is an effect of treatment with a composition of the invention that is characterized by post-treatment detection of an increased abundance of a species not present in the composition by a nonparametric test of abundance.

“Engraftment” of a type of bacterium, e.g., a species, is an effect of treatment with a composition of the invention that is characterized by post-treatment detection of a species from the administered composition, which is not detected in the treated subject pretreatment. Methods of detection are known in the art. In one example, the method is PCR detection of a 16S rDNA sequence using standard parameters for PCR.

“Residual habitat products” refers to material derived from the habitat for microbiota within or on a human or animal. For example, microbiota live in feces in the gastrointestinal tract, on the skin itself, in saliva, mucus of the respiratory tract, or secretions of the genitourinary tract (i.e., biological matter associated with the microbial community). Substantially free of residual habitat products means that the bacterial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms and/or fragments of microorganisms. Substantially free of residual habitat products may also mean that the bacterial composition contains no detectable cells from a human or animal and that only microbial cells are detectable. In one embodiment, substantially free of residual habitat products may also mean that the bacterial composition contains no detectable viral (including bacterial viruses (i.e., phage) or human viruses), fungal, or mycoplasmal contaminants. In another embodiment, it means that fewer than 1×10⁻²%, 1×10⁻³%, 1×10⁻⁴%, 1×10⁻⁶%, 1×10⁻⁶%, 1×10⁻⁷%, 1×10⁻⁸% of the viable cells in the bacterial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g. PCR, DNA sequencing), serology and antigen analysis, enzymatic and metabolic analysis, and methods using instrumentation such as flow cytometry with reagents that distinguish desired constituents from contaminants.

“Phylogenetic tree” refers to a graphical representation of the evolutionary relationships of one genetic sequence to another that is generated using a defined set of phylogenetic reconstruction algorithms (e.g. parsimony, maximum likelihood, or Bayesian). Nodes in the tree represent distinct ancestral sequences and the confidence of any node is provided by a bootstrap or Bayesian posterior probability, which measures branch uncertainty.

“Operational taxonomic unit (OTU, plural OTUs)”, in some embodiments, refers to a terminal leaf in a phylogenetic tree and is defined by a specific genetic sequence and all sequences that share sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rDNA sequence or a portion of the 16S rDNA sequence or it may be a functionally conserved housekeeping gene found broadly across the eubacterial kingdom. OTUs share at least 95%, 96%, 97%, 98%, or 99% sequence identity. OTUs generally defined by comparing sequences between organisms. Sequences with less than 95% sequence identity are not considered to form part of the same OTU. In some embodiments, metagenomics methods, known in the art, are used to identify species and/or OTUs.

“Clade” refers to the set of OTUs or members of a phylogenetic tree downstream of a statistically valid node in a phylogenetic tree. A clade is a group of related organisms representing all of the phylogenetic descendants of a common ancestor. The clade comprises a set of terminal leaves in the phylogenetic tree that is a distinct monophyletic evolutionary unit.

The terms “subject” or “patient” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

The term “pathobiont” refer to specific bacterial species found in healthy hosts that may trigger immune-mediated pathology and/or disease in response to certain genetic or environmental factors. Chow et al., (2011) Curr Op Immunol. Pathobionts of the intestinal microbiota and inflammatory disease. 23: 473-80. Thus, a pathobiont is a pathogen that is mechanistically distinct from an acquired infectious organism. Thus, the term “pathogen” includes both acquired infectious organisms and pathobionts.

As used herein, the term “immunoregulator” refers to an agent or a signaling pathway (or a component thereof) that regulates an immune response. “Regulating,” “modifying” or “modulating” an immune response refers to any alteration of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function or utility as a therapeutic target in a cancer microenvironment.

As used herein, the term “immune evasion” refers to inhibition of a subject's immune system or a component thereof (e.g., endogenous T cell response) by a cancer or tumor cell in order to maximize or allow continued growth or spread of the cancer/tumor.

As used herein, the term “immunotherapy” refers to the treatment or prevention of a disease or condition (e.g., cancer) by a method comprising inducing, enhancing, suppressing or otherwise modifying an immune response.

As used herein, “potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “antibody” refers to a whole antibody molecule or a fragment thereof (e.g., fragments such as Fab, Fab′, and F(ab′)2), it may be a polyclonal or monoclonal antibody, a chimeric antibody, a humanized antibody, a human antibody, etc.

As used herein, “cancer” means all types of cancers. In particular, the cancers can be solid or non-solid cancers. Non-limiting examples of cancers are carcinomas or adenocarcinomas such as breast, prostate, ovary, lung, pancreas or colon cancer, sarcomas, lymphomas, melanomas, leukemias, germ cell cancers and blastomas.

### II. Methods of the Disclosure

Provided herein are compositions and methods for treatment and/or prevention of a cancer by microbiome manipulation. In particular, the amount, identity, presence, and/or ratio of bacteria in the microbiome (e.g., GI microbiome) in a subject is manipulated to facilitate treatment of a cancer. Furthermore, applicants have discovered that the abundance and/or prevalence of certain commensal bacteria in feces, e.g., commensal Ruminococcaceae, can be used to identify fecal donors and/or donations that can improve patient response to a checkpoint inhibitor. Fecal material from such individuals can be used, e.g., in fecal microbiome transplantation or in a processed form derived from such material, for example a preparation enriched in Firmicutes (e.g., Clostridia, Clostridiales, or spore formers), that are in vegetative and/or spore form.

Applicants have identified bacterial species that are useful for increasing the efficacy of cancer treatment, e.g., treatments using checkpoint inhibitors. In some embodiments, the effectiveness of an endogenous immune response, immunotherapy, chemotherapeutic, or other treatment (e.g., surgery, radiation, etc.) in the treatment or prevention of reoccurrence of cancer and/or tumor is dependent upon conditions within the subject (e.g., the tumor microenvironment). In particular, the identity or characteristics (e.g., concentration or level) of the microbiome within a subject can affect the effectiveness of cancer treatments (e.g., generally or specific treatments) and/or the effectiveness of the subject's own response to cancer, e.g., immune response.

In some embodiments, the presence or increased level of one or more species of bacteria in a subject facilitates treatment (e.g., immunotherapy, chemotherapy, etc.) and/or the subject's endogenous immune response to cancer and/or tumor cells. In some embodiments, the absence and/or decreased level of one or more species of bacteria in a subject discourages cancer/tumor growth, spread, and/or evasion of treatment/immune response. In some embodiments, the absence or decreased level of one or more species of bacteria in a subject facilitates treatment (e.g., immunotherapy, chemotherapy, etc.) and/or the subject's endogenous immune response to cancer and/or tumor cells.

In some embodiments, the presence of certain microbes (e.g., microbes that facilitate cancer treatment) in a subject creates an environment or microenvironment (e.g., microbiome) that is conducive to the treatment of cancer and/or inhibits cancer/tumor growth. In some embodiments, the presence of detrimental microbes (e.g., microbes that facilitate cancer/tumor growth and/or prevent treatment) in a subject creates an environment or microenvironment (e.g., microbiome) that is conducive to the treatment of cancer and/or inhibits cancer/tumor growth. Microbes or their products may act locally at the level of the gut epithelium and the lamina propria to alter immunological tone or immune cell trafficking, or they may act distally by the translocation of microbes or their products into circulation to alter peripheral immune responses, e.g. in blood, liver, spleen, lymph nodes or tumor.

Modulation of microflora levels and/or identity may comprise encouraging or facilitating growth of one or more species of beneficial microbes (e.g., microbes that facilitate cancer treatment), discouraging or inhibiting growth of one or more types of detrimental microbes (e.g., species of bacteria that facilitate cancer/tumor growth and/or prevent treatment), administering one or more types of beneficial microbes (e.g., species of bacteria that facilitate cancer treatment) to the subject, and/or combinations thereof. Embodiments within the scope herein are not limited by the mechanisms for introducing one or more microbes (e.g., probiotic administration, fecal transplant, etc.), encouraging growth of beneficial microbes (e.g., administering agents that skew the environment within the subject toward growth conditions for the beneficial microbes), discouraging or inhibiting growth of detrimental microbes (e.g., administering agents that skew the environment within the subject away from growth conditions for the detrimental microbes, administration of antimicrobial(s), etc.), and combinations thereof.

In some embodiments, methods are provided for the treatment or prevention of cancer by the manipulation of the presence, amount, or relative ratio of one or more families, genera, or species of bacteria (e.g., in the gastrointestinal microbiome). In some embodiments, the presence, amount, or relative ratio of particular bacteria, fungi, and/or archaea within a subject is altered. For example, in some embodiments, the presence, amount, or relative ratio of one or more bacteria from the genera Ruminococcus, Gemmiger, Faecalibacterium, Subdoligranulum is manipulated. For example, in some embodiments, the presence, amount, or relative ratio of one or more bacteria from the genera Alistipes, Bacteroides, Barnesiella, Bifidobacterium, Blautia, Clostridium, Eubacterium, Erysipelotrichaceae, Odoribacter, or Parabacteroides is manipulated. In some embodiments, the presence, amount, or relative ratio of one or more bacteria from the genera Barnesiella, Bifidobacterium, Blautia, Erysipelotrichaceae, Odoribacter, or Parabacteroides are manipulated. In some embodiments the presence, amount, or relative ratio of one or more bacteria from the genera Bifidobacterium, Blautia, Parabacteroides, or Subdoligranulum are manipulated. In some embodiments the presence, amount, or relative ratio of one or more bacteria from the genera Blautia, Clostridium, Coprococcus, Faecalibacterium, Fusicatenbacter, Gemmiger, Lachnospiraceae or Subdoligranulum are manipulated.

In some embodiments, the presence, amount or relative ratio of one or more bacterial species that are phylogenetic descendants of the most recent common ancestor (MRCA) of Faecalibacterium prausnitzii and Flavonifractor plautii are manipulated or adjusted. In some embodiments, the presence, amount or relative ratio of one or more bacterial species that have 16S rDNA sequence identity of at least 94.5% to 16S rDNA sequences of species belonging to the family Ruminococcaceae are manipulated or adjusted. In some embodiments, the one or more species may have 16S rDNA sequence identity of at least 98.7% to 16S rDNA sequences of species belonging to the family Ruminococcaceae. In some embodiments, the presence, amount or relative ratio of one or more bacterial species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof, are manipulated or adjusted.

In some embodiments, the methods exclude the administration of, the evaluation of, the detection of, or the determination of the amount or relative ratio of one or more bacterial species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In some embodiments, the presence, amount, or relative ratio of one or more bacterial species Alistipes senegalensis, Bacteroides dorei, Blautia_SC109, Clostridium_SC64, Eubacterium_biforme or Parabacteroides distasonis are manipulated. In some embodiments, the presence, amount, or relative ratio of one or more bacterial species Barnesiella intestinihominis, Bifidobacterium bifidum, Bifidobacterium longum, Blautia_SC102, Blautia_SC109, Clostridium innocuum, Odoribacter splanchnicus or Parabacteroides distasonis are manipulated. In some embodiments, the presence, amount, or relative ratio of one or more bacterial species Bifidobacterium bifidum, Blautia_SC109, Parabacteroides distasonis Gemmiger formicilis or Subdoligranulum variabile are manipulated. In some embodiments, the presence, amount, or relative ratio of one or more bacterial species Blautia_SC109, Gemmiger formicilis or Subdoligranulum variabile, Coprococcus catus, Faecalibacterium prausnitzii, Fusicatenbacter saccharivorans, Gemmiger formicilis, Subdoligranulum variabile, Anaerostipes hadrus, Gemmiger formicilis or Subdoligranulum variabile are manipulated.

### III. Therapeutic Compositions

In some embodiments, the therapeutic compositions comprise an effective amount of an isolated and/or purified population of bacteria belonging to one or more of the genera Ruminococcus, Gemmiger, Faecalibacterium, Subdoligranulum or combinations thereof. In some embodiments, the therapeutic composition may comprise bacteria belonging to at least one, two, three or four of the genera listed.

In some embodiments, the therapeutic compositions comprise an effective amount of an isolated and/or purified population of one or more bacterial species that are phylogenetic descendants of the MRCA of Faecalibacterium prausnitzii and Flavonifractor plautii. In some embodiments, the therapeutic composition may comprise at least one, two, three, four, five, six, seven, eight, nine, ten or more than ten species that are phylogenetic descendants of the MRCA of Faecalibacterium prausnitzii and Flavonifractor plautii.

In some embodiments, the therapeutic compositions comprise an effective amount of an isolated and/or purified population of one or more bacterial species that have 16S rDNA sequence identity of at least 94.5% to 16S rDNA sequences of species belonging to the family Ruminococcaceae. In some embodiments, the therapeutic composition may comprise at least one, two, three, four, five, six, seven, eight, nine, ten or more than ten species that have 16S rDNA sequence identity of at least 94.5% to 16S rDNA sequences of species belonging to the family Ruminococcaceae. In some embodiments, the one or more species may have 16S rDNA sequence identity of at least 98.7% to 16S rDNA sequences of species belonging to the family Ruminococcaceae.

In some embodiments, the therapeutic compositions comprise an effective amount of an isolated and/or purified population of one or more bacterial species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof. In some embodiments, the therapeutic composition may comprise at least one, two, three, four, five, six, seven, eight, nine, ten or more than ten species of the species listed.

In some embodiments, the therapeutic compositions may exclude an isolated and/or purified population comprising one or more bacterial species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

In some embodiments, the therapeutic compositions comprise an effective amount of an isolated and/or purified population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In some embodiments, the therapeutic composition may comprise bacteria belonging to at least one, two, three, four, five, six, seven, eight, nine or ten of the genera listed.

In some embodiments, the therapeutic compositions comprise an effective amount of an isolated and/or purified population of bacteria belonging to one or more of the genera Alistipes, Bacteroides, Blautia, Clostridium, Eubacterium, Parabacteroides or combinations thereof. In some embodiments, the therapeutic composition may comprise bacteria belonging to at least one, two, three, four, five or six of the genera listed.

In some embodiments, the therapeutic compositions comprise an effective amount of an isolated and/or purified population of bacteria belonging to one or more of the genera Barnesiella, Bifidobacterium, Blautia, Erysipelotrichaceae, Odoribacter, Parabacteroides or combinations thereof. In some embodiments, the therapeutic composition may comprise bacteria belonging to at least one, two, three, four, five or six of the genera listed.

In some embodiments, the therapeutic compositions comprise an effective amount of an isolated and/or purified population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In some embodiments, the therapeutic composition may comprise at least one, two, three, four, five, six, seven, eight, nine, ten, eleven or twelve of the species listed.

In some embodiments, the therapeutic compositions comprise an effective amount of an isolated and/or purified population of bacteria species selected from Alistipes senegalensis, Bacteroides dorei, Blautia_SC109, Clostridium_SC64, Eubacterium_biforme, Parabacteroides distasonis or combinations thereof. In some embodiments, the therapeutic composition may comprise at least one, two, three, four, five or six of the species listed.

In some embodiments, the therapeutic compositions comprise an effective amount of an isolated and/or purified population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof. In some embodiments, the therapeutic composition may comprise at least two, three, four, five or more of the species listed. In some embodiments, the therapeutic composition may comprise at least one, two, three, four, five, six, seven or eight of the species listed.

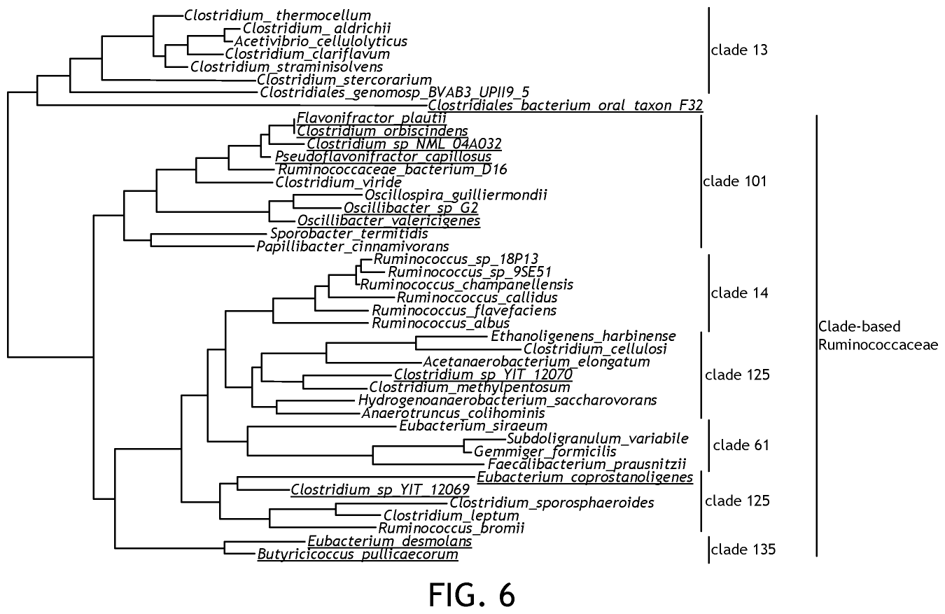

In some embodiments, the therapeutic compositions comprise an effective amount of an isolated and/or purified population of one or more of the bacteria species in one or more of clade 101, clade 14, clade 126, clade 61, clade 125 or clade 135 as shown in the phylogenetic tree in FIG. 6. In some embodiments, clade 101 comprises the bacterial species Flavonifractor plautii, Clostridium orbiscindens, Clostridium sp NML_04A032, Pseudoflavonifractor capillosus, Ruminococcaceae bacterium D16, Clostridium viride, Oscillospira guilliermondii, Oscillibacter sp_G2, Oscillibacter valericigenes, Sporobacter termitidis and Paplillibacter cinnamivorans. In some embodiments, clade 14 comprises the bacterial species Ruminococcus sp_18P13, Ruminococcus sp_9SE51, Ruminococcus champanellensis, Ruminococcus callidus, Ruminococcus flavefaciens and Ruminococcus albus. In some embodiments, clade 126 comprises the bacterial species Ethanoligenens harbinense, Clostridium cellulosi, Acetanaerobacterium elongatum, Clostridium sp_YIT_12070, Clostridium methylpentosum, Hydrogenoanaerobacterium saccharovorans, and Anaerotruncus colihominis. In some embodiments, clade 61 comprises the bacterial species Eubacterium siraeum, Subdoligranulum variabile, Gemmiger formicilis and Faecalibacterium prausnitzii. In some embodiments, clade 125 comprises the bacterial species Eubacterium coprostanoligenes, Clostridium sp_YIT_12069, Clostridium sporosphaeroides, Clostridium leptum and Ruminococcus bromii. In some embodiments, clade 135 comprises the bacterial species Eubacterium desmolans, Butyricicoccus pullicaecorum or combinations thereof.

In some embodiments, the therapeutic compositions comprise an effective amount of one, two, three, four, five, six, seven, eight, nine, ten or eleven species of clade 101. In some embodiments, the therapeutic compositions comprise an effective amount of one, two, three, four, five or six, species of clade 14. In some embodiments, the therapeutic compositions comprise an effective amount of one, two, three, four, five, six or seven species of clade 126. In some embodiments, the therapeutic compositions comprise an effective amount of one, two, three or four species of clade 61. In some embodiments, the therapeutic compositions comprise an effective amount of one, two, three, four or five species of clade 125. In some embodiments, the therapeutic compositions comprise an effective amount of one or two species of clade 135.

In some embodiments, the therapeutic compositions may comprise additional species that are determined to be part of any one of clade 101, clade 14, clade 126, clade 61, clade 125 or clade 135. A person of ordinary skill in the art would be able to use methods known in the art to determine whether a species is part of a clade, including methods described herein.

In some embodiments, the therapeutic compositions comprise an effective amount of an isolated and/or purified population of one or more of the bacteria species listed in Tables 1A and 1B. In some embodiments, the therapeutic compositions comprise an effective amount of an isolated and/or purified population of one or more of the bacteria species listed in Table 11. In other embodiments, the therapeutic compositions comprise an effective amount of an isolated and/or purified population of one or more of the bacteria species listed in any of Tables 1A, 1B, 2A, 2B, 3A, 3B, 4A, 4B, 5A, 5B, 6A, 6B, 7A, 7B, 8A, 8B, 10 and 11.

In some embodiments, a therapeutic composition can reduce the rate of tumor growth in an animal model. In some embodiments, a therapeutic composition can reduce the rate of tumor growth in a human subject. In some embodiments, a therapeutic composition can reduce the rate of tumor growth in an in vitro cell culture model. In some embodiments, a therapeutic composition can reduce the rate of tumor growth in an in situ model.

In some embodiments, the method of treating a cancer may use any of the therapeutic compositions listed herein, including combinations of genera from therapeutic compositions and/or combinations of species from therapeutic compositions. These methods of treatment, including combination treatment with other anti-cancer agents, are described in further detail below.

In some embodiments, the bacteria in the therapeutic compositions may be identified by species, operational taxonomic unit (OTU), whole genome sequence or other methods known in the art for defining different types of bacteria.

Bacterial compositions may comprise two types of bacteria (termed “binary combinations” or “binary pairs”) or greater than two types of bacteria. Bacterial compositions that comprise three types of bacteria are termed “ternary combinations”. For instance, a bacterial composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21, 22, 23, 24, 25, 26, 27, 28, 29 30, 31, 32, 33, 34, 35, 36, 37, 38, 39, or at least 40, at least 50 or greater than 50 types of bacteria, as defined by species or operational taxonomic unit (OTU), or otherwise as provided herein.

In another embodiment, the number of types of bacteria present in a bacterial composition is at or below a known value. For example, in such embodiments the bacterial composition comprises 50 or fewer types of bacteria, such as 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 or fewer, or 9 or fewer types of bacteria, 8 or fewer types of bacteria, 7 or fewer types of bacteria, 6 or fewer types of bacteria, 5 or fewer types of bacteria, 4 or fewer types of bacteria, or 3 or fewer types of bacteria. In another embodiment, a bacterial composition comprises from 2 to no more than 40, from 2 to no more than 30, from 2 to no more than 20, from 2 to no more than 15, from 2 to no more than 10, or from 2 to no more than 5 types of bacteria.

A bacterial composition useful in a method described herein may be prepared comprising at least one type of isolated bacteria, wherein a first type and a second type are independently chosen from the genera or species listed herein. In another embodiment, the first and/or second OTUs may be characterized by one or more of the variable regions of the 16S sequence (V1-V9). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (e.g., Brosius et al., Complete nucleotide sequence of a 16S ribosomal RNA gene from Escherichia coli, Proc Nat Acad Sci 75(10):4801-4805 (1978)). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU.

Methods of the disclosure include administration of a combination of therapeutic agents and compositions. The therapy may be administered in any suitable manner known in the art. For example, the therapies may be administered sequentially (at different times) or concurrently (at the same time). In some embodiments, the therapies are in a separate composition. In some embodiments, the therapies are in the same composition.

Various combinations of the therapies may be employed, for example, one therapy or composition designated “A” and another therapy or composition designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies and compositions of the disclosure may be administered by the same route of administration or by different routes of administration. In some embodiments, the therapy is administered intracolonically, intravenously, intramuscularly, subcutaneously, topically, orally, transdermally, intraperitoneally, intraorbitally, by implantation, intrathecally, intraventricularly, or intranasally. In some embodiments, the microbial modulator is administered intravenously, intramuscularly, subcutaneously, topically, orally, transdermally, intraperitoneally, intraorbitally, by implantation, intrathecally, intraventricularly, or intranasally.

In some embodiments, the compositions of the disclosure are administered in a therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×10³ viable colony forming units (CFU) of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ viable CFU (or any derivable range therein). In some embodiments, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable CFU (or any derivable range therein) of total bacteria. In specific embodiments, the bacteria are provided in spore form or as sporulated bacteria. In particular embodiments, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some embodiments, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

In some embodiments, the therapeutically effective or sufficient amount of each of the at least one isolated or purified population of bacteria or each of the at least two, 3, 4, 5, 6, 7, 8, 9, 10 11, 12, 13, 14, or 15 isolated or purified populations of bacteria of the microbial modulator compositions of the embodiments that is administered to a human will be at least about 1×103 cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein). In some embodiments, a single dose will contain an amount of bacteria (such as a specific bacteria or species, genus, or family described herein) of at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein) of a specified bacteria. In some embodiments, a single dose will contain at least, at most, or exactly 1×10⁴, 1×10⁵, 1×10⁶, 1×10⁷, 1×10⁸, 1×10⁹, 1×10¹⁰, 1χ10¹¹, 1×10¹², 1χ10¹³, 1×10¹⁴, 1×10¹⁵ cells (or any derivable range therein) of total bacteria. In specific embodiments, the bacteria are provided in spore form or as sporulated bacteria. In particular embodiments, the concentration of spores of each isolated or purified population of bacteria, for example of each species, subspecies or strain, is at least, at most, or exactly 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ (or any derivable range therein) viable bacterial spores per gram of composition or per administered dose. In some embodiments, the composition comprises or the method comprises administration of at least, at most, or exactly 1, 2, 3, 4, 5, 6, 7, 8, 9, 10, 11, 12, 13, 14, 15, 20, 25, 30, 40, or 50 (or any derivable range therein) of different bacterial species, different bacterial genus, or different bacterial family.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administerable dose.

The quantity to be administered, both according to number of treatments and unit dose, depends on the treatment effect desired. An effective dose is understood to refer to an amount necessary to achieve a particular effect. In some embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of a therapeutic composition that is administered to a human will be in the range of about 0.01 to about 50 mg/kg of patient body weight whether by one or more administrations. In some embodiments, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the therapeutic agent is administered at 15 mg/kg. However, other dosage regimens may be useful. In one embodiment, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In some embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In some embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Methods for Evaluating Bacteria

A. Determining Bacterial Genera and Species

In some embodiments, the bacterial genera or species for use in a therapeutic composition is as described in the Examples below.

In some embodiments, the bacterial genera or species for use in a therapeutic composition are those genera or species that are found to be prevalent in the microbiome of subjects that respond to an anti-cancer therapy, e.g., subjects who are responders. In some embodiments, the genera or species are more prevalent in the microbiome of a responder compared to the microbiome of a subject who does not respond to an anti-cancer therapy, e.g., a non-responder. In other embodiments, the genera or species are more prevalent in the microbiome of a responder compared to the microbiome of a healthy subject that does not have a cancer and thus has not been treated with an anti-cancer therapy.

In some embodiments, the bacterial genera or species for use in a therapeutic composition are those genera or species that are found to be more abundant in the microbiome of subjects that respond to an anti-cancer therapy, e.g., subjects who are responders. In some embodiments, the genera or species are more abundant in the microbiome of a responder compared to the microbiome of a subject who does not respond to an anti-cancer therapy, e.g., a non-responder. In other embodiments, the genera or species are more abundant in the microbiome of a responder compared to the microbiome of a healthy subject that does not have a cancer and thus has not been treated with an anti-cancer therapy.

In some embodiments, whether a subject is a responder to an anti-cancer therapy is determined as described in the art, for example, by Routy et al. (Science 2018 359(6371):91-97) or Gopalakrishnan et al. (Science 2018; 359(6371):97-103). In some embodiments, the subject is considered a responder if, following treatment with an anti-cancer therapy, the subject shows a complete response to the therapy, e.g., a complete remission of the cancer. In other embodiments, the subject is considered a responder if, following treatment with an anti-cancer therapy, the subject shows a complete response to the therapy or a partial response to the therapy, e.g., a reduction in tumor size or tumor load. In other embodiments, the subject is considered a responder if, following treatment with an anti-cancer therapy, the subject shows a complete response to the therapy, a partial response to the therapy, or a stable response to the therapy, e.g. the subject's tumor size or tumor load does not increase.

B. Methods for Determining Species that are Members of the Family Ruminococcaceae

1. Most Recent Common Ancestor (MRCA)

In some embodiments, a bacterial species is a member of the family Ruminococcaceae if the species is a phylogenetic descendant of the most recent common ancestor (MRCA) of Faecalibacterium prausnitzii and Flavonifractor plautii. In certain aspects, such a group of MRCA phylogenetic descendants is referred to as a “monophyletic” group.

In some embodiments, determining if a bacterial species is a descendant of a MRCA of Faecalibacterium prausnitzii and Flavonifractor plautii may be performed using phylogenetic grouping procedures known in the art. In one embodiment, one may use a rooted phylogenetic tree with F. prausnitzii, F. plautii and a third taxon of interest (e.g. a taxon to be classified), and apply the analysis packages Analyses of Phylogenetics and Evolution (“ape;” https://cran.r-project.org/web/packages/ape/index.html) and Phylogenetic Tools for Comparative Biology (and Other Things) (“phytools;” https://cran.r-project.org/web/packages/phytools/index.html) in order to determine whether the taxon of interest is in the family Ruminococcaceae. Both ape and phytools are packages written in the R language for use in studying molecular evolution and phylogenetics. The ape and phytools packages provide methods for phylogenetic and evolutionary analysis and their use is known to one of skill in the art.

In some embodiments, the following script may be used:

library(“ape”)
library(“phytools”)
input.tree = read.tree(file=″tree_file″)
rumino.node = getMRCA(input.tree,
c(′Faecalibacterium_prausnitzii′,′Flavonifractor_plautii′))
rumino.tree = extract.clade(input.tree, rumino.node)
print(rumino.tree$tip.label)

In some embodiments, after the script is run, if the taxon of interest is in the printed list, it is a descendant of a MRCA of Faecalibacterium prausnitzii and Flavonifractor plautii and, in certain aspects, a member of the family Ruminococcaceae.

In other embodiments, different phylogenetic grouping methods known in the art may be used to determine if a bacterial strain is a descendant of a MRCA of Faecalibacterium prausnitzii and Flavonifractor plautii, including methods that use different analysis packages and are based on different programming languages.

2. 16S rDNA Sequence Identity

In other embodiments, a bacterial species is a member of the family Ruminococcaceae if the species has a 16S rDNA sequence with sequence identity to 16S rDNA sequences from species already idenfied as a member of the family Ruminococcaceae. In an embodiment, identification of whether a bacterial species is a member of the family Ruminococcaceae is performed using the methods described in Yarza et al., 2014, Nature Reviews Microbiology 12:635-645, and Stackebrandt, E. & Ebers, J., 2006, Microbiol. Today 8:6-9, which are hereby incorporated by reference herein.

In some embodiments, the 16S rDNA sequence is obtained or determined for a bacterial species to be classified. This query 16S rDNA sequence is compared to 16S rDNA sequences from bacterial species already classified as members of the family Ruminococcaceae. In some embodiments, the query 16S rDNA sequence is compared to the 16S rDNA sequences listed in Table 11. In some embodiments, the query 16S rDNA sequence is compared to all known 16S rDNA sequences for bacterial species already classified as members of the family Ruminococcaceae. In other embodiments, the query 16S rDNA sequence is compared to a subset of all known 16S rDNA sequences for bacterial species already classified as members of the family Ruminococcaceae. A percent identity between the query sequence and the compared sequences is determined. If the percent identify of the query sequence is determined to be above a defined threshold, then the bacterial species to be classified is classified as member of the family Ruminococcaceae.

In some embodiments, the threshold sequence identity is 94.5%. In some embodiments, the threshold sequence identity is 98.7%. In some embodiments, the threshold sequence identity is 94.8%. In some embodiments, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9% 99.0%, 99.1%, 99.2%, 99.3%, 99.4%, 99.5%. 99.6%, 99.7%, 99.8%, 99.9% or 100%.

3. Clades that are Part of the Family Ruminococcaceae

In some embodiments, bacteria species may be classified in one of clade 101, clade 14, clade 126, clade 61, clade 125 or clade 135 as shown in the phylogenetic tree in FIG. 6. In some embodiments, clade 101 comprises the bacterial species Flavonifractor plautii, Clostridium orbiscindens, Clostridium sp NML_04A032, Pseudoflavonifractor capillosus, Ruminococcaceae bacterium D16, Clostridium viride, Oscillospira guilliermondii, Oscillibacter sp_G2, Oscillibacter valericigenes, Sporobacter termitidis and Paplillibacter cinnamivorans. In some embodiments, clade 14 comprises the bacterial species Ruminococcus sp_18P13, Ruminococcus sp_9SE51, Ruminococcus champanellensis, Ruminococcus callidus, Ruminococcus flavefaciens and Ruminococcus albus. In some embodiments, clade 126 comprises the bacterial species Ethanoligenens harbinense, Clostridium cellulosi, Acetanaerobacterium elongatum, Clostridium sp_YIT_12070, Clostridium methylpentosum, Hydrogenoanaerobacterium saccharovorans, and Anaerotruncus colihominis. In some embodiments, clade 61 comprises the bacterial species Eubacterium siraeum, Subdoligranulum variabile, Gemmiger formicilis and Faecalibacterium prausnitzii. In some embodiments, clade 125 comprises the bacterial species Eubacterium coprostanoligenes, Clostridium sp_YIT_12069, Clostridium sporosphaeroides, Clostridium leptum and Ruminococcus bromii. In some embodiments, clade 135 comprises the bacterial species Eubacterium desmolans, Butyricicoccus pullicaecorum or combinations thereof.

In some embodiments, the clades herein can include additional species that are determined to be part of any one of clade 101, clade 14, clade 126, clade 61, clade 125 or clade 135. In some embodiments, the phylogenetic grouping methods described herein, including the MRCA and 16S rDNA sequence identity methods described above, may be used to determine in an additional species belongs in a clade. In some embodiments, an additional species is classified as part of a clade if the 16S rDNA of the additional species is at least 97% identical to the 16S rDNA of the other species in the clade. A person of ordinary skill in the art would also be able to use methods known in the art to determine whether a species is part of a clade, including methods described herein.

C. Methods for Determining 16S rDNA Sequences

Operational taxonomic units (OTUs) can be identified, for example, by sequencing of the 16S rRNA gene, by sequencing of a specific hypervariable region of this gene (i.e. V1, V2, V3, V4, V5, V6, V7, V8, or V9), or by sequencing of any combination of hypervariable regions from this gene (e.g. V1-3 or V3-5). The bacterial 16S rDNA is approximately 1500 nucleotides in length and is used in reconstructing the evolutionary relationships and sequence similarity of one bacterial isolate to another using phylogenetic approaches. 16S rDNA sequences are used for phylogenetic reconstruction as they are in general highly conserved, but contain specific hypervariable regions that harbor sufficient nucleotide diversity to differentiate genera and species of most microbes. Using well known techniques to determine a full 16S rDNA sequence or the sequence of any hypervariable region of the 16S rDNA sequence, genomic DNA is extracted from a bacterial sample, the 16S rDNA (full region or specific hypervariable regions) amplified using polymerase chain reaction (PCR), the PCR products cleaned, and nucleotide sequences delineated to determine the genetic composition of 16S rDNA gene or subdomain of the gene. If full 16S rDNA sequencing is performed, the sequencing method used may be, but is not limited to, Sanger sequencing. If one or more hypervariable regions are used, such as the V4 region, the sequencing may be, but is not limited to being, performed using the Sanger method or using a next-generation sequencing method, such as an Illumina (sequencing by synthesis) method using barcoded primers allowing for multiplex reactions. In some cases, the 16S rDNA sequence associated with an OTU, species, or strain of bacteria is a composite of multiple 16S rDNA sequences harbored by the OTU, species, or strain.

In some embodiments, bacterial species identified as described herein are identified by sequence identity to 16S rDNA sequences as known in the art and described herein. In some embodiments, the selected species are identified by sequence identity to full length 16S rDNA sequences as shown in Table 10.

In some embodiments, Clostridium_SC64 is identified by at least 97% identity to the full length 16S rDNA sequence provided as SEQ ID NO:1 or at least 97% identity to a variable region such as V4. In some embodiments, Blautia_SC102 is identified by at least 97% to the full length 16S rDNA sequence provided as SEQ ID NO:2 or at least 97% identity to a variable region such as V4. In some embodiments, Blautia_SC109 is identified by its full length 16S rDNA sequence provided as SEQ ID NO:3 or at least 97% identity to a variable region such as V4. In some embodiments, Blautia_SC109 is identified by its full length 16S rDNA sequence provided as SEQ ID NO:4 or at least 97% identity to a variable region such as V4.

### V. Methods for Preparing a Bacterial Composition for Administration to a Subject

Methods for producing bacterial compositions are known in the art. For example, a composition can be produced generally via three main processes, combined with one or more methods of mixing. The steps are: organism banking, organism production, and preservation.

For banking, the strains included in the bacterial composition can be, for example isolated directly from a specimen, obtained from a banked stock, optionally cultured on a nutrient agar or in broth that supports growth to generate viable biomass, and the biomass optionally preserved in multiple aliquots in long-term storage.

Stocks of organisms may prepared for storage, e.g., by adding cryoprotectants, lyoprotectants, and/or osmoprotectants. In general, such methods are known in the art.

### VI. Immuno-Oncology (Immunotherapy) Drugs that can be Used in Conjunction with the Therapeutic Compositions

In some embodiments of the invention, the therapeutic composition is an adjunct treatment administered in combination with an immunotherapy drug, generally an immune checkpoint inhibitor (e.g., an antibody, such as a monoclonal antibody). The terms “immune checkpoint inhibitor,” “immune checkpoint blockade,” and “immune checkpoint therapy” are used interchangeably. Examples of such immunotherapy drugs include PD-1 inhibitors (e.g., nivolumab, and pembrolizumab), PD-L1 inhibitors (e.g., atezolizumab, avelumab, and durvalumab), and CTLA-4 inhibitors (e.g., ipilimumab and tremelimumab). In some embodiments, more than one checkpoint inhibitor is administered. As is known in the art, dosing of checkpoint inhibitors can be repeated at, for example, 2-3 week intervals, for as long as the patient continues to have a response or stable disease, or as otherwise determined to be appropriate by those of skill in the art.

Examples of cancers that can benefit from treatment with the therapeutic compositions in conjunction with a checkpoint inhibitor, e.g., an inhibitor of PD-1, PD-L1, or CTLA-4, include but are not limited to metastatic melanoma, melanoma of the skin, non-small cell lung cancer, kidney cancer, bladder cancer, head and neck cancers, Merkel cell skin cancer (Merkel cell carcinoma), and Hodgkin lymphoma.

### VII. Methods of Treatment

In general, the therapeutic compositions are administered to a patient diagnosed with a cancer, e.g., melanoma, for example, metastasized melanoma in conjunction with an immunotherapy drug such as checkpoint inhibitor, e.g., an inhibitor of PD-1, PD-L1, or CTLA-4. A therapeutic composition can be administered prior to checkpoint inhibitor (e.g., PD-1/PD-L1 inhibitor or CTLA-4 inhibitor) treatment, for example, at least one week, two weeks, or three weeks in advance of the treatment. In some cases, administration of the therapeutic composition is continued after the initiation of checkpoint inhibitor (e.g., PD-1/PD-L1 or CTLA-4 inhibitor) treatment. The therapeutic compositions may be administered daily, weekly, or monthly to induce and/or maintain an appropriate microbiome in the patient's GI tract.

Prior to initiating administration of a therapeutic composition, the patient may be subject to antibiotic treatment (e.g., with vancomycin, neomycin, rifaximin, or other antibiotic) and/or a bowel cleanse. In some cases, the antibiotic is a non-absorbable or minimally absorbable antibiotic. In some cases, no bowel preparation is performed. Such preparation may increase the speed and or efficacy of engraftment of one or more species in the therapeutic compositions that are associated with an improvement in checkpoint inhibitor (e.g., PD-1/PD-L1 inhibitor) efficacy.

### VIII. Models for Testing

Animal models suitable for testing the efficacy of a microbiome composition for use in immunotherapy are known in the art, for example, as described in Cooper et al. (2014, Cancer Immunol Res 2:643-654) and Gopalakrishnan et al (2018, Science 359(6371):97-103) using the BP cell line, and reviewed in Li et al. (2017, Pharmacol & Therapeutics, dx.doi.org/10.1016/j.pharmthera.20170.02.002). Other useful models include germ-free mouse models (e.g., Matson et al. Science 359:104-108 (2018), Routy et al Science 59(6371):91-97 (2018)).

### IX. Formulations

A microbiome immune-oncology therapeutic composition for use as described herein can be prepared and administered using methods known in the art. In general, compositions are formulated for oral, colonoscopic, or nasogastric delivery although any appropriate method can be used.

A formulation containing a therapeutic composition can contain one or more pharmaceutical excipients suitable for the preparation of such formulations. In some embodiments, the formulation is a liquid formulation. In some embodiments, a formulation comprising the therapeutic compositions can comprise one or more of surfactants, adjuvants, buffers, antioxidants, tonicity adjusters, thickeners or viscosity modifiers and the like.

In some embodiments, treatment includes administering the therapeutic compositions in a formulation that includes a pharmaceutically acceptable carrier. In some embodiments, the excipient includes a capsule or other format suitable for providing the therapeutic compositions as an oral dosage form. When an excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, soft or hard capsules, suppositories, or packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, polyethylene glycol, glycerol, and methyl cellulose. The compositions can be formulated so as to provide quick, sustained or delayed release of the active ingredient after administration to the patient by employing procedures known in the art.

In some embodiments, the therapeutic composition can be incorporated into a food product. In some embodiments the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments, the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some embodiments, the therapeutic compositions are incorporated into a therapeutic food. In some embodiments, the therapeutic food is a ready-to-use food that optionally contains some or all essential macronutrients and micronutrients. In some embodiments, the compositions disclosed herein are incorporated into a supplementary food that is designed to be blended into an existing meal. In some embodiments, the supplemental food contains some or all essential macronutrients and micronutrients. In some embodiments, the bacterial compositions disclosed herein are blended with or added to an existing food to fortify the food's protein nutrition. Examples include food staples (grain, salt, sugar, cooking oil, margarine), beverages (juice, coffee, tea, soda, beer, liquor, sports drinks), snacks, sweets and other foods.

The therapeutic compositions can be formulated in a unit dosage form. In general, a dosage comprises about 1×10² to 1×10⁹ viable colony forming units (CFU). The term “unit dosage forms” refers to physically discrete units suitable as unitary dosages for human subjects and/or other mammals, each unit containing a predetermined quantity of active material calculated to produce the desired therapeutic effect, in association with a suitable pharmaceutical excipient. A dosage may be administered in multiple delivery vehicles, e.g., multiple pills, capsules, foodstuffs or beverages.

The amount and frequency of administering the therapeutic compositions to a patient can vary depending upon the specific composition being administered, the purpose of the administration (such as prophylaxis or therapy), the state of the patient, the manner of administration, and the like. In therapeutic applications, compositions can be administered to a patient already suffering from a disease in an amount sufficient to cure or at least partially arrest or mitigate the symptoms of the disease and its complications. An effective dose can depend on the disease condition being treated as well as by the judgment of the attending clinician depending upon factors such as the severity of the disease, the age, weight and general condition of the patient, and the like.

In some embodiments, at least one dose of the therapeutic composition is administered by the attending clinician or a person acting on behalf of the attending clinician. In some embodiments, the subject may self-administer some or all of the subsequent doses. In other embodiments, all doses of the therapeutic composition are administered by the attending clinician or a person acting on behalf of the attending clinician. In these embodiments, prior to the administration of a first dose of the therapeutic composition the attending clinician or a person acting on behalf of the attending clinician may administer an antibiotic treatment and/or a bowel cleanse.

The dosage can refer, for example, to the total number of viable colony forming units (CFUs) of each individual species or strain; or can refer to the total number of microorganisms in the dose. It is understood in the art that determining the number of organisms in a dosage is not exact and can depend on the method used to determine the number of organisms present. If the composition includes spores, for example, the number of spores in a composition may be determined using a dipicolinic acid assay (Fichtel et al, 2007, FEMS Microbiol Ecol, 61:522-32). In some cases, the number of organisms is determined using a culture assay.

Effective doses can be extrapolated from dose-response curves derived from in vitro or animal model test systems.

### X. Methods of Identifying a Candidate for Immune Checkpoint Therapy in Combination with Adjuvant Microbiome Therapy

In some embodiments, methods are provided of identifying a subject as a candidate for immune checkpoint therapy in combination with adjuvant microbiome therapy, the methods comprising: a) obtaining a microbiome sample from the subject, b) determining the prevalence or abundance of the genera or selected genera of bacteria in the microbiome sample, and c) determining that the subject is a candidate for anticancer treatment if the microbiome sample comprises bacteria belonging to one or more of the genera Ruminococcus, Gemmiger, Faecalibacterium, Subdoligranulum or combinations thereof. In some embodiments, methods are provided of identifying a subject as a candidate for immune checkpoint therapy in combination with adjuvant microbiome therapy, the methods comprising: a) obtaining a microbiome sample from the subject, b) determining the prevalence or abundance of the genera or selected genera of bacteria in the microbiome sample, and c) determining that the subject is a candidate for immune checkpoint therapy in combination with adjuvant microbiome therapy if the microbiome sample comprises bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In other embodiments, methods are provided of identifying a mammalian subject as a candidate for immune checkpoint therapy in combination with adjuvant microbiome therapy, the methods comprising: a) obtaining a microbiome sample from the subject, b) determining the prevalence and/or abundance of the genera of bacteria in the microbiome sample, and c) determining that the subject is a candidate for immune checkpoint therapy in combination with adjuvant microbiome therapy if the microbiome sample comprises one or more of the genera Alistipes, Bacteroides, Blautia, Clostridium, Eubacterium, Parabacteroides or combinations thereof. In other embodiments, methods are provided of identifying a mammalian subject as a candidate for immune checkpoint therapy in combination with adjuvant microbiome therapy, the methods comprising: a) obtaining a microbiome sample from the subject, b) determining the prevalence and/or abundance of the genera of bacteria in the microbiome sample, and c) determining that the subject is a candidate for immune checkpoint therapy in combination with adjuvant microbiome therapy if the microbiome sample comprises one or more of the genera Barnesiella, Bifidobacterium, Blautia, Erysipelotrichaceae, Odoribacter, Parabacteroides or combinations thereof.

In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises bacterial species that are phylogenetic descendants of the most recent common ancestor (MRCA) of Faecalibacterium prausnitzii and Flavonifractor plautii.

In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises bacterial species that have 16S rDNA sequence identity of at least 94.5% to 16S rDNA sequences of species belonging to the family Ruminococcaceae. In some embodiments, the bacterial species may have 16S rDNA sequence identity of at least 98.7% to 16S rDNA sequences of species belonging to the family Ruminococcaceae.

In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof. In some embodiments, the subject may be determined to be a candidate for anticancer treatment if at least two, three, four, five or more of the species listed are present in the microbiome sample.

In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises one or more of the bacteria species in one or more of clade 101, clade 14, clade 126, clade 61, clade 125 or clade 135.

In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In other embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Alistipes senegalensis, Bacteroides dorei, Blautia_SC109, Clostridium_SC64, Eubacterium_biforme, Parabacteroides distasonis or combinations thereof. In some embodiments, methods are provided of identifying a mammalian subject as a candidate for anticancer treatment, the methods comprising: a) obtaining a microbiome sample from the subject, b) determining the prevalence and/or abundance of the species of bacteria in the microbiome sample, and c) determining that the subject is a candidate for anticancer treatment if the microbiome sample comprises bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

In some embodiments, subjects that are identified as candidates for anticancer treatment are identified as candidates for treatment with a checkpoint inhibitor. In some embodiments, the checkpoint inhibitor can be an anti-PD-1 antibody, an anti-CTLA-4 antibody an anti-PD-L1 antibody or combinations thereof. In some embodiments, the checkpoint inhibitor can be, e.g., pembrolizumab, nivolumab, atezolizumab, avelumab, durvalumab or ipilimumab, or other checkpoint inhibitors known in the art. In other embodiments, the checkpoint inhibitors can be e.g., pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors STI-A1010, or combinations thereof. In other embodiments, the subject can be candidates for treatment with cyclophosphamide. In some embodiments, the immune checkpoint therapy comprises immune checkpoint blockade monotherapy. In some embodiments, the immune checkpoint therapy comprises immune checkpoint blockade combination therapy.

### XI. Methods of Identifying FMT Donors

Applicants have discovered that certain microbiome profiles, e.g., families, genera, and/or species are associated with improved outcomes in therapy with a checkpoint inhibitor. Accordingly, in some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacteria belonging to one or more of the genera Ruminococcus, Gemmiger, Faecalibacterium, Subdoligranulum or combinations thereof. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof. In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one or more of the genera Alistipes, Bacteroides, Blautia, Clostridium, Eubacterium, Parabacteroides or combinations thereof. In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one or more of the genera Barnesiella, Bifidobacterium, Blautia, Erysipelotrichaceae, Odoribacter, Parabacteroides or combinations thereof.

In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacterial species that are phylogenetic descendants of the most recent common ancestor (MRCA) of Faecalibacterium prausnitzii and Flavonifractor plautii.

In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacterial species that have 16S rDNA sequence identity of at least 94.5% to 16S rDNA sequences of species belonging to the family Ruminococcaceae. In some embodiments, the bacterial species may have 16S rDNA sequence identity of at least 98.7% to 16S rDNA sequences of species belonging to the family Ruminococcaceae.

In other embodiments, methods are provided of selecting donors whose feces are useful for fecal matter transfer, the methods comprising: a) obtaining a microbiome sample from the potential donor, b) determining the prevalence of the species of bacteria in the microbiome sample, and c) determining that the donor's feces is useful for fecal matter transfer if the microbiome sample comprises one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof. In some embodiments, the potential donor may be determined to be a donor for fecal matter transfer if at least two, three, four, five or more of the species listed are present in the microbiome sample.

In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one or more of the bacteria species in one or more of clade 101, clade 14, clade 126, clade 61, clade 125 or clade 135. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one, two, three, four, five, six, seven, eight, nine, ten or eleven species of clade 101. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one, two, three, four, five or six, species of clade 14. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one, two, three, four, five, six or seven species of clade 126. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one, two, three or four species of clade 61. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one, two, three, four or five species of clade 125. In some embodiments, the therapeutic compositions comprise an effective amount of one or two species of clade 135.

In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacterial species that are phylogenetic descendants of the most recent common ancestor (MRCA) of Faecalibacterium prausnitzii and Flavonifractor plautii.

In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacterial species that have 16S rDNA sequence identity of at least 94.5% to 16S rDNA sequences of species belonging to the family Ruminococcaceae. In some embodiments, the bacterial species may have 16S rDNA sequence identity of at least 98.7% to 16S rDNA sequences of species belonging to the family Ruminococcaceae.

In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof. In some embodiments, the potential donor may be determined to be a donor for fecal matter transfer if at least two, three, four, five or more of the species listed are present in the microbiome sample.

In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one or more of the bacteria species in one or more of clade 101, clade 14, clade 126, clade 61, clade 125 or clade 135. In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one, two, three, four, five, six, seven, eight, nine, ten or eleven species of clade 101. In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one, two, three, four, five or six, species of clade 14. In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one, two, three, four, five, six or seven species of clade 126. In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one, two, three or four species of clade 61. In some embodiments, methods are provided of selecting donors whose feces are useful for fecal matter transfer, the methods comprising: a) obtaining a microbiome sample from the potential donor, b) determining the abundance of the species of bacteria in the microbiome sample, and c) determining that the donor's feces is useful for fecal matter transfer if the microbiome sample comprises one, two, three, four or five species of clade 125. In some embodiments, the therapeutic compositions comprise an effective amount of one or two species of clade 135.

In other embodiments, methods are provided of selecting donors whose feces are useful for fecal matter transfer, the methods comprising: a) obtaining a microbiome sample from the potential donor, b) determining the prevalence of the species of bacteria in the microbiome sample, and c) determining that the donor's feces is useful for fecal matter transfer if the microbiome sample comprises bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof. In other embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacteria species selected from Alistipes senegalensis, Bacteroides dorei, Blautia_SC109, Clostridium_SC64, Eubacterium_biforme, Parabacteroides distasonis or combinations thereof. In some embodiments, methods are provided of selecting donors whose feces are useful for fecal matter transfer, the methods comprising: a) obtaining a microbiome sample from the potential donor, b) determining the prevalence and/or abundance of the species of bacteria in the microbiome sample, and c) determining that the donor's feces is useful for fecal matter transfer if the microbiome sample comprises bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

Below are examples of specific embodiments for carrying out the present invention. The examples are offered for illustrative purposes only, and are not intended to limit the scope of the present invention in any way. Efforts have been made to ensure accuracy with respect to numbers used, but some experimental error and deviation should, of course, be allowed for.

### XII. Examples

### Example 1: Taxonomic Profiling

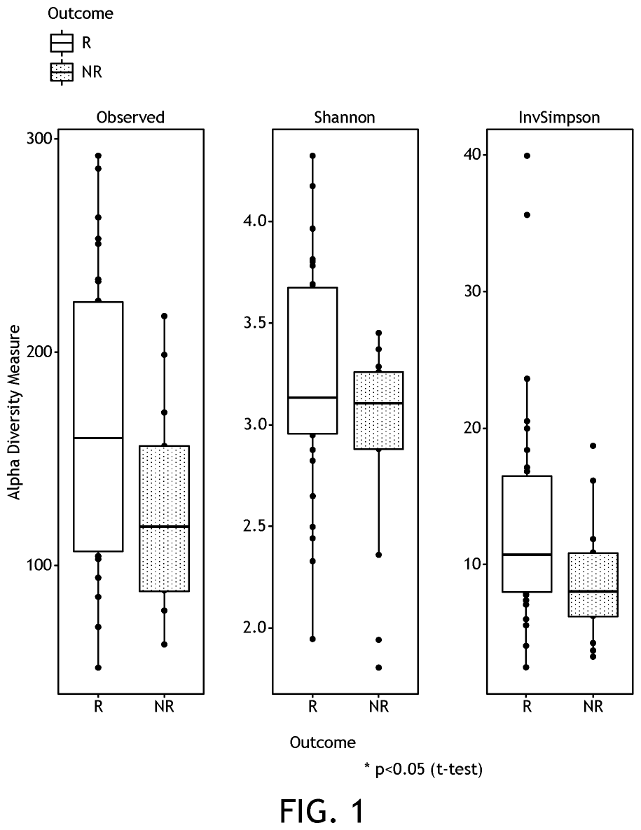

Whole metagenomics sequencing (WMS) raw data from Gopalakrishnan et al. (Science 2018; 359: 97-103) were obtained and analyzed as described herein. As described in Gopalakrishnan et al., supra, the WMS sequences were generated using fecal microbiome samples from metastatic melanoma patients who were classified as responders or non-responders to a checkpoint inhibitor. Responder and non-responder classes of subjects were determined as described in Gopalakrishnan et al. The raw data sets were pre-processed following the guidelines set by the Human Microbiome Project. The pre-processing analysis was used to perform error analysis and removal of low-quality sequences and other undesirable data, such as sequences present from PCR amplification steps. Species-level taxonomic profiles of each WMS sample were obtained using a MetaPhlAn2 software package (e.g., Truong et al., Nature Meth 12:902-903, 2015). In brief, MetaPhlAn2 is a software tool that aligns each sample to a curated reference database of marker genes, each of which is unique to a bacterial species. The reference database contains more than one million marker genes, representing more than seven thousand bacterial species. Alpha diversity, i.e., a measure of species richness, of 16S rDNA for responders (R) and non-responders (NR) is shown in FIG. 1.

### Example 2: Summary of Data Types and Data Analysis Methods

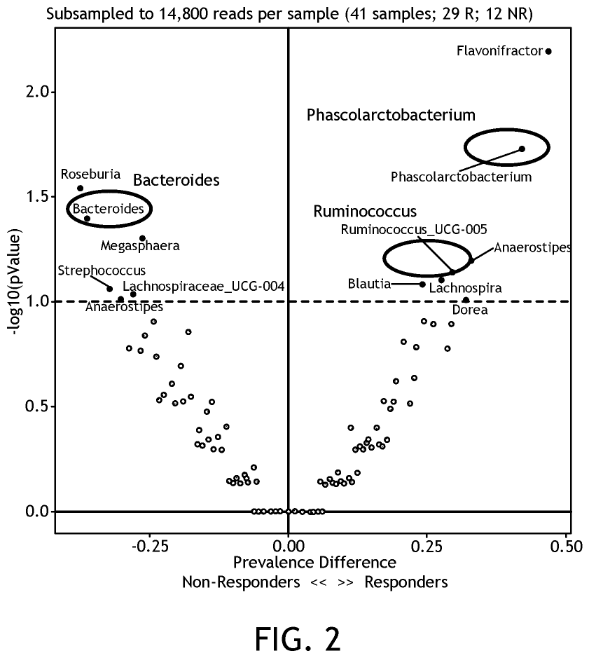

Abundance data were obtained after profiling WMS data. For a given sample, the sum of the abundances of all species sums to 100. Prevalence data are discretized so that species are analyzed only as being either present or absent. This is a population-wide data type, meaning that it can only be assessed for a set of samples and not individually for any given sample. For example, the prevalence of a species that appears in 4 out of 10 responders is 40%. Quantile normalized abundance is a procedure that was used to standardize microarray data. Across data sets, estimated abundance values of a given species may lead to a different interpretation due to a variety of reasons including technical artifacts arising from differences in sample processing. The quantile normalization approach re-assigns abundance values of a species given the distribution of abundances of that species in a set of background samples (in this case, non-responders). The normalized value is the percentage of background samples that have an abundance less than or equal to the abundance of the given species in the given sample. A volcano plot of results from a differential prevalence analysis is shown in FIG. 2.

Using these three data types, four analytic methods were used to generate independent data sets: Fisher's exact test, Lasso regression, Random forest analysis, and Linear discriminant analysis. These analytic approaches are briefly described below. A table summarizing key features of the methods is provided in Table 9.

The Fisher's exact test is a test for a difference in distribution of categorical variables. Applicants applied this analysis to test for differences in species prevalence between responders and non-responders, given the number of samples found in each group. For example, a species that occurs in 8/12 responder samples would have a prevalence of 67%. Statistical significance is calculated between the prevalence of responders and non-responders based on the same size of each group.

The Lasso Regression is different from simple regression, where an effect is assigned to every feature in the data set (such as species abundance and/or prevalence). Instead, Lasso regression attempts to minimize small effects in order to retain the smallest collection of features that have the largest impact on outcome, using an L1 regularization approach. This approach attempts to avoid overfitting the data to all possible variables in the data set, and instead leads to more interpretable results.

The random forest classifier is an algorithm based on the results of many decision trees. In a single decision tree, features are selected iteratively that best separate samples into responder and non-responder categories, until all features are utilized. In the case of prevalence data, these features could be presence or absence of a given species, where presence of a single species might be preferentially associated with responder samples, or vice versa. Since a single decision tree typically overfits data and does not produce robust results, random forests are often used instead. A random forest classifier is based on many different decision trees, where each tree only uses a subset of the available data, for example randomly leaving out 20% of the observed species for each tree. In some cases, a subset of the samples is used for training the random forest. The random forest classifier thus learns which signals are strongest across all possible features and samples.

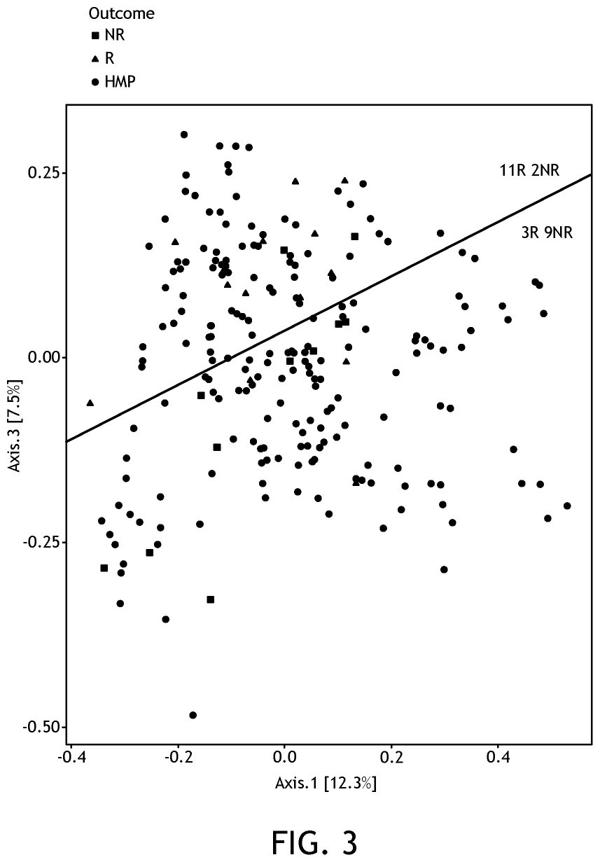

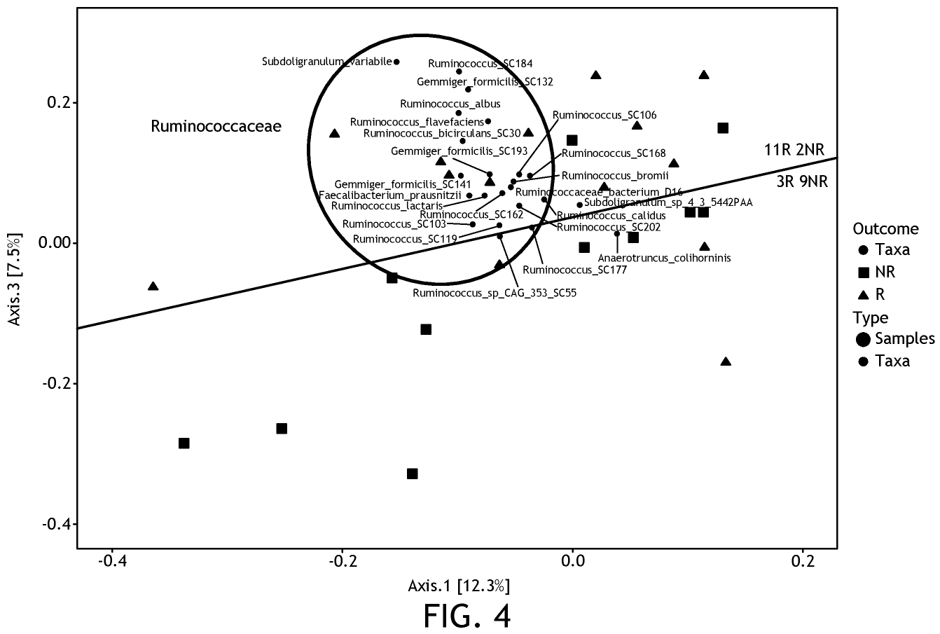

Linear discriminant analysis (LDA) is a method that attempts to find a linear combination of features that separates samples into two or more outcomes. For example, in a multidimensional scaling (MDS) representation of the Bray-Curtis dissimilarity among samples, the method can be applied to identify the species that distinguish responder from non-responder samples. Due to the limited sample size of the available data, and to provide additional information that might be present in a larger set of healthy background samples, this approach was applied to the data as embedded into approximately 200 samples from healthy donors as collected in the Human Microbiome Project (HMP). This was done by calculating the Bray-Curtis dissimilarity among all WMS and HMP samples. LDA was then used to generate a classification line to separate responder and non-responder samples in the data as embedded in the combined MDS plot (FIG. 3). Further, species data mapped onto a beta diversity plot demonstrates that Ruminococcaceae are generally associated with patients classified as responders (FIG. 4).

A ranking of the significance of association of taxa to responder and non-responder status can then be evaluated based on their distance from the classification line, where taxa that are further from the line (e.g. driving the signal of separation between R and NR) are given a higher score. In order to mitigate the significance of rare species which are found in very few samples, the score was modified by multiplying it by the log of the prevalence of the species in the pooled data. The effect of this final modification is that species with very low prevalence are assigned a lower significance score. Due to the fact that this list sets no cutoff threshold for statistical significance, we examined scores in a quantile-quantile style plot and selected the inflection point of scores as the cutoff.

### Example 3: Development of Aggregate Results and Rankings Based on Penalized Geometric Mean Analysis

After obtaining a ranked list of species according to the various methods and data types above, a method of aggregating the rankings was developed that fulfill the following properties: species that are significantly associated with response were assigned higher ranks, species that were found significantly associated with response across multiple methods were assigned higher ranks compared to species that were found significantly associated in only one or two methods, and final species rankings were robust to potential outliers in individual method rankings. The first two properties are intuitive, since species that are identified as significant using multiple algorithms and data types are more likely to represent a real and robust signal. Because different algorithms may return a different number of significantly associated species, the third property was included to minimize the penalty for rankings based solely on significantly associated species. The aggregate results of the ranked lists generated by the alternate analysis methods are in Tables 1-2.

A penalized geometric mean approach was developed to generate the aggregate results. For each species, the geometric mean was calculated from its ranks across all methods in which it was identified. The geometric mean is defined as the product of all n values, followed by taking the n^{th} root. For example, for “Species Example 1” that was identified in three out of the four methods, the geometric mean of (1, 2, 10) would be (1×2×10)^{(1/3)}=2.71. This geometric mean is robust to outliers, but it is susceptible to bias for certain data sets, such as “Species Example 2” instead appearing in all four analysis methods with rankings 1, 2, 2, 20 across the four analysis methods (1, 2, 2, 20), since (1×2×2×20)\hat{ }(¼)=2.99. Using this approach, Species Example 1, with a value of 2.71 would be ranked higher than Species Example 2 due to its lower geometric mean score, yet this approach does not account for the prevalence aspect of the analysis and the fact that Species Example 1 was not identified in one of the four analysis methods.

To account for this discrepancy, scores were penalized by the square of the number of methods in which a given species is not found. These aggregate scores are then ranked from lowest to highest, with lowest scores attributed to species for which we have the most confidence. Thus, better scores were preferentially assigned to those species which are identified as significant by a variety of different methods. Final aggregate rankings can be found in Tables 1-2.

These analyses demonstrate the in silico analysis of human microbiome data can be used to identify bacteria genera and species associated with a response to a checkpoint inhibitor. Accordingly, species identified as provided herein are useful in compositions for improving the efficacy of a checkpoint inhibitor treatment.

### Example 4: Further Validation Studies

Several studies have reported various disparate GI microbiome signatures for individuals having an improved response to a checkpoint inhibitor. Applicants undertook a further analysis of data reported in Gopalakrishnan et al., 2018 to determine whether a signature could be detected that would be useful for identifying donor fecal material likely to be effective for the preparation of a microbiome composition useful as an adjunct therapy for treating patients receiving checkpoint inhibitor therapy.

It is desirable that detection of the signature has a rapid turnaround time and can be implemented, e.g., as a qPCR diagnostic. Validation of the signature using an additional cohort of patients selected by the laboratory of Dr. Jennifer Wargo using the same criteria for patient selection and identification of disease state as in Gopalakrishnan et al (2018) was then performed.

### Terms & Abbreviations

The following terms and abbreviations are used in Example 4:
Clade system: An internal numbered classification system based on the concept of clades, i.e. a group of related organisms representing all of the phylogenetic descendants of a common ancestor.
RECIST: Response evaluation criteria in solid tumors. A set of guidelines to determine the response of tumors to therapy.
refOTU: An internal classification system of 16S rDNA sequences assigned to specific taxonomies that is derived from NCBI and internal sources.
Responders and non-responders: non-responders include patients within the RECIST category progressive disease, while responders include patients in the RECIST categories stable disease, partial response, and complete response.
ROC curve: Receiver operating characteristic curve. A plot that shows the true positive and false positive rate of a binary classifier as the definition of the classifier is varied.
OTU (Operational Taxonomic Unit): An operational definition of a group of closely related organisms outside of traditional Linnaean taxonomy.
Silva: A widely used database of rDNA sequences and their classifications (https://www.arb-silva.de/).
USEARCH: A suite of sequence searching and clustering algorithms developed by R. Edgar.
Wargo Types: Gopalakrishnan et al (2018) divided patients into two microbiome types: Type 1 (enriched in Clostridiales) included only responders while Type 2 (enriched in Bacteroidales) included a mix of responders and non-responders.

A. Materials & Methods

1. Acquisition of Sequence Data

Human fecal 16S NGS sequencing (Illumina MiSeq) data from 43 patients (30 responders and 13 non-responders) from the Gopalakrishnan et al (2018) study were downloaded from the European Nucleotide Archive (ENA) of the European Bioinformatics Institute (EBI) (https://www.ebi.ac.uk/ena/data/view/ERX2218758, Experiment: ERX2218758, Project: PRJEB22894). Additional human fecal 16S NGS sequencing (Illumina MiSeq) data were obtained from the second cohort of 69 patients (39 responders and 30 non-responders).

2. Taxonomic Profiling of 16S Sequence Data Through USEARCH

Both published data and validation data were processed through the Seres USEARCH-based pipeline. Reads were merged using USEARCH v7.0.1090 (Edgar 2010, 2013) allowing four mismatches per ≥50 bases. Taxonomic annotations were assigned to 16S V4 sequence reads using the USEARCH v7.0.1090 (Edgar, 2010, 2013) algorithm. The USEARCH algorithm was parameterized to maximize sequence read data retention and to return the optimal taxonomy. Operational Taxonomic Unit (OTU) assignment based on 16S V4 sequence data is limited by the amount of information in the approximately 254 base pairs comprising this rDNA domain. To gain maximal information content from 16S V4 sequences, applicants developed a proprietary clade mapping system based on the ability of the 16S V4 region to reliably distinguish groups (clades) of related organisms. This system was used to define the phylogenetic clade that can be definitively assigned to any given OTU. As discussed herein, clades provide a resolution that is greater than genus assignment but typically less than species. These clades define the group of bacterial species that are not reliably distinguished from one another using the 16S V4 sequencing assay but can be distinguished from other bacterial species in other clades. Importantly, while the precise assignment of species is often not possible with 16S V4 data, the consistent determination of the number of distinct OTUs within a given clade is robust using the algorithms reported here.

3. Statistical Analysis

Mann-Whitney U tests were conducted on continuous or integer-based data (e.g., relative abundance, species diversity), while Fisher's exact tests were conducted on categorical data (e.g., Wargo Types). All p-values were corrected for multiple comparisons using the Benjamini-Hochberg method.

B. Results & Analysis

1. Type 1 Microbiomes are Enriched in Clostridia while Type 2 Microbiomes are Enriched in Bacteroidia

Gopalakrishnan et al (2018) subdivided patients into two microbiome types: Type 1 (enriched in Clostridiales), which included only patients defined by the authors as responders, and Type 2 (enriched in Bacteroidales), which included a mix of responders and non-responders. A USEARCH-based pipeline and NCBI-based genus-level classification were used to verify these compositional differences in the published 16S sequencing data. Differentially prevalent higher taxa at the levels of class and family were identified between Type 1 and Type 2 patients using a Mann-Whitney U test adjusted for multiple comparisons at each taxonomic level using the Benjamini-Hochberg method. Type 1 patients were enriched for Clostridia, particularly the families Ruminococcaceae, Lachnospiraceae, Clostridiaceae, and Catabacteriaceae, while Type 2 patients were enriched in Bacteroidia (Table 12). This enrichment is similar to that identified in Gopalakrishnan et al (2018) Table S5.

TABLE 12
Type 1 microbiomes are enriched in Clostridia while Type
2 microbiomes are enriched in Bacteroidia. All class- and
family-level taxa significantly enriched in either type
are shown below. Mann-Whitney U tests were conducted for
each taxon, and adjusted for multiple comparisons at each
taxonomic level using the Beniamini-Hochberg method.

| | | | | Adj |
|---|---|---|---|---|
| Level | Taxon | Enrichment | P-value | P-value |
| Class | Bacterioidia | Type 2 | 1.4 × 10⁻⁹ | 2.6 × 10⁻⁸ |
| Class | Clostridia | Type 1 | 2.3 × 10⁻⁷ | 2.2 × 10⁻⁶ |
| Family | Ruminococcaceae | Type 1 | 0.0019 | 0.0068 |
| Family | Lachnospiraceae | Type 1 | 0.00098 | 0.0046 |
| Family | Clostridiaceae | Type 1 | 5.5 × 10⁻⁵ | 0.00076 |
| Family | Catabacteriaceae | Type 1 | 0.00045 | 0.0032 |

2. Relative Abundance of Ruminococcaceae, Clostridia, and Bacteroidia are the Strongest Predictors of Response

Potential correlates of checkpoint efficacy were then evaluated by comparing directly with response rather than type. Both Wargo type and Clostridia species diversity were evaluated based on findings in Gopalakrishnan et al (2018), and the relative abundance of Clostridia, Bacteroidia, and Ruminococcaceae based on the analysis above. The relative abundance of Clostridiaceae and Lachnospiraceae was not evaluated further as their signal appeared to be driven by high abundances in a small number of samples. For each potential correlate, a statistical test was conducted to determine if there was a significant difference between responders and non-responders (Table 13). The specific test was determined by whether the correlate was categorical (Fisher's exact test) or numerical (Mann-Whitney U test). Ruminococcaceae, Clostridia, and Bacteroidia relative abundance, and Wargo type all differed significantly (p<0.05) between responders and non-responders, while Clostridia diversity (in OTUs) did not.

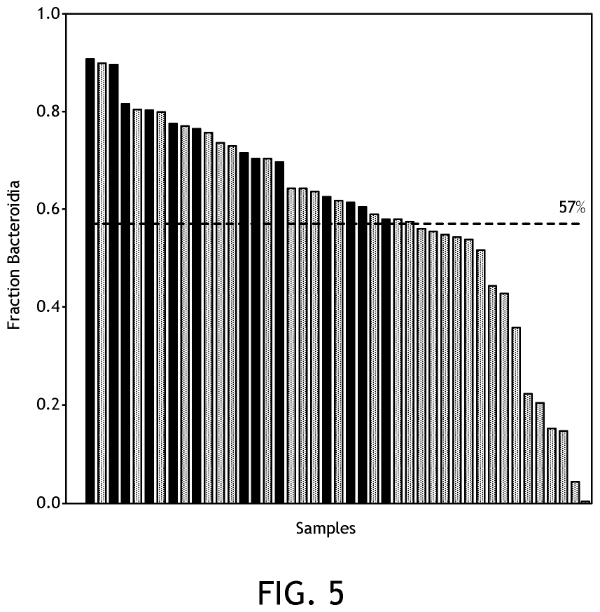

Next, for each potential correlate, a binary classification system was developed where the optimal cut-off was chosen to separate responders from non-responders based on first maximizing specificity (to 100% if possible) and then maximizing sensitivity using bar plots (FIG. 5, Table 13). Relative abundances of Ruminococcaceae, Clostridia, and Bacteroidia were all more sensitive predictors of response than Wargo type (54-57% vs 37%, respectively), showing that classification systems based on relative abundance could capture more responders than those based on Wargo type. Accordingly, the use of relative abundance can be used as an improved metric for identifying samples that are most associated with responders.

TABLE 13
Relative abundance of Ruminococcaceae, Clostridia, and Bacteroidia
were found to be the strongest predictors of response to checkpoint
therapy. Association of each microbiome characteristic analyzed with
response is shown below along with the statistical test used. Sensitivity
and specificity of each as a binary classifier is also shown; the
cut-off for binary classification is shown in parentheses after the
microbiome characteristic. OTUs were based on USEARCH and assigned
taxonomy as described. M-W U: Mann-Whitney U test.

| | Test for | Associ- | | |
|---|---|---|---|---|
| | associ- | ation | Sensi- | Speci- |
| Microbiome | ation | with | tivity as | ficity as |
| Characteristic | with | response | binary | binary |
| (classifier cut-off) | response | (p-value) | classifier | classifier** |
| Wargo Types (I and II) | Fisher's | 0.019* | 37% | 100% |
| Clostridia diversity | M-W U | 0.024* | 47% | 92% |
| (≥and <90 16S OTUs) | | | | |
| Clostridia relative | M-W U | 0.0032* | 50% | 100% |
| abundance | | | | |
| (≥and <32%) | | | | |
| Ruminococcaceae | M-W U | 0.00026* | 60% | 100% |
| relative | | | | |
| abundance | | | | |
| (≥and <9.5%) | | | | |
| Bacteroidia | M-W U | 0.0020* | 50% | 100% |
| relative | | | | |
| abundance | | | | |
| (≤and >57%) | | | | |

*significant at p < 0.05 level
**the classifier threshold was set by first maximizing specificity (to 100% if possible) and then maximizing sensitivity

3. Phylogenetic Definition of Ruminococcaceae Improves Sensitivity to Detect Responders

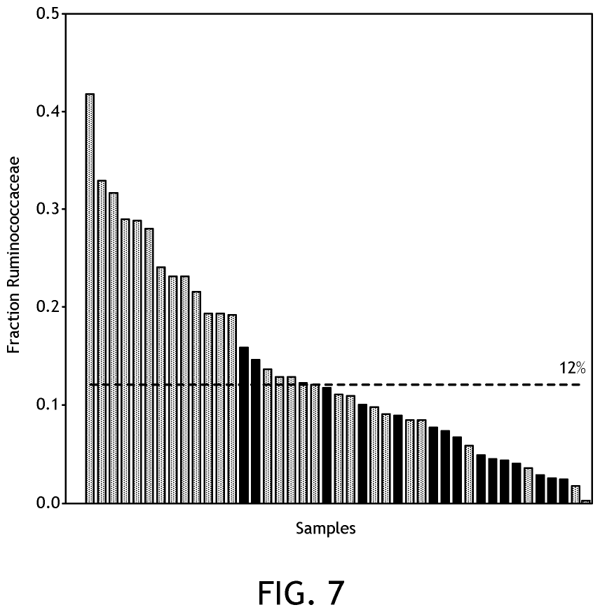

Specific examination of taxa assigned to Ruminococcaceae by NCBI in the context of a phylogenetic tree derived from 16S rDNA sequences indicates that some taxa are misclassified with respect to Ruminococcaceae. FIG. 6 shows a phylogenetic tree of Ruminococcaceae derived from 16S rDNA sequences from NCBI RefSeq and sequenced strains from Seres' strain collection. Taxa in underlined were listed in the NCBI taxonomy as not belonging to Ruminococcaceae; accordingly, NCBI-based classification is clearly not consistent with phylogeny. Applicants therefore undertook the development of a definition of Ruminococcaceae that is more indicative of true evolutionary relationships using an internal phylogenetic-based classification system (specifically, clades 14, 61, 101, 125, 135). Clade 13, traditionally classified as Ruminococcaceae, was left out of the definition of Ruminococcaceae for purposes of analyzing responder and non-responder microbiomes because the clade was highly divergent from the rest of the Ruminococcaceae (FIG. 6). Clade-based relative abundance of Ruminococcaceae was significantly associated with response (p=0.00078, Mann-Whitney U test) and was more sensitive than the NCBI-based definition (67%) while maintaining 100% specificity (Table 14, FIG. 7). Further, the threshold was increased from 9.5% to 12% using the clade-based definition because a greater number of Ruminococcaceae species were detected by the clade-based definition, resulting in higher per sample abundances. Further studies therefore used the phylogenetic, clade-based definition of Ruminococcaceae.

TABLE 14
A clade-based definition of Ruminococcaceae is more sensitive in
classifying responders than an NCBI-based definition. Association
of each microbiome characteristic analyzed with response is shown
below along with the statistical test used. Sensitivity and specificity
of each a binary classifier is also shown; the cut-off for binary
classification is shown in parentheses after the microbiome characteristic.
OTUs were based on USEARCH and assigned taxonomy as described.
M-W U: Mann-Whitney U test.

| | Test for | Associ- | | |
|---|---|---|---|---|
| | associ- | ation | Sensi- | Speci- |
| Microbiome | ation | with | tivity | ficity |
| Characteristic | with | response | as binary | as binary |
| (classifier cut-off) | response | (p-value) | classifier | classifier |
| Ruminococcaceae | M-W U | 0.00026* | 60% | 100% |
| relative | | | | |
| abundance | | | | |
| (≥and <9.5%) | | | | |
| Ruminococcaceae clade- | M-W U | 0.00078* | 67% | 100% |
| based relative abundance | | | | |
| (≥and <12%) | | | | |

*significant at p < 0.05 level

4. Combination of Ruminococcaceae and Bacteroidia Provides Increased Sensitivity while Maintaining Specificity

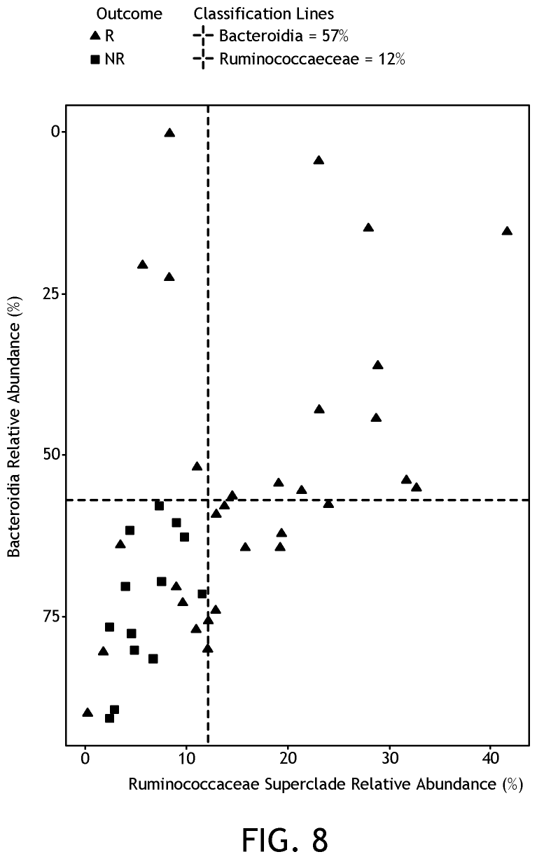

An analysis was performed to determine if a combination of classification systems would provide superior sensitivity and specificity over a single classification system. The union of a number of relative abundance metrics listed above was examined for sensitivity and specificity in detecting responders from the total patient pool (Table 15). While most combinatorial metrics showed 100% specificity, combining a minimum Ruminococcaceae clade-based abundance with a maximum Bacteroidia clade-based abundance showed the highest sensitivity (80%). Details of where each sample fell within this distribution are shown in FIG. 8.

TABLE 15
Combination of Ruminococcaceae and Bacteroidia provides increased
sensitivity while maintaining specificity. Sensitivity and specificity
of combining classification systems is shown below.

| | Sensi- | Speci- |
|---|---|---|
| Classification System | tivity | ficity |
| Clostridia Relative Abundance (above 32%) OR | 67% | 100% |
| Ruminococcaceae Relative Abundance (above 9.5%) | | |
| Ruminococcaceae Relative Abundance (above 9.5%) | 73% | 92% |
| OR Bacteroidia Relative Abundance (below 57%) | | |
| Ruminococcaceae clade-based Relative Abundance | 80% | 100% |
| (above 12%) OR Bacteroidia Relative Abundance | | |
| (below 57%) | | |
| Clostridia Relative Abundance (above 32%) OR | 60% | 100% |
| Bacteroidia Relative Abundance (below 57%) | | |
| Clostridia Relative Abundance (above 32%) OR | 73% | 100% |
| Ruminococcaceae Relative Abundance (above 9.5%) | | |
| OR Bacteroidia Relative Abundance (below 57%) | | |

5. Validation of Ruminococcaceae Metric in Second Cohort

Following development of the combined metric above, a new dataset was generated (n=69), using the same selection criteria for patients as Gopalakrishnan et al (2018) and it was desired to validate the metric using this new dataset. Relative abundance of clade-based Ruminococcaceae was significantly associated with response in the validation dataset (p=0.031, Table 16), while relative abundance of Bacteroidia was not (p=0.5, Table 15). De novo analysis to identify taxa at the (NCBI taxonomy-based) class and family level significantly associated with response identified only Ruminococcaceae and Clostridia (unadjusted p=0.047 and 0.049, respectively), indicating that no strong, conflicting signal existed in the validation dataset that was absent from the original, published dataset.

TABLE 16
Validation test of Ruminococcaceae and Bacteroidia relative abundances.
P-values listed include the original, published data from Gopalakrishnan
et al (2018) (n = 43), the validation cohort (n = 69),
and the combination of the two datasets (n = 112). All p-
values were generated with the Mann-Whitney U test.

| | Original | Validation | Combined |
|---|---|---|---|
| Measure | P-value | P-value | P-value |
| Ruminococcaceae clade- | 0.00078* | 0.031* | 0.00012* |
| based relative | | | |
| abundance | | | |
| Bacteroidia relative | 0.0020* | 0.50 | 0.035* |
| abundance | | | |

*significant at p < 0.05 level

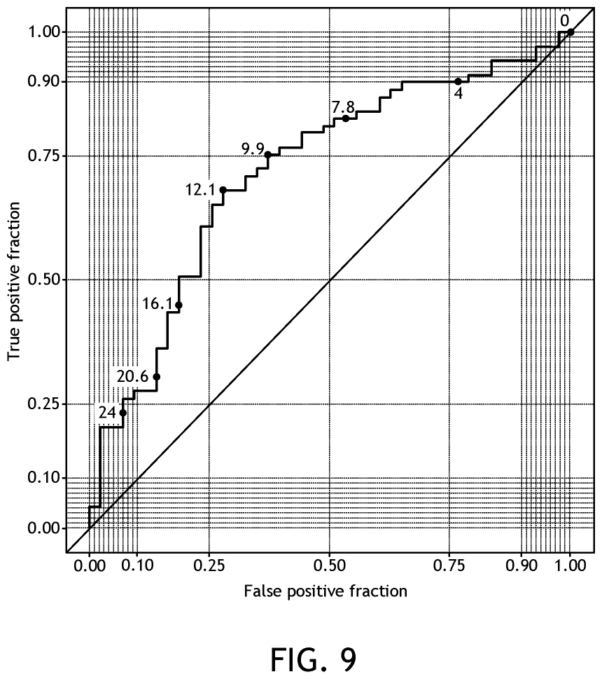

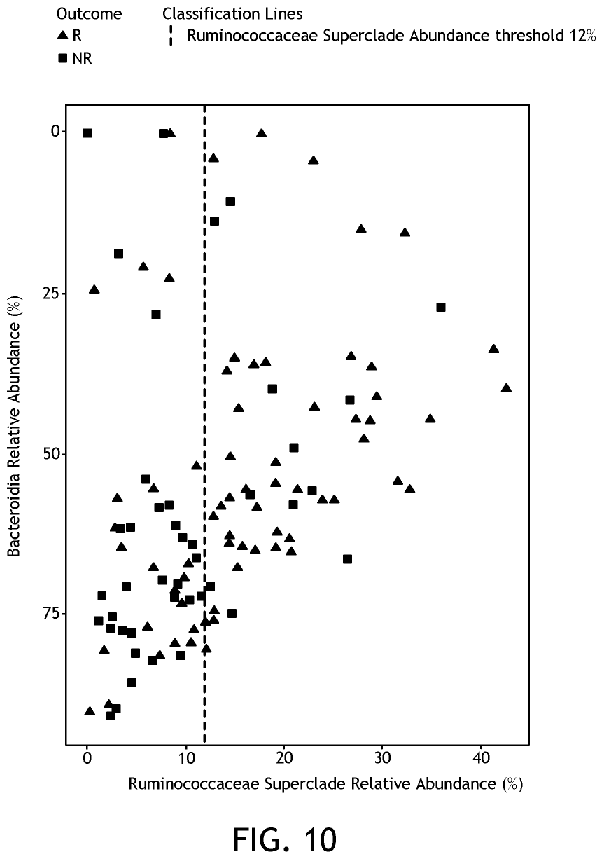

The 12% cutoff for clade-based Ruminococcaceae and the 57% cutoff for Bacteroidia discussed above were both further evaluated with respect to sensitivity and specificity. While specificity of 12% Ruminococcaceae decreased for both the validation and combined datasets, sensitivity remained in the 67-69% range (Table 17). Evaluation of the ROC curve for Ruminococcaceae did not suggest a significantly better cutoff than 12% existed in the combined dataset (FIG. 9). Patients with <12% Ruminococcaceae made up 47% (53/112) of total patients, but 72% of total non-responders. Patients with >=12% Ruminococcaceae, on the other hand, made up 53% of total patients and 68% of total responders (FIG. 10). For Bacteroidia, specificity dropped while sensitivity remained stable; however, sensitivity of Bacteroidia was near 50% in all datasets (Table 16), giving it little power to distinguish responders from non-responders when specificity was low. Based on these analyses, using a minimum 12% Ruminococcaceae clade-based abundance alone has the greatest combined specificity and sensitivity for distinguishing responders from non-responders in the combined dataset.

TABLE 17
Sensitivity and specificity of Ruminococcaceae and Bacteroidia thresholds in all datasets.
Datasets include the original, published data from Gopalakrishnan et al (2018) (n =
43), the validation cohort (n = 69), and the combination of the two datasets (n = 112).

| | Original | Original | Validation | Validation | Combined | Combined |
|---|---|---|---|---|---|---|
| Threshold | Sensitivity | Specificity | Sensitivity | Specificity | Sensitivity | Specificity |
| 12% Ruminococcaceae | 67% | 100% | 69% | 60% | 68% | 73% |
| (clade-based) | | | | | | |
| 57% Bacteroidia relative | 50% | 100% | 54% | 57% | 52% | 70% |
| abundance | | | | | | |

* significant at p < 0.05 level

6. Ruminococcaceae Significantly Different Despite Classification of Stable Disease

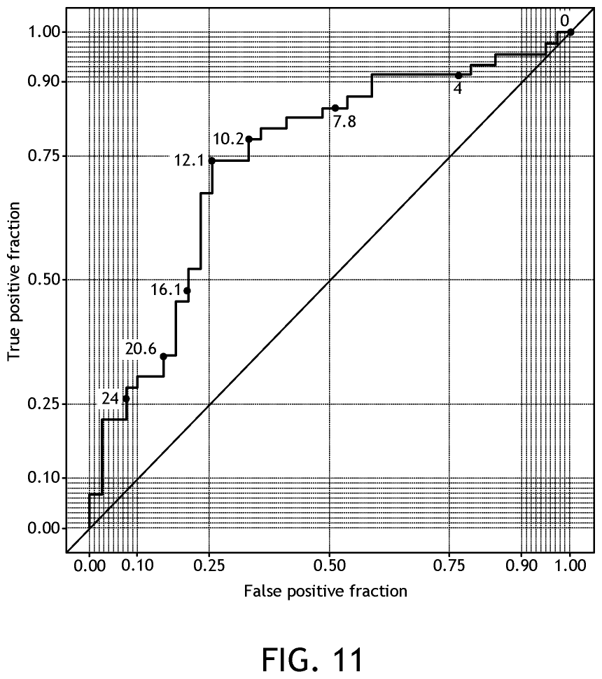

It was also determined whether the signature held if patients with stable disease were excluded from the analysis. Ruminococcaceae clade-based relative abundance maintained an equivalently significant difference between responders and non-responders whether stable disease patients (and the two patients classified as responders but without a specific RECIST classification) were included as responders (p=0.0012, Mann-Whitney U test) or excluded from the analysis altogether (p=0.0010, Mann-Whitney U test). Further, exclusion of stable disease slightly increased sensitivity to detect responders in the combined dataset (68% with all patients to 74% excluding stable disease), while maintaining specificity (73% with all patients to 74% excluding stable disease). Examination of the ROC curve for the combined dataset excluding stable disease patients affirmed choice of the 12% cutoff for Ruminococcaceae (FIG. 11).

C. Summary & Conclusion

A number of recent studies have established a correlation between microbiome composition and response to checkpoint therapy for treatment of cancer. In particular, Gopalakrishnan et al (2018) found that responder microbiomes were enriched for Clostridiales and Ruminococcaceae, while non-responder microbiomes were enriched in Bacteroidales. They further subdivided patients into microbiome “types,” where the Type 1 cluster consisted solely of responders while Type 2 included a mix of responders and non-responders. The study herein sought to verify the findings of Gopalakrishnan et al (2018) and define a signature for the design of a microbiome therapeutic. The signature was validated with a new cohort of patients.

In conclusion, analysis of the validation dataset shows that responders were enriched in Ruminococcaceae, as defined herein, but non-responders were not enriched in Bacteroidia. Using a clade-based relative abundance (12%) of Ruminococcaceae alone achieved the greatest sensitivity and specificity in the validation and combined datasets. Exclusion of stable disease patients from the definition of responder did not reduce the significance of association between Ruminococcaceae and response or alter the 12% threshold. While the association between Ruminococcaceae and responders found in Gopalakrishnan et al (2018) was validated in this analysis, these results contrast with Gopalakrishnan et al (2018) in that non-responders were not found to be enriched in Bacteroidia.

The discoveries disclosed herein therefore demonstrate a method that can be used to identify mircobiomes associated with response to checkpoint inhibitor therapy. Accordingly, this analysis can be used in methods of identifying suitable donors for microbiome compositions to be used, e.g., as adjunct therapies for checkpoint inhibitor therapy or other cancer therapies. In addition to this discovery of a metric for identifying donors with useful GI microbiomes for therapeutic use, the discovery provides early identification of such donors, e.g., so that time and expense wasted on processing donations from unsuitable donors is greatly reduced.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XIII. Tables

Tables 1A-1B: Aggregate Rankings. Aggregate rankings after combining data from all analysis methods are shown. The species rankings are identified in both responder and non-responder patient groups.

TABLE 1A
Aggregate Ranked List - Responders

| Species | Rank |
|---|---|
| Blautia_SC109 | 1 |
| Parabacteroides distasonis | 2 |
| Bilophila_unclassified | 3 |
| Ruminococcus_{—}bicirculans_SC30 | 4 |
| Subdoligranulum_unclassified | 5 |
| Blautia_SC102 | 6 |
| Gemmiger_{—}formicilis_SC193 | 7 |
| Ruminococcus_{—}albus | 8 |
| Bacteroides_{—}dorei | 9 |
| Bifidobacterium_{—}bifidum | 10 |
| Bifidobacterium_{—}longum | 11 |
| Fusicatenibacter_{—}saccharivorans_SC160 | 12 |
| Eubacterium_{—}biforme | 13 |
| Roseburia_{—}faecis_SC53 | 14 |
| Lachnospiraceae_bacterium_5_1_63FAA (Anaerostipes hadrus) | 15 |
| Gemmiger_{—}formicilis_SC141 | 16 |
| Ruminococcus_{—}bromii | 17 |
| Alistipes_{—}senegalensis | 18 |
| Clostridium_SC178 | 19 |
| Odoribacter_{—}splanchnicus | 20 |
| Faecalibacterium_{—}prausnitzii | 21 |
| Clostridium_SC64 | 22 |
| Blautia_{—}wexlerae_SC15 | 23 |
| Coprococcus_{—}catus | 24 |
| Clostridium_SC188 | 25 |
| Streptococcus_{—}parasanguinis | 26 |
| Erysipelotrichaceae_bacterium_21_3 | 27 |
| Barnesiella_{—}intestinihominis | 28 |
| Clostridium_SC26 | 29 |
| Clostridium_{—}lavalense_SC43 | 30 |
| Blautia_{—}faecis_SC4 | 31 |
| Streptococcus_{—}australis | 32 |
| Collinsella_{—}aerofaciens | 33 |
| Clostridium_SC92 | 34 |

TABLE 1B
Aggregate Ranked List - Non-Responders

| Species | Rank |
|---|---|
| Bacteroides_{—}thetaiotaomicron | 1 |
| Collinsella_unclassified | 2 |
| Ruminococcus_{—}torques | 3 |
| Bacteroides_{—}vulgatus | 4 |
| Anaerotruncus_{—}colihominis | 5 |
| Escherichia_{—}coli | 6 |
| Prevotella_{—}copri | 7 |
| Bacteroides_{—}massiliensis | 8 |
| Paraprevotella_{—}clara | 9 |
| Paraprevotella_{—}xylaniphila | 10 |
| Bacteroides_{—}xylanisolvens | 11 |
| Bacteroides_{—}coprocola | 12 |
| Ruminococcus_{—}gnavus | 13 |
| Bilophila_{—}wadsworthia | 14 |
| Parabacteroides_{—}merdae | 15 |
| Collinsella_{—}aerofaciens | 16 |
| Lachnospiraceae_bacterium_2_1_58FAA | 17 |
| Paraprevotella_unclassified | 18 |
| Klebsiella_{—}pneumoniae | 19 |
| Adlercreutzia_{—}equolifaciens | 20 |
| Escherichia_unclassified | 21 |
| Flavonifractor_SC129 | 22 |
| Clostridium_{—}aldenense_SC114 | 23 |
| Lachnospiraceae_bacterium_3_1_46FAA | 24 |
| Holdemania_{—}filiformis | 25 |
| Ruminococcaceae_bacterium_D16 | 26 |
| Blautia_{—}faecis_SC4 | 27 |
| Clostridium_{—}bolteae | 28 |
| Lachnospiraceae_bacterium_1_1_57FAA | 29 |
| Veillonella_{—}parvula | 30 |
| Lachnospiraceae_bacterium_7_1_58FAA | 31 |
| Veillonella_unclassified | 32 |
| Parabacteroides_{—}distasonis | 33 |
| Roseburia_{—}intestinalis | 34 |
| Bacteroides_{—}faecis | 35 |
| Dialister_{—}invisus | 36 |
| Eubacterium_{—}eligens | 37 |

Tables 2A-2B. Differential Prevalence Rankings. Differential prevalence rankings are shown. The species are ranked among responder and non-responder patient groups.

TABLE 2A
Differential Prevalence - Responders

| Species | Rank |
|---|---|
| Parabacteroides distasonis | 1 |
| Blautia_SC109 | 2 |
| Blautia_SC102 | 3 |
| Bacteroides_{—}dorei | 4 |

TABLE 2B
Differential Prevalence - Non-Responders

| Species | Rank |
|---|---|
| Anaerotruncus_{—}colihominis | 1 |
| Parabacteroides_{—}merdae | 2 |

Tables 3A-3B. LDA Abundance Rankings. Linear Discriminant Analysis (LDA) abundance rankings are shown. The species are ranked among responder and non-responder patient groups.

TABLE 3A
LDA Abundance - Responders

| Species | Rank |
|---|---|
| Ruminococcus_{—}bicirculans_SC30 | 1 |
| Ruminococcus_{—}albus | 2 |
| Blautia_SC102 | 3 |
| Gemmiger_{—}formicilis_SC141 | 4 |
| Gemmiger_{—}formicilis_SC193 | 5 |
| Clostridium_SC188 | 6 |
| Subdoligranulum_unclassified | 7 |
| Clostridium_{—}lavalense_SC43 | 8 |
| Blautia_{—}faecis_SC4 | 9 |
| Streptococcus_{—}australis | 10 |
| Clostridium_SC92 | 11 |
| Clostridium_sp_L2_50 | 12 |
| Faecalibacterium_{—}prausnitzii | 13 |
| Eubacterium_{—}siraeum | 14 |
| Clostridium_SC125 | 15 |
| Blautia_SC109 | 16 |
| Ruminococcus_{—}bromii | 17 |
| Lachnospiraceae_bacterium_3_1_46FAA | 18 |
| Coprococcus_{—}comes | 19 |
| Erysipelotrichaceae_bacterium_21_3 | 20 |
| Odoribacter_{—}laneus | 21 |
| Dorea_{—}longicatena | 22 |
| Pseudoflavonifractor_{—}capillosus_SC163 | 23 |
| Eubacterium_{—}rectale | 24 |
| Lachnospiraceae_bacterium_3_1_57FAA_CT1 | 25 |

TABLE 3B
LDA Abundance - Non-Responders

| Species | Rank |
|---|---|
| Bacteroides_{—}vulgatus | 1 |
| Bacteroides_{—}xylanisolvens | 2 |
| Lachnospiraceae_bacterium_2_1_58FAA | 3 |
| Ruminococcus_{—}gnavus | 4 |
| Prevotella_{—}copri | 5 |

Tables 4A-4B. LASSO Prevalence Rankings. LASSO prevalence rankings are shown. The species are ranked among responder and non-responder patient groups.

TABLE 4A
Lasso Prevalence - Responders

| Species | Rank |
|---|---|
| Parabacteroides distasonis | 1 |
| Blautia_SC109 | 2 |
| Bacteroides_{—}dorei | 3 |
| Eubacterium_{—}biforme | 4 |
| Alistipes_{—}senegalensis | 5 |
| Clostridium_SC64 | 6 |

TABLE 4B
Lasso Prevalence - Non-Responders

| Species | Rank |
|---|---|
| Collinsella_unclassified | 1 |
| Bacteroides_{—}coprocola | 2 |
| Anaerotruncus_{—}colihominis | 3 |
| Bacteroides_{—}massiliensis | 4 |
| Adlercreutzia_{—}equolifaciens | 5 |

Tables 5A-5B. LASSO Abundance Rankings. LASSO abundance rankings are shown. The species are ranked among responder and non-responder patient groups.

TABLE 5A
Lasso Abundance - Responders

| Species | Rank |
|---|---|
| Blautia_SC109 | 1 |
| Parabacteroides distasonis | 2 |
| Bifidobacterium_{—}bifidum | 3 |
| Subdoligranulum_unclassified | 4 |

TABLE 5B
Lasso Abundance - Non-Responders

| Species | Rank |
|---|---|
| Bacteroides_{—}thetaiotaomicron | 1 |
| Paraprevotella_{—}clara | 2 |
| Bacteroides_{—}massiliensis | 3 |

Tables 6A-6B. Random Forest Prevalence Rankings. Random Forest prevalence rankings are shown. The species are ranked among responder and non-responder patient groups.

TABLE 6A
Random Forest Prevalence - Responders

| Species | Rank |
|---|---|
| Blautia_SC109 | 1 |
| Parabacteroides distasonis | 2 |
| Bifidobacterium_{—}longum | 3 |
| Blautia_SC102 | 4 |
| Erysipelotrichaceae_bacterium_21_3 | 5 |
| Bifidobacterium_{—}bifidum | 6 |
| Odoribacter_{—}splanchnicus | 7 |
| Barnesiella_{—}intestinihominis | 8 |

TABLE 6B
Random Forest Prevalence - Non-Responders

| Species | Rank |
|---|---|
| Escherichia_{—}coli | 1 |
| Bacteroides_{—}thetaiotaomicron | 2 |
| Collinsella_{—}aerofaciens | 3 |
| Bacteroides_{—}coprocola | 4 |
| Klebsiella_{—}pneumoniae | 5 |
| Parabacteroides_{—}merdae | 6 |
| Clostridium_{—}aldenense_SC114 | 7 |
| Bacteroides_{—}massiliensis | 8 |
| Ruminococcaceae_bacterium_D16 | 9 |

Tables 7A-7B. Random Forest Abundance Rankings. Random Forest abundance rankings are shown. The species are ranked among responder and non-responder patient groups.

TABLE 7A
Random Forest Abundance - Responders

| Species | Rank |
|---|---|
| Bilophila_unclassified | 1 |
| Ruminococcus_{—}bromii | 2 |
| Gemmiger_{—}formicilis_SC193 | 3 |
| Roseburia_{—}faecis_SC53 | 4 |
| Clostridium_SC178 | 5 |
| Blautia_{—}wexlerae_SC15 | 6 |
| Streptococcus_{—}parasanguinis | 7 |
| Bifidobacterium_{—}longum | 8 |
| Odoribacter_{—}splanchnicus | 9 |
| Blautia_SC109 | 10 |
| Collinsella_{—}aerofaciens | 11 |
| Fusicatenibacter_{—}saccharivorans_SC160 | 12 |
| Eubacterium_{—}eligens | 13 |

TABLE 7B
Random Forest Abundance - Non-Responders

| Species | Rank |
|---|---|
| Ruminococcus_{—}torques | 1 |
| Paraprevotella_{—}xylaniphila | 2 |
| Bacteroides_{—}thetaiotaomicron | 3 |
| Paraprevotella_unclassified | 4 |
| Bilophila_{—}wadsworthia | 5 |
| Ruminococcus_{—}gnavus | 6 |
| Flavonifractor_SC129 | 7 |
| Lachnospiraceae_bacterium_3_1_46FAA | 8 |
| Bacteroides_{—}massiliensis | 9 |
| Clostridium_{—}bolteae | 10 |
| Lachnospiraceae_bacterium_1_1_57FAA | 11 |

Tables 8A-8B. Random Forest abunQ Rankings. Random Forest abunQ rankings are shown. The species are ranked among responder and non-responder patient groups.

TABLE 8A
Random Forest abunQ - Responders

| Species | Rank |
|---|---|
| Subdoligranulum_unclassified | 1 |
| Fusicatenibacter_{—}saccharivorans_SC160 | 2 |
| Gemmiger_{—}formicilis_SC193 | 3 |
| Lachnospiraceae_bacterium_5_1_63FAA | 4 |
| Faecalibacterium_{—}prausnitzii | 5 |
| Coprococcus_{—}catus | 6 |
| Blautia_SC109 | 7 |
| Clostridium_SC26 | 8 |

TABLE 8B
Random Forest abunQ - Non-Responders

| Species | Rank |
|---|---|
| Prevotella_{—}copri | 1 |
| Bilophila_{—}wadsworthia | 2 |
| Ruminococcus_{—}gnavus | 3 |
| Escherichia_{—}coli | 4 |
| Escherichia_unclassified | 5 |
| Anaerotruncus_{—}colihominis | 6 |
| Bacteroides_{—}thetaiotaomicron | 7 |
| Holdemania_{—}filiformis | 8 |
| Klebsiella_{—}pneumoniae | 9 |
| Blautia_{—}faecis_SC4 | 10 |
| Veillonella_{—}parvula | 11 |
| Lachnospiraceae_bacterium_7_1_58FAA | 12 |
| Veillonella_unclassified | 13 |
| Parabacteroides_{—}distasonis | 14 |
| Roseburia_{—}intestinalis | 15 |
| Bacteroides_{—}faecis | 16 |
| Dialister_{—}invisus | 17 |
| Eubacterium_{—}eligens | 18 |
| Clostridium_{—}bolteae | 19 |

Table 9. Data Types and Analysis Methods. The three data types and four analysis methods applied to each type of data is shown. Analysis methods applied to a specific data type is marked with an “X”.

TABLE 9

Data type

| | | | Quantile |
|---|---|---|---|
| | | | normalized |
| Method | Prevalence | Abundance | abundance |
| Fisher's exact test | X | — | — |
| Lasso regression | X | X | X |
| Random forest | X | X | X |
| Linear discriminant | — | X | — |
| analysis | | | |

Table 10. Species Call Information. Species calls for bacteria identified in the examples are provided. Bacteria were identified by percent identity to known full length 16S rDNA sequences.

“PCT ID” refers to the percent identity of a 16S rDNA sequence of the species identified to the 16S rDNA sequence of the associated NCBI call (NR Lookup). “Scientific Name” refers to the NCBI name associated with the sequence.

TABLE 10

| Species | PCT ID | NR Lookup | Scientific Name |
|---|---|---|---|
| Parabacteroides_unclassified | 98.9 | NR_074376 | Parabacteroides distasonis |
| Parabacteroides_unclassified | 98.7 | NR_041342 | Parabacteroides distasonis |
| Bifidobacterium_{—}bifidum | 99.9 | NR_118793 | Bifidobacterium bifidum |
| Bifidobacterium_{—}bifidum | 99.9 | NR_044771 | Bifidobacterium bifidum |
| Bifidobacterium_{—}bifidum | 99.9 | NR_117505 | Bifidobacterium bifidum |
| Bifidobacterium_{—}bifidum | 99.9 | NR_113873 | Bifidobacterium bifidum |
| Subdoligranulum_unclassified | 99.3 | NR_104846 | Gemmiger formicilis |
| Subdoligranulum_unclassified | 99.3 | NR_028997 | Subdoligranulum variabile |
| Bacteroides_{—}dorei | 99.9 | NR_041351 | Bacteroides dorei |
| Bacteroides_{—}dorei | 97.2 | NR_074515 | Bacteroides vulgatus |
| Bacteroides_{—}dorei | 97.4 | NR_112946 | Bacteroides vulgatus |
| Eubacterium_{—}biforme | 99.1 | NR_044731 | Holdemanella biformis |
| Alistipes_{—}senegalensis | 100 | NR_118219 | Alistipes senegalensis JC50 |
| Fusicatenibacter_{—}saccharivorans_SC160 | 99.6 | NR_114326 | Fusicatenibacter saccharivorans |
| Lachnospiraceae_bacterium_5_1_63FAA | 99.9 | NR_117138 | Anaerostipes hadrus |
| Lachnospiraceae_bacterium_5_1_63FAA | 99.8 | NR_117139 | Anaerostipes hadrus |
| Lachnospiraceae_bacterium_5_1_63FAA | 99 | NR_104799 | Anaerostipes hadrus |
| Faecalibacterium_{—}prausnitzii | 98 | NR_028961 | Faecalibacterium prausnitzii |
| Coprococcus_{—}catus | 98.1 | NR_024750 | Coprococcus catus |
| Clostridium_SC26 | 98.4 | NR_151982 | Agathobaculum butyriciproducens |
| Clostridium_SC26 | 98.3 | NR_152060 | Butyricicoccus faecihominis |
| Bifidobacterium_{—}longum | 100 | NR_043437 | Bifidobacterium longum subsp. |
| | | | infantis |
| Bifidobacterium_{—}longum | 99.5 | NR_145535 | Bifidobacterium longum subsp. |
| | | | suillum |
| Bifidobacterium_{—}longum | 99.1 | NR_117506 | Bifidobacterium longum |
| Bifidobacterium_{—}longum | 97.6 | NR_040783 | Bifidobacterium breve |
| Bifidobacterium_{—}longum | 98 | NR_044691 | Bifidobacterium longum |
| Bifidobacterium_{—}longum | 97.5 | NR_044693 | Bifidobacterium longum subsp. suis |
| Erysipelotrichaceae_bacterium_21_3 | 98.3 | NR_029164 | Clostridium innocuum |
| Odoribacter_{—}splanchnicus | 100 | NR_074535 | Odoribacter splanchnicus |
| Odoribacter_{—}splanchnicus | 99.9 | NR_113075 | Odoribacter splanchnicus |
| Odoribacter_{—}splanchnicus | 99 | NR_044636 | Odoribacter splanchnicus |
| Barnesiella_{—}intestinihominis | 100 | NR_041668 | Barnesiella intestinihominis YIT 11860 |
| Barnesiella_{—}intestinihominis | 100 | NR_113073 | Barnesiella intestinihominis |

Table 11: Species Call Information. Species calls are provided for bacteria belonging to one or more species that are phylogenetic descendants of the MRCA of Faecalibacterium prausnitzii and Flavonifractor plautii. “Assigned Name” refers to the NCBI name associated with the sequence. Full length 16S rDNA sequences are listed for each species identified.

TABLE 11

| Identifier | Assigned Name | 16S Sequence |
|---|---|---|
| GCF_000154325 | Eubacterium siraeum | CAAAGAGTTTGATCCTGGCTCAGGACGAACGCTGGCGGCGCGCCTAACACATGCAAG |
| | | TCGAACGGTGAAGAGGAGCTTGCTCCTCGGATCAGTGGCGGACGGGTGAGTAACACG |
| | | TGAGCAACCTGGCTCTAAGAGGGGGACAACAGTTGGAAACGACTGCTAATACCGCAT |
| | | AACGTATCGGGATGGCATCTTCCTGATACCAAAGATTTTATCGCTTAGAGATGGGCTC |
| | | GCGTCTGATTAGATAGTTGGCGGGGTAACGGCCCACCAAGTCGACGATCAGTAGCCG |
| | | GACTGAGAGGTTGAACGGCCACATTGGGACTGAGACACGGCCCAGACTCCTACGGGA |
| | | GGCAGCAGTGGGGGATATTGGACAATGGGGGCAACCCTGATCCAGCGACGCCGCGTG |
| | | AGGGAAGAAGGTTTTCGGATTGTAAACCTCTGTTGACGGAGNNNNNNNTGATGGTAT |
| | | CCGTTTAGAAAGCCACGGCTAACTACGTGCCAGCAGCCGCGGTAATACGTAGGTGGC |
| | | AAGCGTTGTCCGGAATTACTGGGTGTAAAGGGAGTGTAGGCGGGATATCAAGTCAGA |
| | | AGTGAAAATTACGGGCTCAACTCGTAACCTGCTTTTGAAACTGACATTCTTGAGTGAA |
| | | GTAGAGGCAAGCGGAATTCCTAGTGTAGCGGTGAAATGCGTAGATATTAGGAGGAAC |
| | | ACCAGTGGCGAAGGCGGCTTGCTGGGCTTTTACTGACGCTGAGGCTCGAAAGCGTGG |
| | | GGAGCAAACAGGATTAGATACCCTGGTAGTCCACGCCGTAAACGATGATTACTAGGT |
| | | GTGGGGGGATTGACCCCTTCCGTGCCGGAGTAAACACAATAAGTAATCCACCTGGGG |
| | | AGTACGACCGCAAGGTTGAAACTCAAAGGAATTGACGGGGGCCCGCACAAGCAGTG |
| | | GAGTATGTGGTTTAATTCGACGCAACGCGAAGAACCTTACCAGGTCTTGACATCGAGT |
| | | GACCGCCTAAGAGATTAGGCTTTCCCTTCGGGGACACAAAGACAGGTGGTGCATGGT |
| | | TGTCGTCAGCTCGTGTCGTGAGATGTTGGGTTAAGTCCCGCAACGAGCGCAACCCTTA |
| | | TCATTAGTTGCTACGCAAGAGCACTCTAATGAGACTGCCGTTGACAAAACGGAGGAA |
| | | GGTGGGGATGACGTCAAATCATCATGCCCTTTATGACCTGGGCTACACACGTACTACA |
| | | ATGGCGTTTAACAAAGAGAAGCAAAGCCGCGAGGCAGAGCAAATCTCCAAAAAACG |
| | | TCTCAGTTCGGATTGTAGGCTGCAACTCGCCTACATGAAGTCGGAATTGCTAGTAATC |
| | | GTAGGTCAGCATACTACGGTGAATACGTTCCCGGGCCTTGTACACACCGCCCGTCAAA |
| | | CCATGAGAGTTGGCAACACCCGAAGTCGGTAGTCTAACCGCAAGGAGGACGCCGCCG |
| | | AAGGTGGGGTTGATGATTAGGGTTAAGTCGTAACAAGGTAGCCGTATCGGAAGGTGC |
| | | GGCTGGATCACCTCCTTT (SEQ ID NO: 108) |
| GCF_000154345 | Clostridium leptum | TTTAGAGAGTTTGATCCTGGCTCAGGACGAACGCTGGCGGCGTGCCTAACACATGCA |
| | | AGTCGAACGGAGTTAAATTCGACACCCGAGTATCCGGCCGGGAGGCGGGGTGCTGGG |
| | | GGTTGGATTTAACTTAGTGGCGGACGGGTGAGTAACGCGTGAGTAACCTGCCTTTCAG |
| | | AGGGGGATAACGTTCTGAAAAGAACGCTAATACCGCATAACATCAATTTATCGCATG |
| | | ATAGGTTGATCAAAGGAGCAATCCGCTGGAAGATGGACTCGCGTCCGATTAGCCAGT |
| | | TGGCGGGGTAACGGCCCACCAAAGCGACGATCGGTAGCCGGACTGAGAGGTTGAAC |
| | | GGCCACATTGGGACTGAGACACGGCCCAGACTCCTACGGGAGGCAGCAGTGGGGGAT |
| | | ATTGCACAATGGGGGAAACCCTGATGCAGCAACGCCGCGTGAGGGAAGAAGGTTTTC |
| | | GGATTGTAAACCTCTGTTCTTAGTGACGATAATGACGGTAGCTAAGGAGAAAGCTCC |
| | | GGCTAACTACGTGCCAGCAGCCGCGGTAATACGTAGGGAGCGAGCGTTGTCCGGATT |
| | | TACTGGGTGTAAAGGGTGCGTAGGCGGCGAGGCAAGTCAGGCGTGAAATCTATGGGC |
| | | TTAACCCATAAACTGCGCTTGAAACTGTCTTGCTTGAGTGAAGTAGAGGTAGGCGGA |
| | | ATTCCCGGTGTAGCGGTGAAATGCGTAGAGATCGGGAGGAACACCAGTGGCGAAGGC |
| | | GGCCTACTGGGCTTTAACTGACGCTGAAGCACGAAAGCATGGGTAGCAAACAGGATT |
| | | AGATACCCTGGTAGTCCATGCCGTAAACGATGATTACTAGGTGTGGGGGGTCTGACC |
| | | CCCTCCGTGCCGCAGTTAACACAATAAGTAATCCACCTGGGGAGTACGGCCGCAAGG |
| | | TTGAAACTCAAAGGAATTGACGGGGGCCCGCACAAGCAGTGGAGTATGTGGTTTAAT |
| | | TCGAAGCAACGCGAAGAACCTTACCAGGTCTTGACATCCGTCTAACGAAGCAGAGAT |
| | | GCATTAGGTGCCCTTCGGGGAAAGGCGAGACAGGTGGTGCATGGTTGTCGTCAGCTC |
| | | GTGTCGTGAGATGTTGGGTTAAGTCCCGCAACGAGCGCAACCCTTGTTTCTAGTTGCT |
| | | ACGCAAGAGCACTCTAGAGAGACTGCCGTTGACAAAACGGAGGAAGGTGGGGACGA |
| | | CGTCAAATCATCATGCCCCTTATGACCTGGGCCACACACGTACTACAATGGCTGTAAA |
| | | CAGAGGGAAGCAAAGCCGCGAGGTGGAGCAAAACCCTAAAAGCAGTCCCAGTTCGG |
| | | ATCGCAGGCTGCAACCCGCCTGCGTGAAGTCGGAATTGCTAGTAATCGCGGATCAGC |
| | | ATGCCGCGGTGAATACGTTCCCGGGCCTTGTACACACCGCCCGTCACACCATGGGAG |
| | | CCGGTAATACCCGAAGCCAGTAGTTCAACCGCAAGGAGAGCGCTGTCGAAGGTAGGA |
| | | TTGGCGACTGGGGTGAAGTCGTAACAAGGTAGCCGTATCGGAAGGTGCGGCTGGATC |
| | | ACCTCCTTT (SEQ ID NO: 109) |
| GCF_000154565 | Anaerotruncus | CAAAGAGTTTGATCCTGGCTCAGGACGAACGCTGGCGGCGCGCCTAACACATGCAAG |
| | colihominis | TCGAACGGAGCTTACGTTTTGAAGTTTTCGGATGGATGAATGTAAGCTTAGTGGCGGA |
| | | CGGGTGAGTAACACGTGAGCAACCTGCCTTTCAGAGGGGGATAACAGCCGGAAACGG |
| | | CTGCTAATACCGCATGATGTTGCGGGGGCACATGCCCCTGCAACCAAAGGAGCAATC |
| | | CGCTGAAAGATGGGCTCGCGTCCGATTAGCCAGTTGGCGGGGTAACGGCCCACCAAA |
| | | GCGACGATCGGTAGCCGGACTGAGAGGTTGAACGGCCACATTGGGACTGAGACACGG |
| | | CCCAGACTCCTACGGGAGGCAGCAGTGGGGGATATTGCACAATGGGCGAAAGCCTGA |
| | | TGCAGCGACGCCGCGTGAGGGAAGACGGTCTTCGGATTGTAAACCTCTGTCTTTGGG |
| | | GAAGAAAATGACGGTACCCAAAGAGGAAGCTCCGGCTAACTACGTGCCAGCAGCCG |
| | | CGGTAATACGTAGGGAGCAAGCGTTGTCCGGAATTACTGGGTGTAAAGGGAGCGTAG |
| | | GCGGGATGGCAAGTAGAATGTTAAATCCATCGGCTCAACCGGTGGCTGCGTTCTAAA |
| | | CTGCCGTTCTTGAGTGAAGTAGAGGCAGGCGGAATTCCTAGTGTAGCGGTGAAATGC |
| | | GTAGATATTAGGAGGAACACCAGTGGCGAAGGCGGCCTGCTGGGCTTTAACTGACGC |
| | | TGAGGCTCGAAAGCGTGGGGAGCAAACAGGATTAGATACCCTGGTAGTCCACGCCGT |
| | | AAACGATGATTACTAGGTGTGGGGGGACTGACCCCTTCCGTGCCGCAGTTAACACAA |
| | | TAAGTAATCCACCTGGGGAGTACGGCCGCAAGGTTGAAACTCAAAGGAATTGACGGG |
| | | GGCCCGCACAAGCAGTGGAGTATGTGGTTTAATTCGAAGCAACGCGAAGAACCTTAC |
| | | CAGGTCTTGACATCGGATGCATAGCCTAGAGATAGGTGAAGCCCTTCGGGGCATCCA |
| | | GACAGGTGGTGCATGGTTGTCGTCAGCTCGTGTCGTGAGATGTTGGGTTAAGTCCCGC |
| | | AACGAGCGCAACCCTTATTATTAGTTGCTACGCAAGAGCACTCTAATGAGACTGCCGT |
| | | TGACAAAACGGAGGAAGGTGGGGATGACGTCAAATCATCATGCCCCTTATGACCTGG |
| | | GCTACACACGTACTACAATGGCACTAAAACAGAGGGCGGCGACACCGCGAGGTGAA |
| | | GCGAATCCCGAAAAAGTGTCTCAGTTCAGATTGCAGGCTGCAACCCGCCTGCATGAA |
| | | GTCGGAATTGCTAGTAATCGCGGATCAGCATGCCGCGGTGAATACGTTCCCGGGCCTT |
| | | GTACACACCGCCCGTCACACCATGGGAGTCGGTAACACCCGAAGCCAGTAGCCTAAC |
| | | CGCAAGGGGGGCGCTGTCGAAGGTGGGATTGATGACTGGGGTGAAGTCGTAACAAG |
| | | GTAGCCGTATCGGAAGGTGCGGCTGGATCACCTCCTTT (SEQ ID NO: 110) |
| GCF_000157955 | Subdoligranulum | NAAGAAGGTTTTCGGATTGTAAACTCCTGTCGTTAGGGACGAATCTTGACGGTACCTA |
| | variabile | ACAAGAAAGCACCGGCTAACTACGTGCCAGCAGCCGCGGTAAAACGTAGGGTGCAA |
| | | GCGTTGTCCGGAATTACTGGGTGTAAAGGGAGCGCAGGCGGACCGGCAAGTTGGAAG |
| | | TGAAATCTATGGGCTCAACCCATAAATTGCTTTCAAAACTGCTGGCCTTGAGTAGTGC |
| | | AGAGGTAGGTGGAATTCCCGGTGTAGCGGTGGAATGCGTAGATATCGGGAGGAACAC |
| | | CAGTGGCGAAGGCGACCTACTGGGCACCAACTGACGCTGAGGCTCGAAAGCATGGGT |
| | | AGCAAACAGGATTAGATACCCTGGTAGTCCATGCCGTAAACGATGATTACTAGGTGT |
| | | TGGAGGATTGACCCCTTCAGTGCCGCAGTTAACACAATAAGTAATCCACCTGGGGAG |
| | | TACGACCGCAAGGTTGAAACTCAAAGGAATTGACGGGGGCCCGCACAAGCAGTGGA |
| | | GTATGTGGTTTAATTCGAAGCAACGCGAAGAACCTTACCAGGTCTTGACATCCGATGC |
| | | ATAGTGCAGAGATGCATGAAGTCCTTCGGGACATCGAGACAGGTGGTGCATGGTTGT |
| | | CGTCAGCTCGTGTCGTGAGATGTTGGGTTAAGTCCCGCAACGAGCGCAACCCTTATTG |
| | | CCAGTTACTACGCAAGAGGACTCTGGCGAGACTGCCGTTGACAAAACGGAGGAAGGT |
| | | GGGGATGACGTCAAATCATCATGCCCTTTATGACCTGGGCTACACACGTACTACAATG |
| | | GCGTTTAACAAAGAGATGCAAGACCGCGAGGTGGAGCAAAACTCAAAAACAACGTC |
| | | TCAGTTCAGATTGCAGGCTGCAACTCGCCTGCATGAAGTCGGAATTGCTAGTAATCGC |
| | | GGATCAGCATGCCGCGGTGAATACGTTCCCGGGCCTTGTACACACCGCCCGTCACACC |
| | | ATGAGAGCCGGGGGGACCCGAAGTCGGTAGTCTAACCGCAAGGAGGACGCCGCCGA |
| | | AGGTAAAACTGGTGATTGGGGTGAAGTCGTAACAAGGTAGCCGTATCGGAAGGTGCG |
| | | GCTGGATCACCTCCTTT (SEQ ID NO: 111) |
| GCF_000158655 | Clostridium | ATTAAGAGTTTGATCCTGGCTCAGGACGAACGCTGGCGGCGCGCCTAACACATGCAA |
| | methylpentosum | GTCGAACGGAGTTGTTTTGGAGAAGCCCTTCGGGGTGGAACTGATTCAACTTAGTGGC |
| | | GGACGGGTGAGTAACACGTGAGCAACCTGCCTTACAGAGGGGAATAACGTTTGGAAA |
| | | CGAACGCTAATACCGCATAACATAACGGAATCGCATGGTTTTGTTATCAAAGATTATA |
| | | TCGCTGTAAGATGGGCTCGCGTCTGATTAGATAGTTGGTGAGGTAATGGCTCACCAAG |
| | | TCGACGATCAGTAGCCGGACTGAGAGGTTGAACGGCCACATTGGGACTGAGACACGG |
| | | CCCAGACTCCTACGGGAGGCAGCAGTGGGGAATATTGCACAATGGGGGAAACCCTGA |
| | | TGCAGCGACGCCGCGTGAAGGAAGAAGGCCTTCGGGTTGTAAACTTCTGTCTTCAGG |
| | | GACGATAATGACGGTACCTGAGGAGGAAGCTCCGGCTAACTACGTGCCAGCAGCCGC |
| | | GGTAATACGTAGGGAGCGAGCGTTGTCCGGAATTACTGGGTGTAAAGGGAGCGTAGG |
| | | CGGGATTGCAAGTTGAATGTGAAATCTATGGGCTTAACCCATAAACTGCGTTCAAAA |
| | | CTGCAGTTCTTGAGTGAAGTAGAGGCAGGCGGAATTCCTAGTGTAGCGGTGAAATGC |
| | | GTAGATATTAGGAGGAACACCAGTGGCGAAGGCGGCCTGCTGGGCTTTAACTGACGC |
| | | TGAGGCTCGAAAGCGTGGGTAGCAAACAGGATTAGATACCCTGGTAGTCCACGCCGT |
| | | AAACGATGATTACTAGGTGTAGGGGGGTCAACCTTCTGTGCCGGAGTTAACACAATA |
| | | AGTAATCCACCTGGGGAGTACGACCGCAAGGTTGAAACTCAAAGGAATTGACGGGGG |
| | | CCCGCACAAGCAGTGGAGTATGTGGTTTAATTCGAAGCAACGCGAAGAACCTTACCA |
| | | GGTCTTGACATCCAACTAACGAAGTAGAGATACATTAGGTGCCCTTCGGGGAAAGTT |
| | | GAGACAGGTGGTGCATGGTTGTCGTCAGCTCGTGTCGTGAGATGTTGGGTTAAGTCCC |
| | | GCAACGAGCGCAACCCTTACATTTAGTTGCTACGCAAGAGCACTCTAGATGGACTGC |
| | | CGTTGACAAAACGGAGGAAGGTGGGGATGACGTCAAATCATCATGCCCCTTATGACC |
| | | TGGGCTACACACGTACTACAATGGCTATTAACAGAGGGAAGCAAAACAGTGATGTGG |
| | | AGCAAACCCCTAAAAATAGTCTCAGTTCGGATTGTAGGCTGCAACTCGCCTACATGA |
| | | AGCCGGAATTGCTAGTAATCGCGGATCAGCATGCCGCGGTGAATACGTTCCCGGGCC |
| | | TTGTACACACCGCCCGTCACACCATGAGAGTTGGCAACACCCGAAGTCAGTAGTCTA |
| | | ACCGCAAGGAGGACGCTGCCGAAGGTGGGGTTGATGATTAGGGTGAAGTCGTAACAA |
| | | GGTAGCCGTATCGGAAGGTGCGGCTGGATCACCTCCTTT (SEQ ID NO: 5) |
| GCF_000169255 | Pseudoflavonifractor | TATTGAGAGTTTGATCCTGGCTCAGGATGAACGCTGGCGGCGTGCTTAACACATGCAA |
| | capillosus | GTCGAACGGAGAGCTCATGACAGAGGATTCGTCCAATGGATTGGGTTTCTTAGTGGC |
| | | GGACGGGTGAGTAACGCGTGAGGAACCTGCCTCGGAGTGGGGAATAACAGTCCGAA |
| | | AGGACTGCTAATACCGCATAATGCAGCTGAGTCGCATGACACTGGCTGCCAAAGATT |
| | | TATCGCTCTGAGATGGCCTCGCGTCTGATTAGCTAGTTGGCGGGGTAACGGCCCACCA |
| | | AGGCGACGATCAGTAGCCGGACTGAGAGGTTGGCCGGCCACATTGGGACTGAGACAC |
| | | GGCCCAGACTCCTACGGGAGGCAGCAGTGGGGAATATTGGGCAATGGGCGCAAGCCT |
| | | GACCCAGCAACGCCGCGTGAAGGATGAAGGCTTTCGGGTTGTAAACTTCTTTTATCAG |
| | | GGACGAAATAAATGACGGTACCTGATGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGACTGCAAGTCAGGTGTGAAAACCACGGGCTCAACCTGTGGCCTGCATTT |
| | | GAAACTGTAGTTCTTGAGTGCTGGAGAGGCAATCGGAATTCCGTGTGTAGCGGTGAA |
| | | ATGCGTAGATATACGGAGGAACACCAGTGGCGAAGGCGGATTGCTGGACAGTAACTG |
| | | ACGCTGAGGCGCGAAAGCGTGGGGAGCAAACAGGATTAGATACCCTGGTAGTCCACG |
| | | CCGTAAACGATGGATACTAGGTGTGGGGGGACTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCGACTAACGAAGCAGAGATGCATTAGGTGCCCTTCGGGG |
| | | AAAGTCGAGACAGGTGGTGCATGGTTGTCGTCAGCTCGTGTCGTGAGATGTTGGGTTA |
| | | AGTCCCGCAACGAGCGCAACCCTTATTGTTAGTTGCTACGCAAGAGCACTCTAGCGA |
| | | GACTGCCGTTGACAAAACGGAGGAAGGTGGGGACGACGTCAAATCATCATGCCCCTT |
| | | ATGTCCTGGGCCACACACGTACTACAATGGTGGTTAACAGAGGGAAGCAATGCCGCG |
| | | AGGTGGAGCAAATCCCTAAAAGCCATCCCAGTTCGGATTGCAGGCTGAAACCCGCCT |
| | | GTATGAAGTTGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACCGCAAGGAGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 6) |
| GCF_000178115 | Ethanoligenens | TTGGAGAGTTTGATCCTGGCTCAGGACGAACGCTGGCGGCGCGCCTAACACATGCAA |
| | harbinense | GTCGAGCGGAGTCCTTCGGGACTTAGCGGCGGACGGGTGAGTAACGCGTGAGCAACC |
| | | TGGCCTTCAGAGGGGGATAACGTCTGGAAACGGACGCTAATACCGCATGACATGGCG |
| | | GAGTCGCATGGCTCTGCCATCAAAGGAGTAATCCGCTGAGGGATGGGCTCGCGTCCG |
| | | ATTAGGTAGTTGGTGAGGTAACGGCTCACCAAGCCCGCGATCGGTAGCCGGACTGAG |
| | | AGGTTGGCCGGCCACATTGGGACTGAGACACGGCCCAGACTCCTACGGGAGGCAGCA |
| | | GTGGGGGATATTGCACAATGGAGGAAACTCTGATGCAGCGACGCCGCGTGAGGGAA |
| | | GAAGGTCTTCGGATTGTAAACCTCTGTCTTTGGGGACGAATCAATGACGGTACCCAAG |
| | | GAGGAAGCCACGGCTAACTACGTGCCAGCAGCCGCGGTAATACGTAGGTGGCAAGCG |
| | | TTGTCCGGAATTACTGGGTGTAAAGGGTGCGCAGGCGGGGCGGCAAGTTGGATGTGA |
| | | AAACTCCGGGCTCAACCCGGAGCCTGCATTCAAAACTGTCGCTCTTGAGTGAAGTAG |
| | | AGGCAGGCGGAATTCCCGGTGTAGCGGTGAAATGCGTAGATATCGGGAGGAACACCA |
| | | GTGGCGAAGGCGGCCTGCTGGGCTTTTACTGACGCTGAGGCACGAAAGCATGGGTAG |
| | | CAAACAGGATTAGATACCCTGGTAGTCCATGCCGTAAACGATGATTGCTAGGTGTGG |
| | | GGGGTCTGACCCCTTCCGTGCCGGAGTTAACACAATAAGCAATCCACCTGGGGAGTA |
| | | CGGCCGCAAGGTTGAAACTCAAAGGAATTGACGGGGGCCCGCACAAGCAGTGGAGT |
| | | ATGTGGTTTAATTCGAAGCAACGCGAAGAACCTTACCAGGTCTTGACATCCACCGAAT |
| | | CCCCCAGAGATGGGGGAGTGCCCTTCGGGGAGCGGTGAGACAGGTGGTGCATGGTTG |
| | | TCGTCAGCTCGTGTCGTGAGATGTTGGGTTAAGTCCCGCAACGAGCGCAACCCTTGTG |
| | | AATAGTTGCTACGAAAGAGCACTCTATTCAGACCGCCGTTGACAAAACGGAGGAAGG |
| | | TGGGGATGACGTCAAATCATCATGCCCCTTATGACCTGGGCTACACACGTACTACAAT |
| | | GGCCATCAACAGAGGGAAGCAAGGCCGCGAGGTGGAGCGAACCCCTAAAAATGGTC |
| | | TCAGTTCAGATTGCAGGCTGAAACCCGCCTGCATGAAGATGGAATTGCTAGTAATCG |
| | | CGGATCAGCATGCCGCGGTGAATACGTTCCCGGGCCTTGTACACACCGCCCGTCACAC |
| | | CATGAGAGCCGGGGACACCCGAAGTCGGTTGGGTAACCGTAAGGAGCCCGCCGCCGA |
| | | AGGTGGAATCGGTAATTGGGGTGAAGTCGTAACAAGGTAGCCGTATCGGAAGGTGCG |
| | | GCTGGATCACCTCCTTT (SEQ ID NO: 7) |
| GCF_000179635 | Ruminococcus albus | TATTAAGAGTTTGATCCTGGCTCAGGACGAACGCTGGCGGCACGCTTAACACATGCA |
| | | AGTCGAACGAGCGAAAGAGTGCTTGCACTCTCTAGCTAGTGGCGGACGGGTGAGTAA |
| | | CACGTGAGCAATCTGCCTTTCGGAGAGGGATACCAATTGGAAACGATTGTTAATACCT |
| | | CATAACATAACGAAGCCGCATGACTTTGTTATCAAATGAATTTCGCCGAAAGATGAG |
| | | CTCGCGTCTGATTAGGTAGTTGGTGAGGTAACGGCCCACCAAGCCGACGATCAGTAG |
| | | CCGGACTGAGAGGTTGAACGGCCACATTGGGACTGAGACACGGCCCAGACTCCTACG |
| | | GGAGGCAGCAGTGGGGAATATTGCACAATGGGCGAAAGCCTGATGCAGCGATGCCG |
| | | CGTGAGGGAAGAAGGTTTTAGGATTGTAAACCTCTGTCTTTGGGGACGATAATGACG |
| | | GTACCCAAGGAGGAAGCTCCGGCTAACTACGTGCCAGCAGCCGCGGTAATACGTAGG |
| | | GAGCGAGCGTTGTCCGGAATTACTGGGTGTAAAGGGAGCGTAGGCGGGATTGCAAGT |
| | | CAGGTGTGAAATTTAGGGGCTTAACCCCTGAACTGCACTTGAAACTGTAGTTCTTGAG |
| | | TGAAGTAGAGGTAAGCGGAATTCCTAGTGTAGCGGTGAAATGCGTAGATATTAGGAG |
| | | GAACATCAGTGGCGAAGGCGGCTTACTGGGCTTTAACTGACGCTGAGGCTCGAAAGC |
| | | GTGGGGAGCAAACAGGATTAGATACCCTGGTAGTCCACGCCGTAAACGATGATTACT |
| | | AGGTGTGGGGGGACTGACCCCTTCCGTGCCGCAGTTAACACAATAAGTAATCCACCT |
| | | GGGGAGTACGGCCGCAAGGCTGAAACTCAAAGGAATTGACGGGGACCCGCACAAGC |
| | | AGTGGAGTATGTGGTTTAATTCGAAGCAACGCGAAGAACCTTACCAGGTCTTGACAT |
| | | CGTACGCATAGCATAGAGATATGTGAAATCCCTTCGGGGACGTATAGACAGGTGGTG |
| | | CATGGTTGTCGTCAGCTCGTGTCGTGAGATGTTGGGGTTAAGTCCCGCAACGAGCGCA |
| | | ACCCTTACTGTTAGTTGCTACGCAAGAGCACTCTAGCAGGACTGCCGTTGACAAAACG |
| | | GAGGAAGGTGGGGATGACGTCAAATCATCATGCCCCTTATGACCTGGGCTACACACG |
| | | TACTACAATGGCTGTTAACAGAGGGAAGCAAAACAGTGATGTGGAGCAAAACCCTAA |
| | | AAGCAGTCTTAGTTCGGATTGTAGGCTGCAACCCGCCTACATGAAGTCGGAATTGCTA |
| | | GTAATCGCGGATCAGCATGCCGCGGTGAATACGTTCCCGGGCCTTGTACACACCGCCC |
| | | GTCACGCCATGGGAGTCGGTAACACCCGAAGCCTGTGTTCTAACCGCAAGGAGGAAG |
| | | CAGTCGAAGGTGGGATTGATGACTGGGGTGAAGTCGTAACAAGGTAGCCGTATCGGA |
| | | AGGTGCGGCTGGATCACCTCCTTT (SEQ ID NO: 8) |
| GCF_000210095 | Ruminococcus | TATGAAGAGTTTGATCCTGGCTCAGGACGAACGCTGGCGGCACGCCTAACACATGCA |
| | champanellensis | AGTCGAACGGAGATAAAGACTTCGGTTTTTATCTTAGTGGCGGACGGGTGAGTAACA |
| | | CGTGAGCAACCTGCCTCTGAGAGAGGGATAGCTTCTGGAAACGGATGGTAATACCTC |
| | | ATAACATAGCGGTACCGCATGATACTGCTATCAAAGATTTATCGCTCAGAGATGGGCT |
| | | CGCGTCTGATTAGCTAGATGGTGAGGTAACGGCTCACCATGGCGACGATCAGTAGCC |
| | | GGACTGAGAGGTTGAACGGCCACATTGGGACTGAGACACGGCCCAGACTCCTACGGG |
| | | AGGCAGCAGTGGGGAATATTGCACAATGGGCGCAAGCCTGATGCAGCGATGCCGCGT |
| | | GGAGGAAGAAGGTTTTCGGATTGTAAACTCCTGTCTTAAGGGACGATAATGACGGTA |
| | | CCTTAGGAGGAAGCTCCGGCTAACTACGTGCCAGCAGCCGCGGTAATACGTAGGGAG |
| | | CGAGCGTTGTCCGGAATTACTGGGTGTAAAGGGAGCGTAGGCGGGATTGCAAGTCAG |
| | | ATGTGAAAACTATGGGCTTAACCCATAGACTGCATTTGAAACTGTAGTTCTTGAGTGA |
| | | AGTAGAGGTAAGCGGAATTCCTAGTGTAGCGGTGAAATGCGTAGATATTAGGAGGAA |
| | | CATCGGTGGCGAAGGCGGCTTACTGGGCTTTTACTGACGCTGAGGCTCGAAAGCGTG |
| | | GGGAGCAAACAGGATTAGATACCCTGGTAGTCCACGCTGTAAACGATGATTACTAGG |
| | | TGTGGGGGGACTGACCCCTTCCGTGCCGCAGTTAACACAATAAGTAATCCACCTGGG |
| | | GAGTACGGCCGCAAGGTTGAAACTCAAAGGAATTGACGGGGGCCCGCACAAGCAGT |
| | | GGAGTATGTGGTTTAATTCGAAGCAACGCGAAAAACCTTACCAGGTCTTGACATCGA |
| | | GTGAATGATCTAGAGATAGATCAGTCCTTCGGGACACAAAGACAGGTGGTGCATGGT |
| | | TGTCGTCAGCTCGTGTCGTGAGATGTTGGGTTAAGTCCCGCAACGAGCGCAACCCTTA |
| | | CCTTTAGTTGCTACGCAAGAGCACTCTAGAGGGACTGCCGTTGACAAAACGGAGGAA |
| | | GGTGGGGATGACGTCAAATCATCATGCCCCTTATGACCTGGGCTACACACGTACTACA |
| | | ATGGCAATGAACAGAGGGAAGCAATACAGTGATGTGGAGCAAATCCCCAAAAATTGT |
| | | CCCAGTTCAGATTGTAGGCTGCAACTCGCCTACATGAAGTCGGAATTGCTAGTAATCG |
| | | CAGATCAGCATGCTGCGGTGAATACGTTCCCGGGCCTTGTACACACCGCCCGTCACAC |
| | | CATGGGAGTCGGTAACACCCGAAGCCAGTAGCCTAACCGCAAGGAGGGCGCTGTCGA |
| | | AGGTGGGATTGATGACTGGGGTGAAGTCGTAACAAGGTAGCCGTATCGGAAGGTGCG |
| | | GCTGGATCACCTCCTTT (SEQ ID NO: 9) |
| GCF_000239295 | Flavonifractor plautii | TATTGAGAGTTTGATCCTGGCTCAGGATGAACGCTGGCGGCGTGCTTAACACATGCAA |
| | | GTCGAACGGGGTGCTCATGACGGAGGATTCGTCCAATGGATTGAGTTACCTAGTGGC |
| | | GGACGGGTGAGTAACGCGTGAGGAACCTGCCTTGGAGAGGGGAATAACACTCCGAA |
| | | AGGAGTGCTAATACCGCATGAAGCAGTTGGGTCGCATGGCTCTGACTGCCAAAGATT |
| | | TATCGCTCTGAGATGGCCTCGCGTCTGATTAGCTAGTAGGCGGGGTAACGGCCCACCT |
| | | AGGCGACGATCAGTAGCCGGACTGAGAGGTTGACCGGCCACATTGGGACTGAGACAC |
| | | GGCCCAGACTCCTACGGGAGGCAGCAGTGGGGAATATTGGGCAATGGGCGCAAGCCT |
| | | GACCCAGCAACGCCGCGTGAAGGAAGAAGGCTTTCGGGTTGTAAACTTCTTTTGTCG |
| | | GGGACGAAACAAATGACGGTACCCGACGAATAAGCCACGGCTAACTACGTGCCAGC |
| | | AGCCGCGGTAATACGTAGGTGGCAAGCGTTATCCGGATTTACTGGGTGTAAAGGGCG |
| | | TGTAGGCGGGATTGCAAGTCAGATGTGAAAACTGGGGGCTCAACCTCCAGCCTGCAT |
| | | TTGAAACTGTAGTTCTTGAGTGCTGGAGAGGCAATCGGAATTCCGTGTGTAGCGGTGA |
| | | AATGCGTAGATATACGGAGGAACACCAGTGGCGAAGGCGGATTGCTGGACAGTAACT |
| | | GACGCTGAGGCGCGAAAGCGTGGGGAGCAAACAGGATTAGATACCCTGGTAGTCCAC |
| | | GCCGTAAACGATGGATACTAGGTGTGGGGGGTCTGACCCCCTCCGTGCCGCAGTTAA |
| | | CACAATAAGTATCCCACCTGGGGAGTACGATCGCAAGGTTGAAACTCAAAGGAATTG |
| | | ACGGGGGCCCGCACAAGCGGTGGAGTATGTGGTTTAATTCGAAGCAACGCGAAGAAC |
| | | CTTACCAGGGCTTGACATCCCACTAACGAGGCAGAGATGCGTTAGGTGCCCTTCGGG |
| | | GAAAGTGGAGACAGGTGGTGCATGGTTGTCGTCAGCTCGTGTCGTGAGATGTTGGGTT |
| | | AAGTCCCGCAACGAGCGCAACCCTTATTGTTAGTTGCTACGCAAGAGCACTCTAGCG |
| | | AGACTGCCGTTGACAAAACGGAGGAAGGTGGGGACGACGTCAAATCATCATGCCCCT |
| | | TATGTCCTGGGCCACACACGTACTACAATGGTGGTTAACAGAGGGAGGCAATACCGC |
| | | GAGGTGGAGCAAATCCCTAAAAGCCATCCCAGTTCGGATTGCAGGCTGAAACCCGCC |
| | | TGTATGAAGTTGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACCGCAAGGAGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 10) |
| GCF_000283575 | Oscillibacter | TATAGAGAGTTTGATCCTGGCTCAGGACGAACGCTGGCGGCGTGCTTAACACATGCA |
| | valericigenes | AGTCGAACGGAGCACCCTTGATTGAGGTTTCGGCCAAATGAGAGGAATGCTTAGTGG |
| | | CGGACTGGTGAGTAACGCGTGAGGAACCTGCCTTTCAGAGGGGGACAACAGTTGGAA |
| | | ACGACTGCTAATACCGCATGATACATTTGGGCGACATCGCTTGAATGTCAAAGATTTA |
| | | TCGCTGAAAGATGGCCTCGCGTCTGATTAGATAGTTGGTGAGGTAACGGCCCACCAA |
| | | GTCGACGATCAGTAGCCGGACTGAGAGGTTGACCGGCCACATTGGGACTGAGATACG |
| | | GCCCAGACTCCTACGGGAGGCAGCAGTGGGGAATATTGGGCAATGGACGCAAGTCTG |
| | | ACCCAGCAACGCCGCGTGAAGGAAGAAGGCTTTCGGGTTGTAAACTTCTTTTAAGGG |
| | | GGAAGAGTAGAAGACGGTACCCCTTGAATAAGCCACGGCTAACTACGTGCCAGCAGC |
| | | CGCGGTAATACGTAGGTGGCAAGCGTTGTCCGGATTTACTGGGTGTAAAGGGCGTGT |
| | | AGCCGGGAAGGTAAGTCAGATGTGAAATCTGGGGGCTCAACCTCCAAACTGCATTTG |
| | | AAACTACTTTTCTTGAGTATCGGAGAGGTAATCGGAATTCCTTGTGTAGCGGTGAAAT |
| | | GCGTAGATATAAGGAAGAACACCAGTGGCGAAGGCGGATTACTGGACGACAACTGA |
| | | CGGTGAGGCGCGAAAGCGTGGGGAGCAAACAGGATTAGATACCCTGGTAGTCCACGC |
| | | TGTAAACGATCAATACTAGGTGTGCGGGGACTGACCCCCTGCGTGCCGCAGTTAACA |
| | | CAATAAGTATTGCACCTGGGGAGTACGATCGCAAGGTTGAAACTCAAAGGAATTGAC |
| | | GGGGGCCCGCACAAGCGGTGGATTATGTGGTTTAATTCGAAGCAACGCGAAGAACCT |
| | | TACCAGGACTTGACATCCTACTAACGAGGTAGAGATACGTCAGGTGCCCTTCGGGGA |
| | | AAGTAGAGACAGGTGGTGCATGGTTGTCGTCAGCTCGTGTCGTGAGATGTTGGGTTAA |
| | | GTCCCGCAACGAGCGCAACCCCTATTGTTAGTTGCTACGCAAGAGCACTCTAGCGAG |
| | | ACTGCCGTTGACAAAACGGAGGAAGGTGGGGACGACGTCAAATCATCATGCCCCTTA |
| | | TGTCCTGGGCTACACACGTAATACAATGGCGGTCAACAGAGGGATGCAAAGCCGTGA |
| | | GGTGGAGCGAACCCCTAAAAGCCGTCTCAGTTCGGATCGCAGGCTGCAACTCGCCTG |
| | | CGTGAAGTCGGAATCGCTAGTAATCGCGGATCAGAATGCCGCGGTGAATACGTTCCC |
| | | GGGCCTTGTACACACCGCCCGTCACACCATGAGAGTCGGGAACACCCGAAGTCCGTA |
| | | GCCTAACAGCAATGAGGGCGCGGCCGAAGGTGGGTTTGATAATTGGGGTGAAGTCGT |
| | | AACAAGGTAGCCGTATCGGAAGGTGCGGCTGGATCACCTCCTTT (SEQ ID NO: 11) |
| GCF_000307265 | Oscillibacter | TATAGAGAGTTTGATCCTGGCTCAGGACGAACGCTGGCGGCGTGCTTAACACATGCA |
| | ruminantium | AGTCGAACGGAACACCCTTGACAGAGGTTTCGGCCAATGAAGAGGAATGTTTAGTGG |
| | | CGGACTGGTGAGTAACGCGTGAGGAACCTGCCTTTCAGAGGGGGACAACAGTTGGAA |
| | | ACGACTGCTAATACCGCATGAAGCAGCGAGGGGACATCCCCTTGCTGTCAAAGATTT |
| | | ATCGCTGAAAGATGGCCTCGCGTCTGATTAGCTAGTTGGTGGGGTAACGGCCCACCA |
| | | AGGCGACGATCAGTAGCCGGACTGAGAGGTTGACCGGCCACATTGGGACTGAGATAC |
| | | GGCCCAGACTCCTACGGGAGGCAGCAGTGGGGAATATTGGGCAATGGACGCAAGTCT |
| | | GACCCAGCAACGCCGCGTGAAGGAAGAAGGCTTTCGGGTTGTAAACTTCTTTTAACA |
| | | GGGAAGAGAAGAAGACGGTACCTGTTGAATAAGCCACGGCTAACTACGTGCCAGCA |
| | | GCCGCGGTAATACGTAGGTGGCAAGCGTTGTCCGGATTTACTGGGTGTAAAGGGCGT |
| | | GTAGCCGGGAAGGCAAGTCAGATGTGAAATCTGGAGGCTCAACCTCCAAACTGCATT |
| | | TGAAACTGCTTTTCTTGAGTATCGGAGAGGTAATCGGAATTCCTTGTGTAGCGGTGAA |
| | | ATGCGTAGATATAAGGAAGAACACCAGTGGCGAAGGCGGATTACTGGACGACAACT |
| | | GACGGTGAGGCGCGAAAGCGTGGGGAGCAAACAGGATTAGATACCCTGGTAGTCCA |
| | | CGCTGTAAACGATCAATACTAGGTGTGCGGGGACTGACCCCCTGCGTGCCGCAGTTA |
| | | ACACAATAAGTATTGCACCTGGGGAGTACGATCGCAAGGTTGAAACTCAAAGGAATT |
| | | GACGGGGGCCCGCACAAGCGGTGGATTATGTGGTTTAATTCGAAGCAACGCGAAGAA |
| | | CCTTACCAGGACTTGACATCCTACTAACGAGGTAGAGATACGTCAGGTGCCCTTCGGG |
| | | GAAAGTAGAGACAGGTGGTGCATGGTTGTCGTCAGCTCGTGTCGTGAGATGTTGGGTT |
| | | AAGTCCCGCAACGAGCGCAACCCCTATTGTTAGTTGCTACGCAAGAGCACTCTAGCG |
| | | AGACTGCCGTTGACAAAACGGAGGAAGGTGGGGACGACGTCAAATCATCATGCCCCT |
| | | TATGTCCTGGGCTACACACGTAATACAATGGCGGTCAACAGAGGGATGCAAAGCCGT |
| | | GAGGCAGAGCGAACCCCTAAAAGCCGTCTCAGTTCGGATCGTAGGCTGCAACTCGCC |
| | | TACGTGAAGTCGGAATCGCTAGTAATCGCGGATCAGAATGCCGCGGTGAATACGTTC |
| | | CCGGGCCTTGTACACACCGCCCGTCACACCATGAGAGTCGGGAACACCCGAAGCCCG |
| | | TAGCCTAACTGCAAAGAGGGCGCGGTCGAAGGTGGGTTCGATAATTGGGGTGAAGTC |
| | | GTAACAAGGTAGCCGTATCGGAAGGTGCGGCTGGATCACCTCCTTT (SEQ ID NO: 12) |
| GCF_000383295 | Clostridium | CAAAGGAGCAATCCGCTGAAAGATGGACTCGCGTCCGATTAGCCAGTTGGCGGGGTA |
| | sporosphaeroides | AAGGCCCACCAAAGCGACGATCGGTAGCCGGGTTGAGAGACTGAACGGCCACATTGG |
| | | GACTGAGACACGGCCCAGACTCCTACGGGAGGCAGCAGTGGGGGATATTGCACAATG |
| | | GAGGAAACTCTGATGCAGCAATGCCGCGTGAGGGAAGACGGTCTTCGGATTGTAAAC |
| | | CTCTGTCCTTGGTGAAGATAATGACGGTAGCCAAGGAGGAAGCTCCGGCTAACTACG |
| | | TGCCAGCAGCCGCGGTAATACGTAGGGAGCAAGCGTTGTCCGGATTTACTGGGTGTA |
| | | AAGGGTGCGTAGGCGGCTCTTTAAGTCGGGCGTGAAAGCTGTGGGCTTAACCCACAA |
| | | ATTGCGTTCGAAACTGGAGGGCTTGAGTGAAGTAGAGGTAGGCGGAATTCCCGGTGT |
| | | AGCGGTGAAATGCGTAGAGATCGGGAGGAACACCAGTGGCGAAGGCGGCCTACTGG |
| | | GCTTTAACTGACGCTGAGGCACGAAAGCATGGGTAGCAAACAGGATTAGATACCCTG |
| | | GTAGTCCATGCCGTAAACGATGATTACTAGGTGTGGGGGGTCTGACCCCTTCCGTGCC |
| | | GGAGTTAACACAATAAGTAATCCACCTGGGGAGTACGGCCGCAAGGTTGAAACTCAA |
| | | AGGAATTGACGGGGGCCCGCACAAGCAGTGGAGTATGTGGTTTAATTCGAAGCAACG |
| | | CGAAGAACCTTACCAGGTCTTGACATCCAACTAACGAGGCAGAGATGCATTAGGTGC |
| | | CCTTCGGGGAAAGTTGAGACAGGTGGTGCATGGTTGTCGTCAGCTCGTGTCGTGAGAT |
| | | GTTGGGTTAAGTCCCGCAACGAGCGCAACCCTTGTGATTAGTTGCTACGCAAGAGCA |
| | | CTCTAATCAGACTGCCGTTGACAAAACGGAGGAAGGTGGGGACGACGTCAAATCATC |
| | | ATGCCCCTTATGACCTGGGCTACACACGTACTACAATGGTCGCCAACAGAGGGAAGC |
| | | CA (SEQ ID NO: 13) |
| GCF_000468015 | Ruminococcus | TAAAGAGTTTGATCCTGGCTCAGGACGAACGCTGGCGGCACGCTTAACACATGCAAG |
| | callidus | TCGAACGGAGAACATTGAGCTTGCTTAATGTTCTTAGTGGCGGACGGGTGAGTAACA |
| | | CGTGAGTAACCTGCCTCTGAGAGTGGGATAGCTTCTGGAAACGGATGGTAATACCGC |
| | | ATAACATCATGGATTCGCATGTTTCTGTGATCAAAGATTTATCGCTTAGAGATGGACT |
| | | CGCGTCTGATTAGCTAGTTGGTAAGGTAACGGCTTACCAAGGCGACGATCAGTAGCC |
| | | GGACTGAGAGGTTGATCGGCCACATTGGGACTGAGACACGGCCCAGACTCCTACGGG |
| | | AGGCAGCAGTGGGGAATATTGCACAATGGGGGAAACCCTGATGCAGCGATGCCGCGT |
| | | GGAGGAAGAAGGTTTTCGGATTGTAAACTCCTGTTGAAGAGGACGATAATGACGGTA |
| | | CTCTTTTAGAAAGCTCCGGCTAACTACGTGCCAGCAGCCGCGGTAATACGTAGGGAG |
| | | CGAGCGTTGTCCGGAATTACTGGGTGTAAAGGGAGCGTAGGCGGGATGGCAAGTCAG |
| | | ATGTGAAAACTATGGGCTCAACCCATAGACTGCATTTGAAACTGTTGTTCTTGAGTGA |
| | | GGTAGAGGTAAGCGGAATTCCTGGTGTAGCGGTGAAATGCGTAGAGATCAGGAGGAA |
| | | CATCGGTGGCGAAGGCGGCTTACTGGGCCTTTACTGACGCTGAGGCTCGAAAGCGTG |
| | | GGGAGCAAACAGGATTAGATACCCTGGTAGTCCACGCCGTAAACGATGATTACTAGG |
| | | TGTGGGGGGACTGACCCCTTCCGTGCCGCAGTTAACACAATAAGTAATCCACCTGGG |
| | | GAGTACGGCCGCAAGGTTGAAACTCAAAGGAATTGACGGGGGCCCGCACAAGCAGT |
| | | GGAGTATGTGGTTTAATTCGAAGCAACGCGAAGAACCTTACCAGGTCTTGACATCGA |
| | | GTGACGTACCTAGAGATAGGTATTTTCTTCGGAACACAAAGACAGGTGGTGCATGGTT |
| | | GTCGTCAGCTCGTGTCGTGAGATGTTGGGTTAAGTCCCGCAACGAGCGCAACCCTTAC |
| | | CATTAGTTGCTACGCAAGAGCACTCTAATGGGACTGCCGTTGACAAAACGGAGGAAG |
| | | GTGGGGATGACGTCAAATCATCATGCCCCTTATGACCTGGGCTACACACGTACTACAA |
| | | TGGCAATATAACAGAGGGAAGCAATACAGCGATGTGGAGCAAATCCCCAAAAATTGT |
| | | CCCAGTTCAGATTGCAGGCTGCAACTCGCCTGCATGAAGTCGGAATTGCTAGTAATCG |
| | | CAGATCAGCATGCTGCGGTGAATACGTTCCCGGGCCTTGTACACACCGCCCGTCACAC |
| | | CATGGGAGTCGGTAACACCCAAAGCCGGTCGTCTAACCTTCGGGAGGATGCCGTCTA |
| | | AGGTGGGATTGATGACTGGGGTGAAGTCGTAACAAGGTAGCCGTATCGGAAGGTGCG |
| | | GCTGGATCACCTCCTT (SEQ ID NO: 14) |
| GCF_000518765 | Ruminococcus | ATAAAGAGTTTGATCCTGGCTCAGGACGAACGCTGGCGGCACGCTTAACACATGCAA |
| | flavefaciens | GTCGAACGGAGATAATTTGAGTTTACTTAGGTTATCTTAGTGGCGGACGGGTGAGTAA |
| | | CACGTGAGCAACCTACCTTAGAGAGAGGGATAGCTTCTGGAAACGGATGGTAATACC |
| | | TCATAACATAACTGAACCGCATGATTTAGTTATCAAAGATTTATCACTCTGAGATGGG |
| | | CTCGCGTCTGATTAGATAGTTGGTGAGGTAACGGCTCACCAAGTCGACGATCAGTAG |
| | | CCGGACTGAGAGGTTGAACGGCCACATTGGGACTGAGACACGGCCCAGACTCCTACG |
| | | GGAGGCAGCAGTGGGGAATATTGCACAATGGGGGAAACCCTGATGCAGCGATGCCG |
| | | CGTGGAGGAAGAAGGTTTTCGGATTGTAAACTCCTGTCTTAAAGGACGATAATGACG |
| | | GTACTTTAGGAGGAAGCTCCGGCTAACTACGTGCCAGCAGCCGCGGTAATACGTAGG |
| | | GAGCGAGCGTTGTCCGGAATTACTGGGTGTAAAGGGAGCGTAGGCGGGAGCGCAAGT |
| | | CAGATGTGAAATACATGGGCTCAACCCATGGGCTGCATTTGAAACTGTGTTTCTTGAG |
| | | TGAAGTAGAGGTAAGCGGAATTCCTGGTGTAGCGGTGAAATGCGTAGATATCAGGAG |
| | | GAACACCGGTGGCGAAGGCGGCTTACTGGGCTTTTACTGACGCTGAGGCTCGAAAGC |
| | | GTGGGGAGCAAACAGGATTAGATACCCTGGTAGTCCACGCTGTAAACGATGATTACT |
| | | AGGTGTGGGGGGACTGACCCCTTCCGTGCCGCAGTTAACACAATAAGTAATCCACCT |
| | | GGGGAGTACGGCCGCAAGGTTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CGTATGCATAGTCTAGAGATAGATGAAATTCCTTCGGGGACATATAGACAGGTGGTG |
| | | CATGGTTGTCGTCAGCTCGTGTCGTGAGATGTTGGGTTAAGTCCCGCAACGAGCGCAA |
| | | CCCTTACCTTTAGTTGCTACGCAAGAGCACTCTAAAGGGACTGCCGTTGACAAAACGG |
| | | AGGAAGGTGGGGATGACGTCAAATCATCATGCCCCTTATGACCTGGGCTACACACGT |
| | | ACTACAATGGCAATTAACAAAGAGAAGCAAGACGGTGACGTGGAGCGAATCTCAAA |
| | | AAATTGTCCCAGTTCAGATTGCAGGCTGCAACTCGCCTGCATGAAGTCGGAATTGCTA |
| | | GTAATCGCGGATCAGCATGCCGCGGTGAATACGTTCCCGGGCCTTGTACACACCGCCC |
| | | GTCACACCATGGGAGTCGGTAACACCCGAAGTCGGTAGTCTAACAGCAATGAGGACG |
| | | CCGCCGAAGGTGGGATTGATGACTGGGGTGAAGTCGTAACAAGGTAGCCGTATCGGA |
| | | AGGTGCGGCTGGATCACCTCCTTT (SEQ ID NO: 15) |
| GCF_000577335 | Clostridium | CAAAGGAGCAATCCGCTGAAAGATGGACTCGCGTCCGATTAGCCAGTTGGCGGGGTA |
| | jeddahense | AAGGCCCACCAAAGCGACGATCGGTAGCCGGGTTGAGAGACTGAACGGCCACATTGG |
| | | GACTGAGACACGGCCCAGACTCCTACGGGAGGCAGCAGTGGGGGATATTGCACAATG |
| | | GAGGAAACTCTGATGCAGCAATGCCGCGTGAGGGAAGACGGTCTTCGGATTGTAAAC |
| | | CTCTGTCCTTGGTGAAGATAATGACGGTAGCCAAGGAGGAAGCTCCGGCTAACTACG |
| | | TGCCAGCAGCCGCGGTAATACGTAGGGAGCAAGCGTTGTCCGGATTTACTGGGTGTA |
| | | AAGGGTGCGTAGGCGGCTTTTTAAGTCGGGCGTGAAAGCTGTGGGCTTAACCCACAA |
| | | ATTGCGTTCGAAACTGGAAGGCTTGAGTGAAGTAGAGGTAGGCGGAATTCCCGGTGT |
| | | AGCGGTGAAATGCGTAGAGATCGGGAGGAACACCAGTGGCGAAGGCGGCCTACTGG |
| | | GCTTTAACTGACGCTGAGGCACGAAAGCATGGGTAGCAAACAGGATTAGATACCCTG |
| | | GTAGTCCATGCCGTAAACGATGATTACTAGGTGTGGGGGGTCTGACCCCTTCCGTGCC |
| | | GGAGTTAACACAATAAGTAATCCACCTGGGGAGTACGGCCGCAAGGTTGAAACTCAA |
| | | AGGAATTGACGGGGGCCCGCACAAGCAGTGGAGTATGTGGTTTAATTCGAAGCAACG |
| | | CGAAGAACCTTACCAGGTCTTGACATCCAACTAACGAGGCAGAGATGCATTAGGTGC |
| | | CCTTCGGGGAAAGTTGAGACAGGTGGTGCATGGTTGTCGTCAGCTCGTGTCGTGAGAT |
| | | GTTGGGTTAAGTCCCGCAACGAGCGCAACCCTTGTGATTAGTTGCTACGCAAGAGCA |
| | | CTCTAATCAGACTGCCGTTGACAAAACGGAGGAAGGTGGGGACGACGTCAAATCATC |
| | | ATGCCCCTTATGACCTGGGCTACACACGTACTACAATGGTCGCTAACAGAGGGAAGC |
| | | CAAGCCGCGAGGTGGAGCAAACCCCCAAAAGCGATCTCAGTTCGGATTGTAGGCTGC |
| | | AACCCGCCTACATGAAGTTGGAATTGCTAGTAATCGCGGATCAGCATGCCGCGGTGA |
| | | ATACGTTCCCGGGCCTTGTACACACCGCCCGTCACACCATGGGAGCCGGTAATACCCG |
| | | AAGCCAATAGTCTAACCGCAAGGGGGACGTTGTCGAAGGTAGGATTGGCGACTGGGG |
| | | TGAAGTCGTAACAAGGTAGCCGTATCGGAAGGTGCGGCTGGATCACCTCCTTT (SEQ |
| | | ID NO: 16) |
| GCF_000620945 | Clostridium viride | GCTTAGTGGCGGACGGGTGAGTAACGCGTGAGTAACCTGCCTTGGAGTGGGGAATAA |
| | | CACATCGAAAGGTGTGCTAATACCGCATGATGCAACGGGATCGCATGGTTCTGTTGCC |
| | | AAAGATTTATCGCTCTGAGATGGACTCGCGTCTGATTAGCTAGTTGGTGAGGTAATGG |
| | | CTCACCAAGGCGACGATCAGTAGCCGGACTGAGAGGTTGACCGGCCACATTGGGACT |
| | | GAGACACGGCCCAGACTCCTACGGGAGGCAGCAGTGGGGAATATTGGGCAATGGGC |
| | | GCAAGCCTGACCCAGCAACGCCGCGTGAAGGAAGAAGGCCCTCGGGTTGTAAACTTC |
| | | TTTTATTCGAGACGAAACAAATGACGGTACCGAATGAATAAGCCACGGCTAACTACG |
| | | TGCCAGCAGCCGCGGTAATACGTAGGTGGCAAGCGTTATCCGGATTTACTGGGTGTA |
| | | AAGGGCGTGTAGGCGGGACTGCAAGTCAGATGTGAAATTCCAGGGCTCAACTCTGGA |
| | | CCTGCATTTGAAACTGTAGTTCTTGAGTGATGGAGAGGCAGGCGGAATTCCGAGTGTA |
| | | GCGGTGAAATGCGTAGATATTCGGAGGAACACCAGTGGCGAAGGCGGCCTGCTGGAC |
| | | ATTAACTGACGCTGAGGCGCGAAAGCGTGGGGAGCAAACAGGATTAGATACCCTGGT |
| | | AGTCCACGCTGTAAACGATGGATACTAGGTGTGGGGGGACTGACCCCTTCCGTGCCG |
| | | CAGTTAACACAATAAGTATCCCACCTGGGGAGTACGATCGCAAGGTTGAAACTCAAA |
| | | GGAATTGACGGGGGCCCGCACAAGCGGTGGAGTATGTGGTTTAATTCGAAGCAACGC |
| | | GAAGAACCTTACCAGGGCTTGACATCCCTCTGACCGGTCTAGAGATAGGCCCTCCCTT |
| | | CGGGGCAGAGGTGACAGGTGGTGCATGGTTGTCGTCAGCTCGTGTCGTGAGATGTTG |
| | | GGTTAAGTCCCGCAACGAGCGCAACCCCTATTGTTAGTTGCTACGCAAGAGCACTCTA |
| | | GCGAGACTGCCGTTGACAAAACGGAGGAAGGTGGGGACGACGTCAAATCATCATGCC |
| | | CCTTATGTCCTGGGCTACACACGTACTACAATGGCGCTTAACAGAGGGAGGCAATAC |
| | | CGCGAGGTGGAGCAAACCCCTAAAAGGCGTCCCAGTTCGGATTGCAGGCTGAAACCC |
| | | GCCTGTATGAAGTTGGAATCGCTAGTAATCGCGGATCAGCATGCCGCGGTGAATACG |
| | | TTCCCGGGCCTTGTACACACCGCCCGTCACACCATGAGAGTCGGGAACACCCGAAGT |
| | | CCGTAGCCTAACAGCAATGAGGGCGCGGCCGAAGGTGGGTTCGATAATTGGGGTGAA |
| | | GTCGTAACAAGGTAGCCGTATCGGAAGGTGCGGCTGGATCACCTCCTTT (SEQ ID |
| | | NO: 17) |
| GCF_000621285 | Ruminococcus albus | TATTAAGAGTTTGATCCTGGCTCAGGACGAACGCTGGCGGCACGCTTAACACATGCA |
| | | AGTCGAACGAGCGAAAGAGTGCTTGCACTCTCTAGCTAGTGGCGGACGGGTGAGTAA |
| | | CACGTGAGCAATCTGCCTTTCGGAGAGGGATACCAATTGGAAACGATTGTTAATACCT |
| | | CATAACATAACGAAGCCGCATGACTTTGTTATCAAATGAATTTCGCCGAAAGATGAG |
| | | CTCGCGTCTGATTAGGTAGTTGGTGAGGTAACGGCCCACCAAGCCGACGATCAGTAG |
| | | CCGGACTGAGAGGTTGAACGGCCACATTGGGACTGAGACACGGCCCAGACTCCTACG |
| | | GGAGGCAGCAGTGGGGAATATTGCACAATGGGCGAAAGCCTGATGCAGCGATGCCG |
| | | CGTGAGGGAAGAAGGTTTTAGGATTGTAAACCTCTGTCTTTGGGGACGATAATGACG |
| | | GTACCCAAGGAGGAAGCTCCGGCTAACTACGTGCCAGCAGCCGCGGTAATACGTAGG |
| | | GAGCGAGCGTTGTCCGGAATTACTGGGTGTAAAGGGAGCGTAGGCGGGATTGCAAGT |
| | | CAGGTGTGAAATTTAGGGGCTTAACCCCTGAACTGCACTTGAAACTGTAGTTCTTGAG |
| | | TGAAGTAGAGGTAAGCGGAATTCCTAGTGTAGCGGTGAAATGCGTAGATATTAGGAG |
| | | GAACATCAGTGGCGAAGGCGGCTTACTGGGCTTTAACTGACGCTGAGGCTCGAAAGC |
| | | GTGGGGAGCAAACAGGATTAGATACCCTGGTAGTCCACGCCGTAAACGATGATTACT |
| | | AGGTGTGGGGGGACTGACCCCTTCCGTGCCGCAGTTAACACAATAAGTAATCCACCT |
| | | GGGGAGTACGGCCGCAAGGCTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CGTACGCATAGCATAGAGATATGTGAAATCCCTTCGGGGACGTATAGACAGGTGGTG |
| | | CATGGTTGTCGTCAGCTCGTGTCGTGAGATGTTGGGTTAAGTCCCGCAACGAGCGCAA |
| | | CCCTTACTGTTAGTTGCTACGCAAGAGCACTCTAGCAGGACTGCCGTTGACAAAACGG |
| | | AGGAAGGTGGGGATGACGTCAAATCATCATGCCCCTTATGACCTGGGCTACACACGT |
| | | ACTACAATGGCTGTTAACAGAGGGAAGCAAAACAGTGATGTGGAGCAAAACCCTAA |
| | | AAGCAGTCTTAGTTCGGATTGTAGGCTGCAACCCGCCTACATGAAGTCGGAATTGCTA |
| | | GTAATCGCGGATCAGCATGCCGCGGTGAATACGTTCCCGGGCCTTGTACACACCGCCC |
| | | GTCACGCCATGGGAGTCGGTAACACCCGAAGCCTGTGTTCTAACCGCAAGGAGGAAG |
| | | CAGTCGAAGGTGGGATTGATGACTGGGGTGAAGTCGTAACAAGGTAGCCGTATCGGA |
| | | AGGTGCGGCTGGATCACCTCCTTT (SEQ ID NO: 18) |
| GCF_000701665 | Agathobaculum | CAAGTTGGGAGTGAAATCCGGGGGCTTAACCCCCGAACTGCTTTCAAAACTGCTGGT |
| | desmolans | CTTGAGTGATGGAGAGGCAGGCGGAATTCCGTGTGTAGCGGTGAAATGCGTAGATAT |
| | | ACGGAGGAACACCAGTGGCGAAGGCGGCCTGCTGGACATTAACTGACGCTGAGGCGC |
| | | GAAAGCGTGGGGAGCAAACAGGATTAGATACCCTGGTAGTCCACGCCGTAAACGATG |
| | | GATACTAGGTGTGGGAGGTATTGACCCCTTCCGTGCCGCAGTTAACACAATAAGTATC |
| | | CCACCTGGGGAGTACGGCCGCAAGGTTGAAACTCAAAGGAATTGACGGGGGCCCGCA |
| | | CAAGCAGTGGAGTATGTGGTTTAATTCGAAGCAACGCGAAGAACCTTACCAGGTCTT |
| | | GACATCCCGGTGACCGTCCTAGAGATAGGACTTCCCTTCGGGGCAACGGTGACAGGT |
| | | GGTGCATGGTTGTCGTCAGCTCGTGTCGTGAGATGTTGGGTTAAGTCCCGCAACGAGC |
| | | GCAACCCTTACGGTTAGTTGATACGCAAGATCACTCTAGCCGGACTGCCGTTGACAAA |
| | | ACGGAGGAAGGTGGGGACGACGTCAAATCATCATGCCCCTTATGACCTGGGCTACAC |
| | | ACGTACTACAATGGCAGTCATACAGAGGGAAGCAAAATCGCGAGGTGGAGCAAATC |
| | | CCTAAAAGCTGTCCCAGTTCAGATTGCAGGCTGCAACCCGCCTGCATGAAGTCGGAA |
| | | TTGCTAGTAATCGCGGATCAGCATGCCGCGGTGAATACGTTCCCGGGCCTTGTACACA |
| | | CCGCCCGTCACACCATGAGAGCCGTCAATACCCGAAGTCCGTAGCCTAACCGCAAGG |
| | | GGGGCGCGGCCGAAGGTAGGGGTGGTAATTAGGGTGAAGTCGTAACAAGGTAGCCG |
| | | TATCGGAAGGTGCGGCTGGATCACCTCCTTT (SEQ ID NO: 19) |
| GCF_000723465 | Ruminococcus | ATTAAGAGTTTGATCCTGGCTCAGGACGAACGCTGGCGGCACGCTTAACACATGCAA |
| | bicirculans | GTCGAACGAGAGAAGAGGAGCTTGCTTTTCTGATCTAGTGGCGGACGGGTGAGTAAC |
| | | ACGTGAGCAATCTGCCTTTCAGAGGGGGATACCGATTGGAAACGATCGTTAATACCG |
| | | CATAACATAATTGAACCGCATGATTTGATTATCAAAGATTTATCGCTGAAAGATGAGC |
| | | TCGCGTCTGATTAGCTAGTTGGTAAGGTAACGGCTTACCAAGGCGACGATCAGTAGC |
| | | CGGACTGAGAGGTTGATCGGCCACATTGGGACTGAGACACGGCCCAGACTCCTACGG |
| | | GAGGCAGCAGTGGGGAATATTGCACAATGGAGGAAACTCTGATGCAGCGATGCCGCG |
| | | TGAGGGAAGAAGGTTTTAGGATTGTAAACCTCTGTCTTCAGGGACGAAAAAAGACGG |
| | | TACCTGAGGAGGAAGCTCCGGCTAACTACGTGCCAGCAGCCGCGGTAATACGTAGGG |
| | | AGCGAGCGTTGTCCGGAATTACTGGGTGTAAAGGGAGCGTAGGCGGGATCGCAAGTC |
| | | AGATGTGAAAACTATGGGCTTAACCCATAAACTGCATTTGAAACTGTGGTTCTTGAGT |
| | | GAAGTAGAGGTAAGCGGAATTCCTAGTGTAGCGGTGAAATGCGTAGATATTAGGAGG |
| | | AACATCAGTGGCGAAGGCGGCTTACTGGGCTTTAACTGACGCTGAGGCTCGAAAGCG |
| | | TGGGGAGCAAACAGGATTAGATACCCTGGTAGTCCACGCCGTAAACGATGATTACTA |
| | | GGTGTGGGGGGACTGACCCCTTCCGTGCCGCAGCAAACGCAATAAGTAATCCACCTG |
| | | GGGAGTACGACCGCAAGGTTGAAACTCAAAGGAATTGACGGGGGCCCGCACAAGCA |
| | | GTGGAGTATGTGGATTAATTCGAAGCAACGCGAAGAACCTTACCAGGTCTTGACATC |
| | | GTATGCATAGCTCAGAGATGAGTGAAATCTCTTCGGAGACATATAGACAGGTGGTGC |
| | | ATGGTTGTCGTCAGCTCGTGTCGTGAGATGTTGGGTTAAGTCCCGCAACGAGCGCAAC |
| | | CCTTACTGTTAGTTGCTACGCAAGAGCACTCTAGCAGGACTGCCGTTGACAAAACGG |
| | | AGGAAGGTGGGGATGACGTCAAATCATCATGCCCCTTATGACCTGGGCCTCACACGT |
| | | ACTACAATGGCTGTCAACAGAGGGAAGCAAAGCCGCGAGGTGGAGCGAACCCCTAA |
| | | AAGCAGTCTTAGTTCGGATTGTAGGCTGCAACCCGCCTACATGAAGTCGGAATTGCTA |
| | | GTAATCGCAGATCAGCATGCTGCGGTGAATACGTTCCCGGGCCTTGTACACACCGCCC |
| | | GTCACGCCATGGGAGTCGGTAACACCCGAAGCCTGTAGTCTAACCGCAAGGAGGACG |
| | | CAGTCGAAGGTGGGATTGATGACTGGGGTGAAGTCGTAACAAGGTAGCCGTATCGGA |
| | | AGGTGCGGCTGGATCACCTCCTTT (SEQ ID NO: 20) |
| GCF_000949455 | Ruthenibacterium | AATGAAGAGTTTGATCCTGGCTCAGGACGAACGCTGGCGGCGCGCCTAACACATGCA |
| | lactatiformans | AGTCGAACGGAGCTGTTTTCTCTGAAGTTTTCGGATGGAAGAGAGTTCAGCTTAGTGG |
| | | CGAACGGGTGAGTAACACGTGAGCAACCTGCCTTTCAGTGGGGGACAACATTTGGAA |
| | | ACGAATGCTAATACCGCATAAGACCACAGTGTCGCATGGCACAGGGGTCAAAGGATT |
| | | TATCCGCTGAAAGATGGGCTCGCGTCCGATTAGCTAGATGGTGAGGTAACGGCCCAC |
| | | CATGGCGACGATCGGTAGCCGGACTGAGAGGTTGAACGGCCACATTGGGACTGAGAC |
| | | ACGGCCCAGACTCCTACGGGAGGCAGCAGTGGGGAATATTGCACAATGGGGGAAAC |
| | | CCTGATGCAGCGACGCCGCGTGGAGGAAGAAGGTCTTCGGATTGTAAACTCCTGTCC |
| | | CAGGGGACGATAATGACGGTACCCTGGGAGGAAGCACCGGCTAACTACGTGCCAGCA |
| | | GCCGCGGTAAAACGTAGGGTGCAAGCGTTGTCCGGAATTACTGGGTGTAAAGGGAGC |
| | | GCAGGCGGATTGGCAAGTTGGGAGTGAAATCTATGGGCTCAACCCATAAATTGCTTT |
| | | CAAAACTGTCAGTCTTGAGTGGTGTAGAGGTAGGCGGAATTCCCGGTGTAGCGGTGG |
| | | AATGCGTAGATATCGGGAGGAACACCAGTGGCGAAGGCGGCCTACTGGGCACTAACT |
| | | GACGCTGAGGCTCGAAAGCATGGGTAGCAAACAGGATTAGATACCCTGGTAGTCCAT |
| | | GCCGTAAACGATGATTACTAGGTGTGGGAGGATTGACCCCTTCCGTGCCGCAGTTAAC |
| | | ACAATAAGTAATCCACCTGGGGAGTACGACCGCAAGGTTGAAACTCAAAGGAATTGA |
| | | CGGGGGCCCGCACAAGCAGTGGAGTATGTGGTTTAATTCGAAGCAACGCGAAGAACC |
| | | TTACCAGGTCTTGACATCGGATGCATACCTAAGAGATTAGGGAAGTCCTTCGGGACAT |
| | | CCAGACAGGTGGTGCATGGTTGTCGTCAGCTCGTGTCGTGAGATGTTGGGTTAAGTCC |
| | | CGCAACGAGCGCAACCCTTATCGTTAGTTACTACGCAAGAGGACTCTAGCGAGACTG |
| | | CCGTTGACAAAACGGAGGAAGGTGGGGATGACGTCAAATCATCATGCCCTTTATGAC |
| | | CTGGGCTACACACGTACTACAATGGCTATTAACAGAGAGAAGCGATACCGCGAGGTG |
| | | GAGCAAACCTCACAAAAATAGTCTCAGTTCGGATCGCAGGCTGCAACCCGCCTGCGT |
| | | GAAGCCGGAATTGCTAGTAATCGCGGATCAGCATGCCGCGGTGAATACGTTCCCGGG |
| | | CCTTGTACACACCGCCCGTCACACCATGAGAGCCGGGGGGACCCGAAGTCGGTAGTC |
| | | TAACCGTAAGGAGGACGCCGCCGAAGGTAAAACTGGTGATTGGGGTGAAGTCGTAAC |
| | | AAGGTAGCCGTATCGGAAGGTGCGGCTGGATCACCTCCTTT (SEQ ID NO: 21) |
| GCF_001244495 | Clostridium | TATTGAGAGTTTGATCCTGGCTCAGGATGAACGCTGGCGGCGTGCTTAACACATGCAA |
| | phoceensis | GTCGAACGGAGTGCCTTAGAAAGAGGATTCGTCCAATTGATAAGGTTACTTAGTGGC |
| | | GGACGGGTGAGTAACGCGTGAGGAACCTGCCTCGGAGTGGGGAATAACAGACCGAA |
| | | AGGCCTGCTAATACCGCATGATGCAGTTGGACCGCATGGTCCTGACTGCCAAAGATTT |
| | | ATCGCTCTGAGATGGCCTCGCGTCTGATTAGCTTGTTGGCGGGGTAATGGCCCACCAA |
| | | GGCGACGATCAGTAGCCGGACTGAGAGGTTGGCCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCTTTCGGGTTGTAAACTTCTTTTCTCAGG |
| | | GACGAACAAATGACGGTACCTGAGGAATAAGCCACGGCTAACTACGTGCCAGCAGCC |
| | | GCGGTAATACGTAGGTGGCAAGCGTTATCCGGATTTACTGGGTGTAAAGGGCGTGTA |
| | | GGCGGGAAGGCAAGTCAGATGTGAAAACTATGGGCTCAACCCATAGCCTGCATTTGA |
| | | AACTGTTTTTCTTGAGTGCTGGAGAGGCAATCGGAATTCCGTGTGTAGCGGTGAAATG |
| | | CGTAGATATACGGAGGAACACCAGTGGCGAAGGCGGATTGCTGGACAGTAACTGACG |
| | | CTGAGGCGCGAAAGCGTGGGGAGCAAACAGGATTAGATACCCTGGTAGTCCACGCTG |
| | | TAAACGATGGATACTAGGTGTGGGGGGTCTGACCCCCTCCGTGCCGCAGTTAACACA |
| | | ATAAGTATCCCACCTGGGGAGTACGATCGCAAGGTTGAAACTCAAAGGAATTGACGG |
| | | GGGCCCGCACAAGCGGTGGAGTATGTGGTTTAATTCGAAGCAACGCGAAGAACCTTA |
| | | CCAGGGCTTGACATCCTACTAACGAAGCAGAGATGCATTAGGTGCCCTTCGGGGAAA |
| | | GTAGAGACAGGTGGTGCATGGTTGTCGTCAGCTCGTGTCGTGAGATGTTGGGTTAAGT |
| | | CCCGCAACGAGCGCAACCCTTATTGTTAGTTGCTACGCAAGAGCACTCTAGCGAGACT |
| | | GCCGTTGACAAAACGGAGGAAGGCGGGGACGACGTCAAATCATCATGCCCCTTATGT |
| | | CCTGGGCTACACACGTACTACAATGGTGGTAAACAGAGGGAAGCAAGACCGCGAGGT |
| | | GGAGCAAATCCCTAAAAGCCATCCCAGTTCGGATTGCAGGCTGAAACCCGCCTGTAT |
| | | GAAGTTGGAATCGCTAGTAATCGCGGATCAGCATGCCGCGGTGAATACGTTCCCGGG |
| | | CCTTGTACACACCGCCCGTCACACCATGAGAGTCGGGAACACCCGAAGTCCGTAGTC |
| | | TAACCGCAAGGGGGACGCGGCCGAAGGTGGGTTCGATAATTGGGGTGAAGTCGTAAC |
| | | AAGGTAGCCGTATCGGAAGGTGCGGCTGGATCACCTCCTTT (SEQ ID NO: 22) |
| GCF_001244995 | Intestinimonas | TATTGAGAGTTTGATCCTGGCTCAGGATGAACGCTGGCGGCGTGCTTAACACATGCAA |
| | massiliensis | GTCGAACGGAACGCCAAGGAAAGAGTTTTCGGACAATGGAATTGGTTGTTTAGTGGC |
| | | GGACGGGTGAGTAACGCGTGAGTAACCTGCCTTGGAGTGGGGAATAACACAGTGAAA |
| | | ATTGTGCTAATACCGCATGATATATTGGTGTCGCATGGCACTGATATCAAAGATTTAT |
| | | CGCTCTGAGATGGACTCGCGTCTGATTAGATAGTTGGCGGGGTAACGGCCCACCAAG |
| | | TCGACGATCAGTAGCCGGACTGAGAGGTTGGCCGGCCACATTGGGACTGAGACACGG |
| | | CCCAGACTCCTACGGGAGGCAGCAGTGGGGAATATTGGGCAATGGGCGCAAGCCTGA |
| | | CCCAGCAACGCCGCGTGAAGGAAGAAGGCTTTCGGGTTGTAAACTTCTTTTAACAGG |
| | | GACGAAGCAAGTGACGGTACCTGTTGAATAAGCCACGGCTAACTACGTGCCAGCAGC |
| | | CGCGGTAATACGTAGGTGGCAAGCGTTATCCGGATTTACTGGGTGTAAAGGGCGTGT |
| | | AGGCGGGACTGCAAGTCAGATGTGAAAACTATGGGCTCAACCCATAGCCTGCATTTG |
| | | AAACTGTAGTTCTTGAGTGTCGGAGAGGCAATCGGAATTCCGTGTGTAGCGGTGAAA |
| | | TGCGTAGATATACGGAGGAACACCAGTGGCGAAGGCGGATTGCTGGACGATAACTGA |
| | | CGCTGAGGCGCGAAAGCGTGGGGAGCAAACAGGATTAGATACCCTGGTAGTCCACGC |
| | | CGTAAACGATGGATACTAGGTGTGGGGGGTCTGACCCCCTCCGTGCCGCAGCTAACG |
| | | CAATAAGTATCCCACCTGGGGAGTACGATCGCAAGGTTGAAACTCAAAGGAATTGAC |
| | | GGGGGCCCGCACAAGCGGTGGAGTATGTGGTTTAATTCGAAGCAACGCGAAGAACCT |
| | | TACCAGGGCTTGACATCCTACTAACGAACCAGAGATGGATTAGGTGCCCTTCGGGGA |
| | | AAGTAGAGACAGGTGGTGCATGGTTGTCGTCAGCTCGTGTCGTGAGATGTTGGGTTAA |
| | | GTCCCGCAACGAGCGCAACCCTTATTGTTAGTTGCTACGCAAGAGCACTCTAGCGAG |
| | | ACTGCCGTTGACAAAACGGAGGAAGGTGGGGACGACGTCAAATCATCATGCCCCTTA |
| | | TGTCCTGGGCCACACACGTACTACAATGGCGGTTAACAGAGGGAGGCAAAGCCGCGA |
| | | GGCAGAGCAAACCCCTAAAAGCCGTCCCAGTTCGGATTGCAGGCTGAAACCCGCCTG |
| | | TATGAAGTCGGAATCGCTAGTAATCGCGGATCAGCATGCCGCGGTGAATACGTTCCC |
| | | GGGCCTTGTACACACCGCCCGTCACACCATGAGAGTCGGGAACACCCGAAGTCCGTA |
| | | GCCTAACTGCAAAGGGGGCGCGGCCGAAGGTGGGTTCGATAATTGGGGTGAAGTCGT |
| | | AACAAGGTAGCCGTATCGGAAGGTGCGGCTGGATCACCTCCTTT (SEQ ID NO: 23) |
| GCF_001261775 | Anaeromassilibacillus | TTTAGAGAGTTTGATCCTGGCTCAGGACGAACGCTGGCGGCGTGCCTAACACATGCA |
| | senegalensis | AGTCGAACGGAGTTAGAAGAGCTTGCTCTTCTAACTTAGTGGCGGACGGGTGAGTAA |
| | | CGCGTGAGTAACCTGCCTTTCAGAGGGGGATAACGTTCTGAAAAGAACGCTAATACC |
| | | GCATGACGTCATAGTACCGCATGGTACAGTGATCAAAGGAGCAATCCGCTGAAAGAT |
| | | GGACTCGCGTCCGATTAGCTAGTTGGTGGGGTAAAGGCTCACCAAGGCGACGATCGG |
| | | TAGCCGGACTGAGAGGTTGAACGGCCACATTGGGACTGAGACACGGCCCAGACTCCT |
| | | ACGGGAGGCAGCAGTGGGGGATATTGCACAATGGGGGAAACCCTGATGCAGCAACG |
| | | CCGCGTGAAGGAAGAAGGTCTTCGGATTGTAAACTTCTGTCCTATGGGAAGATAATG |
| | | ACGGTACCATAGGAGGAAGCTCCGGCTAACTACGTGCCAGCAGCCGCGGTAATACGT |
| | | AGGGAGCAAGCGTTGTCCGGATTTACTGGGTGTAAAGGGTGCGTAGGCGGATCTGCA |
| | | AGTCAGTAGTGAAATCCCGGGGCTTAACCCCGGAACTGCTATTGAAACTGTGGGTCTT |
| | | GAGTGAGGTAGAGGCAGGCGGAATTCCCGGTGTAGCGGTGAAATGCGTAGAGATCG |
| | | GGAGGAACACCAGTGGCGAAGGCGGCCTGCTGGGCCTTAACTGACGCTGAGGCACGA |
| | | AAGCATGGGTAGCAAACAGGATTAGATACCCTGGTAGTCCATGCCGTAAACGATGAT |
| | | TACTAGGTGTGGGTGGTCTGACCCCATCCGTGCCGGAGTTAACACAATAAGTAATCCA |
| | | CCTGGGGAGTACGGCCGCAAGGTTGAAACTCAAAGGAATTGACGGGGGCCCGCACA |
| | | AGCAGTGGAGTATGTGGTTTAATTCGAAGCAACGCGAAGAACCTTACCAGGTCTTGA |
| | | CATCCTACTAACGAAGCAGAGATGCATTAGGTGCCTTTCGGGGAAAGTAGAGACAGG |
| | | TGGTGCATGGTTGTCGTCAGCTCGTGTCGTGAGATGTTGGGTTAAGTCCCGCAACGAG |
| | | CGCAACCCTTGCTATTAGTTGCTACGCAAGAGCACTCTAATAGGACTGCCGTTGACAA |
| | | AACGGAGGAAGGTGGGGACGACGTCAAATCATCATGCCCCTTATGACCTGGGCTACA |
| | | CACGTACTACAATGGTCGTTAACAGAGAGAAGCAATACTGCGAAGTGGAGCAAAACT |
| | | CTAAAAACGGTCTCAGTTCGGATTGTAGGCTGCAACCCGCCTACATGAAGTTGGAATT |
| | | GCTAGTAATCGCGGATCAGCATGCCGCGGTGAATACGTTCCCGGGCCTTGTACACACC |
| | | GCCCGTCACACCATGGGAGCCGGTAATACCCGAAGTCAGTAGTCTAACCGCAAGGAG |
| | | GACGCTGCCGAAGGTAGGATTGGCGACTGGGGTGAAGTCGTAACAAGGTAGCCGTAT |
| | | CGGAAGGTGCGGCTGGATCACCTCCTTT (SEQ ID NO: 24) |
| GCF_001312825 | Ruminococcus | TATGAAGAGTTTGATCCTGGCTCAGGACGAACGCTGGCGGCACGCCTAACACATGCA |
| | champanellensis | AGTCGAACGGAGATAAAGACTTCGGTTTTTATCTTAGTGGCGGACGGGTGAGTAACA |
| | | CGTGAGCAACCTGCCTCTGAGAGAGGGATAGCTTCTGGAAACGGATGGTAATACCTC |
| | | ATAACATAGCGGTACCGCATGATACTGCTATCAAAGATTTATCGCTCAGAGATGGGCT |
| | | CGCGTCTGATTAGCTAGATGGTGAGGTAACGGCTCACCATGGCGACGATCAGTAGCC |
| | | GGACTGAGAGGTTGAACGGCCACATTGGGACTGAGACACGGCCCAGACTCCTACGGG |
| | | AGGCAGCAGTGGGGAATATTGCACAATGGGCGCAAGCCTGATGCAGCGATGCCGCGT |
| | | GGAGGAAGAAGGTTTTCGGATTGTAAACTCCTGTCTTAAGGGACGATAATGACGGTA |
| | | CCTTAGGAGGAAGCTCCGGCTAACTACGTGCCAGCAGCCGCGGTAATACGTAGGGAG |
| | | CGAGCGTTGTCCGGAATTACTGGGTGTAAAGGGAGCGTAGGCGGGATTGCAAGTCAG |
| | | ATGTGAAAACTATGGGCTTAACCCATAGACTGCATTTGAAACTGTAGTTCTTGAGTGA |
| | | AGTAGAGGTAAGCGGAATTCCTAGTGTAGCGGTGAAATGCGTAGATATTAGGAGGAA |
| | | CATCGGTGGCGAAGGCGGCTTACTGGGCTTTTACTGACGCTGAGGCTCGAAAGCGTG |
| | | GGGAGCAAACAGGATTAGATACCCTGGTAGTCCACGCTGTAAACGATGATTACTAGG |
| | | TGTGGGGGGACTGACCCCTTCCGTGCCGCAGTTAACACAATAAGTAATCCACCTGGG |
| | | GAGTACGGCCGCAAGGTTGAAACTCAAAGGAATTGACGGGGGCCCGCACAAGCAGT |
| | | GGAGTATGTGGTTTAATTCGAAGCAACGCGAAAAACCTTACCAGGTCTTGACATCGA |
| | | GTGAATGATCTAGAGATAGATCAGTCCTTCGGGACACAAAGACAGGTGGTGCATGGT |
| | | TGTCGTCAGCTCGTGTCGTGAGATGTTGGGTTAAGTCCCGCAACGAGCGCAACCCTTA |
| | | CCTTTAGTTGCTACGCAAGAGCACTCTAGAGGGACTGCCGTTGACAAAACGGAGGAA |
| | | GGTGGGGATGACGTCAAATCATCATGCCCCTTATGACCTGGGCTACACACGTACTACA |
| | | ATGGCAATGAACAGAGGGAAGCAATACAGTGATGTGGAGCAAATCCCCAAAAATTGT |
| | | CCCAGTTCAGATTGTAGGCTGCAACTCGCCTACATGAAGTCGGAATTGCTAGTAATCG |
| | | CAGATCAGCATGCTGCGGTGAATACGTTCCCGGGCCTTGTACACACCGCCCGTCACAC |
| | | CATGGAGTCGGTAACACCCGAAGCCAGTAGCCTAACCGCAAGGAGGGCGCTGTCGAA |
| | | GGTGGGATTGATGACTGGGGTGAAGTCGTAACAAGGTAGCCGTATCGGAAGGTGCGG |
| | | CTGGATCACCTCCTTT (SEQ ID NO: 25) |
| GCF_001486165 | Bittarella | ATAAAGAGTTTGATCCTGGCTCAGGACGAACGCTGGCGGCGCGCCTAACACATGCAA |
| | massiliensis | GTCGAACGGACACATCCGACGGAATAGCTTGCTAGGAAGATGGATGTTGTTAGTGGC |
| | | GGACGGGTGAGTAACACGTGAGCAACCTGCCTCGGAGTGGGGGACAACAGTTGGAA |
| | | ACGACTGCTAATACCGCATACGGTGGTCGGGGGACATCCCCTGGCTAAGAAAGGATC |
| | | TATGATCCGCTCTGAGATGGGCTCGCGTCTGATTAGCTAGTTGGCGGGGTAACGGCCC |
| | | ACCAAGGCAACGATCAGTAGCCGGACTGAGAGGTTGAACGGCCACATTGGGACTGAG |
| | | ACACGGCCCAGACTCCTACGGGAGGCAGCAGTGGGGAATATTGCACAATGGGGGGA |
| | | ACCCTGATGCAGCGACGCCGCGTGAGGGAAGAAGGTTTTCGGATTGTAAACCTCTGT |
| | | CTTGTGGGACGATAATGACGGTACCACAGGAGGAAGCCATGGCTAACTACGTGCCAG |
| | | CAGCCGCGGTAATACGTAGATGGCGAGCGTTGTCCGGAATTACTGGGTGTAAAGGGA |
| | | GTGTAGGCGGGATCATAAGTTGCGTGTGAAATGCAGGGGCTCAACCCCTGAACTGCG |
| | | CGCAAAACTGTGGTTCTTGAGTGAAGTAGAGGCAGGCGGAATTCCCGGTGTAGCGGT |
| | | GGAATGCGTAGATATCGGGAGGAACACCAGTGGCGAAGGCGGCCTGCTGGGCTTTTA |
| | | CTGACGCTGAGGCTCGAAAGCATGGGGAGCAAACAGGATTAGATACCCTGGTAGTCC |
| | | ATGCCGTAAACGATGATTACTAGGTGTGGGGGGATAACCCCCTCCGTGCCGGAGTTA |
| | | ACACAATAAGTAATCCACCTGGGGAGTACGACCGCAAGGTTGAAACTCAAAGGAATT |
| | | GACGGGGGCCCGCACAAGCAGTGGAGTATGTGGTTTAATTCGAAGCAACGCGAAAAA |
| | | CCTTACCAGGTCTTGACATCTATCGCTATCCCAAGAGATTGGGAGTTCCCTTCGGGGA |
| | | CGGTAAGACAGGTGGTGCATGGTTGTCGTCAGCTCGTGTCGTGAGATGTTGGGTTAAG |
| | | TCCCGCAACGAGCGCAACCCTTACTGTTAGTTGCTACGCAAGAGCACTCTAACGGGA |
| | | CTGCCGTTGACAAAACGGAGGAAGGTGGGGATGACGTCAAATCATCATGCCCTTTAT |
| | | GACCTGGGCTACACACGTACTACAATGGCCGCAAACAACGAGCAGCGAAACCGCGA |
| | | GGTGGAGCGAATCTATAAAAGCGGTCTCAGTTCGGATTGCAGGCTGCAACTCGCCTG |
| | | CATGAAGTCGGAATTGCTAGTAATCGCGGATCAGCATGCCGCGGTGAATACGTTCCC |
| | | GGGCCTTGTACACACCGCCCGTCACACCATGAGAGCCGGTAACACCCGAAGTCAGTA |
| | | GTCTAACCGCAAGGGGGACGCTGCCGAAGGTGGGGCTGGTGATTGGGGTGAAGTCGT |
| | | AACAAGGTAGCCGTATCGGAAGGTGCGGCTGGATCACCTCCTTT (SEQ ID NO: 26) |
| GCF_002157465 | Butyricicoccus | TTTAGAGAGTTTGATCCTGGCTCAGGATGAACGCTGGCGGCGTGCCTAACACATGCA |
| | porcorum | AGTCGAACGGAGCACTGAGACTTCGGTTTTTGTGCTTAGTGGCGGACGGGTGAGTAA |
| | | CGCGTGAGCAATCTGCCTTTCAGAGGGGGATAACGACTGGAAACGGTCGCTAATACC |
| | | GCATAACGTATTTTGCAGGCATCTGCGAGATACCAAAGGAGCAATCCGCTGAAAGAT |
| | | GAGCTCGCGTCTGATTAGATAGTTGGTGAGGTAACGGCCCACCAAGTCGACGATCAG |
| | | TAGCCGGACTGAGAGGTTGAACGGCCACATTGGGACTGAGACACGGCCCAGACTCCT |
| | | ACGGGAGGCAGCAGTGGGGAATATTGCGCAATGGGGGAAACCCTGACGCAGCAACG |
| | | CCGCGTGATCGAAGAAGGTCTTCGGATTGTAAAGATCTTTTATCAGGGACGAAGAAA |
| | | GTGACGGTACCTGATGAATAAGCTCCGGCTAACTACGTGCCAGCAGCCGCGGTAATA |
| | | CGTAGGGAGCGAGCGTTATCCGGATTTACTGGGTGTAAAGGGCGAGTAGGCGGGCTG |
| | | GTAAGTTGGAAGTGAAATGTCGGGGCTTAACCCCGGAACTGCTTTCAAAACTGCTGG |
| | | TCTTGAGTGATGGAGAGGCAGGCGGAATTCCTAGTGTAGCGGTGAAATGCGTAGATA |
| | | TTAGGAGGAACACCAGTGGCGAAGGCGGCCTGCTGGACATTAACTGACGCTGAGGAG |
| | | CGAAAGCGTGGGGAGCAAACAGGATTAGATACCCTGGTAGTCCACGCCGTAAACGAT |
| | | GGATACTAGGTGTGGGAGGTATTGACCCCTTCCGTGCCGCAGTTAACACAATAAGTAT |
| | | CCCACCTGGGGAGTACGACCGCAAGGTTGAAACTCAAAGGAATTGACGGGGGCCCGC |
| | | ACAAGCAGTGGAGTATGTGGTTTAATTCGAAGCAACGCGAAGAACCTTACCTGGTCT |
| | | TGACATCCCGGTGACCGGCATAGAGATATGCCTTTCCCTTCGGGGACAGCGGTGACA |
| | | GGTGGTGCATGGTTGTCGTCAGCTCGTGTCGTGAGATGTTGGGTTAAGTCCCGCAACG |
| | | AGCGCAACCCTTATTGTTAGTTGATACATTTAGTTGATCACTCTAGCGAGACTGCCGT |
| | | TGACAAAACGGAGGAAGGTGGGGACGACGTCAAATCATCATGCCCCTTATGACCAGG |
| | | GCTACACACGTACTACAATGGCAGACATACAGAGGGAAGCAAAGCTGTGAGGCAGA |
| | | GCAAATCCCTAAAAGCTGTCCCAGTTCAGATTGCAGGCTGCAACCCGCCTGCATGAA |
| | | GTCGGAATTGCTAGTAATCGCGGATCAGCATGCCGCGGTGAATACGTTCCCGGGCCTT |
| | | GTACACACCGCCCGTCACACCATGAGAGCCGGTAATGCCCGAAGTCCGTAGTCTAAC |
| | | CGCAAGGAGGACGCGGCCGAAGGCAGGACTGGTAATTAGGGTGAAGTCGTAACAAG |
| | | GTAGCCGTATCGGAAGGTGCGGCTGGATCACCTCCTTT (SEQ ID NO: 27) |
| GCF_002201475 | Acutalibacter muris | TTTAGAGAGTTTGATCCTGGCTCAGGACGAACGCTGGCGGCGTGCCTAATACATGCA |
| | | AGTCGAACGGAGATATTCGCTGATGAAGTACTTCGGTAATGAATCTTGGATATCTTAG |
| | | TGGCGGACGGGTGAGTAACGCGTGAGCAACCTGCCTTTCAGAGGGGGATAACGTTTG |
| | | GAAACGAACGCTAATACCGCATGACATTATCTTATCGCATGGTAGGATAATCAAAGG |
| | | AGCAATCCGCTGAAAGATGGGCTCGCGTCCGATTAGGTAGTTGGTGGGGTAACGGCC |
| | | CACCAAGCCGACGATCGGTAGCCGGACTGAGAGGTTGGACGGCCACATTGGGACTGA |
| | | GACACGGCCCAGACTCCTACGGGAGGCAGCAGTAAGGGATATTGGTCAATGGGGGA |
| | | AACCCTGAACCAGCAACGCCGCGTGAGGGAAGACGGTTTTCGGATTGTAAACCTCTG |
| | | TCCTCTGTGAAGATGATGACGGTAGCAGAGGAGGAAGCTCCGGCTAACTACGTGCCA |
| | | GCAGCCGCGGTAATACGTAGGGAGCGAGCGTTGTCCGGATTTACTGGGTGTAAAGGG |
| | | TGCGTAGGCGGCTTGGCAAGTCAGTAGTGAAATCCATGGGCTTAACCCATGAACTGC |
| | | TATTGAAACTGTCGAGCTTGAGTGAAGTAGAGGTAGGCGGAATTCCCGGTGTAGCGG |
| | | TGAAATGCGTAGAGATCGGGAGGAACACCAGTGGCGAAGGCGGCCTACTGGGCTTTA |
| | | ACTGACGCTGAGGCACGAAAGCGTGGGTAGCAAACAGGATTAGATACCCTGGTAGTC |
| | | CACGCTGTAAACGATGATTACTAGGTGTGGGTGGACTGACCCCATCCGTGCCGGAGTT |
| | | AACACAATAAGTAATCCACCTGGGGAGTACGGCCGCAAGGCTGAAACTCAAAGGAAT |
| | | TGACGGGGGCCCGCACAAGCAGTGGAGTATGTGGATTAATTCGATGCAACGCGAAGA |
| | | ACCTTACCAGGTCTTGACATCCCGCTAACGAGGTAGAGATACGTTAGGTGCCCTTCGG |
| | | GGAAAGCGGAGACAGGTGGTGCATGGTTGTCGTCAGCTCGTGTCGTGAGATGTTGGG |
| | | TTAAGTCCCGCAACGAGCGCAACCCTTACTGTTAGTTGCTACGCAAGAGCACTCTAGC |
| | | AGGACCGCCGTTGACAAAACGGAGGAAGGTGGGGATGATGTCAAATCATCATGCCCC |
| | | TTATGACCTGGGCCTCACACGTACTACAATGGCCATTAACAGAGGGAGGCAAAGCCG |
| | | CGAGGCAGAGCAAAACCCTAAAAATGGTCCCAGTTCGGATCGCAGGCTGCAACCCGC |
| | | CTGCGTGAAGTTGGAATTGCTAGTAATCGCGGATCAGCATGCCGCGGTGAATACGTTC |
| | | CCGGGCCTTGTACACACCGCCCGTCACACCATGGAAGTCGGTAATGCCCGAAGTCAG |
| | | TAGCCTAACCGCAAGGGGGGCGCTGCCGAAGGCAGGATTGATGACTGGGGTGAAGTC |
| | | GTAACAAGGTAGCCGTATCGGAAGGTGCGGCTGGATCACCTCCTTT (SEQ ID NO: 28) |
| GCF_002556665 | Clostridium leptum | TTTAGAGAGTTTGATCCTGGCTCAGGACGAACGCTGGCGGCGTGCCTAACACATGCA |
| | | AGTCGAACGGAGTTAAATTCGACACCCGAGTATCCGGCCGGGAGGCGGGGTGCTGGG |
| | | GGTTGGATTTAACTTAGTGGCGGACGGGTGAGTAACGCGTGAGTAACCTGCCTTTCAG |
| | | AGGGGGATAACGTTCTGAAAAGAACGCTAATACCGCATAACATCAATTTATCGCATG |
| | | ATAGGTTGATCAAAGGAGCAATCCGCTGGAAGATGGACTCGCGTCCGATTAGCCAGT |
| | | TGGCGGGGTAACGGCCCACCAAAGCGACGATCGGTAGCCGGACTGAGAGGTTGAAC |
| | | GGCCACATTGGGACTGAGACACGGCCCAGACTCCTACGGGAGGCAGCAGTGGGGGAT |
| | | ATTGCACAATGGGGGAAACCCTGATGCAGCAACGCCGCGTGAGGGAAGAAGGTTTTC |
| | | GGATTGTAAACCTCTGTTCTTAGTGACGATAATGACGGTAGCTAAGGAGAAAGCTCC |
| | | GGCTAACTACGTGCCAGCAGCCGCGGTAATACGTAGGGAGCGAGCGTTGTCCGGATT |
| | | TACTGGGTGTAAAGGGTGCGTAGGCGGCGAGGCAAGTCAGGCGTGAAATCTATGGGC |
| | | TTAACCCATAAACTGCGCTTGAAACTGTCTTGCTTGAGTGAAGTAGAGGTAGGCGGA |
| | | ATTCCCGGTGTAGCGGTGAAATGCGTAGAGATCGGGAGGAACACCAGTGGCGAAGGC |
| | | GGCCTACTGGGCTTTAACTGACGCTGAAGCACGAAAGCATGGGTAGCAAACAGGATT |
| | | AGATACCCTGGTAGTCCATGCCGTAAACGATGATTACTAGGTGTGGGGGGTCTGACC |
| | | CCCTCCGTGCCGCAGTTAACACAATAAGTAATCCACCTGGGGAGTACGGCCGCAAGG |
| | | TTGAAACTCAAAGGAATTGACGGGGGCCCGCACAAGCAGTGGAGTATGTGGTTTAAT |
| | | TCGAAGCAACGCGAAGAACCTTACCAGGTCTTGACATCCGTCTAACGAAGCAGAGAT |
| | | GCATTAGGTGCCCTTCGGGGAAAGGCGAGACAGGTGGTGCATGGTTGTCGTCAGCTC |
| | | GTGTCGTGAGATGTTGGGTTAAGTCCCGCAACGAGCGCAACCCTTGTTTCTAGTTGCT |
| | | ACGCAAGAGCACTCTAGAGAGACTGCCGTTGACAAAACGGAGGAAGGTGGGGACGA |
| | | CGTCAAATCATCATGCCCCTTATGACCTGGGCCACACACGTACTACAATGGCTGTAAA |
| | | CAGAGGGAAGCAAAGCCGCGAGGTGGAGCAAAACCCTAAAAGCAGTCCCAGTTCGG |
| | | ATCGCAGGCTGCAACCCGCCTGCGTGAAGTCGGAATTGCTAGTAATCGCGGATCAGC |
| | | ATGCCGCGGTGAATACGTTCCCGGGCCTTGTACACACCGCCCGTCACACCATGGGAG |
| | | CCGGTAATACCCGAAGCCAGTAGTTCAACCGCAAGGAGAGCGCTGTCGAAGGTAGGA |
| | | TTGGCGACTGGGGTGAAGTCGTAACAAGGTAGCCGTATCGGAAGGTGCGGCTGGATC |
| | | ACCTCCTTT (SEQ ID NO: 29) |
| GCF_002834225 | Ruminococcus | TTAGAGAGTTTGATCCTGGCTCAGGACGAACGCTGGCGGCGTGCCTAACACATGCAA |
| | bromii | GTCGAACGGAACTGTTTTGAAAGATTTCTTCGGAATGAATTTGATTTAGTTTAGTGGC |
| | | GGACGGGTGAGTAACGCGTGAGTAACCTGCCTTCAAGAGGGGGATAACATTCTGAAA |
| | | AGAATGCTAATACCGCATGACATATCGGAACCACATGGTTCTGATATCAAAGATTTTA |
| | | TCGCTTGAAGATGGACTCGCGTCCGATTAGTTAGTTGGTGAGGTAACGGCTCACCAAG |
| | | ACCGCGATCGGTAGCCGGACTGAGAGGTTGAACGGCCACATTGGGACTGAGACACGG |
| | | CCCAGACTCCTACGGGAGGCAGCAGTGGGGGATATTGCGCAATGGGGGCAACCCTGA |
| | | CGCAGCAACGCCGCGTGAAGGATGAAGGTTTTCGGATTGTAAACTTCTTTTATTAAGG |
| | | ACGAAAAATGACGGTACTTAATGAATAAGCTCCGGCTAACTACGTGCCAGCAGCCGC |
| | | GGTAATACGTAGGGAGCAAGCGTTGTCCGGATTTACTGGGTGTAAAGGGTGCGTAGG |
| | | CGGCTTTGCAAGTCAGATGTGAAATCTATGGGCTCAACCCATAAACTGCATTTGAAAC |
| | | TGTAGAGCTTGAGTGAAGTAGAGGCAGGCGGAATTCCCCGTGTAGCGGTGAAATGCG |
| | | TAGAGATGGGGAGGAACACCAGTGGCGAAGGCGGCCTGCTGGGCTTTAACTGACGCT |
| | | GAGGCACGAAAGCGTGGGTAGCAAACAGGATTAGATACCCTGGTAGTCCACGCTGTA |
| | | AACGATGATTACTAGGTGTGGGGGGTCTGACCCCTTCCGTGCCGGAGTTAACACAAT |
| | | AAGTAATCCACCTGGGGAGTACGGCCGCAAGGTTGAAACTCAAAGGAATTGACGGGG |
| | | GCCCGCACAAGCAGTGGAGTATGTGGTTTAATTCGAAGCAACGCGAAGAACCTTACC |
| | | AGGTCTTGACATCCAACTAACGAAGTAGAGATACATTAGGTGCCCTTCGGGGAAAGT |
| | | TGAGACAGGTGGTGCATGGTTGTCGTCAGCTCGTGTCGTGAGATGTTGGGTTAAGTCC |
| | | CGCAACGAGCGCAACCCTTGCTATTAGTTGCTACGCAAGAGCACTCTAATAGGACTG |
| | | CCGTTGACAAAACGGAGGAAGGTGGGGACGACGTCAAATCATCATGCCCCTTATGAC |
| | | CTGGGCTACACACGTACTACAATGGATGTTAACAGAGGGAAGCAAGACAGTGATGTG |
| | | GAGCAAACCCCTAAAAACATTCTCAGTTCAGATTGCAGGCTGCAACCCGCCTGCATG |
| | | AAGATGGAATTGCTAGTAATCGCGGATCAGAATGCCGCGGTGAATACGTTCCCGGGC |
| | | CTTGTACACACCGCCCGTCACACCATGGGAGCCGGTAATACCCGAAGTCAGTAGTCC |
| | | AACCTCGTGAGGACGCTGCCGAAGGTAGGATTGGCGACTGGGGTGAAGTCGTAACAA |
| | | GGTAGCCGTATCGGAAGGTGCGGCTGGATCACCTCCTTT (SEQ ID NO: 30) |
| GCF_002874775 | Monoglobus | ATCGAGAGTTTGATCCTGGCTCAGGACGAACGCTGGCGGCGTGCCTAACACATGCAA |
| | pectinilyticus | GTCGAGCGAGAAATTTTTAACGGATCCCTTCGGGGAGAAGATAAGGATGGAAAGCGG |
| | | CGGACGGGTGAGTAACGCGTGAGTAACCTGCCTTTAGGAGGGGGACAACATTCCGAA |
| | | AGGGATGCTAATACCGCATAAAATTATTGTATCGCATGGTATAATAATCAAAGATTTA |
| | | TCGCCTAAAGATGGACTCGCGTCCGATTAGCTAGTTGGTGGGGTAAAAGCCTACCAA |
| | | GGCGACGATCGGTAGCCGAACTGAGAGGTTGATCGGCCACATTGGGACTGAGACACG |
| | | GCCCAGACTCCTACGGGAGGCAGCAGTGGGGGATATTGCGCAATGGGGGAAACCCTG |
| | | ACGCAGCAACGCCGCGTGAAGGAAGAAGGCCTTCGGGTTGTAAACTTCTTTAAGTGT |
| | | GGAAGATAATGACGGTACACACAGAATAAGCCACGGCTAACTACGTGCCAGCAGCCG |
| | | CGGTAATACGTAGGTGGCAAGCGTTGTCCGGATTTACTGGGTGTAAAGGGCGTGTAG |
| | | GCGGGTAGACAAGTCAGATGTGAAATACCGGGGCTCAACTCCGGGGCTGCATTTGAA |
| | | ACTGTATATCTTGAGTGTCGGAGAGGAAAGCGGAATTCCTAGTGTAGCGGTGAAATG |
| | | CGTAGATATTAGGAGGAACACCAGTGGCGAAGGCGGCTTTCTGGACGATAACTGACG |
| | | CTGAGGCGCGAAAGCGTGGGGAGCAAACAGGATTAGATACCCTGGTAGTCCACGCCG |
| | | TAAACGATGGATACTAGGTGTAGGAGGTATCGACCCCTTCTGTGCCGCAGTTAACAC |
| | | AATAAGTATCCCACCTGGGGAGTACGGTCGCAAGATTGAAACTCAAAGGAATTGACG |
| | | GGGGCCCGCACAAGCAGTGGAGTATGTGGTTTAATTCGAAGCAACGCGAAGAACCTT |
| | | ACCAGGACTTGACATCCCACGCATAGCCTAGAGATAGGTGAAGTCCTACGGGACGTG |
| | | GAGACAGGTGGTGCATGGTTGTCGTCAGCTCGTGTCGTGAGATGTTGGGTTAAGTCCC |
| | | GCAACGAGCGCAACCCTTACTGTCAGTTACCATCATTAAGTTGGGGACTCTGGCAGG |
| | | ACTGCCGGTGACAAATCGGAGGAAGGTGGGGACGACGTCAAATCATCATGCCCCTTA |
| | | TGTCCTGGGCTACACACGTACTACAATGGCTGTTAACAAAGTGAAGCAAAGCAGTGA |
| | | TGTGGAGCAAAACACAAAAAGCAGTCTCAGTTCAGATTGTAGGCTGAAACTCGCCTA |
| | | TATGAAGTCGGAATTGCTAGTAATCGCAGATCAGCATGCTGCGGTGAATACGTTCCCG |
| | | GGCCTTGTACACACCGCCCGTCACACCATGAGAGTCGATAACACCCGAAGCCTGTAG |
| | | CTTAACCTTAGGGAGAGCGCAGTCGAAGGTGGGATTGATAATTAGGGTGAAGTCGTA |
| | | ACAAGGTAGCCGTATCGGAAGGTGCGGCTGGATCACCTCCTTT (SEQ ID NO: 31) |
| GCF_003020045 | Ethanoligenens | TTGGAGAGTTTGATCCTGGCTCAGGACGAACGCTGGCGGCGCGCCTAACACATGCAA |
| | harbinense | GTCGAGCGGAGTCCTTCGGGACTTAGCGGCGGACGGGTGAGTAACGCGTGAGCAACC |
| | | TGGCCTTCAGAGGGGGATAACGTCTGGAAACGGACGCTAATACCGCATGACATGGCG |
| | | GAGTCGCATGGCTCTGCCATCAAAGGAGTAATCCGCTGAGGGATGGGCTCGCGTCCG |
| | | ATTAGGTAGTTGGTGAGGTAACGGCTCACCAAGCCCGCGATCGGTAGCCGGACTGAG |
| | | AGGTTGGCCGGCCACATTGGGACTGAGACACGGCCCAGACTCCTACGGGAGGCAGCA |
| | | GTGGGGGATATTGCACAATGGAGGAAACTCTGATGCAGCGACGCCGCGTGAGGGAA |
| | | GAAGGTCTTCGGATTGTAAACCTCTGTCTTTGGGGACGAATCAATGACGGTACCCAAG |
| | | GAGGAAGCCACGGCTAACTACGTGCCAGCAGCCGCGGTAATACGTAGGTGGCAAGCG |
| | | TTGTCCGGAATTACTGGGTGTAAAGGGTGCGCAGGCGGGGCGGCAAGTTGGATGTGA |
| | | AAACTCCGGGCTCAACCCGGAGCCTGCATTCAAAACTGTCGCTCTTGAGTGAAGTAG |
| | | AGGCAGGCGGAATTCCCGGTGTAGCGGTGAAATGCGTAGATATCGGGAGGAACACCA |
| | | GTGGCGAAGGCGGCCTGCTGGGCTTTTACTGACGCTGAGGCACGAAAGCATGGGTAG |
| | | CAAACAGGATTAGATACCCTGGTAGTCCATGCCGTAAACGATGATTGCTAGGTGTGG |
| | | GGGGTCTGACCCCTTCCGTGCCGGAGTTAACACAATAAGCAATCCACCTGGGGAGTA |
| | | CGGCCGCAAGGTTGAAACTCAAAGGAATTGACGGGGGCCCGCACAAGCAGTGGAGT |
| | | ATGTGGTTTAATTCGAAGCAACGCGAAGAACCTTACCAGGTCTTGACATCCACCGAAT |
| | | CCCCCAGAGATGGGGGAGTGCCCTTCGGGGAGCGGTGAGACAGGTGGTGCATGGTTG |
| | | TCGTCAGCTCGTGTCGTGAGATGTTGGGTTAAGTCCCGCAACGAGCGCAACCCTTGTG |
| | | AATAGTTGCTACGAAAGAGCACTCTATTCAGACCGCCGTTGACAAAACGGAGGAAGG |
| | | TGGGGATGACGTCAAATCATCATGCCCCTTATGACCTGGGCTACACACGTACTACAAT |
| | | GGCCATCAACAGAGGGAAGCAAGGCCGCGAGGTGGAGCGAACCCCTAAAAATGGTC |
| | | TCAGTTCAGATTGCAGGCTGAAACCCGCCTGCATGAAGATGGAATTGCTAGTAATCG |
| | | CGGATCAGCATGCCGCGGTGAATACGTTCCCGGGCCTTGTACACACCGCCCGTCACAC |
| | | CATGAGAGCCGGGGACACCCGAAGTCGGTTGGGTAACCGTAAGGAGCCCGCCGCCGA |
| | | AGGTGGAATCGGTAATTGGGGTGAAGTCGTAACAAGGTAGCCGTATCGGAAGGTGCG |
| | | GCTGGATCACCTCCTTT (SEQ ID NO: 32) |
| GCF_900048895 | Neglecta timonensis | TTTAGAGAGTTTGATCCTGGCTCAGGACGAACGCTGGCGGCGTGCCTAACACATGCA |
| | | AGTCGAACGGAGATAGACGCTGAAAGGGAGACAGCTTGCTGTAAGAATTTCTTGTTT |
| | | ATCTTAGTGGCGGACGGGTGAGTAACGCGTGAGTAACCTGCCTTTCAGAGGGGGATA |
| | | ACGTCTGGAAACGGACGCTAATACCGCATGAGACCACAGCTTCACATGGAGCGGCGG |
| | | TCAAAGGAGCAATCCGCTGAAAGATGGACTCGCGTCCGATTAGATAGTTGGCGGGGT |
| | | AACGGCCCACCAAGTCGACGATCGGTAGCCGGACTGAGAGGTTGAACGGCCACATTG |
| | | GGACTGAGACACGGCCCAGACTCCTACGGGAGGCAGCAGTGAGGGATATTGGTCAAT |
| | | GGGGGAAACCCTGAACCAGCAACGCCGCGTGAGGGAAGACGGTTTTCGGATTGTAAA |
| | | CCTCTGTCCTCTGTGAAGATAGTGACGGTAGCAGAGGAGGAAGCTCCGGCTAACTAC |
| | | GTGCCAGCAGCCGCGGTAATACGTAGGGAGCGAGCGTTGTCCGGATTTACTGGGTGT |
| | | AAAGGGTGCGTAGGCGGCTCTGCAAGTCAGAAGTGAAATCCATGGGCTTAACCCATG |
| | | AACTGCTTTTGAAACTGTAGAGCTTGAGTGAAGTAGAGGTAGGCGGAATTCCCGGTG |
| | | TAGCGGTGAAATGCGTAGAGATCGGGAGGAACACCAGTGGCGAAGGCGGCCTACTG |
| | | GGCTTTAACTGACGCTGAGGCACGAAAGCATGGGTAGCAAACAGGATTAGATACCCT |
| | | GGTAGTCCATGCCGTAAACGATGATTACTAGGTGTGGGGGGTCTGACCCCCTCCGTGC |
| | | CGGAGTTAACACAATAAGTAATCCACCTGGGGAGTACGACCGCAAGGTTGAAACTCA |
| | | AAGGAATTGACGGGGGCCCGCACAAGCAGTGGAGTATGTGGATTAATTCGAAGCAAC |
| | | GCGAAGAACCTTACCAGGTCTTGACATCCAACTAACGAAGCAGAGATGCATTAGGTG |
| | | CCCTTCGGGGAAAGTTGAGACAGGTGGTGCATGGTTGTCGTCAGCTCGTGTCGTGAG |
| | | ATGTTGGGTTAAGTCCCGCAACGAGCGCAACCCTTACTGTTAGTTGCTACGCAAGAGC |
| | | ACTCTAGCAGGACTGCCGTTGACAAAACGGAGGAAGGTGGGGACGACGTCAAATCAT |
| | | CATGCCCCTTATGACCTGGGCCTCACACGTACTACAATGGCCATTAACAGAGGGAAG |
| | | CAAGCCCGCGAGGTGGAGCAAAACCCTAAAAATGGTCTCAGTTCGGATCGTAGGCTG |
| | | AAACCCGCCTGCGTGAAGTTGGAATTGCTAGTAATCGCGGATCAGCATGCCGCGGTG |
| | | AATACGTTCCCGGGCCTTGTACACACCGCCCGTCACACCATGGGAGCCGGTAATACCC |
| | | GAAGTCAGTAGTCTAACCGCAAGGGGGACGCTGCCGAAGGTAGGATTGGCGACTGGG |
| | | GTGAAGTCGTAACAAGGTAGCCGTATCGGAAGGTGCGGCTGGATCACCTCCTTT (SEQ |
| | | ID NO: 33) |
| GCF_900078395 | Anaerotruncus | AAAGAGTTTGATCCTGGCTCAGGACGAACGCTGGCGGCGCGCCTAACACATGCAAGT |
| | rubiinfantis | CGAACGGAGTTTATCCGACTGAAGTTTTCGGATGGAAGATGGATAAACTTAGTGGCG |
| | | GACGGGTGAGTAACACGTGAGCAACCTGCCTTTCAGAGGGGGATAACGATTGGAAAC |
| | | GATCGCTAATACCGCATAACATTATGAGGAGACATCTTCTTATAATCAAAGGAGCAA |
| | | TCCGCTGAAAGATGGGCTCGCGGCCGATTAGCTAGATGGTGGGGTAACGGCCCACCA |
| | | TGGCGACGATCGGTAGCCGGACTGAGAGGTTGAACGGCCACATTGGGACTGAGACAC |
| | | GGCCCAGACTCCTACGGGAGGCAGCAGTGGGGGATATTGCACAATGGAGGAAACTCT |
| | | GATGCAGCGACGCCGCGTGAGGGAAGACGGTCTTCGGATTGTAAACCTCTGTCTTAG |
| | | GGGAAGAAAATGACGGTACCCTAAGAGGAAGCTCCGGCTAACTACGTGCCAGCAGCC |
| | | GCGGTAATACGTAGGGAGCGAGCGTTGTCCGGAATTACTGGGTGTAAAGGGAGCGTA |
| | | GGCGGGATGGCAAGTTGGATGTTTAAACTAACGGCTCAACTGTTAGGTGCATCCAAA |
| | | ACTGCTGTTCTTGAGTGAAGTAGAGGCAGGCGGAATTCCTAGTGTAGCGGTGAAATG |
| | | CGTAGATATTAGGAGGAACACCAGTGGCGAAGGCGGCCTGCTGGGCTTTAACTGACG |
| | | CTGAGGCTCGAAAGCGTGGGGAGCAAACAGGATTAGATACCCTGGTAGTCCACGCTG |
| | | TAAACGATGATTACTAGGTGTGGGGGGACTGACCCCTTCCGTGCCGCAGTTAACACA |
| | | ATAAGTAATCCACCTGGGGAGTACGGCCGCAAGGTTGAAACTCAAAGGAATTGACGG |
| | | GGGCCCGCACAAGCAGTGGAGTATGTGGTTTAATTCGAAGCAACGCGAAGAACCTTA |
| | | CCAGGTCTTGACATCGGATGCATACCATAGAGATATGGGAAGTCCTTCGGGACATCC |
| | | AGACAGGTGGTGCATGGTTGTCGTCAGCTCGTGTCGTGAGATGTTGGGTTAAGTCCCG |
| | | CAACGAGCGCAACCCTTATTATTAGTTGCTACGCAAGAGCACTCTAATGAGACTGCCG |
| | | TTGACAAAACGGAGGAAGGTGGGGATGACGTCAAATCATCATGCCCCTTATGACCTG |
| | | GGCTACACACGTACTACAATGGCACTTAAACAAAGGGCAGCAACGTCGCGAGGCGAA |
| | | GCGAATCCCGAAAAAGTGTCTCAGTTCGGATCGCAGGCTGCAACCCGCCTGCGTGAA |
| | | GTCGGAATTGCTAGTAATCGCGGATCAGCATGCCGCGGTGAATACGTTCCCGGGCCTT |
| | | GTACACACCGCCCGTCACACCATGGGAGTCGGTAACACCCGAAGCCAGTAGTCTAAC |
| | | TGCAAAGAGGACGCTGTCGAAGGTGGGATTGATGACTGGGGTGAAGTCGTAACAAGG |
| | | TAGCCGTATCGGAAGGTGCGGCTGGATCACCTCCTTT (SEQ ID NO: 34) |
| GCF_900095865 | Massilioclostridium | ATTAAGAGTTTGATCCTGGCTCAGGACGAACGCTGGCGGCGCGCCTAACACATGCAA |
| | coli | GTCGAACGGAGATACCTGTTAGATCCCTTCGGGGTGACGATGGACTATCTTAGTGGCG |
| | | GACGGGTGAGTAACACGTGAGCAACCTGCCTTACAGAGTGGGATAACGTTTGGAAAC |
| | | GAACGCTAATACCGCATAACATTAACTTATCGCATGGTAAGATAATCAAAGAAATTC |
| | | GCTGTAAGATGGGCTCGCGTCTGATTAGATAGTTGGTGAGGTAACGGCTCACCAAGT |
| | | CGACGATCAGTAGCCGGACTGAGAGGTTGAACGGCCACATTGGGACTGAGACACGGC |
| | | CCAGACTCCTACGGGAGGCAGCAGTGGGGAATATTGCACAATGGGGGAAACCCTGAT |
| | | GCAGCGACGCCGCGTGAGGGAAGAAGGTTTTCGGATTGTAAACCTCTGTCTTCAGGG |
| | | ACGATAGTGACGGTACCTGAGGAGGAAGCTCCGGCTAACTACGTGCCAGCAGCCGCG |
| | | GTAATACGTAGGGAGCGAGCGTTGTCCGGAATTACTGGGTGTAAAGGGAGCGTAGGC |
| | | GGGACAGCAAGTTGAATGTGAAATCTATGGGCTCAACCCATAAACTGCGTTCAAAAC |
| | | TGTTGTTCTTGAGTGAAGTAGAGGTAGGCGGAATTCCTAGTGTAGCGGTGAAATGCGT |
| | | AGATATTAGGAGGAACACCAGTGGCGAAGGCGGCCTACTGGGCTTTAACTGACGCTG |
| | | AGGCTCGAAAGCGTGGGTAGCAAACAGGATTAGATACCCTGGTAGTCCACGCTGTAA |
| | | ACGATGATTACTAGGTGTNNNNNNNTCAACCTTCCGTGCCGGAGTTAACACAATAAG |
| | | TAATCCACCTGGGGAGTACGACCGCAAGGTTGAAACTCAAAGGAATTGACGGGGGCC |
| | | CGCACAAGCAGTGGAGTATGTGGTTTAATTCGAAGCAACGCGAAGAACCTTACCAGG |
| | | TCTTGACATCCAACTAACGAGATAGAGATATGTTAGGTGCCCTTCGGGGAAAGTTGA |
| | | GACAGGTGGTGCATGGTTGTCGTCAGCTCGTGTCGTGAGATGTTGGGTTAAGTCCCGC |
| | | AACGAGCGCAACCCTTACCATTAGTTGCTACGCAAGAGCACTCTAATGGGACTGCCG |
| | | TTGACAAAACGGAGGAAGGTGGGGATGACGTCAAATCATCATGCCCCTTATGACCTG |
| | | GGCCACACACGTACTACAATGGCTATTAACAGAGGGAAGCAATACCGCGAGGAGGA |
| | | GCAAACCCCTAAAAATAGTCTCAGTTCGGATTGCAGGCTGCAACCCGCCTGCATGAA |
| | | GCCGGAATTGCTAGTAATCGCGGATCAGCATGCCGCGGTGAATACGTTCCCGGGCCTT |
| | | GTACACACCGCCCGTCACACCATGAGAGTTGGCAACACCCGAAGCCAGTAGCCTAAC |
| | | CGCAAGGAGGGCGCTGTCGAAGGTGGGGTTGATGATTAGGGTGAAGTCGTAACAAGG |
| | | TAGCCGTATCGGAAGGTGCGGCTGGATCACCTCCTTT (SEQ ID NO: 35) |
| GCF_900104675 | Angelakisella | AATGAAGAGTTTGATCCTGGCTCAGGACGAACGCTGGCGGCGCGCCTAACACATGCA |
| | massiliensis | AGTCGAACGGAGTAAGATGAGCTTGCTTATCTTACTTAGTGGCGGACGGGTGAGTAA |
| | | CACGTGAGCAACCTGCCTTCGAGTGGGGAATAACAGTCGGAAACGACTGCTAATACC |
| | | GCATAACACATTGGGATGGCATCATCCTGATGTCAAAGATTTATCGCTCGAAGATGG |
| | | GCTCGCGTCCGATTAGCTAGTTGGCGGGGTAACGGCCCACCAAGGCGACGATCGGTA |
| | | GCCGGACTGAGAGGTTGATCGGCCACATTGGGACTGAGACACGGCCCAGACTCCTAC |
| | | GGGAGGCAGCAGTGGGGGATATTGCACAATGGGGGAAACCCTGATGCAGCGACGCC |
| | | GCGTGTAGGAAGACGGTCCTCTGGATTGTAAACTACTGTCTTCAGGGACGATAATGA |
| | | CGGTACCTGAGGAGGAAGCTCCGGCTAACTACGTGCCAGCAGCCGCGGTAATACGTA |
| | | GGGAGCGAGCGTTGTCCGGAATTACTGGGTGTAAAGGGAGCGTAGGCGGGGAGGCA |
| | | AGTTGGATGTGAAAACTATCGGCTCAACTGATAGACTGCATTCAAAACTGTTTCTCTT |
| | | GAGTGAAGTAGAGGCAGGCGGGATTCCTAGTGTAGCGGTGAAATGCGTAGATATTAG |
| | | GAGGAACACCAGTGGCGAAGGCGGCCTGCTGGGCTTTTACTGACGCTGAGGCTCGAA |
| | | AGTGTGGGGAGCAAACAGGATTAGATACCCTGGTAGTCCACACCGTAAACGATGATT |
| | | ACTAGGTGTGGGGGGTCTGACCCCTTCCGTGCCGGAGTTAACACAATAAGTAATCCA |
| | | CCTGGGGAGTACGGCCGCAAGGTTGAAACTCAAAGGAATTGACGGGGGCCCGCACA |
| | | AGCAGTGGAGTATGTGGTTTAATTCGAAGCAACGCGAAGAACCTTACCAGGTCTTGA |
| | | CATCTCCTGCATAACCTAGAGATAGGTGAAGTCCTTCGGGACAGGAAGACAGGTGGT |
| | | GCATGGTTGTCGTCAGCTCGTGTCGTGAGATGTTGGGTTAAGTCCCGCAACGAGCGCA |
| | | ACCCTTGTTTTTAGTTGCTACGCAAGAGCACTCTAAAGAGACTGCCGTTGACAAAACG |
| | | GAGGAAGGTGGGGATGACGTCAAATCATCATGCCCCTTATGACCTGGGCTACACACG |
| | | TACTACAATGGCAATTAACAGAGGGAAGCGACACCGCGAGGTGGAGCAAAACCCTA |
| | | AAAATTGTCCCAGTTCAGATTGCAGGCTGCAACTCGCCTGCATGAAGTCGGAATTGCT |
| | | AGTAATCGCGGATCAGCATGCCGCGGTGAATACGTTCCCGGGCCTTGTACACACCGC |
| | | CCGTCACACCATGGGAGTCGGTAACACCCGAAGTCAGTAGCCTAACCGCAAGGAGGG |
| | | CGCTGCCGAAGGTGGGATTGATGACTGGGGTGAAGTCGTAACAAGGTAGCCGTATCG |
| | | GAAGGTGCGGCTGGATCACCTCCTTT (SEQ ID NO: 36) |
| GCF_900130065 | Sporobacter | TATTGAGAGTTTGATCCTGGCTCAGGACGAACGCTGGCGGCGTGCCTAACACATGCA |
| | termitidis | AGTCGAACGGAGACAATTGGTTCGCTGATTGTCTTAGTGGCGGACGGGTGAGTAACG |
| | | CGTGAGCAATCTGCCCTTCGGAGGGGGACAACAGCTGGAAACGGCTGCTAATACCGC |
| | | ATAATGTATATTCAAGGCATCTTGGATATACCAAAGATTTATCGCCGAAGGATGAGCT |
| | | CGCGTCTGATTAGCTAGTTGGTGAGGTAAAGGCTCACCAAGGCTGCGATCAGTAGCC |
| | | GGACTGAGAGGTTGAACGGCCACATTGGGACTGAGATACGGCCCAGACTCCTACGGG |
| | | AGGCAGCAGTGGGGAATATTGGGCAATGGGGGCAACCCTGACCCAGCAACGCCGCGT |
| | | GAAGGAAGAAGGCCTTCGGGTTGTAAACTTCTTTGACCAGGGACGAAACAAATGACG |
| | | GTACCTGGAAAACAAGCCACGGCTAACTACGTGCCAGCAGCCGCGGTAATACGTAGG |
| | | TGGCAAGCGTTGTCCGGATTTACTGGGTGTAAAGGGCGCGTAGGCGGGAGTACAAGT |
| | | CAGATGTGAAATCTGGGGGCTTAACCCTCAAACTGCATTTGAAACTGTATTTCTTGAG |
| | | TATCGGAGAGGCAGGCGGAATTCCTAGTGTAGCGGTGAAATGCGTAGATATTAGGAG |
| | | GAACACCAGTGGCGAAGGCGGCCTGCTGGACGACAACTGACGCTGAGGCGCGAAAG |
| | | CGTGGGGAGCAAACAGGATTAGATACCCTGGTAGTCCACGCTGTAAACGATGAATAC |
| | | TAGGTGTGGGGGGACTGACCCCCTCCGTGCCGGAGTTAACACAATAAGTATTCCACCT |
| | | GGGGAGTACGGCCGCAAGGTTGAAACTCAAAGGAATTGACGGGGGCCCGCACAAGC |
| | | AGTGGATTATGTGGTTTAATTCGAAGCAACGCGAAGAACCTTACCAGGGCTTGACAT |
| | | CGTACTAACGAAGCAGAGATGCATTAGGTGCCCTTCGGGGAAAGTATAGACAGGTGG |
| | | TGCATGGTTGTCGTCAGCTCGTGTCGTGAGATGTTGGGTTAAGTCCCGCAACGAGCGC |
| | | AACCCCTATTGTTAGTTGCTACGCGAGAGCACTCTAGCGAGACTGCCGTTGACAAAAC |
| | | GGAGGAAGGTGGGGACGACGTCAAATCATCATGCCCCTTATGTCCTGGGCTACACAC |
| | | GTAATACAATGGCGCTCAACAGAGGGAAGCAAGACCGCGAGGTGGAGCAAATCCCT |
| | | AAAAGGCGTCTCAGTTCAGATTGCAGGCTGCAACTCGCCTGCATGAAGTCGGAATTG |
| | | CTAGTAATCGCGGATCAGCATGCCGCGGTGAATACGTTCCCGGGCCTTGTACACACCG |
| | | CCCGTCACACCATGAGAGCCGGGAACACCCGAAGTCCGTAGTCTAACCGCAA (SEQ |
| | | ID NO: 37) |
| GCF_900148495 | Negativibacillus | ACAAAGAGTTTGATCCTGGCTCAGGACGAACGCTGGCGGCGCGCCTAACACATGCAA |
| | massiliensis | GTCGAACGGAGTTGTGTTGAAAGCTTGCTGGATATACAACTTAGTGGCGGACGGGTG |
| | | AGTAACACGTGAGTAACCTGCCTCTCAGAGTGGAATAACGTTTGGAAACGAACGCTA |
| | | ATACCGCATAACGTGAGAAGAGGGCATCCTCTTTTTACCAAAGATTTATCGCTGAGAG |
| | | ATGGGCTCGCGGCCGATTAGGTAGTTGGTGAGATAACAGCCCACCAAGCCGACGATC |
| | | GGTAGCCGGACTGAGAGGTTGATCGGCCACATTGGGACTGAGACACGGCCCAGACTC |
| | | CTACGGGAGGCAGCAGTGGGGGATATTGCACAATGGGGGAAACCCTGATGCAGCGA |
| | | CGCCGCGTGAGGGAAGACGGTTTTCGGATTGTAAACCTCTGTCTTTAGGGACGAAAA |
| | | AATGACGGTACCTAAGGAGGAAGCCACGGCTAACTACGTGCCAGCAGCCGCGGTAAT |
| | | ACGTAGGTGGCAAGCGTTGTCCGGAATTACTGGGTGTAAAGGGAGCGTAGGCGGGGA |
| | | GACAAGTTGAATGTCTAAACTATCGGCTTAACTGATAGTCGCGTTCAAAACTATCACT |
| | | CTTGAGTGCAGTAGAGGTAGGCGGAATTCCTAGTGTAGCGGTGAAATGCGTAGATAT |
| | | TAGGAGGAACACCAGTGGCGAAGGCGGCCTACTGGGCTGTAACTGACGCTGAGGCTC |
| | | GAAAGCGTGGGTAGCAAACAGGATTAGATACCCTGGTAGTCCACGCCGTAAACGATG |
| | | ATTACTAGGTGTGGGGGGACTGACCCCTTCCGTGCCGGAGTTAACACAATAAGTAAT |
| | | CCACCTGGGGAGTACGACCGCAAGGTTGAAACTCAAAGGAATTGACGGGGGCCCGCA |
| | | CAAGCAGTGGAGTATGTGGTTTAATTCGAAGCAACGCGAAGAACCTTACCAGGTCTT |
| | | GACATCGAGCGACGAACCAAGAGATTGGTTCTTCCTTCGGGACGCGAAGACAGGTGG |
| | | TGCATGGTTGTCGTCAGCTCGTGTCGTGAGATGTTGGGTTAAGTCCCGCAACGAGCGC |
| | | AACCCTTATCATTAGTTGCTACGCAAGAGCACTCTAATGAGACTGCCGTTGATAAAAC |
| | | GGAGGAAGGTGGGGATGACGTCAAATCATCATGCCCCTTATGACCTGGGCTACACAC |
| | | GTACTACAATGGTGATCAAACAGAGGGAAGCAACACAGCGATGTGAAGCAAATCCC |
| | | GAAAAATCATCTCAGTTCAGATTGCAGGCTGCAACTCGCCTGCATGAAGTCGGAATT |
| | | GCTAGTAATCGCGGATCAGCATGCCGCGGTGAATACGTTCCCGGGCCTTGTACACACC |
| | | GCCCGTCACACCATGGGAGTCGGTAACACCCGAAGCCAGTAGCCTAACCGCAAGGAG |
| | | GGCGCTGTCGAAGGTGGGATTGATGACTGGGGTGAAGTCGTAACAAGGTAGCCGTAT |
| | | CGGAAGGTGCGGCTGGATCACCTCCTTT (SEQ ID NO: 38) |
| GCF_900155615 | Massilimaliae | AAAGAGTTTGATCCTGGCTCAGGACGAACGCTGTCGGCGCGCCTAACACATGCAAGT |
| | massiliensis | CGAACGAAGCTGCATCGAACGAATTCTTCGGAAAGAGATTGGTACAGCTTAGTGGCG |
| | | GACGGGTGAGTAACGCGTGAGTAACCTGCCTTTCAGAGGGGGATAACGTTTGGAAAC |
| | | GAACGCTAATACCGCATAACATATTAAATTCGCATGGATTTGATATCAAAGGAGCAA |
| | | TCCGCTGAAAGATGGACTCGCGTCCAATTAGCTAGTTGGTGAGGTAACGGCCCACCA |
| | | AGGCGACGATTGGTAGCCGGACTGAGAGGTTGAACGGCCACATTGGGACTGAGACAC |
| | | GGCCCAGACTCCTACGGGAGGCAGCAGTGGGGAATATTGCACAATGGGGGAAACCCT |
| | | GATGCAGCGACGCCGAGTGAGGGAAGAAGGTTTTCGGATTGTAAACCTCTGTCCTTG |
| | | GTGAAGATAATGACGGTAGCCAAGGAGGAAGCTACGGCTAACTACGTGCCAGCAGCC |
| | | GCGGTAATACGTAGGTAGCGAGCGTTGTCCGGAATTACTGGGTGTAAAGGGAGCGTA |
| | | GGCGGGATTGCAAGTTGAATGTCAAATCTACGGGCTTAACCCGTAGCCGCGTTCAAA |
| | | ACTGCAGTTCTTGAGTGAAGTAGAGGCAGGCGGAATTCCTAGTGTAGCGGTGAAATG |
| | | CGTAAATATTAGGAGGAACACCAGTGGCGAAGGCGGCCTGCTGGGCTTTAACTGACG |
| | | CTGAGGCTCGAAAGCGTGGGTAGCAAACAGGATTAGATACCCTGGTAGTCCACGCTG |
| | | TAAACGATGATTACTAGGTGTNNNNNNNACTGACCCCTTCCGTGCCGGAGTTAACAC |
| | | AATAAGTAATCCACCTGGGGAGTACGACCGCAAGGTTGAAACTCAAAGGAATTGACG |
| | | GGGGCCCGCACAAGCAGTGGAGTATGTGGTTTAATTCGAAGCAACGCGAAGAACCTT |
| | | ACCAGGTCTTGACATCGTGCGCATAGCCTAGAGATAGGTGAAGCCCTTCGGGGCGCA |
| | | TAGACAGGTGGTGCATGGTTGTCGTCAGCTCGTGTCGTGAGATGTTGGGTTAAGTCCC |
| | | GCAACGAGCGCAACCCTTACGTTTAGTTGCTACGCAAGAGCACTCTAGACGGACTGC |
| | | CGTTGACAAAACGGAGGAAGGTGGGGATGACGTCAAATCATCATGCCCCTTATGACC |
| | | TGGGCTACACACGTACTACAATGGCTATTAACAGAGGGAAGCAAGATGGTGACATGG |
| | | AGCAAACCCCTAAAAATAGTCTCAGTTCGGATTGCAGGCTGCAACCCGCCTGCATGA |
| | | AGCCGGAATTGCTAGTAATCGCGGATCAGCATGCCGCGGTGAATACGTTCCCGGGCC |
| | | TTGTACACACCGCCCGTCACACCATGAGAGTTGGCAACACCCGAAGCCGATAGTCTA |
| | | ACCGCAAGGGGGACGTCGTCGAAGGTGGGGTTGATGATTGGGGTGAAGTCGTAACAA |
| | | GGTAGCCGTATCGGAAGGTGCGGCTGGATCACCTCCTTT (SEQ ID NO: 39) |
| GCF_900155735 | Intestinibacillus | TAGTGGCGGACGGGTGAGTAACGCGTGAGCAATCTGCCTTTAGGAGGGGGATAACGA |
| | massiliensis | CCGGAAACGGTCGCTAATACCGCATGAAGTGCCGGGTGGGCATCCACCTGGCACCAA |
| | | AGGAGCAATCCGCCTTTAGATGAGCTCGCGTCCCATTAGCTAGTTGGTGAGGTAACG |
| | | GCCCACCAAGGCGACGATGGGTAGCCGGACTGAGAGGTTGAACGGCCACATTGGGAC |
| | | TGAGACACGGCCCAGACTCCTACGGGAGGCAGCAGTGGGGAATATTGCGCAATGGGG |
| | | GAAACCCTGACGCAGCAACGCCGCGTGATTGAAGAAGGCCTTCGGGTTGTAAAGATC |
| | | TTTAATGAGGGACGAAAAATGACGGTACCTCAAGAATAAGCTCCGGCTAACTACGTG |
| | | CCAGCAGCCGCGGTAATACGTAGGGAGCAAGCGTTATCCGGATTTACTGGGTGTAAA |
| | | GGGCGAGTAGGCGGGCTGGCAAGTTGGGAGTGAAATCCGGGGGCTTAACCCCCGAAC |
| | | TGCTTTCAAAACTGCTGGCCTTGAGTGATGGAGAGGCAGGCGGAATTCCGTGTGTAG |
| | | CGGTGAAATGCGTAGATATACGGAGGAACACCAGTGGCGAAGGCGGCCTGCTGGAC |
| | | ATTAACTGACGCTGAGGCGCGAAAGCGTGGGGAGCAAACAGGATTAGATACCCTGGT |
| | | AGTCCACGCCGTAAACGATGGATACTAGGTGTGGGAGGTATTGACCCCTTCCGTGCC |
| | | GGAGTTAACACAATAAGTATCCCACCTGGGGAGTACGGCCGCAAGGTTGAAACTCAA |
| | | AGGAATTGACGGGGGCCCGCACAAGCAGTGGAGTATGTGGTTTAATTCGAAGCAACG |
| | | CGAAGAACCTTACCAGGTCTTGACATCCCTCTGACCGGTACAGAGATGTACCTTCCCT |
| | | TCGGGGCAGGGGTGACAGGTGGTGCATGGTTGTCGTCAGCTCGTGTCGTGAGATGTTG |
| | | GGTTAAGTCCCGCAACGAGCGCAACCCTTATTGTTAGTTGATACATTCAGTTGATCAC |
| | | TCTAGCGAGACTGCCGTTGACAAAACGGAGGAAGGTGGGGACGACGTCAAATCATCA |
| | | TGCCCCTTATGACCTGGGCTACACACGTACTACAATGGCAGTCATACAGAGGGAAGC |
| | | AAAGCCGCGAGGTGGAGCAAATCCCTAAAAGCTGTCCCAGTTCAGATTGCAGGCTGC |
| | | A (SEQ ID NO: 40) |
| GCF_900167205 | Eubacterium | TGTACCAAAGCTATTGCGCTGAAGGATGGGCTCGCGTCTGATTAGATAGTTGGTGGGG |
| | coprostanoligenes | TAACGGCCTACCAAGTCGACGATCAGTAGCCGGACTGAGAGGTTGAACGGCCACATT |
| | | GGGACTGAGACACGGCCCAGACTCCTACGGGAGGCAGCAGTGGGGAATATTGCACA |
| | | ATGGGCGCAAGCCTGATGCAGCAACGCCGCGTGGAGGAAGACGGTTTTCGGATTGTA |
| | | AACTCCTGTTCTTAGTGAAGAAAAATGACGGTAGCTAAGGAGCAAGCCACGGCTAAC |
| | | TACGTGCCAGCAGCCGCGGTAATACGTAGGTGGCAAGCGTTGTCCGGAATTACTGGG |
| | | TGTAAAGGGAGCGCAGGCGGGGGAGCAAGTCAGCTGTGAAATCTATGGGCTTAACCC |
| | | ATAAACTGCAGTTGAAACTGTTCTTCTTGAGTGAAGTAGAGGTTGGCGGAATTCCGAG |
| | | TGTAGCGGTGAAATGCGTAGATATTCGGAGGAACACCGGTGGCGAAGGCGGCCAACT |
| | | GGGCTTTTACTGACGCTGAGGCTCGAAAGTGTGGGGAGCAAACAGGATTAGATACCC |
| | | TGGTAGTCCACACTGTAAACGATGATAACTAGGTGTAGGGGGTCTGACCCCTTCTGTG |
| | | CCGCAGCTAACGCAATAAGTTATCCACCTGGGGAGTACGACCGCAAGGTTGAAACTC |
| | | AAAGGAATTGACGGGGACCCGCACAAGCAGTGGATTATGTGGTTTAATTCGATGCAA |
| | | CGCGAAGAACCTTACCAGCACTTGACATCCAACTAACGAAATAGAGATATATTAGGT |
| | | GCCCCTCGGGGAAAGTTGAGACAGGTGGTGCATGGTTGTCGTCAGCTCGTGTCGTGA |
| | | GATGTTGGGTTAAGTCCCGCAACGAGCGCAACCCCTGCCATTAGTTGCTACGCAAGA |
| | | GCACTCTAATGGGACCGCTACCGACAAGGTGGAGGAAGGTGGGGATGACGTCAAATC |
| | | ATCATGCCCCTTATGTGCTGGGCTACACACGTAATACAATGGTCGTTAACAAAGAGA |
| | | AGCAATACCGCGAGGTGGAGCAAAACTTCAAAAACGATCTCAGTTCGGACTGTAGGC |
| | | TGAAACTCGCCTGCACGAAGTTGGAATTGCTAGTAATCGTGGATCAGCATGCCACGG |
| | | TGAATACGTTCCCGGGTCTTGTACACACCGCCCGTCACACCATGGGAGCCGGTAATAC |
| | | CCGAAGTCAGTAGTCTAACCTTAATGGAGGACGCTGCCGAAGGTAGGATTGGCGACT |
| | | GGGGTGAAGTCGTAACAAGGTAGCCGTAGGAGAACCTGCGGCTGGATCACCTCCTTT |
| | | (SEQ ID NO: 41) |
| GCF_900169495 | Provencibacterium | CTAAAGAGTTTGATCCTGGCTCAGGACGAACGCTGGCGGCGCGCCTAACACATGCAA |
| | massiliense | GTCGAACGGAGAAATGCTGAGCTTGCTTTGCATTTTTTAGTGGCGGACGGGTGAGTAA |
| | | CACGTGAGCAACCTGCCTTTGTGAGGGGAATAACGTCTGGAAACGGACGCTAATACC |
| | | GCATAACGTCAAGGAACCGCATGGTTTTTTGACCAAAGATTTTATCGCAAAAAGATG |
| | | GGCTCGCGGCTGATTAGCTAGTTGGCGGGGTAACGGCCCACCAAGGCGACGATCAGT |
| | | AGCCGGACTGAGAGGTTGATCGGCCACATTGGGACTGAGACACGGCCCAGACTCCTA |
| | | CGGGAGGCAGCAGTGGGGGATATTGCACAATGGGGGAAACCCTGATGCAGCGACGC |
| | | CGCGTGAGGGAAGACGGTTTTCGGATTGTAAACCTCTGTCTTCAGGGACGAAATCAA |
| | | TGACGGTACCTGAGGAGGAAGCCACGGCTAACTACGTGCCAGCAGCCGCGGTAATAC |
| | | GTAGGTGGCAAGCGTTGTCCGGAATTACTGGGTGTAAAGGGAGCGTAGGCGGGAATG |
| | | CAAGTTGAATGTTTAAACTATCGGCTCAACTGATAATCGCGTTCAAAACTGCATTTCT |
| | | TGAGTGGAGTAGAGGCAGGCGGAATTCCTAGTGTAGCGGTGAAATGCGTAGATATTA |
| | | GGAGGAACACCAGTGGCGAAGGCGGCCTGCTGGGCTCTAACTGACGCTGAGGCTCGA |
| | | AAGCGTGGGTAGCAAACAGGATTAGATACCCTGGTAGTCCACGCCGTAAACGATGAT |
| | | TACTAGGTGTGGGGGGACTGACCCCTTCCGTGCCGGAGTTAACACAATAAGTAATCC |
| | | ACCTGGGGAGTACGGTCGCAAGACTGAAACTCAAAGGAATTGACGGGGGCCCGCAC |
| | | AAGCAGTGGAGTATGTGGTTTAATTCGAAGCAACGCGAAGAACCTTACCAGGTCTTG |
| | | ACATCGTGCGCATACCGTAGAGATACGGGAAGTCCTTCGGGACGCATAGACAGGTGG |
| | | TGCATGGTTGTCGTCAGCTCGTGTCGTGAGATGTTGGGTTAAGTCCCGCAACGAGCGC |
| | | AACCCTTATTATTAGTTGCTACGCAAGAGCACTCTAATGAGACTGCCGTTGACAAAAC |
| | | GGCGGAAGGTGGGGATGACGTCAAATCATCATGCCCCTTATGACCTGGGCTACACAC |
| | | GTACTACAATGGCACTTAACAGAGGGAAGCAAGACCGCGAGGTGGAGCAAACCCCC |
| | | AAAAAGTGTCTCAGTTCGGATTGCAGGCTGCAACCCGCCTGTATGAAGTCGGAATTG |
| | | CTAGTAATCGCGGATCAGCATGCCGCGGTGAATACGTTCCCGGGCCTTGTACACACCG |
| | | CCCGTCACACCATGAGAGCCGGTAACACCCGAAGTCAGTAGCCTAACCGCAAGGAGG |
| | | GCGCTGCCGAAGGTGGGATTGGTGATTAGGGTGAAGTCGTAACAAGGTAGCCGTATC |
| | | GGAAGGTGCGGCTGGATCACCTCCTTT (SEQ ID NO: 42) |
| GCF_900176335 | Papillibacter | TATTGAGAGTTTGATCCTGGCTCAGGACGAACGCTGGCGGCGTGCCTAACACATGCA |
| | cinnamivorans | AGTCGAACGAAAATACCAAAGCAGCAATGCGGGGGTATTTTAGTGGCGGACGGGTGA |
| | | GTAACGCGTGAGCAATCTGCCTTTTGGAGGGGGATACCGACTGGAAACGGTCGTTAA |
| | | TACCGCATAACGTATATGGACGACATCGTCCGTATACCAAAGGAGCAATCCGCCGAA |
| | | AGATGAGCTCGCGTCTGATTAGCTAGTTGGCGGGGTAAAGGCCCACCAAGGCGACGA |
| | | TCAGTAGCCGGACTGAGAGGTTGAACGGCCACATTGGGACTGAGATACGGCCCAGAC |
| | | TCCTACGGGAGGCAGCAGTGGGGAATATTGGGCAATGGGCGAAAGCCTGACCCAGCA |
| | | ACGCCGCGTGAAGGAAGAAGGCCTTCGGGTTGTAAACTTCTTTGACCAGGGAAGAAG |
| | | AAGTGACGGTACCTGGAAAACAAGCCACGGCTAACTACGTGCCAGCAGCCGCGGTAA |
| | | TACGTAGGTGGCAAGCGTTGTCCGGATTTACTGGGTGTAAAGGGCGTGTAGGCGGGA |
| | | TTGCAAGTCAGATGTGAAATGCCGGGGCTTAACCCCGGAGCTGCATTTGAAACTGTA |
| | | GTTCTTGAGTGATGGAGAGGCAGGCGGAATTCCTAGTGTAGCGGTGAAATGCGTAGA |
| | | TATTAGGAGGAACACCAGTGGCGAAGGCGGCCTGCTGGACATTAACTGACGCTGAGG |
| | | CGCGAAAGCGTGGGGAGCAAACAGGATTAGATACCCTGGTAGTCCACGCTGTAAACG |
| | | ATGGATACTAGGTGTGGGAGGTCTGACCCCTTCCGTGCCGGAGTTAACACAATAAGT |
| | | ATCCCACCTGGGGAGTACGATCGCAAGGTTGAAACTCAAAGGAATTGACGGGGGCCC |
| | | GCACAAGCAGTGGAGTATGTGGTTTAATTCGAAGCAACGCGAAGAACCTTACCAGGA |
| | | TTTGACATCCTACTAACGAGGTAGAGATACGTCAGGTGCCCTTCGGGGAAAGTAGAG |
| | | ACAGGTGGTGCATGGTTGTCGTCAGCTCGTGTCGTGAGATGTTGGGTTAAGTCCCGCA |
| | | ACGAGCGCAACCCTTATTGCTAGTTGCTACGCAAGAGCACTCTAGCGAGACTGCCGTT |
| | | GACAAAACGGAGGAAGGCGGGGACGACGTCAAATCATCATGCCCCTTATGTCCTGGG |
| | | CTACACACGTACTACAATGGCGGTTAACAGAGGGAAGCAAGACAGTGATGTGGAGCA |
| | | AATCCCTAAAAACCGTCTCAGTTCGGATCGCAGGCTGCAACCCGCCTGCGTGAAGTC |
| | | GGAATTGCTAGTAATCGCGGATCAGCATGCCGCGGTGAATACGTTCCCGGGCCTTGTA |
| | | CACACCGCCCGTCACACCATGAGAGTCGGGAATACCCGAAGTCCGTAGTCTAACCGC |
| | | AAGGGGGACGCGGCCGAAGGTAGGTTCGATAATTGGGGTGAAGTCGTAACAAGGTA |
| | | GCCGTATCGGAAGGTGCGGCTGGATCACCTCCTTT (SEQ ID NO: 43) |
| GCF_900176635 | Clostridium merdae | TTTAGAGAGTTTGATCCTGGCTCAGGACGAACGCTGGCGGCATGCCTAACACATGCA |
| | | AGTCGAACGGAGTAAGAGAGAAGCTTGCTTAGCTCTTACTTAGTGGCGGACGGGTGA |
| | | GTAACGCGTGAGTAACCTGCCTTTCAGAGGGGGATAACGTTCTGAAAAGAACGCTAA |
| | | TACCGCATAACATATTGGTGTCGCATGGCACTGGTATCAAAGGAGCAATCCGCTGAA |
| | | AGATGGACTCGCGTCCGATTAGCTAGTTGGTGGGGTAAAGGCCTACCAAGGCGACGA |
| | | TCGGTAGCCGGGTTGAGAGACTGAACGGCCACATTGGGACTGAGACACGGCCCAGAC |
| | | TCCTACGGGAGGCAGCAGTGGGGGATATTGCACAATGGGCGAAAGCCTGATGCAGCA |
| | | ATGCCGCGTGAGGGAAGACGGTTTTCGGATTGTAAACCTCTGTCCTTGGTGAAGATAA |
| | | TGACGGTAGCCAAGGAGGAAGCTCCGGCTAACTACGTGCCAGCAGCCGCGGTAATAC |
| | | GTAGGGAGCAAGCGTTGTCCGGATTTACTGGGTGTAAAGGGTGCGTAGGCGGCTCTTT |
| | | AAGTCGGGCGTGAAAGCTGTGGGCTCAACCCACAAATTGCGTTCGAAACTGGAGAGC |
| | | TTGAGTGAAGTAGAGGTAGGCGGAATTCCCGGTGTAGCGGTGAAATGCGTAGAGATC |
| | | GGGAGGAACACCAGTGGCGAAGGCGGCCTACTGGGCTTTAACTGACGCTGAGGCACG |
| | | AAAGCATGGGTAGCAAACAGGATTAGATACCCTGGTAGTCCATGCCGTAAACGATGA |
| | | TTACTAGGTGTGGGGGGTCTGACCCCTTCCGTGCCGGAGTTAACACAATAAGTAATCC |
| | | ACCTGGGGAGTACGGCCGCAAGGTTGAAACTCAAAGGAATTGACGGGGGCCCGCAC |
| | | AAGCAGTGGAGTATGTGGTTTAATTCGAAGCAACGCGAAGAACCTTACCAGGTCTTG |
| | | ACATCCAACTAACGAAGCAGAGATGCATTAGGTGCCCTTCGGGGAAAGTTGAGACAG |
| | | GTGGTGCATGGTTGTCGTCAGCTCGTGTCGTGAGATGTTGGGTTAAGTCCCGCAACGA |
| | | GCGCAACCCCTGTGATTAGTTGCTACGCAAGAGCACTCTAATCAGACTGCCGTTGACA |
| | | AAACGGAGGAAGGTGGGGACGACGTCAAATCATCATGCCCCTTATGACCTGGGCTAC |
| | | ACACGTACTACAATGGTCGCTAACAGAGGGAAGCCAAGCCGCGAGGTGGAGCAAAC |
| | | CCCCAAAAGCGGTCTCAGTTCGGATTGTAGGCTGCAACCCGCCTACATGAAGTTGGA |
| | | ATTGCTAGTAATCGCGGATCAGCATGCCGCGGTGAATACGTTCCCGGGCCTTGTACAC |
| | | ACCGCCCGTCACACCATGGGAGCCGGTAATACCCGAAGCCAATAGTCTAACCGCAAG |
| | | GAGGACGTTGTCGAAGGTAGGATTGGCGACTGGGGTGAAGTCGTAACAAGGTAGCCG |
| | | TATCGGAAGGTGCGGCTGGATCACCTCCTTT (SEQ ID NO: 44) |
| GCF_900186535 | Marasmitruncus | AAAGAGTTTGATCCTGGCTCAGGACGAACGCTGGCGGCGCGCCTAACACATGCAAGT |
| | massiliensis | CGAACGGACAGAAGAGAAGCTTGCTTAGCTTCTGTTAGTGGCGGACGGGTGAGTAAC |
| | | ACGTGAGTAACCTGCCTTTCAGAGGGGGATAACGATTGGAAACGATCGCTAATACCG |
| | | CATGATGTTGCGATGGGACATCCTATTGCAACCAAAGGAGTAATCCGCTGAAAGATG |
| | | GGCTCGCGGCCGATTAGATAGTTGGTGAGGTAACGGCCCACCAAGTCAGCGATCGGT |
| | | AGCCGGACTGAGAGGTTGATCGGCCACATTGGGACTGAGACACGGCCCAGACTCCTA |
| | | CGGGAGGCAGCAGTGGGGGATATTGCACAATGGAGGAAACTCTGATGCAGCGACGC |
| | | CGCGTGAGGGAAGACGGTCTTCGGATTGTAAACCTCTGTCTTAGGGGAAGAAAATGA |
| | | CGGTACCCTAAGAGGAAGCTCCGGCTAACTACGTGCCAGCAGCCGCGGTAATACGTA |
| | | GGGAGCGAGCGTTGTCCGGAATTACTGGGTGTAAAGGGAGCGTAGGCGGGGCAGCA |
| | | AGTTGGATGTTTAAACTACCGGCTTAACCGGTAACTGCATCCAAAACTGCAGTTCTTG |
| | | AGTGAAGTAGAGGCAGGCGGAATTCCTAGTGTAGCGGTGAAATGCGTAGATATTAGG |
| | | AGGAACACCAGTGGCGAAGGCGGCCTGCTGGGCTTTAACTGACGCTGAGGCTCGAAA |
| | | GCGTGGGGAGCAAACAGGATTAGATACCCTGGTAGTCCACGCTGTAAACGATGATTA |
| | | CTAGGTGTGGGGGGACTGACCCCTTCCGTGCCGCAGTTAACACAATAAGTAATCCAC |
| | | CTGGGGAGTACGGCCGCAAGGTTGAAACTCAAAGGAATTGACGGGGGCCCGCACAA |
| | | GCAGTGGAGTATGTGGTTTAATTCGAAGCAACGCGAAGAACCTTACCAGGTCTTGAC |
| | | ATCGTGCGCATACCATAGAGATATGGGAAGCCCTTCGGGGCGCATAGACAGGTGGTG |
| | | CATGGTTGTCGTCAGCTCGTGTCGTGAGATGTTGGGTTAAGTCCCGCAACGAGCGCAA |
| | | CCCTTATTACTAGTTGCTACGCAAGAGCACTCTAGTGAGACTGCCGTTGACAAAACGG |
| | | AGGAAGGTGGGGATGACGTCAAATCATCATGCCCCTTATGACCTGGGCTACACACGT |
| | | ACTACAATGGCACTTAAACAGAGGGCTGCTACATCGCGAGATGAAGCGAATCCCGAA |
| | | AAAGTGTCTCAGTTCGGATTGCAGGCTGCAACTCGCCTGCATGAAGTCGGAATTGCTA |
| | | GTAATCGCGGATCAGCATGCCGCGGTGAATACGTTCCCGGGCCTTGTACACACCGCCC |
| | | GTCACACCATGGGAGTCGGTAACACCCGAAGCCAGTAGTCTAACCGCAAGGGGGACG |
| | | CTGTCGAAGGTGGGATTGATGACTGGGGTGAAGTCGTAACAAGGTAGCCGTATCGGA |
| | | AGGTGCGGCTGGATCACCTCCTTT (SEQ ID NO: 45) |
| GCF_900186585 | Massilimaliae | TAAAGAGTTTGATCCTGGCTCAGGACGAACGCTGTCGGCGCGCCTAACACATGCAAG |
| | timonensis | TCGAACGAAGTTGCTTTGAATGAATTCTTCGGAAGGAATTTGATTCAACTTAGTGGCG |
| | | GACGGGTGAGTAACGCGTGAGTAACCTGCCTTTCAGAGGGGGATAACGTCTGGAAAC |
| | | GGACGCTAATACCGCATAACATATTGGTTTCGCATGGAGCTGATATCAAAGGAGCAA |
| | | TCCGCTGAAAGATGGACTCGCGTCCAATTAGCTAGTTGGTGAGGTAACGGCCCACCA |
| | | AGGCGACGATTGGTAGCCGGACTGAGAGGTTGAACGGCCACATTGGGACTGAGACAC |
| | | GGCCCAGACTCCTACGGGAGGCAGCAGTGGGGAATATTGCACAATGGGGGAAACCCT |
| | | GATGCAGCGACGCCGAGTGAGGGAAGAAGGTTTTCGGATTGTAAACCTCTGTCCTTG |
| | | GTGAAGATAATGACGGTAACCAAGGAGGAAGCTACGGCTAACTACGTGCCAGCAGCC |
| | | GCGGTAATACGTAGGTAGCGAGCGTTGTCCGGAATTACTGGGTGTAAAGGGAGCGTA |
| | | GGCGGGATTGCAAGTTGAATGTTAAATCTATGGGCTCAACCCATAGCCGCGTTCAAA |
| | | ACTGCAGTTCTTGAGTGAAGTAGAGGCAGGCGGAATTCCTAGTGTAGCGGTGAAATG |
| | | CGTAAATATTAGGAGGAACACCAGTGGCGAAGGCGGCCTGCTGGGCTTTAACTGACG |
| | | CTGAGGCTCGAAAGCGTGGGTAGCAAACAGGATTAGATACCCTGGTAGTCCACGCTG |
| | | TAAACGATGATTACTAGGTGTGGGGGGACTGACCCCTTCCGTGCCGGAGTTAACACA |
| | | ATAAGTAATCCACCTGGGGAGTACGACCGCAAGGTTGAAACTCAAAGGAATTGACGG |
| | | GGGCCCGCACAAGCAGTGGAGTATGTGGTTTAATTCGAAGCAACGCGAAGAACCTTA |
| | | CCAGGTCTTGACATCCGGTGCATAGCCTAGAGATAGGTGAAGCCCTTCGGGGCACCG |
| | | AGACAGGTGGTGCATGGTTGTCGTCAGCTCGTGTCGTGAGATGTTGGGTTAAGTCCCG |
| | | CAACGAGCGCAACCCTTACGTTTAGTTGCTACGCAAGAGCACTCTAGACGGACTGCC |
| | | GTTGACAAAACGGAGGAAGGTGGGGATGACGTCAAATCATCATGCCCCTTATGACCT |
| | | GGGCTACACACGTACTACAATGGCTATTAACAGAGGGAAGCAAGATGGTGACATGGA |
| | | GCAAACCCCTAAAAATAGTCTCAGTTCGGATTGCAGGCTGCAACCCGCCTGCATGAA |
| | | GCCGGAATTGCTAGTAATCGCGGATCAGCATGCCGCGGTGAATACGTTCCCGGGCCTT |
| | | GTACACACCGCCCGTCACACCATGAGAGTTGGCAACACCCGAAGCCGATAGTCTAAC |
| | | CGCAAGGGGGACGTCGTCGAAGGTGGGGTTGATGATTGGGGTGAAGTCGTAACAAGG |
| | | TAGCCGTATCGGAAGGTGCGGCTGGATCACCTCCTTT (SEQ ID NO: 46) |
| GCF_900199435 | Pygmaiobacter | ATTAAGAGTTTGATCCTGGCTCAGGACGAACGCTGGCGGCGCGCCTAACACATGCAA |
| | massiliensis | GTCGAACGGAGCTTGCACTTCTGAAGTTTTCGGATGGACGAGGTACAAGCTTAGTGG |
| | | CGAACGGGTGAGTAACACGTGAAGAACCTGCCCTTCAGTGGGGGACAACAGTTGGAA |
| | | ACGACTGCTAATACCGCATAAGACCACAGTACCGCATGGTACAGTGATCAAAGGATT |
| | | TATTCGCTGAAGGATGGCTTCGCGTCCGATTAGGTAGTTGGTGAGGTAACGGCCCACC |
| | | AAGCCTACGATCGGTAGCCGGACTGAGAGGTTGATCGGCCACATTGGGACTGAGACA |
| | | CGGCCCAGACTCCTACGGGAGGCAGCAGTGGGGAATATTGCACAATGGAGGAAACTC |
| | | TGATGCAGCGACGCCGCGTGAGGGAAGAAGGTCTTCGGATTGTAAACCTCTGTCTTC |
| | | AGGGACGATAATGACGGTACCTGAGGAGGAAGCACCGGCTAACTACGTGCCAGCAG |
| | | CCGCGGTAAAACGTAGGGTGCAAGCGTTGTCCGGAATTACTGGGTGTAAAGGGAGCG |
| | | CAGGCGGGAAGATAAGTTGGATGTTTAATCTACGGGCTCAACCCGTATCAGCATTCA |
| | | AAACTATTTTTCTTGAGTAGTGCAGAGGTAGGCGGAATTCCCGGTGTAGCGGTGGAAT |
| | | GCGTAGATATCGGGAGGAACACCAGTGGCGAAGGCGGCCTACTGGGCACTAACTGAC |
| | | GCTGAGGCTCGAAAGCATGGGTAGCAAACAGGATTAGATACCCTGGTAGTCCATGCC |
| | | GTAAACGATGATTACTAGGTGTGGGAGGATTGACCCCTTCCGTGCCGCAGTTAACAC |
| | | AATAAGTAATCCACCTGGGGAGTACGACCGCAAGGTTGAAACTCAAAGGAATTGACG |
| | | GGGGCCCGCACAAGCAGTGGAGTATGTGGTTTAATTCGAAGCAACGCGAAGAACCTT |
| | | ACCAGGTCTTGACATCCCGTGCATAGTGTAGAGATACATGAAGTCCTTCGGGACACG |
| | | GTGACAGGTGGTGCATGGTTGTCGTCAGCTCGTGTCGTGAGATGTTGGGTTAAGTCCC |
| | | GCAACGAGCGCAACCCTTATTGCTAGTTACTACGAAAGAGGACTCTAGCAAGACTGC |
| | | CGTTGACAAAACGGAGGAAGGTGGGGATGACGTCAAATCATCATGCCCTTTATGACC |
| | | TGGGCCACACACGTACTACAATGGCTATTAACAAAGAGATGCTAAGCCGCGAGGTGG |
| | | AGCGAACCTCATAAAAATAGTCTCAGTTCGGATTGCAGGCTGCAACTCGCCTGCATG |
| | | AAGCCGGAATTGCTAGTAATCGCGGATCAGCATGCCGCGGTGAATACGTTCCCGGGC |
| | | CTTGTACACACCGCCCGTCACACCATGAGAGCCGGGGGGACCCGAAGTCAGTAGTCT |
| | | AACCGCAAGGAGGACGCTGCCGAAGGTAAAACTGGTGATTGGGGTGAAGTCGTAAC |
| | | AAGGTAGCCGTATCGGAAGGTGCGGCTGGATCACCTCCTTT (SEQ ID NO: 47) |
| GCF_900240385 | Clostridium | TTTAGAGAGTTTGATCCTGGCTCAGGACGAACGCTGGCGGCATGCCTAACACATGCA |
| | minihomine | AGTCGAACGGAGTAAGAGATAAGCTTGCTTAACTCTTACTTAGTGGCGGACGGGTGA |
| | | GTAACGCGTGAGTAACCTGCCTTTCAGAGGGGGATAACGTTCTGAAAAGAACGCTAA |
| | | TACCGCATGATATATCGGTGTCGCATGGCACTGATATCAAAGGAGCAATCCGCTGAA |
| | | AGATGGACTCGCGTCCGATTAGCCAGTTGGCGGGGTAATGGCCCACCAAAGCGACGA |
| | | TCGGTAGCCGGGTTGAGAGACTGGACGGCCACATTGGGACTGAGACACGGCCCAGAC |
| | | TCCTACGGGAGGCAGCAGTGGGGGATATTGCACAATGGAGGAAACTCTGATGCAGCA |
| | | ATGCCGCGTGAGGGAAGACGGTCTTCGGATTGTAAACCTCTGTCCTTGGTGAAGATA |
| | | ATGACGGTAGCCAAGGAGGAAGCTCCGGCTAACTACGTGCCAGCAGCCGCGGTAATA |
| | | CGTAGGGAGCAAGCGTTGTCCGGATTTACTGGGTGTAAAGGGTGCGTAGGCGGCTTTT |
| | | CAAGTCGGGCGTGAAAGCTGTGGGCTTAACCCACAAATTGCGTTCGAAACTGGAGAG |
| | | CTTGAGTGAAGTAGAGGTAGGCGGAATTCCCGGTGTAGCGGTGAAATGCGTAGAGAT |
| | | CGGGAGGAACACCAGTGGCGAAGGCGGCCTACTGGGCTTTAACTGACGCTGAGGCAC |
| | | GAAAGCATGGGTAGCAAACAGGATTAGATACCCTGGTAGTCCATGCCGTAAACGATG |
| | | ATTACTAGGTGTGGGGGGTCTGACCCCTTCCGTGCCGGAGTTAACACAATAAGTAATC |
| | | CACCTGGGGAGTACGGCCGCAAGGCTGAAACTCAAAGGAATTGACGGGGGCCCGCA |
| | | CAAGCAGTGGAGTATGTGGTTTAATTCGAAGCAACGCGAAGAACCTTACCAGGTCTT |
| | | GACATCCAACTAACGAAGCAGAGATGCATTAGGTGCCCTTCGGGGAAAGTTGAGACA |
| | | GGTGGTGCATGGTTGTCGTCAGCTCGTGTCGTGAGATGTTGGGTTAAGTCCCGCAACG |
| | | AGCGCAACCCCTGTGATTAGTTGCTACGCAAGAGCACTCTAATCAGACTGCCGTTGAC |
| | | AAAACGGAGGAAGGTGGGGACGACGTCAAATCATCATGCCCCTTATGACCTGGGCTA |
| | | CACACGTACTACAATGGTCGTTAACAACGGGAAGCTAAGCCGCGAGGTGGCGCAAAT |
| | | CCCCAAAAACGGTCTCAGTTCGGATTGTAGGCTGCAACCCGCCTACATGAAGTTGGA |
| | | ATTGCTAGTAATCGCGGATCAGCATGCCGCGGTGAATACGTTCCCGGGCCTTGTACAC |
| | | ACCGCCCGTCACACCACGGGAGCCGGTAATACCCGAAGCCGATAGTCTAACCGCAAG |
| | | GAGGACGTCGTCGAAGGTAGGATTGGCGACTGGGGTGAAGTCGTAACAAGGTAGCCG |
| | | TATCGGAAGGTGCGGCTGGATCACCTCCTTT (SEQ ID NO: 48) |
| GCF_900289145 | Neobitarella | TAAAGAGTTTGATCCTGGCTCAGGACGAACGCTGGCGGCGCGCTTAACACATGCAAG |
| | massiliensis | TCGAACGGACACATCCGACGGAATAGCTTGCTAGGAAGATGGATGTTGTTAGTGGCG |
| | | GACGGGTGAGTAACACGTGAGCAACCTACCTCAGAGTGGGGGACAACAGTTGGAAA |
| | | CGACTGCTAATACCGCATAAGATGGCAGGGTCGCATGGCCTGGTCATAAAAGGAGCA |
| | | ATTCGCTCTGAGATGGGCTCGCGTCTGATTAGCTAGTTGGTGAGGTAACGGCTCACCA |
| | | AGGCAACGATCAGTAGCCGGACTGAGAGGTTGAACGGCCACATTGGGACTGAGACAC |
| | | GGCCCAGACTCCTACGGGAGGCAGCAGTGGGGAATATTGCACAATGGGGGAAACCCT |
| | | GATGCAGCGACGCCGCGTGAGGGAAGACGGTTTTCGGATTGTAAACCTCTGTCTTGTG |
| | | GGACGATAGTGACGGTACCACAGGAGGAAGCCATGGCTAACTACGTGCCAGCAGCCG |
| | | CGGTAATACGTAGATGGCGAGCGTTGTCCGGAATTACTGGGTGTAAAGGGAGTGTAG |
| | | GCGGGCTGGTAAGTTGAATGTGAAACCTTCGGGCTCAACCCGGAGCGTGCGTTCAAA |
| | | ACTGCTGGTCTTGAGTGAAGTAGAGGCAGGCGGAATTCCCGGTGTAGCGGTGGAATG |
| | | CGTAGATATCGGGAGGAACACCAGTGGCGAAGGCGGCCTGCTGGGCTTTTACTGACG |
| | | CTGAGGCTCGAAAGCATGGGTAGCAAACAGGATTAGATACCCTGGTAGTCCATGCCG |
| | | TAAACGATGATTACTAGGTGTGGGGGGATTGACCCCCTCCGTGCCGGAGTTAACACA |
| | | ATAAGTAATCCACCTGGGGAGTACGACCGCAAGGTTGAAACTCAAAGGAATTGACGG |
| | | GGGCCCGCACAAGCAGTGGAGTATGTGGTTTAATTCGAAGCAACGCGAAAAACCTTA |
| | | CCAGGTCTTGACATCCATCGCCAGGCTAAGAGATTAGCTGTTCCCTTCGGGGACGATG |
| | | AGACAGGTGGTGCATGGTTGTCGTCAGCTCGTGTCGTGAGATGTTGGGTTAAGTCCCG |
| | | CAACGAGCGCAACCCTTACTATTAGTTGCTACGCAAGAGCACTCTAATGGGACTGCC |
| | | GTTGACAAAACGGAGGAAGGTGGGGATGACGTCAAATCATCATGCCCCTTATGACCT |
| | | GGGCTACACACGTACTACAATGGCCGTTAACAGAGAGCAGCGATACCGCGAGGTGGA |
| | | GCGAATCTAGAAAAACGGTCTCAGTTCGGATTGCAGGCTGAAACTCGCCTGCATGAA |
| | | GTCGGAATTGCTAGTAATCGCGGATCAGCATGCCGCGGTGAATACGTTCCCGGGCCTT |
| | | GTACACACCGCCCGTCACACCATGAGAGCCGGTAACACCCGAAGTCAGTAGCCTAAC |
| | | CGCAAGGAGGGCGCTGCCGAAGGTGGGGCTGGTAATTGGGGTGAAGTCGTAACAAG |
| | | GTAGCCGTATCGGAAGGTGCGGCTGGATCACCTCCTTT (SEQ ID NO: 49) |
| GCF_000154385 | Faecalibacterium | TATAAAGAGTTTGATCCTGGCTCAGGACGAACGCTGGCGGCGCGCCTAACACATGCA |
| | prausnitzii | AGTCGAACGAGCGAGAGAGAGCTTGCTTTCTTGAGCGAGTGGCGAACGGGTGAGTAA |
| | | CGCGTGAGGAACCTGCCTCAAAGAGGGGGACAACAGTTGGAAACGACTGCTAATACC |
| | | GCATAAGCCCACGGCTCGGCATCGAGCAGAGGGAAAAGGAGCAATCCGCTTTGAGAT |
| | | GGCCTCGCGTCCGATTAGCTGGTTGGTGAGGTAACGGCCCACCAAGGCGACGATCGG |
| | | TAGCCGGACTGAGAGGTTGAACGGCCACATTGGGACTGAGACACGGCCCAGACTCCT |
| | | ACGGGAGGCAGCAGTGGGGAATATTGCACAATGGGGGAAACCCTGATGCAGCGACG |
| | | CCGCGTGGAGGAAGAAGGTCTTCGGATTGTAAACTCCTGTTGTTGAGGAAGATAATG |
| | | ACGGTACTCAACAAGGAAGTGACGGCTAACTACGTGCCAGCAGCCGCGGTAAAACGT |
| | | AGGTCACAAGCGTTGTCCGGAATTACTGGGTGTAAAGGGAGCGCAGGCGGGAAGAC |
| | | AAGTTGGAAGTGAAATCCATGGGCTCAACCCATGAACTGCTTTCAAAACTGTTTTTCT |
| | | TGAGTAGTGCAGAGGTAGGCGGAATTCCCGGTGTAGCGGTGGAATGCGTAGATATCG |
| | | GGAGGAACACCAGTGGCGAAGGCGGCCTACTGGGCACCAACTGACGCTGAGGCTCG |
| | | AAAGTGTGGGTAGCAAACAGGATTAGATACCCTGGTAGTCCACACTGTAAACGATGA |
| | | TTACTAGGTGTTGGAGGATTGACCCCTTCAGTGCCGCAGTTAACACAATAAGTAATCC |
| | | ACCTGGGGAGTACGACCGCAAGGTTGAAACTCAAAGGAATTGACGGGGGCCCGCAC |
| | | AAGCAGTGGAGTATGTGGTTTAATTCGACGCAACGCGAAGAACCTTACCAAGTCTTG |
| | | ACATCCCTTGACGATGCTGGAAACAGTATTTCTCTTCGGAGCAAGGAGACAGGTGGT |
| | | GCATGGTTGTCGTCAGCTCGTGTCGTGAGATGTTGGGTTAAGTCCCGCAACGAGCGCA |
| | | ACCCTTATGGTCAGTTACTACGCAAGAGGACTCTGGCCAGACTGCCGTTGACAAAAC |
| | | GGAGGAAGGTGGGGATGACGTCAAATCATCATGCCCTTTATGACTTGGGCTACACAC |
| | | GTACTACAATGGCGTTAAACAAAGAGAAGCAAGACCGCGAGGTGGAGCAAAACTCA |
| | | GAAACAACGTCCCAGTTCGGACTGCAGGCTGCAACTCGCCTGCACGAAGTCGGAATT |
| | | GCTAGTAATCGCAGATCAGCATGCTGCGGTGAATACGTTCCCGGGCCTTGTACACACC |
| | | GCCCGTCACACCATGAGAGCCGGGGGGACCCGAAGTCGGTAGTCTAACCGCAAGGAG |
| | | GACGCCGCCGAAGGTAAAACTGGTGATTGGGGTGAAGTCGTAACAAGGTAGCCGTAG |
| | | GAGAACCTGCGGCTGGATCACCTCCTTT (SEQ ID NO: 50) |
| GCF_000174895 | Ruminococcus | TAAAGAGTTTGATCCTGGCTCAGGACGAACGCTGGCGGCACGCTTAACACATGCAAG |
| | flavefaciens | TCGAACGGAGATAATTTGAGTTTACTTGGATTATCTTAGTGGCGGACGGGTGAGTAAC |
| | | ACGTGAGCAACCTGCCTTTGAGAGAGGGATAGCTTCTGGAAACGGATGGTAATACCT |
| | | CATAACATAATTGAAGGGCATCCTTTAATTATCAAAGATTTATCACTCAAAGATGGGC |
| | | TCGCATCTGATTAGATAGTTGGTGAGGTAACGGCTCACCAAGTCGACGATCAGTAGC |
| | | CGGACTGAGAGGTTGAACGGCCACATTGGGACTGAGACACGGCCCAGACTCCTACGG |
| | | GAGGCAGCAGTGGGGAATATTGCACAATGGGGGAAACCCTGATGCAGCGATGCCGC |
| | | GTGGAGGAAGAAGGTTTTCGGATTGTAAACTCCTGTCTTAAAGGACGATAATGACGG |
| | | TACTTTAGGAGGAAGCTCCGGCTAACTACGTGCCAGCAGCCGCGGTAATACGTAGGG |
| | | AGCGAGCGTTGTCCGGAATTACTGGGTGTAAAGGGAGCGTAGGCGGGATTGCAAGTC |
| | | AGATGTGAAATACATGGGCTCAACCCATGGGCTGCATTTGAAACTGTAGTTCTTGAGT |
| | | GAAGTAGAGGTAAGCGGAATTCCTGGTGTAGCGGTGAAATGCGTAGATATCAGGAGG |
| | | AACACCGGTGGCGAAGGCGGCTTACTGGGCTTTTACTGACGCTGAGGCTCGAAAGCG |
| | | TGGGGAGCAAACAGGATTAGATACCCTGGTAGTCCACGCTGTAAACGATGATTACTA |
| | | GGTGTGGGGGGACTGACCCCTTCCGTGCCGCAGTTAACACAATAAGTAATCCACCTG |
| | | GGGAGTACGGCCGCAAGGTTGAAACTCAAAGGAATTGACGGGGGCCCGCACAAGCA |
| | | GTGGAGTATGTGGTTTAATTCGAAGCAACGCGAAGAACCTTACCAGGTCTTGACATC |
| | | GTATGCATAACTTAGAGATAAGTGAAATCCCTTCGGGGACATATAGACAGGTGGTGC |
| | | ATGGTTGTCGTCAGCTCGTGTCGTGAGATGTTGGGTTAAGTCCCGCAACGAGCGCAAC |
| | | CCTTACCTTTAGTTGCTACGCAAGAGCACTCTAAAGGGACTGCCGTTGACAAAACGG |
| | | AGGAAGGTGGGGATGACGTCAAATCATCATGCCCCTTATGACCTGGGCTACACACGT |
| | | ACTACAATGGCAATTAACAAAGAGAAGCAAGACAGCGATGTGGAGCAAATCTCGAA |
| | | AAATTGTCCCAGTTCAGATTGCAGGCTGCAACTCGCCTGCATGAAGTCGGAATTGCTA |
| | | GTAATCGTGGATCAGCATGCCACGGTGAATACGTTCCCGGGCCTTGTACACACCGCCC |
| | | GTCACACCATGGGAGTCGGTAACACCCGAAGTCGGTAGTCTAACAGCAATGAGGACG |
| | | CCGCCGAAGGTGGGATTGATGACTGGGGTGAAGTCGTAACAAGGTAGCCGTATCGGA |
| | | AGGTGCGGCTGGATCACCTCCTTT (SEQ ID NO: 51) |
| GCF_000177015 | Ruminococcaceae | TATTGAGAGTTTGATCCTGGCTCAGGATGAACGCTGGCGGCGTGCTTAACACATGCAA |
| | bacterium D16 | GTCGAACGGAGTGCCTTTGAAAGAGGATTCGTCCAATTGATAAGGTTACTTAGTGGCG |
| | | GACGGGTGAGTAACGCGTGAGGAACCTGCCTTGGAGTGGGGAATAACACAGTGAAA |
| | | ATTGTGCTAATACCGCATAATGCAGTTGGGCCGCATGGCTCTGACTGCCAAAGATTTA |
| | | TCGCTCTGAGATGGCCTCGCGTCTGATTAGCTAGTTGGTGGGGTAACGGCCCACCAAG |
| | | GCGACGATCAGTAGCCGGACTGAGAGGTTGGCCGGCCACATTGGGACTGAGACACGG |
| | | CCCAGACTCCTACGGGAGGCAGCAGTGGGGAATATTGGGCAATGGGCGCAAGCCTGA |
| | | CCCAGCAACGCCGCGTGAAGGAAGAAGGCTTTCGGGTTGTAAACTTCTTTTCTTAGGG |
| | | ACGAAGCAAGTGACGGTACCTAAGGAATAAGCCACGGCTAACTACGTGCCAGCAGCC |
| | | GCGGTAATACGTAGGTGGCAAGCGTTATCCGGATTTACTGGGTGTAAAGGGCGTGTA |
| | | GGCGGGATTGCAAGTCAGATGTGAAAACCACGGGCTCAACCTGTGGCCTGCATTTGA |
| | | AACTGTAGTTCTTGAGTACTGGAGAGGCAGACGGAATTCCTAGTGTAGCGGTGAAAT |
| | | GCGTAGATATTAGGAGGAACACCAGTGGCGAAGGCGGTCTGCTGGACAGCAACTGAC |
| | | GCTGAGGCGCGAAAGCGTGGGGAGCAAACAGGATTAGATACCCTGGTAGTCCACGCT |
| | | GTAAACGATGGATACTAGGTGTGGGGGGTCTGACCCCTTCCGTGCCGCAGTTAACAC |
| | | AATAAGTATCCCACCTGGGGAGTACGATCGCAAGGTTGAAACTCAAAGGAATTGACG |
| | | GGGGCCCGCACAAGCGGTGGAGTATGTGGTTTAATTCGAAGCAACGCGAAGAACCTT |
| | | ACCAGGGCTTGACATCCCGAGGCCCGGTCTAGAGATAGACCTTTCTCTTCGGAGACCT |
| | | CGGTGACAGGTGGTGCATGGTTGTCGTCAGCTCGTGTCGTGAGATGTTGGGTTAAGTC |
| | | CCGCAACGAGCGCAACCCCTATTGTTAGTTGCTACGCAAGAGCACTCTAGCGAGACT |
| | | GCCGTTGACAAAACGGAGGAAGGTGGGGACGACGTCAAATCATCATGCCCCTTATGT |
| | | CCTGGGCCACACACGTACTACAATGGTGGTTAACAGAGGGAGGCAATACCGCGAGGT |
| | | GGAGCAAACCCCTAAAAGCCATCCCAGTTCGGATTGCAGGCTGCAACCCGCCTGCAT |
| | | GAAGTTGGAATCGCTAGTAATCGCGGATCAGCATGCCGCGGTGAATACGTTCCCGGG |
| | | CCTTGTACACACCGCCCGTCACACCATGAGAGTCGGGAACACCCGAAGTCCGTAGCC |
| | | TAACCGCAAGGGGGGCGCGGCCGAAGGTGGGTTCGATAATTGGGGTGAAGTCGTAAC |
| | | AAGGTAGCCGTATCGGAAGGTGCGGCTGGATCACCTCCTTT (SEQ ID NO: 52) |
| GCF_000178155 | Ruminococcus albus | GGCCCACCAAGCCGACGATCAGTAGCCGGACTGAGAGGTTGAACGGCCACATTGGGA |
| | | CTGAGACACGGCCCAGACTCCTACGGGAGGCAGCAGTGGGGAATATTGCACAATGGG |
| | | CGAAAGCCTGATGCAGCGATGCCGCGTGAGGGAAGAAGGTTTTAGGATTGTAAACCT |
| | | CTGTCTTCGGGGACGATAATGACGGTACCCGAGGAGGAAGCTCCGGCTAACTACGTG |
| | | CCAGCAGCCGCGGTAATACGTAGGGAGCGAGCGTTGTCCGGAATTACTGGGTGTAAA |
| | | GGGAGCGTAGGCGGGACTGCAAGTCAGGTGTGAAATGTAGGGGCTTAACCCCTACCC |
| | | TGCACTTGAAACTGTGGTTCTTGAGTGAAGTAGAGGTAAGCGGAATTCCTAGTGTAGC |
| | | GGTGAAATGCGTAGATATTAGGAGGAACATCAGTGGCGAAGGCGGCTTACTGGGCTT |
| | | TAACTGACGCTGAGGCTCGAAAGCGTGGGGAGCAAACAGGATTAGATACCCTGGTAG |
| | | TCCACGCCGTAAACGATGATTACTAGGTGTGGGGGGACTGACCCCTTCCGTGCCGCA |
| | | GTTAACACAATAAGTAATCCACCTGGGGAGTACGACCGCAAGGTTGAAACTCAAAGG |
| | | AATTGACGGGGGCCCGCACAAGCAGTGGAGTATGTGGTTTAATTCGAAGCAACGCGA |
| | | AGAACCTTACCAGGTCTTGACATCGTGAGCATAGCTTAGAGATAAGTGAAATCCCTTC |
| | | GGGGACTCATAGACAGGTGGTGCATGGTTGTCGTCAGCTCGTGTCGTGAGATGTTGGG |
| | | TTAAGTCCCGCAACGAGCGCAACCCTTACTGTTAGTTGCTACGCAAGAGCACTCTAGC |
| | | AGGACTGCCGTTGACAAAACGGAGGAAGGTGGGGATGACGTCAAATCATCATGCCCC |
| | | TTATGACCTGGGCTACACACGTACTACAATGGCTGTTAACAGAGGGAAGCAAAGCAG |
| | | TGATGCAGAGCAAAACCCTAAAAGCAGTCTTAGTTCGGATTGTAGGCTGCAACCCGC |
| | | CTACATGAAGTCGGAATTGCTAGTAATCGCGGATCAGCATGCCGCGGTGAATACGTT |
| | | CCCGGGCCTTGTACACACCGCCCGTCACGCCATGGGAGTCGGTAACACCCGAAGCCT |
| | | GTGTTCTAACCGCAAGGAGGAAGCAGTCGAAGGTGGGATTGATGACTGGGGTGAAGT |
| | | CGTAACAAGGTAGCCGTATCGGAAGGTGCGGCTGGATCACCTCCTTT (SEQ ID NO: 53) |
| GCF_000403395 | Anaerotruncus sp | AGATGGGCTCGCGGCCGATTAGCTAGTTGGTGGGGCAACGGCCCACCAAGGCGACGA |
| | G3 2012 | TCGGTAGCCGGACTGAGAGGTTGATCGGCCACATTGGGACTGAGACACGGCCCAGAC |
| | | TCCTACGGGAGGCAGCAGTGGGGGATATTGCACAATGGAGGAAACTCTGATGCAGCG |
| | | ACGCCGCGTGAGGGAAGACGGTCTTCGGATTGTAAACCTCTGTCTTTGGGGAAGAAA |
| | | ATGACGGTACCCAAAGAGGAAGCTCCGGCTAACTACGTGCCAGCAGCCGCGGTAATA |
| | | CGTAGGGAGCGAGCGTTGTCCGGAATTACTGGGTGTAAAGGGAGCGTAGGCGGGCGA |
| | | GAAAGTTGAATGTTAAATCTACCGGCTTAACTGGTAGCTGCGTTCAAAACTTCTTGTC |
| | | TTGAGTGAAGTAGAGGCAGGCGGAATTCCTAGTGTAGCGGTGAAATGCGTAGATATT |
| | | AGGAGGAACACCAGTGGCGAAGGCGGCCTGCTGGGCTTTAACTGACGCTGAGGCTCG |
| | | AAAGCGTGGGGAGCAAACAGGATTAGATACCCTGGTAGTCCACGCTGTAAACGATGA |
| | | TTACTAGGTGTGGGGGGACTGACCCCTTCCGTGCCGCAGTTAACACAATAAGTAATCC |
| | | ACCTGGGGAGTACGGCCGCAAGGTTGAAACTCAAAGGAATTGACGGGGGCCCGCAC |
| | | AAGCAGTGGAGTATGTGGTTTAATTCGAAGCAACGCGAAGAACCTTACCAGGTCTTG |
| | | ACATCGTGCGCATAGCCTAGAGATAGGTGAAGCCCTTCGGGGCGCACAGACAGGTGG |
| | | TGCATGGTTGTCGTCAGCTCGTGTCGTGAGATGTTGGGTTAAGTCCCGCAACGAGCGC |
| | | AACCCTTATTATTAGTTGCTACGCAAGAGCACTCTAATGAGACTGCCGTTGACAAAAC |
| | | GGAGGAAGGTGGGGATGACGTCAAATCATCATGCCCCTTATGACCTGGGCTACACAC |
| | | GTACTACAATGGCACTGAAACAGAGGGAAGCGACATCGCGAGGTGAAGCGAATCCC |
| | | AAAAAAGTGTCCCAGTTCGGATTGCAGGCTGCAACTCGCCTGCATGAAGTCGGAATT |
| | | GCTAGTAATCGCGGATCAGCATGCCGCGGTGAATACGTTCCCGGGCCTTGTACACACC |
| | | GCCCGTCACACCATGGGAGTCGGTAACACCCGAAGCCAGTAGCCTAACCGCAAGGAG |
| | | GGCGCTGTCGAAGGTGGGATTGATGACTGGGGTGAAGTCGTAACAAGGTAGCCGTAT |
| | | CGGAAGGTGCGGCTGGATCACCTCCTTT (SEQ ID NO: 54) |
| GCF_000403435 | Oscillibacter sp 1 3 | TATAGAGAGTTTGATCCTGGCTCAGGACGAACGCTGGCGGCGTGCTTAACACATGCA |
| | | AGTCGAACGGAGCACCCCTGAAGGAGTTTTCGGACAACGGAAGGGAATGCTTAGTGG |
| | | CGGACTGGTGAGTAACGCGTGAGGAACCTGCCTTCCAGAGGGGGACAACAGTTGGAA |
| | | ACGACTGCTAATACCGCATGAAACATTTGAACCGCATGGTTTGAATGTCAAAGATTTA |
| | | TCGCTGGAAGATGGCCTCGCGTCTGATTAGCTAGTAGGCGGGGTAACGGCCCACCTA |
| | | GGCGACGATCAGTAGCCGGACTGAGAGGTTGACCGGCCACATTGGGACTGAGATACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCTTTCGGGTTGTAAACTTCTTTTAAGAG |
| | | GGAAGAGAAGAAGACGGTACCTCTTGAATAAGCCACGGCTAACTACGTGCCAGCAGC |
| | | CGCGGTAATACGTAGGTGGCAAGCGTTGTCCGGATTTACTGGGTGTAAAGGGCGTGC |
| | | AGCCGGGAAGACAAGTCAGATGTGAAATCCCGCGGCTCAACCGCGGAACTGCATTTG |
| | | AAACTGTTTTTCTTGAGTACCGGAGAGGTCATCGGAATTCCTTGTGTAGCGGTGAAAT |
| | | GCGTAGATATAAGGAAGAACACCAGTGGCGAAGGCGGATGACTGGACGGCAACTGA |
| | | CGGTGAGGCGCGAAAGCGTGGGGAGCAAACAGGATTAGATACCCTGGTAGTCCACGC |
| | | TGTAAACGATGGATACTAGGTGTGCGGGGACTGACCCCCTGCGTGCCGCAGTTAACA |
| | | CAATAAGTATCCCACCTGGGGAGTACGATCGCAAGGTTGAAACTCAAAGGAATTGAC |
| | | GGGGGCCCGCACAAGCGGTGGATTATGTGGTTTAATTCGAAGCAACGCGAAGAACCT |
| | | TACCAGGGCTTGACATGGAGAGGACCGCTCTAGAGATAGGGTTTTCCCTTCGGGGAC |
| | | CTCTCACACAGGTGGTGCATGGTTGTCGTCAGCTCGTGTCGTGAGATGTTGGGTTAAG |
| | | TCCCGCAACGAGCGCAACCCCTATTGTTAGTTGCTACGCAAGAGCACTCTAGCGAGA |
| | | CTGCCGTTGACAAAACGGAGGAAGGTGGGGACGACGTCAAATCATCATGCCCCTTAT |
| | | GTCCTGGGCTACACACGTAATACAATGGCGGTCAACAGAGGGATGCAAATCCGCGAG |
| | | GAGGAGCGAACCCCCAAAAGCCGTCCCAGTTCGGATCGCAGGCTGCAACCCGCCTGC |
| | | GTGAAGTCGGAATCGCTAGTAATCGCGGATCAGCATGCCGCGGTGAATACGTTCCCG |
| | | GGCCTTGTACACACCGCCCGTCACACCATGAGAGTCGGGAACACCCGAAGTCCGTAG |
| | | CCTAACAGCAATGAGGGCGCGGCCGAAGGTGGGTTCGATAATTGGGGTGAAGTCGTA |
| | | ACAAGGTAGCCGTTCGAGAACGAGCGGCTGGATCACCTCCTTT (SEQ ID NO: 55) |
| GCF_000421005 | Clostridiales | ATTAGCTAGTTGGTGAGGTAACGGCCCACCAAGGCGACGATCAGTAGCCGGACTGAG |
| | bacterium NK3B98 | AGGTTGACCGGCCACATTGGGACTGAGACACGGCCCAGACTCCTACGGGAGGCAGCA |
| | | GTGGGGAATATTGCACAATGGGGGAAACCCTGATGCAGCAACGCCGCGTGAGTGATG |
| | | ACGGCCTTCGGGTTGTAAAGCTCTGTCTTCAGGGACGATAATGACGGTACCTGAGGA |
| | | GGAAGCCACGGCTAACTACGTGCCAGCAGCCGCGGTAATACGTAGGTGGCGAGCGTT |
| | | ATCCGGATTTACTGGGCGTAAAGGATGCGTAGGTGGAATTTTAAGTGGGATGTGAAA |
| | | TACCCGGGCTCAACCTGGGAACTGCATTCCAAACTGGAATTCTAGAGTGCAGGAGAG |
| | | GAAAGCGGAATTCCTAGTGTAGCGGTGAAATGCGTAGAGATTAGGAAGAACACCAGT |
| | | GGCGAAGGCGGCTTGCTGGACAGTAACTGACGCTAAGGCGCGAAAGCGTGGGGAGC |
| | | AAACAGGATTAGATACCCTGGTAGTCCACGCCGTAAACGATGGGTACTAGGTGTAGG |
| | | GGTTTCGATACCTCTGTGCCGCCGTAAACACAATAAGTACCCCGCCTGGGGAGTACG |
| | | GTCGCAAGATTAAAACTCAAAGGAATTGACGGGGGCCCGCACAAGTAGCGGAGCAT |
| | | GTGGTTTAATTCGAAGCAACGCGAAGAACCTTACCAGGTCTTGACATCCCGGCGACC |
| | | GGTGTAGAGATACACCTTCTTCTTCGGAAGCGCCGGTGACAGGTGGTGCATGGTTGTC |
| | | GTCAGCTCGTGTCGTGAGATGTTGGGTTAAGTCCCGCAACGAGCGCAACCCCTATAGT |
| | | TAGTTGCTAACAGTAAGATGAGCACTCTAGCTAGACTGCCGTGGTTAACGCGGAGGA |
| | | AGGTGGGGATGACGTCAAATCATCATGCCCCTTATGTCTAGGGCTACACACGTGCTAC |
| | | AATGGCGAGAACAAAGAGAAGCAAGACCGCGAGGTGGAGCAAAACTCATAAAACTC |
| | | GTCCCAGTTCGGATTGCAGGCTGAAACCCGCCTGTATGAAGTTGGAATCGCTAGTAAT |
| | | CGCGGATCAGCATGCCGCGGTGAATACGTTCCCGGGCCTTGTACACACCGCCCGTCAC |
| | | ACCATGAGAGTCGGGAACACCCGAAGTCCGTAGCCTAACCGCAAGGGGGGCGCGGC |
| | | CGAAGGTGGGTTCGATAATTGGGGTGAAGTCGTAACAAGGTAGCCGT (SEQ ID NO: 56) |
| GCF_000469425 | Oscillibacter sp | TATAGAGAGTTTGATCCTGGCTCAGGACGAACGCTGGCGGCGTGCTTAACACATGCA |
| | KLE 1728 | AGTCGAACGGAGCACCCTTGACTGAGGTTTCGGCCAAATGATAGGAATGCTTAGTGG |
| | | CGGACTGGTGAGTAACGCGTGAGGAACCTACCTTCCAGAGGGGGACAACAGTTGGAA |
| | | ACGACTGCTAATACCGCATGACGCATGACCGGGGCATCCCGGGCATGTCAAAGATTT |
| | | TATCGCTGGAAGATGGCCTCGCGTCTGATTAGCTAGATGGTGGGGTAACGGCCCACC |
| | | ATGGCGACGATCAGTAGCCGGACTGAGAGGTTGACCGGCCACATTGGGACTGAGATA |
| | | CGGCCCAGACTCCTACGGGAGGCAGCAGTGGGGAATATTGGGCAATGGACGCAAGTC |
| | | TGACCCAGCAACGCCGCGTGAAGGAAGAAGGCTTTCGGGTTGTAAACTTCTTTTGTCA |
| | | GGGAAGAGTAGAAGACGGTACCTGACGAATAAGCCACGGCTAACTACGTGCCAGCA |
| | | GCCGCGGTAATACGTAGGTGGCAAGCGTTGTCCGGATTTACTGGGTGTAAAGGGCGT |
| | | GCAGCCGGGCCGGCAAGTCAGATGTGAAATCTGGAGGCTTAACCTCCAAACTGCATT |
| | | TGAAACTGTAGGTCTTGAGTACCGGAGAGGTTATCGGAATTCCTTGTGTAGCGGTGAA |
| | | ATGCGTAGATATAAGGAAGAACACCAGTGGCGAAGGCGGATAACTGGACGGCAACT |
| | | GACGGTGAGGCGCGAAAGCGTGGGGAGCAAACAGGATTAGATACCCTGGTAGTCCA |
| | | CGCTGTAAACGATGGATACTAGGTGTGCGGGGACTGACCCCCTGCGTGCCGCAGTTA |
| | | ACACAATAAGTATCCCACCTGGGGAGTACGATCGCAAGGTTGAAACTCAAAGGAATT |
| | | GACGGGGGCCCGCACAAGCGGTGGATTATGTGGTTTAATTCGAAGCAACGCGAAGAA |
| | | CCTTACCAGGGCTTGACATCCTACTAACGAAGTAGAGAT (SEQ ID NO: 57) |
| GCF_000492175 | Firmicutes bacterium | TATTGAGAGTTTGATCCTGGCTCAGGATGAACGCTGGCGGCGTGCTTAACACATGCAA |
| | ASF500 | GTCGAACGGAGGACCCCTGAAGGAGTTTTCGGACAACTGAAGGGAATCCTTAGTGGC |
| | | GGACGGGTGAGTAACGCGTGAGTAACCTGCCTTGGAGTGGGGAATAACAGCTGGAAA |
| | | CAGCTGCTAATACCGCATGATATGTCTGTGTCGCATGGCACTGGACATCAAAGATTTA |
| | | TCGCTCTGAGATGGACTCGCGTCTGATTAGCTAGTTGGCGGGGTAACGGCCCACCAA |
| | | GGCGACGATCAGTAGCCGGACTGAGAGGTTGGCCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCTTTCGGGTTGTAAACTTCTTTTCTCAGG |
| | | GACGAAGCAAGTGACGGTACCTGAGGAATAAGCCACGGCTAACTACGTGCCAGCAGC |
| | | CGCGGTAATACGTAGGTGGCAAGCGTTATCCGGATTTACTGGGTGTAAAGGGCGTGT |
| | | AGGCGGGACTGCAAGTCAGATGTGAAAACCACGGGCTCAACCTGTGGCCTGCATTTG |
| | | AAACTGTAGTTCTTGAGTACTGGAGAGGCAGACGGAATTCCTAGTGTAGCGGTGAAA |
| | | TGCGTAGATATTAGGAGGAACACCAGTGGCGAAGGCGGTCTGCTGGACAGCAACTGA |
| | | CGCTGAGGCGCGAAAGCGTGGGGAGCAAACAGGATTAGATACCCTGGTAGTCCACGC |
| | | TGTAAACGATGGATACTAGGTGTGGGGGGACTGACCCCCTCCGTGCCGCAGTTAACA |
| | | CAATAAGTATCCCACCTGGGGAGTACGATCGCAAGGTTGAAACTCAAAGGAATTGAC |
| | | GGGGGCCCGCACAAGCGGTGGAGTATGTGGTTTAATTCGAAGCAACGCGAAGAACCT |
| | | TACCAGGGCTTGACATCCCGGCGACCGGTGTAGAGATACACTTTCTTCTTCGGAAGCG |
| | | CCGGTGACAGGTGGTGCATGGTTGTCGTCAGCTCGTGTCGTGAGATGTTGGGTTAAGT |
| | | CCCGCAACGAGCGCAACCCTTATTGTTAGTTGCTACGCAAGAGCACTCTAGCGAGACT |
| | | GCCGTTGACAAAACGGAGGAAGGTGGGGACGACGTCAAATCATCATGCCCCTTATGT |
| | | CCTGGGCCACACACGTACTACAATGGTGGTCAACAGAGGGAAGCAAAACCGCGAGGT |
| | | GGAGCAAATCCCTAAAAGCCATCCCAGTTCGGATCGCAGGCTGCAACCCGCCTGCGT |
| | | GAAGTTGGAATCGCTAGTAATCGCGGATCAGCATGCCGCGGTGAATACGTTCCCGGG |
| | | CCTTGTACACACCGCCCGTCACACCATGAGAGTCGGGAACACCCGAAGTCCGTAGCC |
| | | TAACAGCAATGGGGGCGCGGCCGAAGGTGGGTTCGATAATTGGGGTGAAGTCGTAAC |
| | | AAGGTAGCCGTATCGGAAGGTGCGGCTGGATCACCTCCTTT (SEQ ID NO: 58) |
| GCF_000621805 | Ruminococcus sp | AATTAAGAGTTTGATCCTGGCTCAGGACGAACGCTGGCGGCACGCTTAACACATGCA |
| | FC2018 | AGTCGAACGGGGTTACAAGATAAGCTTGCTTAATTTGTAACCTAGTGGCGGACGGGT |
| | | GAGTAACACGTGAGCAATCTGCCCTTAAGAGGGGGATACCAGTTAGAAATGACTGTT |
| | | AATACCGCATAAGATAGTAGTACCGCATGGTACAGCTATAAAAGATTTATCGCTTAA |
| | | GGATGAGCTCGCGTCTGATTAGCTAGTTGGTGAGGTAACGGCCCACCAAGGCAACGA |
| | | TCAGTAGCCGGACTGAGAGGTTGGACGGCCACATTGGGACTGAGACACGGCCCAGAC |
| | | TCCTACGGGAGGCAGCAGTGGGGAATATTGCACAATGGAGGAAACTCTGATGCAGCG |
| | | ATGCCGCGTGAGGGAAGAAGGTTTTAGGATTGTAAACCTCTGTTGACAGGGACGATA |
| | | ATGACGGTACCTGTTGAGGAAGCTCCGGCTAACTACGTGCCAGCAGCCGCGGTAATA |
| | | CGTAGGGAGCGAGCGTTGTCCGGAATTACTGGGTGTAAAGGGAGCGTAGGCGGGATC |
| | | GCAAGTCAGGTGTGAAATGCGGGGGCTCAACCCCCGAACTGCACTTGAAACTGTGGT |
| | | TCTTGAGTGAAGTAGAGGTAAGCGGAATTCCTAGTGTAGCGGTGAAATGCGTAGATA |
| | | TTAGGAGGAACATCAGTGGCGAAGGCGGCTTACTGGGCTTTAACTGACGCTGAGGCT |
| | | CGAAAGCGTGGGGAGCAAACAGGATTAGATACCCTGGTAGTCCACGCCGTAAACGAT |
| | | GATTACTAGGTGTGGGGGGACTGACCCCTTCCGTGCCGCAGCTAACGCAATAAGTAA |
| | | TCCACCTGGGGAGTACGACCGCAAGGTTGAAACTCAAAGGAATTGACGGGGGCCCGC |
| | | ACAAGCAGTGGAGTATGTGGATTAATTCGAAGCAACGCGAAGAACCTTACCAGGTCT |
| | | TGACATCGTACGCATAGCATAGAGATATGTGAAATCCCTTCGGGGACGTATAGACAG |
| | | GTGGTGCATGGTTGTCGTCAGCTCGTGTCGTGAGATGTTGGGTTAAGTCCCGCAACGA |
| | | GCGCAACCCTTACTGTTAGTTGCTACGCAAGAGCACTCTAGCAGGACTGCCGTTGACA |
| | | AAACGGAGGAAGGTGGGGATGACGTCAAATCATCATGCCCCTTATGACCTGGGCCTC |
| | | ACACGTACTACAATGGCTGCCAACAGAGGGAAGCAAAGCAGTGATGCAGAGCAAAG |
| | | CCCCAAAAGCAGTCTTAGTTCGGATTGCAGGCTGAAACCCGCCTGCATGAAGTCGGA |
| | | ATTGCTAGTAATCGCAGATCAGCATGCTGCGGTGAATACGTTCCCGGGCCTTGTACAC |
| | | ACCGCCCGTCACGCCATGGGAGTCGGTAACACCCGAAGCCTGTAGCCCAACCGCAAG |
| | | GAGGACGCAGTCGAAGGTGGGATTGATGACTGGGGTGAAGTCGTAACAAGGTAGCC |
| | | GTATCGGAAGGTGCGGCTGGATCACCTCCTTT (SEQ ID NO: 59) |
| GCF_000686125 | Ruminococcus sp | TTAAAGAGTTTGATCCTGGCTCAGGACGAACGCTGGCGGCACGCTTAACACATGCAA |
| | NK3A76 | GTCGAACGGAGTTTTAGAGAGCTTGCTTTTTAAAACTTAGTGGCGGACGGGTGAGTAA |
| | | CACGTGAGCAATCTGCCTTTCAGAGGGGGATAGCAGTTGGAAACGACTGATAATACC |
| | | GCATAATATAGTAGGATCGCATGGTTCAACTATCAAAGATTTATCGCTGAAAGATGA |
| | | GCTCGCGTCTGATTAGATAGTTGGTGAGGTAACGGCTCACCAAGTCGACGATCAGTA |
| | | GCCGGACTGAGAGGTTGAACGGCCACATTGGGACTGAGACACGGCCCAGACTCCTAC |
| | | GGGAGGCAGCAGTGGGGAATATTGCACAATGGGCGCAAGCCTGATGCAGCGATGCC |
| | | GCGTGAGGGAAGAAGGTTTTAGGATTGTAAACCTCTGTCTTCAGGGACGATAATGAC |
| | | GGTACCTGAGGAGGAAGCTCCGGCTAACTACGTGCCAGCAGCCGCGGTAATACGTAG |
| | | GGAGCGAGCGTTGTCCGGAATTACTGGGTGTAAAGGGAGTGCAGGCGGGACTGCAAG |
| | | TCAGATGTGAAATGTAGGGGCTTAACCCCTGAACTGCATTTGAAACTGTAGTTCTTGA |
| | | GTGAAGTAGAGGTAAGCGGAATTCCTAGTGTAGCGGTGAAATGCGTAGATATTAGGA |
| | | GGAACATCAGTGGCGAAGGCGGCTTACTGGGCTTTTACTGACGCTGAGGCTCGAAAG |
| | | CGTGGGGAGCAAACAGGATTAGATACCCTGGTAGTCCACGCTGTAAACGATGATTAC |
| | | TAGGTGTGGGGGGACTGACCCCTTCCGTGCCGCAGTTAACACAATAAGTAATCCACCT |
| | | GGGGAGTACGGCCGCAAGGCTGAAACTCAAAGGAATTGACGGGGGCCCGCACAAGC |
| | | AGTGGAGTATGTGGATTAATTCGAAGCAACGCGAAGAACCTTACCAGGTCTTGACAT |
| | | CGTACGCATAGCATAGAGATATGTGAAATCCCTTCGGGGACGGACAGACAGGTGGTG |
| | | CATGGTTGTCGTCAGCTCGTGTCGTGAGATGTTGGGTTAAGTCCCGCAACGAGCGCAA |
| | | CCCTTACTGTTAGTTGCTACGCAAGAGCACTCTAGCAGGACTGCCGTTGACAAAACGG |
| | | AGGAAGGTGGGGATGACGTCAAATCATCATGCCCCTTATGACCTGGGCCTCACACGT |
| | | ACTACAATGGCTGTTAACAGAGAGAAGCGACATAGTGATATGAAGCAAAACCCTAAA |
| | | AGCAGTCTCAGTTCGGATTGCAGGCTGAAACCCGCCTGCATGAAGTCGGAATTGCTA |
| | | GTAATCGCGGATCAGCATGCCGCGGTGAATACGTTCCCGGGCCTTGTACACACCGCCC |
| | | GTCACACCATGGGAGTCGGTAACACCCGAAGTCAGTAGCCTAACCGTAAGGAGGGCG |
| | | CTGCCGAAGGTGGGATTGATGACTGGGGTGAAGTCGTAACAAGGTAGCCGTATCGGA |
| | | AGGTGCGGCTGGATCACCTCCTTT (SEQ ID NO: 60) |
| GCF_000701945 | Ruminococcus | CAAAGATTTATCACTCAGAGATGGGCTCGCGTCTGATTAGATAGTTGGTGAGGTAACG |
| | flavefaciens | GCTCACCAAGTCGACGATCAGTAGCCGGACTGAGAGGTTGAACGGCCACATTGGGAC |
| | | TGAGACACGGCCCAGACTCCTACGGGAGGCAGCAGTGGGGAATATTGCACAATGGGG |
| | | GGAACCCTGATGCAGCGATGCCGCGTGGAGGAAGAAGGTTTTCGGATTGTAAACTCC |
| | | TGTCTTAAAGGACGATAATGACGGTACTTTAGGAGGAAGCTCCGGCTAACTACGTGC |
| | | CAGCAGCCGCGGTAATACGTAGGGAGCGAGCGTTGTCCGGAATTACTGGGTGTAAAG |
| | | GGAGCGTAGGCGGGACTGCAAGTCAGATGTGAAATGCCGGGGCTTAACCCCGGAGCT |
| | | GCATTTGAAACTGTGGTTCTTGAGTGAAGTAGAGGCAAGCGGAATTCCTGGTGTAGC |
| | | GGTGAAATGCGTAGATATCAGGAGGAACACCGGTGGCGAAGGCGGCTTGCTGGGCTT |
| | | TTACTGACGCTGAGGCTCGAAAGCGTGGGTAGCAAACAGGATTAGATACCCTGGTAG |
| | | TCCACGCTGTAAACGATGATTACTAGGTGTGGGGGGACTGACCCCTTCCGTGCCGCAG |
| | | TTAACACAATAAGTAATCCACCTGGGGAGTACGGCCGCAAGGTTGAAACTCAAAGGA |
| | | ATTGACGGGGGCCCGCACAAGCAGTGGAGTATGTGGTTTAATTCGAAGCAACGCGAA |
| | | GAACCTTACCAGGTCTTGACATCGTATGCATAGCATAGAGATATGTGAAATCTCTTCG |
| | | GAGACATATAGACAGGTGGTGCATGGTTGTCGTCAGCTCGTGTCGTGAGATGTTGGGT |
| | | TAAGTCCCGCAACGAGCGCAACCCTTACCTTTAGTTGCTACGCAAGAGCACTCTAAAG |
| | | GGACTGCCGTTGACAAAACGGAGGAAGGTGGGGATGACGTCAAATCATCATGCCCCT |
| | | TATGACCTGGGCTACACACGTACTACAATGGCAATCAACAAAGAGAAGCAAGACAGT |
| | | GATGTGGAGCGAATCTCAAAAAATTGTCCCAGTTCGGATTGCAGGCTGCAACTCGCCT |
| | | GCATGAAGTCGGAATTGCTAGTAATCGCGGATCAGCATGCCGCGGTGAATACGTTCC |
| | | CGGGCCTTGTACACACCGCCCGTCACACCATGGGAGTCGGTAACACCCGAAGTCAGT |
| | | AGTCTAACAGCAATGAGGACGCTGCCGAAGGTGGGATTGATGACTGGGGTGAAGTCG |
| | | TAACAAGGTAGCCGTATCGGAAGGTGCGGCTGGATCACCTCCTTT (SEQ ID NO: 61) |
| GCF_000712055 | Ruminococcus sp | ATAAAGAGTTTGATCCTGGCTCAGGATGAACGCTGGCGGCACGCCTAACACATGCAA |
| | HUN007 | GTCGAACGGAGTTTAAGAGAGCTTGCTCTTTTAAACTTAGTGGCGGACGGGTGAGTA |
| | | ACACGTGAGCAACCTGCCTTTCAGAGAGGGATAGCTTCTGGAAACGGATGGTAATAC |
| | | CTCATAACATATTGATACGGCATCGTATTGATATCAAAGATTTATCGCTGAAAGATGG |
| | | GCTCGCGTCTGATTAGCTGGTTGGTGAGGTAACGGCCCACCAAGGCAACGATCAGTA |
| | | GCCGGACTGAGAGGTTGAACGGCCACATTGGGACTGAGACACGGCCCAGACTCCTAC |
| | | GGGAGGCAGCAGTGGGGAATATTGCACAATGGGCGCAAGCCTGATGCAGCGATGCC |
| | | GCGTGAGGGAAGAAGGTTTTCGGATTGTAAACCTCTGTCATCGGGGACGAAAATGAC |
| | | GGTACCCGAGAAGGAAGCTCCGGCTAACTACGTGCCAGCAGCCGCGGTAATACGTAG |
| | | GGAGCAAGCGTTATCCGGAATTACTGGGTGTAAAGGGAGTGTAGGCGGGACTGCAAG |
| | | TCAGATGTGAAATATGCCGGCTCAACTGGCAGACTGCATTTGAAACTGTGGTTCTTGA |
| | | GTGAAGTAGAGGTAAGCGGAATTCCTAGTGTAGCGGTGAAATGCGTAGATATTAGGA |
| | | GGAACATCAGTGGCGAAGGCGGCTTACTGGGCTTTAACTGACGCTGAGGCTCGAAAG |
| | | CGTGGGGAGCAAACAGGATTAGATACCCTGGTAGTCCACGCTGTAAACGATGATTAC |
| | | TAGGTGTGGGGGGACTGACCCCTTCCGTGCCGCAGTTAACACAATAAGTAATCCACCT |
| | | GGGGAGTACGGCCGCAAGGCTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CGAGTGAAGTATCAAGAGATTGATATGTCTTCGGACACAAAGACAGGTGGTGCATGG |
| | | TTGTCGTCAGCTCGTGTCGTGAGATGTTGGGTTAAGTCCCGCAACGAGCGCAACCCTT |
| | | ACCATTAGTTGCTACGCAAGAGCACTCTAATGGGACTGCCGTTGACAAAACGGAGGA |
| | | AGGTGGGGATGACGTCAAATCATCATGCCCCTTATGACCTGGGCTACACACGTACTAC |
| | | AATGGCAATCGAACAGAGGGAAGCAATACAGCGATGTAAAGCAAAACCCGAAAAAA |
| | | TTGTCTCAGTTCGGATTGCAGGCTGCAACCCGCCTGCATGAAGTCGGAATTGCTAGTA |
| | | ATCGCAGATCAGCATGCTGCGGTGAATACGTTCCCGGGCCTTGTACACACCGCCCGTC |
| | | ACACCATGGGAGTCGGTAACACCCGAAGCCAGTAGTCCAACCGCAAGGAGGACGCTG |
| | | TCGAAGGTGGGATTGATGACTGGGGTGAAGTCGTAACAAGGTAGCCGTATCGGAAGG |
| | | TGCGGCTGGATCACCTCCTTT (SEQ ID NO: 62) |
| GCF_000752215 | bacterium M54 | TTTAGAGAGTTTGATCCTGGCTCAGGACGAACGCTGGCGGCGTGCCTAACACATGCA |
| | | AGTCGAACGGAATTAAGTTTAACACCGAACACTTTGTTTGGTGGGGACACCTGACCG |
| | | AGTGGTGGGTGTTGAGCTTAATTTAGTGGCGGACGGGTGAGTAACGCGTGAGTAACC |
| | | TGCCTTTCAGAGGGGGATAACGTCTGGAAACGGACGCTAATACCGCATGACATATTT |
| | | GGGCTGCATGGTCTGAATATCAAAGGAGCAATCCGCTGAAAGATGGACTCGCGTCCG |
| | | ATTAGCTAGTTGGTGAGATAAAGGCCCACCAAGGCGACGATCGGTAGCCGGACTGAG |
| | | AGGTTGAACGGCCACATTGGGACTGAGACACGGCCCAGACTCCTACGGGAGGCAGCA |
| | | GTGGGGGATATTGCACAATGGAGGAAACTCTGATGCAGCAACGCCGCGTGAGGGAA |
| | | GACGGTTTTCGGATTGTAAACCTCTGTCCTTGGTGACGAAACAAATGACGGTAGCCAA |
| | | GGAGGAAGCTCCGGCTAACTACGTGCCAGCAGCCGCGGTAATACGTAGGGAGCAAGC |
| | | GTTGTCCGGATTTACTGGGTGTAAAGGGTGCGTAGGCGGCTCTGCAAGTCAGGCGTG |
| | | AAATATATGGGCTTAACCCATAGACTGCGTTTGAAACTGTGGAGCTTGAGTGAAGTA |
| | | GAGGTAGGCGGAATTCCCGGTGTAGCGGTGAAATGCGTAGAGATCGGGAGGAACAC |
| | | CAGTGGCGAAGGCGGCTTACTGGGCTTTAACTGACGCTGAGGCACGAAAGCATGGGT |
| | | AGCAAACAGGATTAGATACCCTGGTAGTCCATGCCGTAAACGATGATTACTAGGTGT |
| | | GGGGGGTCTGACCCCTTCCGTGCCGGAGTTAACACAATAAGTAATCCACCTGGGGAG |
| | | TACGGCCGCAAGGTTGAAACTCAAAGGAATTGACGGGGGCCCGCACAAGCAGTGGA |
| | | GTATGTGGTTTAATTCGAAGCAACGCGAAGAACCTTACCAGGTCTTGACATCCAACTA |
| | | ACGAAGCAGAGATGCATCAGGTGCCCTTCGGGGAAAGTTGAGACAGGTGGTGCATGG |
| | | TTGTCGTCAGCTCGTGTCGTGAGATGTTGGGTTAAGTCCCGCAACGAGCGCAACCCCT |
| | | GTGATTAGTTGCTACGCAAGAGCACTCTAATCAGACTGCCGTTGACAAAACGGAGGA |
| | | AGGTGGGGACGACGTCAAATCATCATGCCCTTTATGACCTGGGCTACACACGTACTAC |
| | | AATGGCTGTTAACAAAGGGAAGCAAGACCGCGAGGTGGAGCAAAACCTAAAAAACA |
| | | GTCTCAGTTCGGATCGCAGGCTGCAACCCGCCTGCGTGAAGTTGGAATTGCTAGTAAT |
| | | CGCGGATCATCATGCCGCGGTGAATACGTTCCCGGGCCTTGTACACACCGCCCGTCAC |
| | | ACCATGGGAGCCGGTAATACCCGAAGTCAGTAGCCTAACCGCAAGGGAGGCGCTGCC |
| | | GAAGGTAGGATTGGCGACTGGGGTGAAGTCGTAACAAGGTAGCCGTATCGGAAGGTG |
| | | CGGCTGGATCACCTCCTTT (SEQ ID NO: 63) |
| GCF_000765135 | Intestinimonas | TATTGAGAGTTTGATCCTGGCTCAGGATGAACGCTGGCGGCGTGCTTAACACATGCAA |
| | butyriciproducens | GTCGAACGGAGCACCCCTGACGGAGTTTTCGGACAACGAAAGGGAATGCTTAGTGGC |
| | | GGACGGGTGAGTAACGCGTGAGTAACCTGCCTTGGAGTGGGGAATAACAGCCGGAA |
| | | ACGGCTGCTAATACCGCATGATGTATCTGGATCGCATGGTTCTGGATACCAAAGATTT |
| | | ATCGCTCTGAGATGGACTCGCGTCTGATTAGCTAGTTGGTGAGGTAATGGCTCACCAA |
| | | GGCGACGATCAGTAGCCGGACTGAGAGGTTGGCCGGCCACATTGGGACTGAGACACG |
| | | GCCCAGACTCCTACGGGAGGCAGCAGTGGGGAATATTGGGCAATGGGCGAAAGCCTG |
| | | ACCCAGCAACGCCGCGTGAAGGAAGAAGGCCCTCGGGTTGTAAACTTCTTTTGTCAG |
| | | GGACGAAGCAAGTGACGGTACCTGACGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGAGTGCAAGTCAGATGTGAAAACTATGGGCTCAACCCATAGCCTGCATTT |
| | | GAAACTGTACTTCTTGAGTGATGGAGAGGCAGGCGGAATTCCCTGTGTAGCGGTGAA |
| | | ATGCGTAGATATAGGGAGGAACACCAGTGGCGAAGGCGGCCTGCTGGACATTAACTG |
| | | ACGCTGAGGCGCGAAAGCGTGGGGAGCAAACAGGATTAGATACCCTGGTAGTCCACG |
| | | CCGTAAACGATGGATACTAGGTGTGGGGGGTCTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGACTTGACATCCTACTAACGAAGCAGAGATGCATAAGGTGCCCTTCGGGG |
| | | AAAGTAGAGACAGGTGGTGCATGGTTGTCGTCAGCTCGTGTCGTGAGATGTTGGGTTA |
| | | AGTCCCGCAACGAGCGCAACCCTTATTGTTAGTTGCTACGCAAGAGCACTCTAGCGA |
| | | GACTGCCGTTGACAAAACGGAGGAAGGTGGGGACGACGTCAAATCATCATGCCCCTT |
| | | ATGTCCTGGGCCACACACGTACTACAATGGCGGTCAACAGAGGGAAGCAAAGCCGCG |
| | | AGGTGGAGCAAATCCCTAAAAGCCGTCCCAGTTCGGATTGCAGGCTGAAACTCGCCT |
| | | GTATGAAGTCGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACAGCAATGGGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 64) |
| GCF_000765235 | Oscillibacter sp ER4 | TATTGAGAGTTTGATCCTGGCTCAGGACGAACGCTGGCGGCGTGCTTAACACATGCA |
| | | AGTCGAACGAGAATCTACTGAAAGAGTTTTCGGACAATGGATGTAGAGGAAAGTGGC |
| | | GGACGGGTGAGTAACGCGTGAGGAACCTGCCTTGAAGAGGGGGACAACAGTTGGAA |
| | | ACGACTGCTAATACCGCATGATGCATAGGGGTCGCATGATCTTTATGCCAAAGATTTA |
| | | TCGCTTCAAGATGGCCTCGCGTCTGATTAGCTAGTTGGCGGGGTAACGGCCCACCAAG |
| | | GCGACGATCAGTAGCCGGACTGAGAGGTTGAACGGCCACATTGGGACTGAGATACGG |
| | | CCCAGACTCCTACGGGAGGCAGCAGTGGGGAATATTGGGCAATGGGCGCAAGCCTGA |
| | | CCCAGCAACGCCGCGTGAAGGAAGAAGGCTTTCGGGTTGTAAACTTCTTTTAAGAGG |
| | | GAAGAGCAGAAGACGGTACCTCTAGAATAAGCCACGGCTAACTACGTGCCAGCAGCC |
| | | GCGGTAATACGTAGGTGGCAAGCGTTGTCCGGATTTACTGGGTGTAAAGGGCGTGCA |
| | | GCCGGGTCTGCAAGTCAGATGTGAAATCCATGGGCTCAACCCATGAACTGCATTTGA |
| | | AACTGTAGATCTTGAGTGTCGGAGGGGCAATCGGAATTCCTAGTGTAGCGGTGAAAT |
| | | GCGTAGATATTAGGAGGAACACCAGTGGCGAAGGCGGATTGCTGGACGATAACTGAC |
| | | GGTGAGGCGCGAAAGTGTGGGGAGCAAACAGGATTAGATACCCTGGTAGTCCACACT |
| | | GTAAACGATGAATACTAGGTGTGCGGGGACTGACCCCCTGCGTGCCGCAGTAAACAC |
| | | AATAAGTATTCCACCTGGGGAGTACGATCGCAAGGTTGAAACTCAAAGGAATTGACG |
| | | GGGGCCCGCACAAGCGGTGGATTATGTGGTTTAATTCGAAGCAACGCGAAGAACCTT |
| | | ACCAGGGTTTGACATCCTGCTAACGAAGTAGAGATACATTAGGTGCCCTTCGGGGAA |
| | | AGCAGAGACAGGTGGTGCATGGTTGTCGTCAGCTCGTGTCGTGAGATGTTGGGTTAA |
| | | GTCCCGCAACGAGCGCAACCCCTATTGTTAGTTGCTACGCAAGAGCACTCTAGCGAG |
| | | ACTGCCGTTGACAAAACGGAGGAAGGTGGGGACGACGTCAAATCATCATGCCCCTTA |
| | | TATCCTGGGCTACACACGTAATACAATGGCGGTCAACAGAGGGAAGCAAAGCCGCGA |
| | | GGCAGAGCAAACCCCCAAAAGCCGTCCCAGTTCGGATTGTAGGCTGCAACTCGCCTG |
| | | CATGAAGTCGGAATCGCTAGTAATCGCGGATCAGCATGCCGCGGTGAATACGTTCCC |
| | | GGGCCTTGTACACACCGCCCGTCACACCATGAGAGTCGGGAACACCCGAAGTCCGTA |
| | | GCCTAACCTGAAAAGGAGGGCGCGGCCGAAGGTGGGTTCGATAATTGGGGTGAAGTC |
| | | GTAACAAGGTAGCCGTTCGAGAACGAGCGGCTGGATCACCTCCTTT (SEQ ID NO: 65) |
| GCF_000820765 | Candidatus | ATTAAGAGTTTGATCCTGGCTCAGGACGAACGCTGGCGGCGCGCTTAACACATGCAA |
| | Soleaferrea | GTCGAACGGGGTTGTTTCTGACACTCAGTGGGTAATCGGTAGATTGCTGATTGAGTGT |
| | massiliensis | TGGGAATAACCTAGTGGCGGACGGGTGAGTAACACGTGAGCAACCTACCTTTCAGAG |
| | | GGGGATAACGTTTGGAAACGAACGCTAATACCGCATGATATAATTGGATGGCATCAT |
| | | CTGATTATCAAAGGAGCAATCCGCTGAAAGATGGGCTCGCGGCCGATTAGGTAGTTG |
| | | GAGTGGTAACGGCACACCAAGCCGACGATCGGTAGCCGGACTGAGAGGTTGAACGG |
| | | CCACATTGGGACTGAGACACGGCCCAGACTCCTACGGGAGGCAGCAGTGGGGAATAT |
| | | TGCACAATGGGCGAAAGCCTGATGCAGCGACGCCGCGTGAGGGAAGACGGTTTTCGG |
| | | ATTGTAAACCTCTGTCTTATGTGACGATAATGACGGTAGCATAGGAGGAAGCCACGG |
| | | CTAACTACGTGCCAGCAGCCGCGGTAATACGTAGGTGGCAAGCGTTGTCCGGAATTA |
| | | CTGGGTGTAAAGGGAGCGTAGGCGGGAATGCAAGTTGAATGTTAAATCTACCGGCTC |
| | | AACCGGTAGCTGCGTTCAAAACTGTATTTCTTGAGTGAAGTAGAGGCAGGCGGAATT |
| | | CCTAGTGTAGCGGTGAAATGCGTAGATATTAGGAGGAACACCAGTGGCGAAGGCGGC |
| | | CTGCTGGGCTTTTACTGACGCTGAGGCTCGAAAGCGTGGGGAGCAAACAGGATTAGA |
| | | TACCCTGGTAGTCCACGCTGTAAACGATGATTACTAGGTGTGGGGGGTCTGACCCCTT |
| | | CCGTGCCGGAGTTAACACAATAAGTAATCCACCTGGGGAGTACGACCGCAAGGTTGA |
| | | AACTCAAAGGAATTGACGGGGACCCGCACAAGCAGTGGAGTATGTGGTTTAATTCGA |
| | | AGCAACGCGAAGAACCTTACCAGGTCTTGACATCCAACTAACGAGGCAGAGATGCGT |
| | | TAGGTGCCCTTCGGGGAAAGTTGAGACAGGTGGTGCATGGTTGTCGTCAGCTCGTGTC |
| | | GTGAGATGTTGGGTTAAGTCCCGCAACGAGCGCAACCCTTACTATTAGTTGCTACGCA |
| | | AGAGCACTCTAATGGGACTGCCGTTGACAAAACGGAGGAAGGTGGGGATGACGTCA |
| | | AATCATCATGCCCCTTATGACCTGGGCCACACACGTACTACAATGGTGTTCAACAGAG |
| | | GGAAGCAAAACTGTGAAGTGGAGCAAACCCCTAAAAGACATCCCAGTTCGGATCGTA |
| | | GGCTGCAACCCGCCTACGTGAAGTTGGAATTGCTAGTAATCGCGGATCAGCATGCCG |
| | | CGGTGAATACGTTCCCGGGTCTTGTACACACCGCCCGTCACACCATGAGAGTCGGTAA |
| | | CACCCGAAGTCAGTAGCCTAACCGCAAGGAGGGCGCTGCCGAAGGTGGGATTGATGA |
| | | TTAGGGTGAAGTCGTAACAAGGTAGCCGTATCGGAAGGTGCGGCTGGATCACCTCCT |
| | | TT (SEQ ID NO: 66) |
| GCF_000953215 | Clostridium cellulosi | TTAGAGAGTTTGATCCTGGCTCAGGACGAACGCTGGCGGCGCGCCTAACACATGCAA |
| | | GTCGAGCGGAGATAGTACTTCGGTTCTATCTTAGCGGCGGACGGGTGAGTAACGCGT |
| | | GAGCAACCTGCCCTTGAGCGGGGGATAGCGTCTGGAAACGGACGGTAATACCGCATA |
| | | ATGTACGTTGGAGGCATCTCCGATGTACCAAAGGAGAAATCCACTCAAGGATGGGCT |
| | | CGCGTCCGATTAGGTAGTTGGTGAGGTAATGGCCCACCAAGCCTGCGATCGGTAGCC |
| | | GGACTGAGAGGTTGTACGGCCACATTGGGACTGAGACACGGCCCAGACTCCTACGGG |
| | | AGGCAGCAGTGGGGGATATTGCACAATGGAGGAAACTCTGATGCAGCGACGCCGCGT |
| | | GAGGGAAGAAGGTCTTCGGATTGTAAACCTCTGTCTTTCGGGACGAAGGAAGTGACG |
| | | GTACCGAAAGAGGAAGCCACGGCTAACTACGTGCCAGCAGCCGCGGTAATACGTAGG |
| | | TGGCGAGCGTTGTCCGGAATTACTGGGTGTAAAGGGTGCGTAGGCGGGTTGTCAAGT |
| | | TGGATGTGAAATCTCTGGGCTTAACTCAGAGGTTGCATTCAAAACTGGCGATCTTGAG |
| | | TGAGGTAGAGGCAGGCGGAATTCCCGGTGTAGCGGTGAAATGCGTAGATATCGGGAG |
| | | GAACACCAGTGGCGAAGGCGGCCTGCTGGGCCTTAACTGACGCTGAGGCACGAAAGC |
| | | ATGGGGAGCAAACAGGATTAGATACCCTGGTAGTCCATGCTGTAAACGATGATTGCT |
| | | AGGTGTGGGTGGACTGACCCCATCCGTGCCGGAGTTAACACAATAAGCAATCCACCT |
| | | GGGGAGTACGGCCGCAAGGTTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CCACCGAATCCGGAAGAGATTCTGGAGTGCCCTTCGGGGAGCGGTGAGACAGGTGGT |
| | | GCATGGTTGTCGTCAGCTCGTGTCGTGAGATGTTGGGTTAAGTCCCGCAACGAGCGCA |
| | | ACCCTTGTTAATAGTTGCTACGCAAGAGCACTCTATTAAGACTGCCGTTGATAAAACG |
| | | GAGGAAGGTGGGGATGACGTCAAATCATCATGCCCCTTATGACCTGGGCTACACACG |
| | | TACTACAATGGCCGCCAACAAAGGGAAGCAATACCGCGAGGTGGAGCGAATCCCCA |
| | | AAAGCGGTCCCAGTTCAGATTGCAGGCTGCAACCCGCCTGCATGAAGACGGAATTGC |
| | | TAGTAATCGCGGATCAGCATGCCGCGGTGAATACGTTCCCGGGCCTTGTACACACCGC |
| | | CCGTCACACCATGAGAGCCGGAAACACCCGAAGTCGTTTGCGTAACCGAAAGGAGCG |
| | | CGGCGCCGAAGGTGGGATCGGTGATTGGGGTGAAGTCGTAACAAGGTAGCCGTATCG |
| | | GAAGGTGCGGCTGGATCACCTCCTTT (SEQ ID NO: 67) |
| GCF_001305095 | Clostridia bacterium | TCTAAGAGTTTGATCCTGGCTCAGGACGAACGCTGGCGGCGCGCCTAACACATGCAA |
| | UCS 1 2F7 | GTCGAACGAGCCGAGGGGAGCTTGCTCCCCAGAGCTAGTGGCGGACGGGTGAGTAAC |
| | | ACGTGAGCAACCTGCCTTTCAGAGGGGGATAACGTTTGGAAACGAACGCTAATACCG |
| | | CATAACATACCGGGACCGCATGATTCTGGTATCAAAGGAGCAATCCGCTGAAAGATG |
| | | GGCTCGCGTCCGATTAGCTAGTTGGCGGGGTAACGGCCCACCAAGGCGACGATCGGT |
| | | AGCCGGACTGAGAGGTTGATCGGCCACATTGGGACTGAGACACGGCCCAGACTCCTA |
| | | CGGGAGGCAGCAGTGGGGGATATTGCACAATGGAGGAAACTCTGATGCAGCGACGC |
| | | CGCGTGAGGGAAGACGGTCTTCGGATTGTAAACCTCTGTCTTTGGGGACGATAATGA |
| | | CGGTACCCAAGGAGGAAGCTCCGGCTAACTACGTGCCAGCAGCCGCGGTAATACGTA |
| | | GGGAGCGAGCGTTGTCCGGAATTACTGGGTGTAAAGGGAGCGTAGGCGGGGTCTCAA |
| | | GTCGAATGTTAAATCTACCGGCTCAACTGGTAGCTGCGTTCGAAACTGGGGCTCTTGA |
| | | GTGAAGTAGAGGCAGGCGGAATTCCTAGTGTAGCGGTGAAATGCGTAGATATTAGGA |
| | | GGAACACCAGTGGCGAAGGCGGCCTGCTGGGCTTTTACTGACGCTGAGGCTCGAAAG |
| | | CGTGGGGAGCAAACAGGATTAGATACCCTGGTAGTCCACGCCGTAAACGATGATTAC |
| | | TAGGTGTGGGGGACTGACCCCTTCCGTGCCGGAGTTAACACAATAAGTAATCCACCT |
| | | GGGGAGTACGACCGCAAGGTTGAAACTCAAAGGAATTGACGGGGGCCCGCACAAGC |
| | | AGTGGATTATGTGGTTTAATTCGAAGCAACGCGAAGAACCTTACCAGGTCTTGACATC |
| | | GAGTGACGGCTCTAGAGATAGAGCTTTCCTTCGGGACACAAAGACAGGTGGTGCATG |
| | | GTTGTCGTCAGCTCGTGTCGTGAGATGTTGGGTTAAGTCCCGCAACGAGCGCAACCCT |
| | | TATTATTAGTTGCTACATTCAGTTGAGCACTCTAATGAGACTGCCGTTGACAAAACGG |
| | | AGGAAGGTGGGGATGACGTCAAATCATCATGCCCCTTATGACCTGGGCTACACACGT |
| | | AATACAATGGCGATCAACAGAGGGAAGCAAGACCGCGAGGTGGAGCAAACCCCTAA |
| | | AAGTCGTCTCAGTTCGGATTGCAGGCTGCAACTCGCCTGCATGAAGTCGGAATTGCTA |
| | | GTAATCGCGGATCAGCATGCCGCGGTGAATACGTTCCCGGGCCTTGTACACACCGCCC |
| | | GTCACACCATGGGAGTCGGTAACACCCGAAGTCAGTAGCCTAACCGCAAAGAGGGCG |
| | | CTGCCGAAGGTGGGATTGATGACTGGGGTGAAGTCGTAACAAGGTAGCCGTATCGGA |
| | | AGGTGCGGCTGGATCACCTCCTTT (SEQ ID NO: 68) |
| GCF_001305115 | Clostridia bacterium | TTTAGAGAGTTTGATCCTGGCTCAGGACGAACGCTGGCGGCGTGCCTAACACATGCA |
| | UC5 1 1E11 | AGTCGAACGGAGTTAAGAGAGCTTGCTCTTTTAACTTAGTGGCGGACGGGTGAGTAA |
| | | CGCGTGAGTAACCTGCCTTTCAGAGGGGAATAACATTCTGAAAAGAATGCTAATACC |
| | | GCATGAGATCGTAGTATCGCATGGTACAGCGACCAAAGGAGCAATCCGCTGAAAGAT |
| | | GGACTCGCGTCCGATTAGCTAGTTGGTGAGATAAAGGCCCACCAAGGCGACGATCGG |
| | | TAGCCGGACTGAGAGGTTGAACGGCCACATTGGGACTGAGACACGGCCCAGACTCCT |
| | | ACGGGAGGCAGCAGTGGGGGATATTGCACAATGGGGGAAACCCTGATGCAGCAACG |
| | | CCGCGTGAAGGAAGAAGGTCTTCGGATTGTAAACTTCTGTCCTCAGGGAAGATAATG |
| | | ACGGTACCTGAGGAGGAAGCTCCGGCTAACTACGTGCCAGCAGCCGCGGTAATACGT |
| | | AGGGAGCAAGCGTTGTCCGGATTTACTGGGTGTAAAGGGTGCGTAGGCGGATCTGCA |
| | | AGTCAGTAGTGAAATCCCAGGGCTTAACCCTGGAACTGCTATTGAAACTGTGGGTCTT |
| | | GAGTGAGGTAGAGGCAGGCGGAATTCCCGGTGTAGCGGTGAAATGCGTAGAGATCG |
| | | GGAGGAACACCAGTGGCGAAGGCGGCCTGCTGGGCCTTAACTGACGCTGAGGCACGA |
| | | AAGCATGGGTAGCAAACAGGATTAGATACCCTGGTAGTCCATGCCGTAAACGATGAT |
| | | TACTAGGTGTGGGTGGTCTGACCCCATCCGTGCCGGAGTTAACACAATAAGTAATCCA |
| | | CCTGGGGAGTACGGCCGCAAGGTTGAAACTCAAAGGAATTGACGGGGGCCCGCACA |
| | | AGCAGTGGAGTATGTGGTTTAATTCGAAGCAACGCGAAGAACCTTACCAGGTCTTGA |
| | | CATCCTGCTAACGAGGTAGAGATACGTTAGGTGCCCTTCGGGGAAAGCAGAGACAGG |
| | | TGGTGCATGGTTGTCGTCAGCTCGTGTCGTGAGATGTTGGGTTAAGTCCCGCAACGAG |
| | | CGCAACCCCTGCTATTAGTTGCTACGCAAGAGCACTCTAATAGGACTGCCGTTGACAA |
| | | AACGGAGGAAGGTGGGGACGACGTCAAATCATCATGCCCCTTATGACCTGGGCTACA |
| | | CACGTACTACAATGGCCGTCAACAGAGAGAAGCAAAGCCGCGAGGTGGAGCAAAAC |
| | | TCTAAAAACGGTCCCAGTTCGGATCGTAGGCTGCAACCCGCCTACGTGAAGTTGGAA |
| | | TTGCTAGTAATCGCGGATCATCATGCCGCGGTGAATACGTTCCCGGGCCTTGTACACA |
| | | CCGCCCGTCACACCATGGGAGCCGGTAATACCCGAAGTCAGTAGTCTAACCGCAAGG |
| | | GGGACGCTGCCGAAGGTAGGATTGGCGACTGGGGTGAAGTCGTAACAAGGTAGCCGT |
| | | ATCGGAAGGTGCGGCTGGATCACCTCCTTT (SEQ ID NO: 69) |
| GCF_001305135 | Clostridia bacterium | TTTAGAGAGTTTGATCCTGGCTCAGGATGAACGCTGGCGGCGTGCCTAACACATGCA |
| | UC5 1 1D1 | AGTCGAACGGGGTTATTTTGGAAATCTCTTCGGAGATGGAATTCTTAACCTAGTGGCG |
| | | GACGGGTGAGTAACGCGTGAGCAATCTGCCTTTAGGAGGGGGATAACAGTCGGAAAC |
| | | GGCTGCTAATACCGCATAATACGTTTGGGAGGCATCTCTTGAACGTCAAAGATTTTAT |
| | | CGCCTTTAGATGAGCTCGCGTCTGATTAGCTGGTTGGCGGGGTAACGGCCCACCAAG |
| | | GCGACGATCAGTAGCCGGACTGAGAGGTTGAACGGCCACATTGGGACTGAGACACGG |
| | | CCCAGACTCCTACGGGAGGCAGCAGTGGGGAATATTGCGCAATGGGGGAAACCCTGA |
| | | CGCAGCAACGCCGCGTGATTGAAGAAGGCCTCGGGTTGTAAAGATCTTTAATCAGGG |
| | | ACGAAAAATGACGGTACCTGAAGAATAAGCTCCGGCTAACTACGTGCCAGCAGCCGC |
| | | GGTAATACGTAGGGAGCAAGCGTTATCCGGATTTACTGGGTGTAAAGGGCGCGCAGG |
| | | CGGGCCGGCAAGTTGGGAGTGAAATCCCGGGGCTTAACCCCGGAACTGCTTTCAAAA |
| | | CTGCTGGTCTTGAGTGATGGAGAGGCAGGCGGAATTCCGTGTGTAGCGGTGAAATGC |
| | | GTAGATATACGGAGGAACACCAGTGGCGAAGGCGGCCTGCTGGACATTAACTGACGC |
| | | TGAGGCGCGAAAGCGTGGGGAGCAAACAGGATTAGATACCCTGGTAGTCCACGCCGT |
| | | AAACGATGGATACTAGGTGTGGGAGGTATTGACCCCTTCCGTGCCGCAGTTAACACA |
| | | ATAAGTATCCCACCTGGGGAGTACGGCCGCAAGGTTGAAACTCAAAGGAATTGACGG |
| | | GGGCCCGCACAAGCAGTGGAGTATGTGGTTTAATTCGAAGCAACGCGAAGAACCTTA |
| | | CCAGGTCTTGACATCCCGATGACCGGCGTAGAGATACGCCCTCTCTTCGGAGCATCGG |
| | | TGACAGGTGGTGCATGGTTGTCGTCAGCTCGTGTCGTGAGATGTTGGGTTAAGTCCCG |
| | | CAACGAGCGCAACCCTTACGGTTAGTTGATACGCAAGATCACTCTAGCCGGACTGCC |
| | | GTTGACAAAACGGAGGAAGGTGGGGACGACGTCAAATCATCATGCCCCTTATGACCT |
| | | GGGCTACACACGTACTACAATGGCAGTCATACAGAGGGAAGCAATACCGCGAGGTGG |
| | | AGCAAATCCCTAAAAGCTGTCCCAGTTCAGATTGCAGGCTGCAACCCGCCTGCATGA |
| | | AGTCGGAATTGCTAGTAATCGCGGATCAGCATGCCGCGGTGAATACGTTCCCGGGCC |
| | | TTGTACACACCGCCCGTCACACCATGAGAGCCGTCAATACCCGAAGTCCGTAGCCTA |
| | | ACCGCAAGGGGGGCGCGGCCGAAGGTAGGGGTGGTAATTAGGGTGAAGTCGTAACA |
| | | AGGTAGCCGTATCGGAAGGTGCGGCTGGATCACCTCCTTT (SEQ ID NO: 70) |
| GCF_001486665 | Fournierella | TATGAAGAGTTTGATCCTGGCTCAGGACGAACGCTGGCGGCGCGCCTAACACATGCA |
| | massiliensis | AGTCGAACGGAGCTTGCTTGTCAGATCCTTTCGGGGTGACGACTTGTAAGCTTAGTGG |
| | | CGAACGGGTGAGTAACACGTGAGTAACCTGCCCCAGAGTGGGGGACAACAGTTGGA |
| | | AACGACTGCTAATACCGCATAAGCCCACGGAACCGCATGGTTCAGAGGGAAAAGGA |
| | | GCAATTCGCTTTGGGATGGACTCGCGTCCGATTAGCTAGATGGTGAGGTAACGGCCC |
| | | ACCATGGCGACGATCGGTAGCCGGACTGAGAGGTTGATCGGCCACATTGGGACTGAG |
| | | ACACGGCCCAGACTCCTACGGGAGGCAGCAGTGGGGAATATTGCACAATGGGGGAA |
| | | ACCCTGATGCAGCGACGCCGCGTGGAGGAAGAAGGCCTTCGGGTTGTAAACTCCTGT |
| | | CGTAAGGGACGATAGTGACGGTACCTTACAAGAAAGCCACGGCTAACTACGTGCCAG |
| | | CAGCCGCGGTAAAACGTAGGTGGCAAGCGTTGTCCGGAATTACTGGGTGTAAAGGGA |
| | | GCGCAGGCGGGTCTGCAAGTTGGAAGTGAAACCCATGGGCTCAACCCATGAACTGCT |
| | | TTCAAAACTGCGGATCTTGAGTGGTGTAGAGGTAGGCGGAATTCCCGGTGTAGCGGT |
| | | GGAATGCGTAGATATCGGGAGGAACACCAGTGGCGAAGGCGGCCTACTGGGCACTA |
| | | ACTGACGCTGAGGCTCGAAAGCATGGGTAGCAAACAGGATTAGATACCCTGGTAGTC |
| | | CATGCCGTAAACGATGATTACTAGGTGTGGGAGGATTGACCCCTTCCGTGCCGCAGTT |
| | | AACACAATAAGTAATCCACCTGGGGAGTACGACCGCAAGGTTGAAACTCAAAGGAAT |
| | | TGACGGGGGCCCGCACAAGCAGTGGAGTATGTGGTTTAATTCGAAGCAACGCGAAGA |
| | | ACCTTACCAGGTCTTGACATCCCGTGCATAGCATAGAGATATGTGAAGTCCTTCGGGA |
| | | CACGGAGACAGGTGGTGCATGGTTGTCGTCAGCTCGTGTCGTGAGATGTTGGGTTAAG |
| | | TCCCGCAACGAGCGCAACCCTTATCGTTAGTTACTACGCAAGAGGACTCTAGCGAGA |
| | | CTGCCGTTGACAAAACGGAGGAAGGTGGGGATGACGTCAAATCATCATGCCCTTTAT |
| | | GACCTGGGCTACACACGTACTACAATGGCAATTAACAAAGAGAAGCAAAGCCGCGA |
| | | GGTGGAGCAAACCTCATAAAAATTGTCTCAGTTCAGATTGCAGGCTGCAACTCGCCTG |
| | | CATGAAGTCGGAATTGCTAGTAATCGCGGATCAGCATGCCGCGGTGAATACGTTCCC |
| | | GGGCCTTGTACACACCGCCCGTCACACCATGAGAGCCGGGGGGACCCGAAGTCCGTA |
| | | GCCTAACCGCAAGGAGGGCGCGGCCGAAGGTAAAACTGGTGATTGGGGTGAAGTCGT |
| | | AACAAGGTAGCCGTATCGGAAGGTGCGGCTGGATCACCTCCTTT (SEQ ID NO: 71) |
| GCF_001695555 | Clostridium sp | TTTAGAGAGTTTGATCCTGGCTCAGGACGAACGCTGGCGGCGTGCCTAACACATGCA |
| | W14A | AGTCGAACGGAAACAGATTGAAGCTTGCTTTGAACTGTTTTAGTGGCGGACGGGTGA |
| | | GTAACGCGTGAGGAACCTGCCTTTCAGAGGGGGATAACGTCTGGAAACGGACGCTAA |
| | | TACCGCATGACATTTTGTTGCCGCATGGTGATAAAATCAAAGGAGCAATCCGCTGAG |
| | | AGATGGACTCGCGTCCGATTAGCCGGTTGGCGGGGTAACGGCCCACCAAAGCAACGA |
| | | TCGGTAGCCGGGCTGAGAGGCTGAACGGCCACATTGGGACTGAGACACGGCCCAGAC |
| | | TCCTACGGGAGGCAGCAGTGGGGGATATTGCACAATGGAGGAAACTCTGATGCAGCA |
| | | ACGCCGCGTGAGGGAAGAAGGTTTTCGGATTGTAAACCTCTGTCCTCAGGGACGATA |
| | | ATGACGGTACCTGAGGAGGAAGCTCCGGCTAACTACGTGCCAGCAGCCGCGGTAATA |
| | | CGTAGGGAGCAAGCGTTGTCCGGATTTACTGGGTGTAAAGGGTGCGTAGGCGGCACT |
| | | GCAAGTCAGGTGTGAAAACCATGGGCTTAACTTATGGATTGCACTTGAAACTGTGGTG |
| | | CTTGAGTGAAGTAGAGGCAGGCGGAATTCCCGGTGTAGCGGTGAAATGCGTAGAGAT |
| | | CGGGAGGAACACCAGTGGCGAAGGCGGCCTGCTGGGCTTTAACTGACGCTGAGGCAC |
| | | GAAAGCATGGGTAGCAAACAGGATTAGATACCCTGGTAGTCCATGCCGTAAACGATG |
| | | ATTACTAGGTGTGGGGGGTCTGACCCCTTCCGTGCCGGAGTTAACACAATAAGTAATC |
| | | CACCTGGGAAGTACGACCGCAAGGTTGAAACTCAAAGGAATTGACGGGGGCCCGCAC |
| | | AAGCAGTGGAGTATGTGGTTTAATTCGAAGCAACGCGAAGAACCTTACCAGGTCTTG |
| | | ACATCCAACTAACGAAGCAGAGATGCATCAGGTGCCCTTCGGGGAAAGTTGAGACAG |
| | | GTGGTGCATGGTTGTCGTCAGCTCGTGTCGTGAGATGTTGGGTTAAGTCCCGCAACGA |
| | | GCGCAACCCTTGTGATTAGTTGCTACGCTAAGAGCACTCTAATCAGACTGCCGTTGAC |
| | | AAAACGGAGGAAGGTGGGGACGACGTCAAATCATCATGCCCCTTATGACCTGGGCTA |
| | | CACACGTACTACAATGGCCGTTAACAACGGGAAGCGAAGCCGCGAGGCGGAGCAAA |
| | | ACCCCAAAAACGGTCTCAGTTCGGATCGCAGGCTGCAACCCGCCTGCGTGAAGCTGG |
| | | AATTGCTAGTAATCGCGGATCATCATGCCGCGGTGAATACGTTCCCGGGCCTTGTACA |
| | | CACCGCCCGTCACACCATGGGAGCCGGTAATACCCGAAGTCGGTAGCCTAACCGCAA |
| | | GGAAGGCGCCGCCGAAGGTAGGATTGGCGACTGGGGTGAAGTCGTAACAAGGTAGC |
| | | CGTATCGGAAGGTGCGGCTGGATCACCTCCTTT (SEQ ID NO: 72) |
| GCF_002119605 | Ruminococcaceae | TTTAGAGAGTTTGATCCTGGCTCAGGACGAACGCTGGCGGCGTGCCTAACACATGCA |
| | bacterium CPB6 | AGTCGAACGAAACTTTTTGCTTCGGTAGAAAGTTTAGTGGCGGACGGGTGAGTAACG |
| | | CGTGAGGAACCTGCCTTTCAGAGGGGGATAATGTCTGGAAACGGACACTAATACCGC |
| | | ATGACATTTTCTGTTCACATGGACAGAAAATCAAAGGAGCAATCTGCTGAAAGATGG |
| | | ACTCGCGTCCGATTAGCTAGATGGTGAGATAATAGCCCACCATGGCGACGATCGGTA |
| | | GCCGGACTGAGAGGTTGAACGGCCACATTGGGACTGAGACACGGCCCAGACTCCTAC |
| | | GGGAGGCAGCAGTGGGGGATATTGCACAATGGAGGAAACTCTGATGCAGCAACGCC |
| | | GCGTGAAGGAAGACGGTCTTCGGATTGTAAACTTTTGTACCTAGGGACGATAATGAC |
| | | GGTACCTAGGCAGCAAGCTCCGGCTAACTACGTGCCAGCAGCCGCGGTAATACGTAG |
| | | GGAGCGAGCGTTGTCCGGATTTACTGGGTGTAAAGGGTGCGTAGGCGGCCAAGCAAG |
| | | TCAGCTGTGAAAACTATGGGCTTAACCCATAGCCTGCAATTGAAACTGTTTGGCTTGA |
| | | GTGAAGTAGAGGTAGGTGGAATTCCCGGTGTAGCGGTGAAATGCGTAGAGATCGGGA |
| | | GGAACACCAGTGGCGAAGGCGACCTACTGGGCTTTAACTGACGCTGAAGCACGAAAG |
| | | CATGGGTAGCAAACAGGATTAGATACCCTGGTAGTCCATGCTGTAAACGATGATTAC |
| | | TAGGTGTGGGGGGTCTGACCCCTTCCGTGCCGGAGTTAACACAATAAGTAATCCACCT |
| | | GGGGAGTACGACCGCAAGGTTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CCAACTAACGAAGCAGAGATGCATTAGGTGCCCTTCGGGGAAAGTTGAGACAGGTGG |
| | | TGCATGGTTGTCGTCAGCTCGTGTCGTGAGATGTTGGGTTAAGTCCCGCAACGAGCGC |
| | | AACCCTTACTGTTAGTTGCTACGCAAGAGCACTCTAGCAGGACTGCCGTTGACAAAAC |
| | | GGAGGAAGGTGGGGACGACGTCAAATCATCATGCCCCTTATGACCTGGGCTACACAC |
| | | GTACTACAATGGCCGTTAACAGAGAGAAGCGATACCGCGAGGTGGAGCGAACCTCAA |
| | | AAAGCGGTCTCAGTTCGGATTGCAGGCTGAAACCCGCCTGCATGAAGTTGGAATTGC |
| | | TAGTAATCGCGGATCATAATGCCGCGGTGAATACGTTCCCGGGCCTTGTACACACCGC |
| | | CCGTCACACCATGGGAGCCGGTAATACCCGAAGTCAGTAGTCTAACCGCAAGGAGGA |
| | | CGCTGCCGAAGGTAGGATTGGCGACTGGGGTGAAGTCGTAACAAGGTAGCCGTATCA |
| | | GAAGGTGCGGCTGGATCACCTCCTTT (SEQ ID NO: 73) |
| GCF_002159175 | Flavonifractor sp | TATTGAGAGTTTGATCCTGGCTCAGGATGAACGCTGGCGGCGTGCTTAACACATGCAA |
| | An92 | GTCGAACGGAGTGCTCATGACGGAGTTTTCGGACAACGGATTGAGTTACTTAGTGGC |
| | | GGACGGGTGAGTAACGCGTGAGGAACCTGCCTTGGAGTGGGGAATAACAGTTGGAA |
| | | ACAGCTGCTAATACCGCATAATGCAGTTGGGTCGCATGGCCCTGACTGCCAAAGATTT |
| | | ATCGCTCTGAGATGGCCTCGCGTCTGATTAGCTGGTTGGCGGGGTAACGGCCCACCAA |
| | | GGCGACGATCAGTAGCCGGACTGAGAGGTTGGCCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGATGAAGGCTTTCGGGTTGTAAACTTCTTTTGTCAGG |
| | | GACGAAACAAATGACGGTACCTGACGAATAAGCCACGGCTAACTACGTGCCAGCAGC |
| | | CGCGGTAATACGTAGGTGGCAAGCGTTATCCGGATTTACTGGGTGTAAAGGGCGTGT |
| | | AGGCGGGATTGCAAGTCAGATGTGAAAACCAGGGGCTCAACCTCTGGCCTGCATTTG |
| | | AAACTGTAGTTCTTGAGTGCTGGAGAGGCAATCGGAATTCCGTGTGTAGCGGTGAAA |
| | | TGCGTAGATATACGGAGGAACACCAGTGGCGAAGGCGGATTGCTGGACAGTAACTGA |
| | | CGCTGAGGCGCGAAAGCGTGGGGAGCAAACAGGATTAGATACCCTGGTAGTCCACGC |
| | | CGTAAACGATGGATACTAGGTGTGGGGGGACTGACCCCCTCCGTGCCGCAGCTAACG |
| | | CAATAAGTATCCCACCTGGGGAGTACGATCGCAAGGTTGAAACTCAAAGGAATTGAC |
| | | GGGGGCCCGCACAAGCGGTGGAGTATGTGGTTTAATTCGAAGCAACGCGAAGAACCT |
| | | TACCAGGGCTTGACATCCTACTAACGAAGCAGAGATGCATAAGGTGCCCTTCGGGGA |
| | | AAGTAGAGACAGGTGGTGCATGGTTGTCGTCAGCTCGTGTCGTGAGATGTTGGGTTAA |
| | | GTCCCGCAACGAGCGCAACCCCTATTGTTAGTTGCTACGCAAGAGCACTCTAGCGAG |
| | | ACTGCCGTTGACAAAACGGAGGAAGGTGGGGACGACGTCAAATCATCATGCCCCTTA |
| | | TGTCCTGGGCCACACACGTACTACAATGGTGGTTAACAGAGGGAGGCAAAACCGCGA |
| | | GGTGGAGCAAATCCCTAAAAGCCATCCCAGTTCGGATTGCAGGCTGCAACCCGCCTG |
| | | TATGAAGTTGGAATCGCTAGTAATCGCGGATCAGCATGCCGCGGTGAATACGTTCCC |
| | | GGGCCTTGTACACACCGCCCGTCACACCATGAGAGTCGGGAACACCCGAAGTCCGTA |
| | | GCCTAACCGCAAGGAGGGCGCGGCCGAAGGTGGGTTCGATAATTGGGGTGAAGTCGT |
| | | AACAAGGTAGCCGTATCGGAAGGTGCGGCTGGATCACCTCCTTT (SEQ ID NO: 74) |
| GCF_002159225 | Flavonifractor sp | TATTGAGAGTTTGATCCTGGCTCAGGATGAACGCTGGCGGCGTGCTTAACACATGCAA |
| | An91 | GTCGAACGGAGTGCTCATGACGGAGGATTCGTCCAACGGATTGAGTTACTTAGTGGC |
| | | GGACGGGTGAGTAACGCGTGAGGAACCTGCCTCGGAGTGGGGAATAACAGCCCGAA |
| | | AGGGTTGCTAATACCGCATGATGCAGTTGGGCCGCATGGCTCTGACTGCCAAAGATTT |
| | | ATCGCTCTGAGATGGCCTCGCGTCTGATTAGCTGGTTGGCGGGGTAACGGCCCACCAA |
| | | GGCGACGATCAGTAGCCGGACTGAGAGGTTGACCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGATGAAGGCTTTCGGGTTGTAAACTTCTTTTATTCGG |
| | | GACGAAGAAAATGACGGTACCGAATGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGACTGCAAGTCAGATGTGAAAACTATGGGCTCAACCCATAGCCTGCATTT |
| | | GAAACTGTAGTTCTTGAGTGCTGGAGAGGCAATCGGAATTCCGTGTGTAGCGGTGAA |
| | | ATGCATAGATATACGGAGGAACACCAGTGGCGAAGGCGGATTGCTGGACAGTAACTG |
| | | ACGCTGAGGCGCGAAAGCGTGGGGAGCAAACAGGATTAGATACCCTGGTAGTCCACG |
| | | CCGTAAACGATGGATACTAGGTGTGGGGGGTCTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCC (SEQ ID NO: 75) |
| GCF_002159455 | Flavonifractor sp | TATTGAGAGTTTGATCCTGGCTCAGGATGAACGCTGGCGGCGTGCTTAACACATGCAA |
| | An306 | GTCGAACGGAGTGCTCATGACAGAGGATTCGTCCAATGGATTGAGTTACTTAGTGGC |
| | | GGACGGGTGAGTAACGCGTGAGGAACCTGCCTCGGAGTGGGGAATAACAGACCGAA |
| | | AGGCCTGCTAATACCGCATGATACAGTTGGGTCGCATGGCTCTGACTGTCAAAGATTT |
| | | ATCGCTCTGAGATGGCCTCGCGTCTGATTAGCTAGTTGGCGGGGTAACGGCCCACCAA |
| | | GGCGACGATCAGTAGCCGGACTGAGAGGTTGACCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCTTTCGGGTTGTAAACTTCTTTTCTCGGG |
| | | GACGAAACAAATGACGGTACCTGAGGAATAAGCCACGGCTAACTACGTGCCAGCAGC |
| | | CGCGGTAATACGTAGGTGGCGAGCGTTATCCGGATTTACTGGGTGTAAAGGGCGTGT |
| | | AGGCGGGATTGCAAGTCAGACGTGAAAACTATGGGCTCAACCCATAGCCTGCGTTTG |
| | | AAACTGTAGTTCTTGAGTGCTGGAGAGGCAATCGGAATTCCGTGTGTAGCGGTGAAA |
| | | TGCGTAGATATACGGAGGAACACCAGTGGCGAAGGCGGATTGCTGGACAGTAACTGA |
| | | CGCTGAGGCGCGAAAGCGTGGGGAGCAAACAGGATTAGATACCCTGGTAGTCCACGC |
| | | CGTAAACGATGGATACTAGGTGTGGGGGGTCTGACCCCCTCCGTGCCGCAGTTAACA |
| | | CAATAAGTATCCCACCTGGGGAGTACGATCGCAAGGTTGAAACTCAAAGGAATTGAC |
| | | GGGGGCCCGCACAAGCGGTGGAGTATGTGGTTTAATTCGAAGCAACGCGAAGAACCT |
| | | TACCAGGGCTTGACATCCCACTAACGAAGCAGAGATGCATTAGGTGCCCTTCGGGGA |
| | | AAGTGGAGACAGGTGGTGCATGGTTGTCGTCAGCTCGTGTCGTGAGATGTTGGGTTAA |
| | | GTCCCGCAACGAGCGCAACCCTTATTGTTAGTTGCTACGCAAGAGCACTCTAGCGAG |
| | | ACTGCCGTTGACAAAACGGAGGAAGGTGGGGACGACGTCAAATCATCATGCCCCTTA |
| | | TGTCCTGGGCCACACACGTACTACAATGGTGGTTAACAGAGGGAAGCAATACCGCGA |
| | | GGTGGAGCAAATCCCTAAAAGCCATCCCAGTTCGGATTGCAGGCTGAAACCCGCCTG |
| | | TATGAAGTTGGAATCGCTAGTAATCGCGGATCAGCATGCCGCGGTGAATACGTTCCC |
| | | GGGCCTTGTACACACCGCCCGTCACACCATGAGAGTCGGGAACACCCGAAGTCCGTA |
| | | GCCTAACAGCAATGGGGGCGCGGCCGAAGGTGGGTTCGATAATTGGGGTGAAGTCGT |
| | | AACAAGGTAGCCGTATCGGAAGGTGCGGCTGGATCACCTCCTTT (SEQ ID NO: 76) |
| GCF_002160015 | Anaerofilum sp | TATAAAGAGTTTGATCCTGGCTCAGGACGAACGCTGGCGGCGCGCCTAACACATGCA |
| | An201 | AGTCGAACGGAGCTATTTCGATAGATCCCTTCGGGGTGACATTGGCTTAGCTTAGTGG |
| | | CGAACGGGTGAGTAACACGTGAGGAACCTGCCCTTCAGAGGGGGACAACAGTTGGA |
| | | AACGACTGCTAATACCGCATAAGACCACAGAGCCGCATGGCTCAGGGGTCAAAGGAG |
| | | AAATCCGCTGAAGGATGGCCTCGCGTCCGATTAGGTAGTTGGCGGGGTAACGGCCCA |
| | | CCAAGCCGACGATCGGTAGCCGGACTGAGAGGTTGAACGGCCACATTGGGACTGAGA |
| | | CACGGCCCAGACTCCTACGGGAGGCAGCAGTGGGGAATATTGCACAATGGGGGAAA |
| | | CCCTGATGCAGCGACGCCGCGTGAGGGAAGAAGATTTTCGGATTGTAAACCTCTGTCT |
| | | TCGGGGACGATAATGACGGTACCCGAGGAGGAAGCCACGGCTAACTACGTGCCAGCA |
| | | GCCGCGGTAATACGTAGGTGGCAAGCGTTGTCCGGAATTACTGGGTGTAAAGGGAGC |
| | | GCAGGCGGGTTTGCAAGTTGGATGTTTAATCGAGGGGCTCAACCCCTTTCCGCATTCA |
| | | AAACTGCAGATCTTGAGTGGTGCAGAGGTAGGCGGAATTCCCGGTGTAGCGGTGGAA |
| | | TGCGTAGATATCGGGAGGAACACCAGTGGCGAAGGCGGCCTACTGGGCACTAACTGA |
| | | CGCTGAGGCTCGAAAGCATGGGTAGCAAACAGGATTAGATACCCTGGTAGTCCATGC |
| | | CGTAAACGATGATTACTAGGTGTGGGGGGATTGACCCCCTCCGTGCCGCAGTTAACA |
| | | CAATAAGTAATCCACCTGGGGAGTACGACCGCAAGGTTGAAACTCAAAGGAATTGAC |
| | | GGGGGCCCGCACAAGCAGTGGAGTATGTGGTTTAATTCGAAGCAACGCGAAGAACCT |
| | | TACCAGGTCTTGACATCCCGTGCATAGCATAGAGATATGTGAAGTCCTTCGGGACACG |
| | | GAGACAGGTGGTGCATGGTTGTCGTCAGCTCGTGTCGTGAGATGTTGGGTTAAGTCCC |
| | | GCAACGAGCGCAACCCTTACTGATAGTTACTACGCAAGAGGACTCTATCGGGACTGC |
| | | CGTTGACAAAACGGAGGAAGGTGGGGATGACGTCAAATCATCATGCCCTATATGACC |
| | | TGGGCTACACACGTACTACAATGGCTATGAACAAAGAGAAGCGAAGCCGCGAGGCA |
| | | GAGCAAACCTCATAAAAATAGTCTCAGTTCGGACTGCAGGCTGCAACTCGCCTGCAC |
| | | GAAGCCGGAATTGCTAGTAATCGCGGATCAGCATGCCGCGGTGAATACGTTCCCGGG |
| | | CCTTGTACACACCGCCCGTCACACCATGAGAGCCGGGGGGACCCGAAGTCGGTAGTC |
| | | TAACCGCAAGGAGGACGCCGCCGAAGGTAAAACTGGTGATTGGGGTGAAGTCGTAAC |
| | | AAGGTAGCCGTATCGGAAGGTGCGGCTGGATCACCTCCTTT (SEQ ID NO: 77) |
| GCF_002160025 | Anaeromassilibacillus | TCTTGTTGCTTAGTGGCGGACGGGTGAGTAACACGTGAGTAACCTGCCTCTCAGAGGG |
| | sp An200 | GGATAACGTCTTGAAAAGGACGCTAATACCGCATGATATCTCTTGACCGCATGGTCG |
| | | GGAGATCAAAGGAGCAATCCGCTGAGAGATGGACTCGCGTCCGATTAGCCAGTTGGC |
| | | GGGGTAACGGCCCACCAAAGCAACGATCGGTAGCCGGACTGAGAGGTTGAACGGCC |
| | | ACATTGGGACTGAGACACGGCCCAGACTCCTACGGGAGGCAGCAGTGGGGGATATTG |
| | | CACAATGGGGGAAACCCTGATGCAGCAACGCCGCGTGAAGGATGAAGGTCTTCGGAT |
| | | TGTAAACTTTTGTCCTATGGGAAGAAGAAAGTGACGGTACCATAGGAGGAAGCTCCG |
| | | GCTAACTACGTGCCAGCAGCCGCGGTAATACGTAGGGAGCAAGCGTTGTCCGGATTT |
| | | ACTGGGTGTAAAGGGTGCGTAGGCGGAAGAGCAAGTCAGTAGTGAAATCTGGGGGCT |
| | | TAACCCCCAAACTGCTATTGAAACTGTTTTTCTTGAGTGGAGTAGAGGTAGGCGGAAT |
| | | TCCCGGTGTAGCGGTGAAATGCGTAGAGATCGGGAGGAACACCAGTGGCGAAGGCG |
| | | GCCTACTGGGCTCTAACTGACGCTGAGGCACGAAAGTGTGGGTAGCAAACAGGATTA |
| | | GATACCCTGGTAGTCCACACCGTAAACGATGATTACTAGGTGTGGGGGGTCTGACCC |
| | | CCTCCGTGCCGGAGTTAACACAATAAGTAATCCACCTGGGGAGTACGGCCGCAAGGT |
| | | TGAAACTCAAAGGAATTGACGGGGGCCCGCACAAGCAGTGGAGTATGTGGTTTAATT |
| | | CGAAGCAACGCGAAGAACCTTACCAGGTCTTGACATCCAA (SEQ ID NO: 78) |
| GCF_002160275 | Pseudoflavonifractor | AAGTGGCGGACGGGTGAGTAACGCGTGAGGAACCTGCCTCGGAGTGGGGAATAACA |
| | sp An187 | GTTGGAAACAGCTGCTAATACCGCATAATGCAACGGAATCGCATGACTCTGTTGCCA |
| | | AAGATTTATCGCTCTGAGATGGCCTCGCGTCTGATTAGCTGGTTGGCGGGGTAACGGC |
| | | CCACCAAGGCGACGATCAGTAGCCGGACTGAGAGGTTGGCCGGCCACATTGGGACTG |
| | | AGACACGGCCCAGACTCCTACGGGAGGCAGCAGTGGGGAATATTGGGCAATGGGCG |
| | | CAAGCCTGACCCAGCAACGCCGCGTGAAGGAAGAAGGCTTTCGGGTTGTAAACTTCT |
| | | TTTGTCAGGGACGAACAAATGACGGTACCTGACGAATAAGCCACGGCTAACTACGTG |
| | | CCAGCAGCCGCGGTAATACGTAGGTGGCAAGCGTTATCCGGATTTATTGGGTGTAAA |
| | | GGGCGTGTAGGCGGGACTGCAAGTCAGATGTGAAAACCACGGGCTCAACCTGTGGCC |
| | | TGCATTTGAAACTGTAGTTCTTGAGTGTCGGAGAGGCAATCGGAATTCCGTGTGTAGC |
| | | GGTGAAATGCGTAGATATACGGAGGAACACCAGTGGCGAAGGCGGATTGCTGGACG |
| | | ATAACTGACGCTGAGGCGCGAAAGCGTGGGGAGCAAACAGGATTAGATACCCTGGTA |
| | | GTCCACGCCGTAAACGATGGATACTAGGTGTGGGGGGACTGACCCCCTCCGTGCCGC |
| | | AGTTAACACAGTAAGTATCCCACCTGGGGAGTACGATCGCAAGGTTGAAACTCAAAG |
| | | GAATTGACGGGGGCCCGCACAAGCGGTGGAGTATGTGGTTTAATTCGAAGCAACGCG |
| | | AAGAACCTTACCAGGACTTGACATCCTACTAACGAAGCAGAGATGCATTAGGTGCCC |
| | | TTCGGGGAAAGTAGAGACAGGTGGTGCATGGTTGTCGTCAGCTCGTGTCGTGAGATG |
| | | TTGGGTTAAGTCCCGCAACGAGCGCAACCCCTATTGTTAGTTGCTACGCAAGAGCACT |
| | | CTAGCGAGACTGCCGTTGACAAAACGGAGGAAGGTGGGGACGACGTCAAATCATCAT |
| | | GCCCCTTATGTCCTGGGCCACACACGTACTACAATGGCGGTTAACAAAGAGAGGCAA |
| | | TACCGCGAGGTGGAGCAAATCTCAAAAAGCCGTCCCAGTTCGGATTGCAGGCTGCAA |
| | | CCCGCCTGCATGAAGTTGGAATCGCTAGTAATCGCGGATCAGCATGCCGCGGTGAAT |
| | | ACGTTCCCGGGCCTTGTACACACCGCCCGTCACACCATGAGAGTCGGGAACACCCGA |
| | | AGTCCGTAGCCTAACCGCAAGGGGGGCGCGGCCGAAGGTGGGTTCGATAATTGGGGT |
| | | GAAGTCGTAACAAGGTAGCCGTATCGGAAGGTGCGGCTGGATCACCTCCTTT (SEQ ID |
| | | NO: 79) |
| GCF_002160305 | Pseudoflavonifractor | TATTGAGAGTTTGATCCTGGCTCAGGATGAACGCTGGCGGCGTGCTTAACACATGCAA |
| | sp An184 | GTCGAACGGAGAGCGTATGACAGAGGATTCGTCCAATGGATTGCGTTTCTTAGTGGC |
| | | GGACGGGTGAGTAACGCGTGAGGAACCTGCCTCGGAGTGGGGAATAACACAACGAA |
| | | AGCTGTGCTAATACCGCATGATGCAGCTGGGTCGCATGACTCTGGCTGCCAAAGATTT |
| | | ATCGCTCTGAGATGGCCTCGCGTCTGATTAGCTGGTTGGCGGGGTAACGGCCCACCAA |
| | | GGCGACGATCAGTAGCCGGACTGAGAGGTTGGCCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCCCTCGGGTTGTAAACTTCTTTTGTCAG |
| | | GGACGAAGCAAGTGACGGTACCTGACGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGATTGCAAGTCAGATGTGAAAACCACGGGCTCAACCTGTGGCCTGCATTT |
| | | GAAACTGCAGTTCTTGAGTACTGGAGAGGCAGACGGAATTCCTAGTGTAGCGGTGAA |
| | | ATGCGTAGATATTAGGAGGAACACCAGTGGCGAAGGCGGTCTGCTGGACAGCAACTG |
| | | ACGCTGAGGCGCGAAAGCGTGGGGAGCAAACAGGATTAGATACCCTGGTAGTCCACG |
| | | CTGTAAACGATGGATACTAGGTGTGGGGGGTCTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCCGACGACCGGTGTAGAGATACACTTTTCTCTTCGGAGAC |
| | | GTCGGTGACAGGTGGTGCATGGTTGTCGTCAGCTCGTGTCGTGAGATGTTGGGTTAAG |
| | | TCCCGCAACGAGCGCAACCCCTATTGTTAGTTGCTACGCAAGAGCACTCTAGCGAGA |
| | | CTGCCGTTGACAAAACGGAGGAAGGTGGGGACGACGTCAAATCATCATGCCCCTTAT |
| | | GTCCTGGGCCACACACGTACTACAATGGTGGTCAACAGAGGGAGGCAAAACCGCGAG |
| | | GTGGAGCAAACCCCTAAAAGCCATCCCAGTTCGGATTGCAGGCTGCAACCCGCCTGC |
| | | ATGAAGTTGGAATCGCTAGTAATCGCGGATCAGCATGCCGCGGTGAATACGTTCCCG |
| | | GGCCTTGTACACACCGCCCGTCACACCATGAGAGTCGGGAACACCCGAAGTCCGTAG |
| | | CCTAACCGCAAGGGGGGCGCGGCCGAAGGTGGGTTCGATAATTGGGGTGAAGTCGTA |
| | | ACAAGGTAGCCGTATCGGAAGGTGCGGCTGGATCACCTCCTTT (SEQ ID NO: 80) |
| GCF_002160515 | Anaeromassilibacillus | TTTAGTGGCGGACGGGTGAGTAACGCGTGAGTAACCTGCCTTCAAGAGGGGAATAAC |
| | sp An172 | GTTCTGAAAAGAACGCTAATACCGCATAACATACGGATGTCGCATGGCAACCGTATC |
| | | AAAGATTTTATCGCTTGAAGATGGACTCGCGTCCGATTAGCCAGTTGGCGGGGTAAC |
| | | GGCCCACCAAAGCGACGATCGGTAGCCGGACTGAGAGGTTGAACGGCCACATTGGGA |
| | | CTGAGACACGGCCCAGACTCCTACGGGAGGCAGCAGTGGGGGATATTGCGCAATGGG |
| | | GGCAACCCTGACGCAGCAACGCCGCGTGAACGATGAAGGTCTTCGGATTGTAAAGTT |
| | | CTTTTATTAAGGACGAAGAAGTGACGGTACTTAATGAATAAGCTCCGGCTAACTACGT |
| | | GCCAGCAGCCGCGGTAATACGTAGGGAGCAAGCGTTGTCCGGATTTACTGGGTGTAA |
| | | AGGGTGCGTAGGCGGCAGAGCAAGTCAGATGTGAAATCCGTGGGCTTAACCCACGAA |
| | | CTGCATTTGAAACTGTTTTGCTTGAGTGAAGTAGAGGCAGGCGGAATTCCCTGTGTAG |
| | | CGGTGAAATGCGTAGAGATAGGGAGGAACACCAGTGGCGAAGGCGGCCTGCTGGGC |
| | | TTTAACTGACGCTGAGGCACGAAAGCGTGGGTAGCAAACAGGATTAGATACCCTGGT |
| | | AGTCCACGCCGTAAACGATGATTACTAGGTGTGGGGGGTCTGACCCCCTCCGTGCCGC |
| | | AGTTAACACAATAAGTAATCCACCTGGGGAGTACGGCCGCAAGGTTGAAACTCAAAG |
| | | GAATTGACGGGGGCCCGCACAAGCAGTGGAGTATGTGGTTTAATTCGAAGCAACGCG |
| | | AAGAACCTTACCAGGTCTTGACATCCAACTAACGAGGTAGAGATACATTAGGTGCCC |
| | | TTCGGGGAAAGTTGAGACAGGTGGTGCATGGTTGTCGTCAGCTCGTGTCGTGAGATGT |
| | | TGGGTTAAGTCCCGCAACGAGCGCAACCCTTGCTATTAGTTGCTACGCAAGAGCACTC |
| | | TAATAGGACTGCCGTTGACAAAACGGAGGAAGGTGGGGACGACGTCAAATCATCATG |
| | | CCCCTTATGACCTGGGCTACACACGTACTACAATGGCCATCAACAGAGGGAAGCAAA |
| | | GCAGCGATGCAGAGCAAACCCCTAAAAATGGTCCCAGTTCAGATTGCAGGCTGCAAC |
| | | TCGCCTGTATGAAGTCGGAATTGCTAGTAATCGCGGATCAGCATGCCGCGGTGAATA |
| | | CGTTCCCGGGCCTTGTACACACCGCCCGTCACACCATGGGAGCCGGTAATACCCGAA |
| | | GTCAGTAGTCTAACCGCAAGGAGGACGCTGCCGAAGGTAGGATTGGCGACTGGGGTG |
| | | AAGTCGTAACAAGGTAGCCGTATCGGAAGGTGCGGCTGGATCACCTCCTTT (SEQ ID |
| | | NO: 81) |
| GCF_002160955 | Gemmiger sp | TAAAGAGTTTGATCCTGGCTCAGGACGAACGCTGGCGGCGCGCCTAACACATGCAAG |
| | An120 | TCGAACGGAGTTATTTTGGCTGAAGTTTTCGGATGGACGCCGGGATAACTTAGTGGCG |
| | | AACGGGTGAGTAACACGTGAGGAACCTGCCCTTGAGTGGGGGACAACAGTTGGAAAC |
| | | GACTGCTAATACCGCATAAGCCCACAGAGCCGCATGGCTCAGGGGGAAAAGGAGCA |
| | | ATTCGCTTAAGGATGGACTCGCGTCCAATTAGGTAGATGGTGAGGTAACGGCCCACC |
| | | ATGCCGACGATTGGTAGCCGGACTGAGAGGTTGAACGGCCACATTGGGACTGAGACA |
| | | CGGCCCAGACTCCTACGGGAGGCAGCAGTGGGGAATATTGCACAATGGGGGAAACCC |
| | | TGATGCAGCGACGCCGCGTGAAGGAAGAAGGCCTTCGGGTTGTAAACTTCTGTCGTA |
| | | AGGGACGATAATGACGGTACCTTACAAGAAAGCCACGGCTAACTACGTGCCAGCAGC |
| | | CGCGGTAAAACGTAGGTGGCAAGCGTTGTCCGGAATTACTGGGTGTAAAGGGAGCGC |
| | | AGGCGGGGAGGCAAGTTGGAAGTGAAAAGCGTGGGCTCAACCCACGACCTGCTTTCA |
| | | AAACTGTCTCTCTTGAGTAGTGCAGAGGTAAGCGGAATTCCCGGTGTAGCGGTGGAA |
| | | TGCGTAGATATCGGGAGGAACACCAGTGGCGAAGGCGGCTTACTGGGCACCAACTGA |
| | | CGCTGAGGCTCGAAAGCATGGGTAGCAAACAGGATTAGATACCCTGGTAGTCCATGC |
| | | CGTAAACGATGATTACTAGGTGTGGGGAGATTGACCCTCTCCGTGCCGCAGTTAACAC |
| | | AATAAGTAATCCACCTGGGGAGTACGACCGCAAGGTTGAAACTCAAAGGAATTGACG |
| | | GGGGCCCGCACAAGCAGTGGAGTATGTGGTTTAATTCGAAGCAACGCGAAGAACCTT |
| | | ACCAGGTCTTGACATCCGATGCATAGTGCAGAGATGCATGAAGTCCTTCGGGACATC |
| | | GAGACAGGTGGTGCATGGTTGTCGTCAGCTCGTGTCGTGAGATGTTGGGTTAAGTCCC |
| | | GCAACGAGCGCAACCCTTATCGTCAGTTACTACGCAAGAGGACTCTGGCGAGACTGC |
| | | CGTTGACAAAACGGAGGAAGGTGGGGATGACGTCAAATCATCATGCCCTTTATGACC |
| | | TGGGCTACACACGTACTACAATGGCGATCAACAAAGAGAAGCGAAGCCGCGAGGCG |
| | | GAGCAAACCTCATAAACATCGTCCCAGTTCAGATTGCAGGCTGCAACTCGCCTGCATG |
| | | AAGTCGGAATTGCTAGTAATCGCGGATCAGCATGCCGCGGTGAATACGTTCCCGGGC |
| | | CTTGTACACACCGCCCGTCACACCATGAGAGCCGGGGGGACCCGAAGTCCGTAGCCT |
| | | AACCGCAAGGAGGGCGCGGCCGAAGGTAAAACTGGTGATTGGGGTGAAGTCGTAAC |
| | | AAGGTAGCCGTATCGGAAGGTGCGGCTGGATCACCTCCTTT (SEQ ID NO: 82) |
| GCF_002161175 | Flavonifractor sp | TATTGAGAGTTTGATCCTGGCTCAGGATGAACGCTGGCGGCGTGCTTAACACATGCAA |
| | An100 | GTCGAACGGAGTGCTCATGACAGAGGATTCGTCCAATGGAATGAGTTACTTAGTGGC |
| | | GGACGGGTGAGTAACGCGTGAGTAACCTGCCTTGGAGTGGGGAATAACACAACGAA |
| | | AGCTGTGCTAATACCGCATAATGCAGCTGAGTCGCATGGCTCTGGCTGCCAAAGATTT |
| | | ATCGCTCTGAGATGGACTCGCGTCTGATTAGCTAGTTGGCGGGGTAACGGCCCACCA |
| | | AGGCGACGATCAGTAGCCGGACTGAGAGGTTGGCCGGCCACATTGGGACTGAGACAC |
| | | GGCCCAGACTCCTACGGGAGGCAGCAGTGGGGAATATTGGGCAATGGGCGCAAGCCT |
| | | GACCCAGCAACGCCGCGTGAAGGAAGAAGGCTTTCGGGTTGTAAACTTCTTTTCTCAG |
| | | GGACGAAGCAAGTGACGGTACCTGAGGAATAAGCCACGGCTAACTACGTGCCAGCA |
| | | GCCGCGGTAATACGTAGGTGGCAAGCGTTATCCGGATTTACTGGGTGTAAAGGGCGT |
| | | GTAGGCGGGATTGCAAGTCAGATGTGAAAACCATGGGCTCAACTCATGGCCTGCATT |
| | | TGAAACTGTAGTTCTTGAGTACTGGAGAGGCAGACGGAATTCCTAGTGTAGCGGTGA |
| | | AATGCGTAGATATTAGGAGGAACACCAGTGGCGAAGGCGGTCTGCTGGACAGCAACT |
| | | GACGCTGAGGCGCGAAAGCGTGGGGAGCAAACAGGATTAGATACCCTGGTAGTCCAC |
| | | GCTGTAAACGATGGATACTAGGTGTGGGGGGTCTGACCCCCTCCGTGCCGCAGTTAA |
| | | CACAATAAGTATCCCACCTGGGGAGTACGATCGCAAGGTTGAAACTCAAAGGAATTG |
| | | ACGGGGGCCCGCACAAGCGGTGGAGTATGTGGTTTAATTCGAAGCAACGCGAAGAAC |
| | | CTTACCAGGGCTTGACATCCCGGTGACCGGCTTAGAGATAGGCTTTTCCCTTCGGGGA |
| | | CACCGGTGACAGGTGGTGCATGGTTGTCGTCAGCTCGTGTCGTGAGATGTTGGGTTAA |
| | | GTCCCGCAACGAGCGCAACCCTTATTGTTAGTTGCTACGCAAGAGCACTCTAGCGAG |
| | | ACTGCCGTTGACAAAACGGAGGAAGGCGGGGACGACGTCAAATCATCATGCCCCTTA |
| | | TGTCCTGGGCCACACACGTACTACAATGGTGGTTAACAGAGGGAAGCAATGCCGCGA |
| | | GGCGGAGCAAACCCCTAAAAGCCATCCCAGTTCGGATCGCAGGCTGCAACCCGCCTG |
| | | CGTGAAGTTGGAATCGCTAGTAATCGCGGATCAGCATGCCGCGGTGAATACGTTCCC |
| | | GGGCCTTGTACACACCGCCCGTCACACCATGAGAGTCGGGAACACCCGAAGTCCGTA |
| | | GCTTAACCGCAAGGAGGGCGCGGCCGAAGGTGGGTTCGATAATTGGGGTGAAGTCGT |
| | | AACAAGGTAGCCGTATCGGAAGGTGCGGCTGGATCACCTCCTTT (SEQ ID NO: 83) |
| GCF_002161215 | Flavonifractor sp | TATTGAGAGTTTGATCCTGGCTCAGGATGAACGCTGGCGGCGTGCTTAACACATGCAA |
| | An10 | GTCGAACGGAGAACCCCTGATAGAGGATTCGTCCAATTGAAGGGAATTCTTAGTGGC |
| | | GGACGGGTGAGTAACGCGTGAGGAACCTGCCTTGGAGTGGGGAATAACAGTCCGAA |
| | | AGGACTGCTAATACCGCATAATGCAGTTGGGCCGCATGGCTCTGACTGCCAAAGATTT |
| | | ATCGCTCTGAGATGGCCTCGCGTCTGATTAGCTAGTAGGCGGGGTAACGGCCCACCTA |
| | | GGCGACGATCAGTAGCCGGACTGAGAGGTTGACCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCCCTCGGGTTGTAAACTTCTTTTGACAG |
| | | GGACGAAGAAAATGACGGTACCTGTCGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGCTGGCAAGTCAGATGTGAAAACCATGGGCTCAACCCATGGCCTGCATTT |
| | | GAAACTGTTGGTCTTGAGTGCTGGAGAGGCAATCGGAATTCCGTGTGTAGCGGTGAA |
| | | ATGCGTAGATATACGGAGGAACACCAGTGGCGAAGGCGGATTGCTGGACAGTAACTG |
| | | ACGCTGAGGCGCGAAAGCGTGGGGAGCAAACAGGATTAGATACCCTGGTAGTCCACG |
| | | CCGTAAACGATGGATACTAGGTGTGGGGGGACTGACCCCCTCCGTGCCGCAGCTAAC |
| | | GCAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCTGCTAACGAAGTAGAGATACATTAGGTGCCCTTCGGGG |
| | | AAAGCAGAGACAGGTGGTGCATGGTTGTCGTCAGCTCGTGTCGTGAGATGTTGGGTT |
| | | AAGTCCCGCAACGAGCGCAACCCTTATTGTTAGTTGCTACGCAAGAGCACTCTAGCG |
| | | AGACTGCCGTTGACAAAACGGAGGAAGGTGGGGACGACGTCAAATCATCATGCCCCT |
| | | TATGTCCTGGGCCACACACGTACTACAATGGCGGTTAACAGAGGGAAGCAAAACCGC |
| | | GAGGTGGAGCAAATCCCTAAAAGCCGTCCCAGTTCGGATTGCAGGCTGAAACCCGCC |
| | | TGTATGAAGTTGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACCGCAAGGGGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 84) |
| GCF_900067065 | Eubacteriaceae | TTTAGAGAGTTTGATCCTGGCTCAGGACGAACGCTGGCGGCGTGCCTAACACATGCA |
| | bacterium | AGTCGAACGGACGAGAAGGTGCTTGCACCTTCAAGTTAGTGGCGGACGGGTGAGTAA |
| | CHKCI005 | CGCGTGAGCAACCTGCCTCAAAGAGGGGGATAACGTCTGGAAACGGACGCTAATACC |
| | | GCATGACGTATTCGATAGGCATCTATTGAATACCAAAGGAGCAATCCGCTTTGAGAT |
| | | GGGCTCGCGTCTGATTAGCTAGTTGGTGGGGTAAAGGCCTACCAAGGCGACGATCAG |
| | | TAGCCGGACTGAGAGGTTGAACGGCCACATTGGGACTGAGACACGGCCCAGACTCCT |
| | | ACGGGAGGCAGCAGTGGGGGATATTGCACAATGGGGGAAACCCTGATGCAGCAACG |
| | | CCGCGTGAAGGAAGACGGTTTTCGGATTGTAAACTTCTGTTCTTAGTGACGATAATGA |
| | | CGGTAGCTAAGGAGAAAGCTCCGGCTAACTACGTGCCAGCAGCCGCGGTAATACGTA |
| | | GGGAGCGAGCGTTGTCCGGAATTACTGGGTGTAAAGGGAGCGTAGGCGGGAGATCA |
| | | AGTCAGATGTGAAAACTATGGGCTCAACCCATAACCTGCATTTGAAACTGGTTTTCTT |
| | | GAGTGAAGTAGAGGCAGGCGGAATTCCGAGTGTAGCGGTGAAATGCGTAGATATTCG |
| | | GAGGAACACCAGTGGCGAAGGCGGCCTGCTGGGCTTTTACTGACGCTGAGGCTCGAA |
| | | AGCATGGGGAGCAAACAGGATTAGATACCCTGGTAGTCCATGCCGTAAACGATGATT |
| | | ACTAGGTGTGGGGTGGCTGACCCATTCCGTGCCGGAGTTAACACAATAAGTAATCCA |
| | | CCTGGGGAGTACGGCCGCAAGGTTGAAACTCAAAGGAATTGACGGGGGCCCGCACA |
| | | AGCAGTGGAGTATGTGGTTTAATTCGAAGCAACGCGAAGAACCTTACCAGGTCTTGA |
| | | CATCCGACTAACGAAGTAGAGATACATTAGGTGCCCTTCGGGGAAAGTCGAGACAGG |
| | | TGGTGCATGGTTGTCGTCAGCTCGTGTCGTGAGATGTTGGGTTAAGTCCCGCAACGAG |
| | | CGCAACCCTTGTCATTAGTTGCTACGCAAGAGCACTCTAATGAGACTGCCGTTGACAA |
| | | AACGGAGGAAGGTGGGGACGACGTCAAATCATCATGCCCCTTATGACCTGGGCTACA |
| | | CACGTACTACAATGGCCGTTAACAGAGGGAAGCAATACTGTGAAGTGGAGCAAACCC |
| | | CTAAAAACGGTCCCAGTTCAGATTGCAGGCTGCAACCCGCCTGCATGAAGTCGGAAT |
| | | TGCTAGTAATCGCGGATCAGCATGCCGCGGTGAATACGTTCCCGGGCCTTGTACACAC |
| | | CGCCCGTCACACCATGGGAGCCGGTAATACCCGAAGTCGGTAGTCTAACCGCAAGGA |
| | | GGGCGCCGCCGAAGGTAGGATTGGCGACTGGGGTGAAGTCGTAACAAGGTAGCCGTA |
| | | TCGGAAGGTGCGGCTGGATCACCTCCTTT (SEQ ID NO: 85) |
| GCF_900100595 | Ruminococcaceae | TTTAGAGAGTTTGATCCTGGCTCAGGACGAACGCTGGCGGCGTGCCTAACACATGCA |
| | bacterium P7 | AGTCGAACGGAGTTGAGGAGCTTGCTCCTTAACTTAGTGGCGGACGGGTGAGTAACG |
| | | CGTGAGTAACCTGCCTCTGAGAGGGGAATAACGTTCTGAAAAGAACGCTAATACCGC |
| | | ATGACACATATTTGCCGCATGACAGATATGTCAAAGATTTTATCGCTCAGAGATGGAC |
| | | TCGCGTCCGATTAGTTAGTTGGTGAGGTAACGGCTCACCAAGACCGCGATCGGTAGC |
| | | CGGACTGAGAGGTTGAACGGCCACATTGGGACTGAGACACGGCCCAGACTCCTACGG |
| | | GAGGCAGCAGTGGGGGATATTGCGCAATGGGGGCAACCCTGACGCAGCAACGCCGC |
| | | GTGAAGGATGAAGGTTTTCGGATTGTAAACTTCTTTTCTCAGGGACGAAATTTGACGG |
| | | TACCTGAGGAATAAGCTCCGGCTAACTACGTGCCAGCAGCCGCGGTAATACGTAGGG |
| | | AGCAAGCGTTGTCCGGATTTACTGGGTGTAAAGGGTGCGTAGGCGGCTTTGTAAGTCA |
| | | GATGTGAAATCTATGGGCTCAACCCATAAACTGCATTTGAAACTACAGAGCTTGAGT |
| | | GAAGTAGAGGCAGGCGGAATTCCCTGTGTAGCGGTGAAATGCGTAGAGATAGGGAG |
| | | GAACACCAGTGGCGAAGGCGGCCTGCTGGGCTTTAACTGACGCTGAGGCACGAAAGC |
| | | GTGGGTAGCAAACAGGATTAGATACCCTGGTAGTCCACGCTGTAAACGATGATTACT |
| | | AGGTGTGGGGGGACTGACCCCTTCCGTGCCGGAGTTAACACAATAAGTAATCCACCT |
| | | GGGGAGTACGGCCGCAAGGTTGAAACTCAAAGGAATTGACGGGGGCCCGCACAAGC |
| | | AGTGGAGTATGTGGTTTAATTCGAAGCAACGCGAAGAACCTTACCAGGTCTTGACAT |
| | | CCGACTAACGAAGTAGAGATACATCAGGTGCCCTTCGGGGAAAGTCGAGACAGGTGG |
| | | TGCATGGTTGTCGTCAGCTCGTGTCGTGAGATGTTGGGTTAAGTCCCGCAACGAGCGC |
| | | AACCCTTGCTATTAGTTGCTACGCAAGAGCACTCTAATAGGACTGCCGTTGACAAAAC |
| | | GGAGGAAGGTGGGGACGACGTCAAATCATCATGCCCCTTATGACCTGGGCTACACAC |
| | | GTACTACAATGGCCATCAACAGAGGGAAGCAAAACAGCGATGTGGAGCAAACCCCT |
| | | AAAAATGGTCTCAGTTCAGATTGCAGGCTGCAACCCGCCTGCATGAAGTCGGAATTG |
| | | CTAGTAATCGCGGATCAGCATGCCGCGGTGAATACGTTCCCGGGCCTTGTACACACCG |
| | | CCCGTCACACCATGGGAGCCGGTAATACCCGAAGTCAGTAGCTTAACCT (SEQ ID |
| | | NO: 86) |
| GCF_900101355 | Ruminococcus | TTAGAGAGTTTGATCCTGGCTCAGGACGAACGCTGGCGGCGTGCCTAACACATGCAA |
| | bromii | GTCGAACGGAACTGCTTCGAAGGATTTCTTCGGAATGACATTGATTCAGTTTAGTGGC |
| | | GGACGGGTGAGTAACGCGTGAGTAACCTGCCTTCAAGAGGGGGATAACATTCTGAAA |
| | | AGAATGCTAATACCGCATGACATATGATTGTCGCATGGCAGACATATCAAAGATTTAT |
| | | CGCTTGAAGATGGACTCGCGTCCGATTAGTTAGTTGGTGAGGTAACGGCCCACCAAG |
| | | ACCGCGATCGGTAGCCGGACTGAGAGGTTGAACGGCCACATTGGGACTGAGACACGG |
| | | CCCAGACTCCTACGGGAGGCAGCAGTGGGGGATATTGCGCAATGGGGGCAACCCTGA |
| | | CGCAGCAACGCCGCGTGAAGGATGAAGGTTTTCGGATTGTAAACTTCTTTTATTAAGG |
| | | ACGAATAATGACGGTACTTAATGAATAAGCTCCGGCTAACTACGTGCCAGCAGCCGC |
| | | GGTAATACGTAGGGAGCAAGCGTTGTCCGGATTTACTGGGTGTAAAGGGTGCGTAGG |
| | | CGGCTAAGCAAGTCAGATGTGAAATCTATGGGCTCAACCCATAAACTGCATTTGAAA |
| | | CTGCATAGCTTGAGTGAAGTAGAGGCAGGCGGAATTCCCCGTGTAGCGGTGAAATGC |
| | | GTAGAGATGGGGAGGAACACCAGTGGCGAAGGCGGCCTGCTGGGCTTTAACTGACGC |
| | | TGAGGCACGAAAGCGTGGGTAGCAAACAGGATTAGATACCCTGGTAGTCCACGCTGT |
| | | AAACGATGATTACTAGGTGTGGGGGGTCTGACCCCTTCCGTGCCGGAGTTAACACAA |
| | | TAAGTAATCCACCTGGGGAGTACGGCCGCAAGGTTGAAACTCAAAGGAATTGACGGG |
| | | GGCCCGCACAAGCAGTGGAGTATGTGGTTTAATTCGAAGCAACGCGAAGAACCTTAC |
| | | CAGGTCTTGACATCCAACTAACGAGATAGAGATATGTTAGGTGCCCTTCGGGGAAAG |
| | | TTGAGACAGGTGGTGCATGGTTGTCGTCAGCTCGTGTCGTGAGATGTTGGGTTAAGTC |
| | | CCGCAACGAGCGCAACCCTTGCTATTAGTTGCTACGCAAGAGCACTCTAATAGGACT |
| | | GCCGTTGACAAAACGGAGGAAGGTGGGGACGACGTCAAATCATCATGCCCCTTATGA |
| | | CCTGGGCTACACACGTACTACAATGGGCGTTAACAGAGGGAAGCAAAATAGCGATAT |
| | | GGAGCAAACCCCTAAAAACGTTCTCAGTTCAGATTGCAGGCTGCAACCCGCCTGCAT |
| | | GAAGTCGGAATTGCTAGTAATCGCGGATCAGCATGCCGCGGTGAATACGTTCCCGGG |
| | | CCTTGTACACACCGCCCGTCACACCATGGGAGCCGGTAATACCCGAAGTCAGTAGTTC |
| | | AACCGCAAGGAGAGCGCTGCCGAAGGTAGGATTGGCGACTGGGGTGAAGTCGTAAC |
| | | AAGGTAGCCGTATCGGAAGGTGCGGCTGGATCACCTCCTTT (SEQ ID NO: 87) |
| GCF_900103235 | Ruminococcus sp | TCAGTGGCGGACGGGTGAGTAACACGTGAGCAATCTGCCTTTAAGAGGGGAATAACG |
| | YE78 | ACTGGAAACGGTCGGTAATACCGCATAACATATCGAAGCCGCATGACTTTGATATCA |
| | | AAGATTTATCGCTTAAAGATGAGCTCGCGTCTGATTAGCTAGTTGGTGAGGTAACGGC |
| | | CCACCAAGGCGACGATCAGTAGCCGGACTGAGAGGTTGAACGGCCACATTGGGACTG |
| | | AGACACGGCCCAGACTCCTACGGGAGGCAGCAGTGGGGAATATTGCACAATGGGCGC |
| | | AAGCCTGATGCAGCGATGCCGCGTGAGGGAAGAAGGTTTTCGGATTGTAAACCTCTG |
| | | TTGACAGGGACGATAATGACGGTACCTGTTCAGAAAGCTCCGGCTAACTACGTGCCA |
| | | GCAGCCGCGGTAATACGTAGGGAGCGAGCGTTGTCCGGAATTACTGGGTGTAAAGGG |
| | | AGTGTAGGCGGGACTGCAAGTCAGATGTGAAATGTAGGGGCTCAACCCCTGACCTGC |
| | | ATTTGAAACTGTAGTTCTTGAGTGAAGTAGAGGTAAGCGGAATTCCCAGTGTAGCGGT |
| | | GAAATGCGTAGATATTGGGAGGAACATCAGTGGCGAAGGCGGCTTACTGGGCTTTAA |
| | | CTGACGCTGAGGCTCGAAAGCGTGGGGAGCAAACAGGATTAGATACCCTGGTAGTCC |
| | | ACGCCGTAAACGATGATTACTAGGTGTGGGGGGATTGACCCCTTCCGTGCCGCAGTTA |
| | | ACACAATAAGTAATCCACCTGGGGAGTACGGTCGCAAGACTGAAACTCAAAGGAATT |
| | | GACGGGGGCCCGCACAAGCAGTGGAGTATGTGGATTAATTCGAAGCAACGCGAAGA |
| | | ACCTTACCAGGTCTTGACATCGTACGCATAGTGTAGAGATACATGAAGTCCTTCGGGA |
| | | CGTATAGACAGGTGGTGCATGGTTGTCGTCAGCTCGTGTCGTGAGATGTTGGGTTAAG |
| | | TCCCGCAACGAGCGCAACCCTTACTGTTAGTTGCTACGCAAGAGCACTCTAGCAGGA |
| | | CTGCCGTTGACAAAACGGAGGAAGGTGGGGATGACGTCAAATCATCATGCCCCTTAT |
| | | GACCTGGGCCTCACACGTACTACAATGGCTGTTAACAGAGGGAAGCGAAGCCGCGAG |
| | | GTGGAGCAAATCCCCAAAAGCAGTCTTAGTTCGGATTGTAGGCTGCAACCCGCCTAC |
| | | ATGAAGTCGGAATTGCTAGTAATCGCAGATCAGCATGCTGCGGTGAATACGTTCCCG |
| | | GGCCTTGTACACACCGCCCGTCACACCATGGGAGTTGGTAACACCCGAAGTCAGTAG |
| | | CCTAACCGCAAGGAGGGCGCTGCCGAAGGTGGGATCGATGACTGGGGTGAAGTCGTA |
| | | ACAAGGTAGCCGTATCGGAAGGTGCGGCTGGATCACCTCCTTT (SEQ ID NO: 88) |
| GCF_900104495 | Ruminococcaceae | ATTAAGAGTTTGATCCTGGCTCAGGACGAACGCTGGCGGCACGCTTAACACATGCAA |
| | bacterium FB2012 | GTCGAACGGAGTTATTTGAGCTTGCTTAAATAACTTAGTGGCGGACGGGTGAGTAAC |
| | | ACGTGAGCAATCTGCCTTTCAGAGGGGGATAGCAGTTGGAAACGACTGATAATACCG |
| | | CATAATATAACGAAACCGCATGACCCTGCTATCAAAGATTTATCGCTGAAAGATGAG |
| | | CTCGCGTCTGATTAGGTAGTTGGTGAGGTAACGGCTCACCAAGCCGACGATCAGTAG |
| | | CCGGACTGAGAGGTTGAACGGCCACATTGGGACTGAGACACGGCCCAGACTCCTACG |
| | | GGAGGCAGCAGTGGGGAATATTGCACAATGGGCGCAAGCCTGATGCAGCGATGCCGC |
| | | GTGAGGGAAGAAGGTTTTAGGATTGTAAACCTCTGTCCTATGGAAAGATAATGACGG |
| | | TACCATAGGAGGAAGCTCCGGCTAACTACGTGCCAGCAGCCGCGGTAATACGTAGGG |
| | | AGCGAGCGTTGTCCGGAATTACTGGGTGTAAAGGGAGTGTAGGCGGGACTGCAAGTC |
| | | AGATGTGAAAACTATGGGCTTAACCCATAGACTGCATTTGAAACTGCAGTTCTTGAGT |
| | | GAAGTAGAGGTAAGCGGAATTCCTAGTGTAGCGGTGAAATGCGTAGATATTAGGAGG |
| | | AACATCAGTGGCGAAGGCGGCTTACTGGGCTTTAACTGACGCTGAGGCTCGAAAGCG |
| | | TGGGGAGCAAACAGGATTAGATACCCTGGTAGTCCACGCCGTAAACGATGATTACTA |
| | | GGTGTGGGGGGACTGACCCCTTC (SEQ ID NO: 89) |
| GCF_900104565 | Ruminococcaceae | CTAAGAGTTTGATCCTGGCTCAGGACGAACGCTGGCGGCACGCCTAACACATGCAAG |
| | bacterium Marseille | TCGAACGGAGCTATTTTAGCGGAAGCCTTCGGGCAGAAGCTGGCTTAGCTTAGTGGC |
| | P2935 | GGACGGGTGAGTAACACGTGAGCAACCTGCCTTTGCGAGGGGGATAACGTTTGGAAA |
| | | CGAACGCTAATACCGCATAATGTCAGAAGGTCGCATGATTTTCTGACCAAAGATTTAT |
| | | CGCGCAAAGATGGGCTCGCGTCCGATTAGATAGTTGGTGAGGTAACGGCCCACCAAG |
| | | TCTGCGATCGGTAGCCGGACTGAGAGGTTGAACGGCCACATTGGGACTGAGACACGG |
| | | CCCAGACTCCTACGGGAGGCAGCAGTGGGGGATATTGCACAATGGAGGGAACTCTGA |
| | | TGCAGCGATGCCGCGTGAGGGAAGACGGTCTTCGGATTGTAAACCTCTGTCTTCAGG |
| | | GACGAACACAATGACGGTACCTGAGGAGGAAGCTCCGGCTAACTACGTGCCAGCAGC |
| | | CGCGGTAATACGTAGGGAGCAAGCGTTGTCCGGAATTACTGGGTGTAAAGGGAGTGT |
| | | AGGCGGGTCTCCAAGTCCGTTGTCAAATCTATCGGCTCAACCGATAGCCGCGGCGGA |
| | | AACTGGAGGTCTTGAGTGAAGTAGAGGCAGGCGGAATTCCTAGTGTAGCGGTGAAAT |
| | | GCGTAGATATTAGGAGGAACACCAGTGGCGAAGGCGGCCTGCTGGGCTTTAACTGAC |
| | | GCTGAGGCTCGAAAGTGTGGGGAGCAAACAGGATTAGATACCCTGGTAGTCCACACT |
| | | GTAAACGATGATTACTAGGTGTGGGGGGACTGACCCCTTCCGTGCCGGAGTTAACAC |
| | | AATAAGTAATCCACCTGGGGAGTACGGCCGCAAGGTTGAAACTCAAAGGAATTGACG |
| | | GGGGCCCGCACAAGCAGTGGAGTATGTGGTTTAATTCGAAGCAACGCGAAGAACCTT |
| | | ACCAGGTCTTGACATCGGATGCATACCATAGAGATATGGGAAGCCCTTCGGGGCATC |
| | | CAGACAGGTGGTGCATGGTTGTCGTCAGCTCGTGTCGTGAGATGTTGGGTTAAGTCCC |
| | | GCAACGAGCGCAACCCTTATCCTTAGTTGCTACGCAAGAGCACTCTAAAGAGACTGC |
| | | CGTTGACAAAACGGAGGAAGGTGGGGATGACGTCAAATCATCATGCCCCTTATGACC |
| | | TGGGCTACACACGTACTACAATGGCGATTAACAAAGGGATGCAACACGGCGACGTGA |
| | | AGCGGAACCCAAAAAATCGTCTCAGTTCAGATTGCAGGCTGCAACCCGCCTGCATGA |
| | | AGTCGGAATTGCTAGTAATCGCGGATCAGCATGCCGCGGTGAATACGTTCCCGGGCC |
| | | TTGTACACACCGCCCGTCACACCATGGGAGTCGGTAACACCCGAAGTCAGTAGCCTA |
| | | ACCGCAAGGAGGGCGCTGCCGAAGGTGGGATTGATGACTGGGGTGAAGTCGTAACA |
| | | AGGTAGCCGTATCGGAAGGTGCGGCTGGATCACCTCCTTT (SEQ ID NO: 90) |
| GCF_900110045 | Hydrogenoanaero- | AGTTTAGTGGCGGACGGGTGAGTAACACGTGAGCAACCTGCCTTTCAGAGGGGAATA |
| | bacterium | ACATTCGGAAACGAATGCTAATACCGCATAATGCAACGAGATGGCATCATCTTGCTG |
| | saccharovorans | CCAAAGATTTATCGCTGAAAGATGGGCTCGCGCCCGATTAGCTAGTTGGTGAGGTAA |
| | | TGGCCCACCAAGGCAACGATCGGTAGCCGGACTGAGAGGTTGATCGGCCACATTGGG |
| | | ACTGAGACACGGCCCAGACTCCTACGGGAGGCAGCAGTGGGGGATATTGCACAATGG |
| | | GCGAAAGCCTGATGCAGCGACGCCGCGTGAGGGAAGACGGTTTTCGGATTGTAAACC |
| | | TCTGTCTTCAGGGACGATAATGACGGTACCTGAGGAGGAAGCACCGGCTAACTACGT |
| | | GCCAGCAGCCGCGGTAATACGTAGGGTGCAAGCGTTGTCCGGAATTACTGGGTGTAA |
| | | AGGGAGCGTAGGCGGGATTGTAAGTTGGATGTGTAATGTACCGGCTCAACCGGTAAC |
| | | TTGCATTCAAAACTGCAGTTCTTGAGTGAAGTAGAGGCAGGCGGAATTCCTAGTGTAG |
| | | CGGTGAAATGCGTAGATATTAGGAGGAACACCAGTGGCGAAGGCGGCCTGCTGGGCT |
| | | TTTACTGACGCTGAGGCTCGAAAGCATGGGTAGCAAACAGGATTAGATACCCTGGTA |
| | | GTCCATGCCGTAAACGATGATTACTAGGTGTGGGTGTGCAAGCATCCGTGCCGCAGCT |
| | | AACGCAATAAGTAATCCACCTGGGGAGTACGGCCGCAAGGCTGAAACTCAAAGGAAT |
| | | TGACGGGGGCCCGCACAAGCAGTGGATTATGTGGTTTAATTCGAAGCAACGCGAAGA |
| | | ACCTTACCAGGTCTTGACATCCCTTGCATACCATAGAGATATGGGAAGCCCTTCGGGG |
| | | CAAGGAGACAGGTGGTGCATGGTTGTCGTCAGCTCGTGTCGTGAGATGTTGGGTTAA |
| | | GTCCCGCAACGAGCGCAACCCTTACTATTAGTTGCTACGCAAGAGCACTCTAATAGG |
| | | ACTGCCGTTGACAAAACGGAGGAAGGTGGGGATGACGTCAAATCATCATGCCCCTTA |
| | | TGACCTGGGCTACACACGTAATACAATGACGATAAACAGAGGGTAGCGAAGCCGCGA |
| | | GGTGGAGCCAATCCCCAAAAGTCGTCTCAGTTCGGATTGCAGGCTGCAACTCGCCTGC |
| | | ATGAAGTCGGAATTGCTAGTAATCGCAGGTCAGCATACTGCGGTGAATACGTTCCCG |
| | | GGCCTTGTACACACCGCCCGTCACACCATGGGAGTCGGTAACACCCGAAGCCAGTAG |
| | | TCTAACCGCAAGGAGGACGCTGTCGAAGGTGGGATTGATGACTGGGGTGAAGTCGTA |
| | | ACAAGGTAGCCGTATCGGAAGGTGCGGCTGGATCACCTCCTTT (SEQ ID NO: 91) |
| GCF_900113995 | Ruminococcaceae | CAAAGATTTATCGCTGTGAGATGGATTCGCGTCCGATTAGATAGTTGGTGAGGTAACG |
| | bacterium D5 | GCCCACCAAGTCGACGATCGGTAGCCGGACTGAGAGGTTGAACGGCCACATTGGGAC |
| | | TGAGACACGGCCCAGACTCCTACGGGAGGCAGCAGTGGGGGATATTGCACAATGGGC |
| | | GCAAGCCTGATGCAGCGACGCCGCGTGTGGGAAGACGGCCCTCGGGTTGTAAACCAC |
| | | TGGCTTTGGGGACGATAATGACGGTACCCAAGGAGGAAGCTCCGGCTAACTACGTGC |
| | | CAGCAGCCGCGGTAATACGTAGGGAGCGAGCGTTGTCCGGAATTACTGGGTGTAAAG |
| | | GGAGCGTAGGCGGGAGTGCAAGTTGAATGTTTAATCTATGGGCTCAACCCATATCAG |
| | | CGTTCAAAACTGCATTTCTTGAGTGAAGTAGAGGTTGGCGGAATTCCTAGTGTAGCGG |
| | | TGAAATGCGTAGATATTAGGAGGAACACCAGTGGCGAAGGCGGCCAACTGGGCTTTT |
| | | ACTGACGCTGAGGCTCGAAAGCGTGGGGAGCAAACAGGATTAGATACCCTGGTAGTC |
| | | CACGCCGTAAACGATGAATACTAGGTGTGGGGGGACTGACCCCTTCCGTGCCGCAGT |
| | | TAACACAATAAGTATTCCACCTGGGGAGTACGGCCGCAAGGCTGAAACTCAAAGGAA |
| | | TTGACGGGGGCCCGCACAAGCAGTGGATTATGTGGTTTAATTCGAAGCAACGCGAAG |
| | | AACCTTACCAGGCCTTGACATCTCCTGAGTAGCCTAGAGATAGGTGATGCCCTTCGGG |
| | | GCAGGAAGACAGGTGGTGCATGGTTGTCGTCAGCTCGTGTCGTGAGATGTTGGGTTA |
| | | AGTCCCGCAACGAGCGCAACCCTTACGGATAGTTGCTACGCAAGAGCACTCTATCAG |
| | | GACTGCCGTTGACAAAACGGAGGAAGGTGGGGATGACGTCAAATCATCATGCCCCTT |
| | | ATGGCCTGGGCTACACACGTAATACAATGGCGTTTAACAGAGGGAAGCAAGACCGCG |
| | | AGGTGGAGCGAATCCTCAAAAGGCGTCTCAGTTCAGATTGCAGGCTGCAACCCGCCT |
| | | GCATGAAGTCGGAATTGCTAGTAATCGCGGATCAGCATGCCGCGGTGAATACGTTCT |
| | | CGGGCCTTGTACACACCGCCCGTCACACCATGGAAGTCGGTAACACCCGAAGTCAGT |
| | | AGCCTAACCGCAAGGGGGGCGCTGCCGAAGGTGGGATTGGTAACTGGGGTGAAGTCG |
| | | TAACAAGGTAGCCGTATCGGAAGGTGCGGCTGGATCACCTCCTTT (SEQ ID NO: 92) |
| GCF_900115635 | Oscillibacter sp | TGCCAAAGATTTATCGCTGAAAGATGGCCTCGCGTCTGATTAGCTAGTTGGTGGGGTA |
| | PC13 | ACGGCCCACCAAGGCGACGATCAGTAGCCGGACTGAGAGGTTGACCGGCCACATTGG |
| | | GACTGAGATACGGCCCAGACTCCTACGGGAGGCAGCAGTGGGGAATATTGGGCAATG |
| | | GACGCAAGTCTGACCCAGCAACGCCGCGTGAAGGAAGAAGGCTTTCGGGTTGTAAAC |
| | | TTCTTTTAAGTGGGAAGAGCAGAAGACGGTACCACTTGAATAAGCCACGGCTAACTA |
| | | CGTGCCAGCAGCCGCGGTAATACGTAGGTGGCAAGCGTTGTCCGGATTTACTGGGTG |
| | | TAAAGGGCGTGTAGCCGGGTGTGCAAGTCAGATGTGAAATCTGGAGGCTCAACCTCC |
| | | AAACTGCATTTGAAACTGTGCATCTTGAGTATCGGAGAGGTAATCGGAATTCCTTGTG |
| | | TAGCGGTGAAATGCGTAGATATAAGGAAGAACACCAGTGGCGAAGGCGGATTACTG |
| | | GACGACAACTGACGGTGAGGCGCGAAAGCGTGGGGAGCAAACAGGATTAGATACCC |
| | | TGGTAGTCCACGCTGTAAACGATCAATACTAGGTGTGCGGGGACTGATCCCCTGCGTG |
| | | CCGCAGTTAACACAATAAGTATTGCACCTGGGGAGTACGATCGCAAGGTTGAAACTC |
| | | AAAGGAATTGACGGGGGCCCGCACAAGCGGTGGATTATGTGGTTTAATTCGAAGCAA |
| | | CGCGAAGAACCTTACCAGGGCTTGACATCCTACTAATGAAGCAGAGATGCATTAAGT |
| | | GCCCTTCGGGGAAAGTAGAGACAGGTGGTGCATGGTTGTCGTCAGCTCGTGTCGTGA |
| | | GATGTTGGGTTAAGTCCCGCAACGAGCGCAACCCCTATTGTTAGTTGCTACGCAAGAG |
| | | CACTCTAGCGAGACTGCCGTTGACAAAACGGAGGAAGGTGGGGACGACGTCAAATCA |
| | | TCATGCCCCTTATGTCCTGGGCTACACACGTAATACAATGGCGGTTAACAGAGGGATG |
| | | CAAATCCGCGAGGAGGAGCGAACCCCGAAAAGCCGTCTCAGTTCGGATCGCAGGCTG |
| | | CAACCCGCCTGCGTGAAGTCGGAATCGCTAGTAATCGCGGATCAGCATGCCGCGGTG |
| | | AATACGTTCCCGGGCCTTGTACACACCGCCCGTCACACCATGAGAGTCGGGAACACC |
| | | CGAAGTCCGTAGCCTAACCGCAAGGAGGGCGCGGCCGAAGGTGGGTTCGATAATTGG |
| | | GGTGAAGTCGTAACAAGGTAGCCGTATCGGAAGGTGCGGCTGGATCACCTCCTTT |
| | | (SEQ ID NO: 93) |
| GCF_900169975 | Pseudoflavonifractor | TATTGAGAGTTTGATCCTGGCTCAGGATGAACGCTGGCGGCGTGCTTAACACATGCAA |
| | sp Marseille P3106 | GTCGAACGGAGAGCCAATGACGGAGTTTTCGGACAACGGATTTGGTTTCTTAGTGGC |
| | | GGACGGGTGAGTAACGCGTGAGCAACCTGCCTTGGAGTGGGGAATAACAGCTGGAA |
| | | ACAGTTGCTAATACCGCATAATGCAGCGAGGGGACATCCTCTTGCTGCCAAAGATTTA |
| | | TCGCTCTGAGATGGACTCGCGTCTGATTAGCTGGTTGGCGGGGTAACGGCCCACCAA |
| | | GGCGACGATCAGTAGCCGGACTGAGAGGTTGACCGGCCACATTGGGACTGAGATACG |
| | | GCCCAGACTCCTACGGGAGGCAGCAGTGGGGAATATTGGGCAATGGGCGAAAGCCTG |
| | | ACCCAGCAACGCCGCGTGAAGGAAGAAGGCCCTCGGGTTGTAAACTTCTTTTATCAG |
| | | GGACGAAACAAATGACGGTACCTGATGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGTCTGCAAGTCAGGTGTGAAATTCCAGGGCTCAACCCTGGAACTGCACTT |
| | | GAAACTGTGGGTCTTGAGTGATGGAGAGGCAGGCGGAATTCCGTGTGTAGCGGTGAA |
| | | ATGCGTAGATATACGGAGGAACACCAGTGGCGAAGGCGGCCTGCTGGACATTAACTG |
| | | ACGCTGAGGCGCGAAAGCGTGGGGAGCAAACAGGATTAGATACCCTGGTAGTCCACG |
| | | CTGTAAACGATGGATACTAGGTGTGGGGGGTCTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCTACTAACGAAGCAGAGATGCATTAGGTGCCCTTCGGGG |
| | | AAAGTAGAGACAGGTGGTGCATGGTTGTCGTCAGCTCGTGTCGTGAGATGTTGGGTTA |
| | | AGTCCCGCAACGAGCGCAACCCTTATTGCTAGTTGCTACGCAAGAGCACTCTAGCGA |
| | | GACTGCCGTTGACAAAACGGAGGAAGGTGGGGACGACGTCAAATCATCATGCCCCTT |
| | | ATGTCCTGGGCCACACACGTACTACAATGGCGGTCAACAGAGGGAAGCAATACCGCG |
| | | AGGTGGAGCGAATCCCTAAAAGCCGTCCCAGTTCGGATTGCAGGCTGAAACCCGCCT |
| | | GCATGAAGTTGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACCGCAAGGGGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 94) |
| GCF_900197595 | Neglecta sp | TTAGAGAGTTTGATCCTGGCTCAGGACGAACGCTGGCGGCGTGCCTAACACATGCAA |
| | Marseille P3890 | GTCGAACGGAGTTAAGAGAAGCTTGCTTTTATTAACTTAGTGGCGGACGGGTGAGTA |
| | | ACGCGTGAGCAATCTGCCTTTCAGTGGGGAATAACGTTCTGAAAAGAACGCTAATAC |
| | | CGCATAATATTGTTGAGCCGCATGGTTTGATAATCAAAGGATTTATTCGCTGAAAGAT |
| | | GAGCTCGCGTCCGATTAGATAGTTGGTGAGGTAACGGCTCACCAAGTCGACGATCGG |
| | | TAGCCGGACTGAGAGGTTGAACGGCCACATTGGGACTGAGACACGGCCCAGACTCCT |
| | | ACGGGAGGCAGCAGTGAGGGATATTGGTCAATGGGGGAAACCCTGAACCAGCAACG |
| | | CCGCGTGAGGGAAGACGGTTTTCGGATTGTAAACCTCTGTCCTCTGTGAAGATAATGA |
| | | CGGTAGCAGAGGAGGAAGCTCCGGCTAACTACGTGCCAGCAGCCGCGGTAATACGTA |
| | | GGGAGCAAGCGTTGTCCGGATTTACTGGGTGTAAAGGGTGCGTAGGCGGCTATGCAA |
| | | GTCAGGAGTGAAATCTATGGGCTTAACCCATAAACTGCTCTTGAAACTGTATAGCTTG |
| | | AGTGAAGTAGAGGTAGGCGGAATTCCCGGTGTAGCGGTGGAATGCGTAGAGATCGGG |
| | | AGGAACACCAGTGGCGAAGGCGGCCTACTGGGCTTTAACTGACGCTGAAGCACGAAA |
| | | GCGTGGGTAGCAAACAGGATTAGATACCCTGGTAGTCCACGCCGTAAACGATGATTA |
| | | CTAGGTGTGGGGGGTCTGACCCCCTCCGTGCCGGAGTTAACACAATAAGTAATCCAC |
| | | CTGGGGAGTACGGTCGCAAGACTGAAACTCAAAGGAATTGACGGGGGCCCGCACAA |
| | | GCAGTGGAGTATGTGGATTAATTCGAAGCAACGCGAAGAACCTTACCAGGTCTTGAC |
| | | ATCCCTCTGACCGCTCTAGAGATAGAGCTTCTCTTCGGAGCAGAGGTGACAGGTGGTG |
| | | CATGGTTGTCGTCAGCTCGTGTCGTGAGATGTTGGGTTAAGTCCCGCAACGAGCGCAA |
| | | CCCCTATGATTAGTTGCTACGCAAGAGCACTCTAATCAGACTGCCGTTGACAAAACGG |
| | | AGGAAGGTGGGGATGACGTCAAATCATCATGCCCCTTATGACCTGGGCCTCACACGT |
| | | ACTACAATGGCCGTTAACAACGGGATGCAATATAGCGATATGGAGCAAAACCCCAAA |
| | | AACGGTCTCAGTTCGGATTGTAGGCTGAAACTCGCCTGCATGAAGCTGGAATTGCTAG |
| | | TAATCGCAGATCAGAATGCTGCGGTGAATACGTTCCCGGGCCTTGTACACACCGCCCG |
| | | TCACACCATGGGAGCCGGTAATACCCGAAGTCAGTAGCCTAACCGTAAGGAGGGCGC |
| | | TGCCGAAGGTAGGGTTGGCGACTGGGGTGAAGTCGTAACAAGGTAGCCGTATCGGAA |
| | | GGTGCGGCTGGATCACCTCCTTT (SEQ ID NO: 95) |
| GCF_900199495 | Clostridium sp | TATTGAGAGTTTGATCCTGGCTCAGGATGAACGCTGGCGGCGTGCTTAACACATGCAA |
| | SN20 | GTCGAACGGAGTGCTCATGACGGAGTTTTCGGACAACGGATTGGGTTACTTAGTGGC |
| | | GGACGGGTGAGTAACGCGTGAGGAACCTGCCTCGGAGTGGGGAATAACATACCGAA |
| | | AGGTGTGCTAATACCGCATAATGCAGTTGGGTCGCATGACTCTGACTGCCAAAGATTT |
| | | ATCGCTCTGAGATGGCCTCGCGTCTGATTAGCTAGTTGGCGGGGTAACGGCCCACCAA |
| | | GGCGACGATCAGTAGCCGGACTGAGAGGTTGACCGGCCACATTGGGACTGAGACACG |
| | | GCCCAGACTCCTACGGGAGGCAGCAGTGGGGAATATTGGGCAATGGGCGCAAGCCTG |
| | | ACCCAGCAACGCCGCGTGAAGGAAGAAGGCTTTCGGGTTGTAAACTTCTTTTGTCAG |
| | | GGACGAAACAAATGACGGTACCTGACGAATAAGCCACGGCTAACTACGTGCCAGCAG |
| | | CCGCGGTAATACGTAGGTGGCAAGCGTTATCCGGATTTACTGGGTGTAAAGGGCGTG |
| | | TAGGCGGGACTGCAAGTCAGGTGTGAAAACCAGGGGCTCAACCTCTGGCCTGCATTT |
| | | GAAACTGTAGTTCTTGAGTGCTGGAGAGGCAATCGGAATTCCGTGTGTAGCGGTGAA |
| | | ATGCGTAGATATACGGAGGAACACCAGTGGCGAAGGCGGATTGCTGGACAGTAACTG |
| | | ACGCTGAGGCGCGAAAGCGTGGGGAGCAAACAGGATTAGATACCCTGGTAGTCCACG |
| | | CCGTAAACGATGGATACTAGGTGTGGGGGGACTGACCCCCTCCGTGCCGCAGTTAAC |
| | | ACAATAAGTATCCCACCTGGGGAGTACGATCGCAAGGTTGAAACTCAAAGGAATTGA |
| | | CGGGGGCCCGCACAAGCGGTGGAGTATGTGGTTTAATTCGAAGCAACGCGAAGAACC |
| | | TTACCAGGGCTTGACATCCTACTAACGAAGCAGAGATGCATTAGGTGCCCTTCGGGG |
| | | AAAGTAGAGACAGGTGGTGCATGGTTGTCGTCAGCTCGTGTCGTGAGATGTTGGGTTA |
| | | AGTCCCGCAACGAGCGCAACCCCTATTGTTAGTTGCTACGCAAGAGCACTCTAGCGA |
| | | GACTGCCGTTGACAAAACGGAGGAAGGTGGGGACGACGTCAAATCATCATGCCCCTT |
| | | ATGTCCTGGGCCACACACGTACTACAATGGTGGTTAACAGAGGGAAGCAATACCGCG |
| | | AGGTGGAGCAAATCCCTAAAAGCCATCCCAGTTCGGATTGCAGGCTGAAACCCGCCT |
| | | GTATGAAGTTGGAATCGCTAGTAATCGCGGATCAGCATGCCGCGGTGAATACGTTCC |
| | | CGGGCCTTGTACACACCGCCCGTCACACCATGAGAGTCGGGAACACCCGAAGTCCGT |
| | | AGCCTAACCGCAAGGAGGGCGCGGCCGAAGGTGGGTTCGATAATTGGGGTGAAGTCG |
| | | TAACAAGGTAGCCGTATCGGAAGGTGCGGCTGGATCACCTCCTTT (SEQ ID NO: 96) |
| GCF_900199635 | Anaerotruncus sp | CAAAGAGTTTGATCCTGGCTCAGGACGAACGCTGGCGGCGCGCCTAACACATGCAAG |
| | AT3 | TCGAACGGAGTGTTTTCACGGAAGTTTTCGGATGGAAGTGGTTACACTTAGTGGCGGA |
| | | CGGGTGAGTAACACGTGAGCAACCTGCCTTTCAGAGGGGGATAACAGTTGGAAACGA |
| | | CTGCTAATACCGCATGATATTACCGGGTCACATGGCCTGGCAATCAAAGGAGCAATC |
| | | CGCTGAAAGATGGGCTCGCGTCCGATTAGCCAGTTGGCGGGGTAATGGCCCACCAAA |
| | | GCGACGATCGGTAGCCGGACTGAGAGGTTGAACGGCCACATTGGGACTGAGACACGG |
| | | CCCAGACTCCTACGGGAGGCAGCAGTGGGGGATATTGCACAATGGGCGAAAGCCTGA |
| | | TGCAGCGACGCCGCGTGAGGGAAGACGGTCTTCGGATTGTAAACCTCTGTCTTAGGG |
| | | GAAGAAAATGACGGTACCCTAAGAGGAAGCTCCGGCTAACTACGTGCCAGCAGCCGC |
| | | GGTAATACGTAGGGAGCGAGCGTTGTCCGGAATTACTGGGTGTAAAGGGAGCGTAGG |
| | | CGGGATGCCAAGTAGAATGTTAAATCCATCGGCTCAACTGGTGGCAGCGTTCTAAAC |
| | | TGGCGTTCTTGAGTGAGGTAGAGGCAGGCGGAATTCCTAGTGTAGCGGTGAAATGCG |
| | | TAGATATTAGGAGGAACACCAGTGGCGAAGGCGGCCTGCTGGGCCTTAACTGACGCT |
| | | GAGGCTCGAAAGCGTGGGGAGCAAACAGGATTAGATACCCTGGTAGTCCACGCCGTA |
| | | AACGATGAATCCTAGGTGTGGGGGGACTGACACCTTCCGTGCCGCAGTTAACACAAT |
| | | AAGTAATCCACCTGGGGAGTACGGCCGCAAGGTTGAAACTCAAAGGAATTGACGGGG |
| | | GCCCGCACAAGCAGTGGAGTATGTGGTTTAATTCGAAGCAACGCGAAGAACCTTACC |
| | | AGGTCTTGACATCGGATGCATACCATAGAGATATGGGAAGCCCTTCGGGGCATCCAG |
| | | ACAGGTGGTGCATGGTTGTCGTCAGCTCGTGTCGTGAGATGTTGGGTTAAGTCCCGCA |
| | | ACGAGCGCAACCCTTATTATTAGTTGCTACGCAAGAGCACTCTAATGAGACTGCCGTT |
| | | GACAAAACGGAGGAAGGTGGGGATGACGTCAAATCATCATGCCCCTTATGACCTGGG |
| | | CTACACACGTACTACAATGGCACTCAAACAGAGGGAAGCGACACCGCGAGGTGAAG |
| | | CGGATCCCAAAAAAGTGTCTCAGTTCGGATCGCAGGCTGCAACCCGCCTGCGTGAAG |
| | | TCGGAATTGCTAGTAATCGCGGATCAGCATGCCGCGGTGAATACGTTCCCGGGCCTTG |
| | | TACACACCGCCCGTCACACCATGGGAGTCGGTAACACCCGAAGCCAGTAGCCTAACC |
| | | GCAAGGAGGGCGCTGTCGAAGGTGGGATTGATGACTGGGGTGAAGTCGTAACAAGGT |
| | | AGCCGTATCGGAAGGTGCGGCTGGATCACCTCCTTT (SEQ ID NO: 97) |
| GCF_900291955 | Anaeromassilibacillus | TTTTGAGAGTTTGATCCTGGCTCAGGACGAACGCTGGCGGCGTGCCTAACACATGCAA |
| | sp Marseille | GTCGAACGAAGCTTTGAGGAGCTTGCTTTTTAAAGCTTAGTGGCGGACGGGTGAGTA |
| | P3876 | ACGCGTGAGCAACCTGCCTCTCAGAGGGGGATAACGTTTTGAAAAGAACGCTAATAC |
| | | CGCATAACATATCGGAACCGCATGATTCTGATATCAAAGGAGCAATCCGCTGAGAGA |
| | | TGGGCTCGCGTCCGATTAGTTAGTTGGTGAGGTAACGGCTCACCAAGACTACGATCG |
| | | GTAGCCGGACTGAGAGGTTGATCGGCCACATTGGGACTGAGACACGGCCCAGACTCC |
| | | TACGGGAGGCAGCAGTGGGGGATATTGCGCAATGGGGGAAACCCTGACGCAGCAAC |
| | | GCCGCGTGAAGGAAGAAGGTCTTCGGATTGTAAACTTCTTTTGTCAGGGACGAAGAA |
| | | AGTGACGGTACCTGACGAATAAGCTCCGGCTAACTACGTGCCAGCAGCCGCGGTAAT |
| | | ACGTAGGGAGCGAGCGTTGTCCGGATTTACTGGGTGTAAAGGGTGCGTAGGCGGCCG |
| | | AGCAAGTCAGTTGTGAAAACTATGGGCTTAACCCATAACGTGCAATTGAAACTGTCC |
| | | GGCTTGAGTGAAGTAGAGGTAGGCGGAATTCCCGGTGTAGCGGTGAAATGCGTAGAG |
| | | ATCGGGAGGAACACCAGTGGCGAAGGCGGCCTACTGGGCTTTAACTGACGCTGAGGC |
| | | ACGAAAGCATGGGTAGCAAACAGGATTAGATACCCTGGTAGTCCATGCCGTAAACGA |
| | | TGATTACTAGGTGTGGGGGGACTGACCCCTTCCGTGCCGCAGTTAACACAATAAGTA |
| | | ATCCACCTGGGGAGTACGGCCGCAAGGTTGAAACTCAAAGGAATTGACGGGGGCCCG |
| | | CACAAGCAGTGGAGTATGTGGTTTAATTCGAAGCAACGCGAAGAACCTTACCAGGTC |
| | | TTGACATCCTGAGAATCCTTAAGAGATTAGGGAGTGCCTTCGGGAACTCAGAGACAG |
| | | GTGGTGCATGGTTGTCGTCAGCTCGTGTCGTGAGATGTTGGGTTAAGTCCCGCAACGA |
| | | GCGCAACCCTTGCTATTAGTTGCTACGCAAGAGCACTCTAATAGGACTGCCGTTGACA |
| | | AAACGGAGGAAGGTGGGGACGACGTCAAATCATCATGCCCCTTATGACCTGGGCTAC |
| | | ACACGTACTACAATGGCCATTAACAGAGGGAAGCAAAACCGCGAGGCAGAGCAAAC |
| | | CCCTAAAAATGGTCCCAGTTCGGATTGTAGGCTGCAACCCGCCTACATGAAGTTGGA |
| | | ATTGCTAGTAATCGCGGATCAGCATGCCGCGGTGAATACGTTCCCGGGCCTTGTACAC |
| | | ACCGCCCGTCACACCATGGGAGCCGGTAATACCCGAAGTCAGTAGTCTAACAGCAAT |
| | | GAGGACGCTGCCGAAGGTAGGATTGGCGACTGGGGTGAAGTCGTAACAAGGTAGCC |
| | | GTATCGGAAGGTGCGGCTGGATCACCTCCTTT (SEQ ID NO: 98) |
| STS00001 | Gemmiger formicilis | TATAAAGAGTTTGATCCTGGCTCAGGACGAACGCTGGCGGCGCGCCTAACACATGCA |
| | | AGTCGAACGGAACTTGAGAGAGCTTGCTTTTTCAAGTTTAGTGGCGAACGGGTGAGT |
| | | AACGCGTGAGTAACCTGCCCTGGAGTGGGGGACAACAGTTGGAAACGACTGCTAATA |
| | | CCGCATAAGCCCACGGCACCGCATGGTACTGAGGGAAAAGGATTTATTCGCTTCAGG |
| | | ATGGACTCGCGTCCAATTAGCTAGTTGGTGAGGTAACGGCCCACCAAGGCGACGATT |
| | | GGTAGCCGGACTGAGAGGTTGAACGGCCACATTGGGACTGAGACACGGCCCAGACTC |
| | | CTACGGGAGGCAGCAGTGGGGGATATTGCACAATGGGGGAAACCCTGATGCAGCGA |
| | | CGCCGCGTGGAGGAAGAAGGTTTTCGGATTGTAAACTCCTGTCGTACGGGACGATAA |
| | | TGACGGTACCGTACAAGAAAGCCACGGCTAACTACGTGCCAGCAGCCGCGGTAAAAC |
| | | GTAGGTGGCAAGCGTTGTCCGGAATTACTGGGTGTAAAGGGAGCGCAGGCGGACCGG |
| | | CAAGTTGGAAGTGAAATCTATGGGCTCAACCCATAAATTGCTTTCAAAACTGCTGGCC |
| | | TTGAGTAGTGCAGAGGTAGGCGGAATTCCCGGTGTAGCGGTGGAATGCGTAGATATC |
| | | GGGAGGAACACCAGTGGCGAAGGCGGCCTACTGGGCACCAACTGACGCTGAGGCTC |
| | | GAAAGCATGGGTAGCAAACAGGATTAGATACCCTGGTAGTCCATGCCGTAAACGATG |
| | | ATTACTAGGTGTTGGAGGATTGACCCCTTCAGTGCCGCAGTTAACACAATAAGTAATC |
| | | CACCTGGGGAGTACGACCGCAAGGTTGAAACTCAAAGGAATTGACGGGGGCCCGCAC |
| | | AAGCAGTGGAGTATGTGGTTTAATTCGAAGCAACGCGAAGAACCTTACCAGGTCTTG |
| | | ACATCCGATGCATAGTGCAGAGATGCATGAAGTCCTTCGGGACATCGAGACAGGTGG |
| | | TGCATGGTTGTCGTCAGCTCGTGTCGTGAGATGTTGGGTTAAGTCCCGCAACGAGCGC |
| | | AACCCTTATTGCCAGTTACTACGCAAGAGGACTCTGGCGAGACTGCCGTTGACAAAA |
| | | CGGAGGAAGGTGGGGATGACGTCAAATCATCATGCCCTTTATGACCTGGGCTACACA |
| | | CGTACTACAATGGCGTTTAACAAAGAGAAGCAATACCGCGAGGTGGAGCAAAACTCA |
| | | AAAACAACGTCTCAGTTCAGATTGCAGGCTGCAACTCGCCTGCATGAAGTCGGAATT |
| | | GCTAGTAATCGCGGATCAGCATGCCGCGGTGAATACGTTCCCGGGCCTTGTACACACC |
| | | GCCCGTCACACCATGAGAGCCGGGGGGACCCGAAGTCCGTAGTCTAACCGCAAGGAG |
| | | GACGCGGCCGAAGGTAAAACTGGTGATTGGGGTGAAGTCGTAACAAGGTAGCCGTAT |
| | | CGGAAGGTGCGGCTGGATCACCTCCTTT (SEQ ID NO: 99) |
| STS00002 | Ruminococcaceae | TTAGAGAGTTTGATCCTGGCTCAGGACGAACGCTGGCGGCGTGCCTAACACATGCAA |
| | unnamed sp 1 | GTCGAACGGAACTTCTTTAAAGGATTTCTTCGGAATGAATTTGATTAAGTTTAGTGGC |
| | | GGACGGGTGAGTAACGCGTGAGTAACCTGCCTCTAAGAGGGGAATAACATTCTGAAA |
| | | AGAATGCTAATACCGCATAATATATATTTATCGCATGGTAGATATATCAAAGATTTAT |
| | | CGCTTAGAGATGGACTCGCGTCCGATTAGTTAGTTGGTGAGGTAACGGCTCACCAAG |
| | | ACCGCGATCGGTAGCCGGACTGAGAGGTTGAACGGCCACATTGGGACTGAGACACGG |
| | | CCCAGACTCCTACGGGAGGCAGCAGTGGGGGATATTGCGCAATGGGGGAAACCCTGA |
| | | CGCAGCAACGCCGCGTGAAGGATGAAGGTCTTCGGATTGTAAACTTCTTTTATTAAGG |
| | | ACGAAGAAAGTGACGGTACTTAATGAATAAGCTCCGGCTAACTACGTGCCAGCAGCC |
| | | GCGGTAATACGTAGGGAGCAAGCGTTGTCCGGATTTACTGGGTGTAAAGGGTGCGTA |
| | | GGCGGCTTTGCAAGTCAGATGTGAAATCTATGGGCTCAACCCATAGCCTGCATTTGAA |
| | | ACTGCAGAGCTTGAGTGAAGTAGAGGCAGGCGGAATTCCCCGTGTAGCGGTGAAATG |
| | | CGTAGAGATGGGGAGGAACACCAGTGGCGAAGGCGGCCTGCTGGGCTTTAACTGACG |
| | | CTGAGGCACGAAAGCGTGGGTAGCAAACAGGATTAGATACCCTGGTAGTCCACGCTG |
| | | TAAACGATGATTACTAGGTGTGGGGGGTCTGACCCCTTCCGTGCCGGAGTTAACACA |
| | | ATAAGTAATCCACCTGGGGAGTACGGCCGCAAGGTTGAAACTCAAAGGAATTGACGG |
| | | GGGCCCGCACAAGCAGTGGAGTATGTGGTTTAATTCGAAGCAACGCGAAGAACCTTA |
| | | CCAGGTCTTGACATCCTACTAACGAGATAGAGATATGTTAGGTGCCCTTCGGGGAAA |
| | | GTAGAGACAGGTGGTGCATGGTTGTCGTCAGCTCGTGTCGTGAGATGTTGGGTTAAGT |
| | | CCCGCAACGAGCGCAACCCTTGCTATTAGTTGCTACGCAAGAGCACTCTAATAGGACT |
| | | GCCGTTGACAAAACGGAGGAAGGTGGGGACGACGTCAAATCATCATGCCCCTTATGA |
| | | CCTGGGCTACACACGTACTACAATGGACATTAACAGAGGGAAGCAATACAGTGATGT |
| | | GGAGCAAACCCCTAAAAATGTTCTCAGTTCAGATTGCAGGCTGCAACCCGCCTGTATG |
| | | AAGATGGAATTGCTAGTAATCGCAGATCAGCATGCTGCGGTGAATACGTTCCCGGGC |
| | | CTTGTACACACCGCCCGTCACACCATGGGAGCCGGTAATACCCGAAGTCAGTAGTCT |
| | | AACCGCAAGGAGGACGCTGCCGAAGGTAGGATTGGCGACTGGGGTGAAGTCGTAAC |
| | | AAGGTAGCCGTATCGGAAGGTGCGGCTGGATCACCTCCTTT (SEQ ID NO: 100) |
| STS00003 | Ruminococcaceae | GAACGGCCACATTGGGACTGAGACACGGCCCAGACTCCTACGGGAGGCAGCAGTGG |
| | unnamed sp 2 | GGAATATTGCGCAATGGGGGAAACCCTGACGCAGCAACGCCGCGTGATTGAAGAAG |
| | | GCCTTCGGGTTGTAAAGATCTTTAATTGGGGACGAAAAATGACGGTACCCAAAGAAT |
| | | AAGCTCCGGCTAACTACGTGCCAGCAGCCGCGGTAATACGTAGGGAGCAAGCGTTAT |
| | | CCGGATTTACTGGGTGTAAAGGGCGAGTAGGCGGGCTGGCAAGTTGGGAGTGAAATC |
| | | CCGGGGCTTAACCCCGGAACTGCTTTCAAAACTGCTGGTCTTGAGTGATGGAGAGGC |
| | | AGGCGGAATTCCGTGTGTAGCGGTGAAATGCGTAGATATACGGAGGAACACCAGTGG |
| | | CGAAGGCGGCCTGCTGGACATTAACTGACGCTGAGGAGCGAAAGCGTGGGGAGCAA |
| | | ACAGGATTAGATACCCTGGTAGTCCACGCCGTAAACGATGGATACTAGGTGTGGGAG |
| | | GTATTGACCCCTTCCGTGCCGGAGTTAACACAATAAGTATCCCACCTGGGGAGTACGG |
| | | CCGCAAGGTTGAAACTCAAAGGAATTGACGGGGGCCCGCACAAGCAGTGGAGTATGT |
| | | GGTTTAATTCGAAGCAACGCGAAGAACCTTACCAGGTCTTGACATCCCTCTGACCGCC |
| | | CTAGAGATAGGGTTTCCCTTCGGGGCAGAGGTGACAGGTGGTGCATGGTTGTCGTCA |
| | | GCTCGTGTCGTGAGATGTTGGGTTAAGTCCCGCAACGAGCGCAACCCTTACGGTTAGT |
| | | TGATACGCAAGATCACTCTAGCCGGACTGCCGTTGACAAAACGGAGGAAGGTGGGGA |
| | | CGACGTCAAATCATCATGCCCCTTATGACCTGGGCTACACACGTACTACAATGGCAGT |
| | | CATACAGAGGGAAGCAAAACAGTGATGTGGAGCAAATCCCTAAAAGCTGTCCCAGTT |
| | | CAGATTGCAGGCTGCAACTCGCCTGCATGAAGTCGGAATTGCTAGTAATCGCGGATC |
| | | AGCATGCCGCGGTGAATACGTTCCCGGGCCTTGTACACACCGCCCGTCACACCATGA |
| | | GAGCCGGTAATACCCGAAGTCCGTAGCCTAACCGCAAGGAGGGCGCGGCCGAAGGT |
| | | AGGACTGGTAATTAGGGTGAAGTCGTAACAAGGTAGCCGTATCGGAAGGTGCGGCTG |
| | | GATCACCTCCTTT (SEQ ID NO: 101) |
| STS00004 | Gemmiger formicilis | AAAAGGATTTATTCGCTTTAGGATGGACTCGCGTCCAATTAGCTAGTTGGTGAGGTAA |
| | | CGGCCCACCAAGGCGACGATTGGTAGCCGGACTGAGAGGTTGAACGGCCACATTGGG |
| | | ACTGAGACACGGCCCAGACTCCTACGGGAGGCAGCAGTGGGGGATATTGCACAATGG |
| | | GGGAAACCCTGATGCAGCGACGCCGCGTGGAGGAAGAAGGTTTTCGGATTGTAAACT |
| | | CCTGTCGTTAGGGACGATAATGACGGTACCTAACAAGAAAGCACCGGCTAACTACGT |
| | | GCCAGCAGCCGCGGTAAAACGTAGGGTGCAAGCGTTGTCCGGAATTACTGGGTGTAA |
| | | AGGGAGCGCAGGCGGGAAGACAAGTTGGAAGTGAAAACCATGGGCTCAACCCATGA |
| | | ATTGCTTTCAAAACTGTTTTTCTTGAGTAGTGCAGAGGTAGATGGAATTCCCGGTGTA |
| | | GCGGTGGAATGCGTAGATATCGGGAGGAACACCAGTGGCGAAGGCGGTCTACTGGGC |
| | | ACCAACTGACGCTGAGGCTCGAAAGCATGGGTAGCAAACAGGATTAGATACCCTGGT |
| | | AGTCCATGCCGTAAACGATGATTACTAGGTGTTGGGGGATTGACCCCCTCAGTGCCGC |
| | | AGTTAACACAATAAGTAATCCACCTGGGGAGTACGACCGCAAGGTTGAAACTCAAAG |
| | | GAATTGACGGGGGCCCGCACAAGCAGTGGAGTATGTGGTTTAATTCGAAGCAACGCG |
| | | AAGAACCTTACCAGGTCTTGACATCCGATGCATAGCACAGAGATGTGTGAAATCCTTC |
| | | GGGACATCGAGACAGGTGGTGCATGGTTGTCGTCAGCTCGTGTCGTGAGATGTTGGGT |
| | | TAAGTCCCGCAACGAGCGCAACCCTTATTGCCAGTTACTACGTTAAGAGGACTCTGGC |
| | | GAGACTGCCGTTGACAAAACGGAGGAAGGTGGGGATGACGTCAAATCATCATGCCCT |
| | | TTATGACCTGGGCTACACACGTACTACAATGGCGTTAAACAAAGAGAAGCAAGACCG |
| | | CGAGGTGGAGCAAAACTCAAAAACAACGTCTCAGTTCAGATTGCAGGCTGCAACTCG |
| | | CCTGCATGAAGTCGGAATTGCTAGTAATCGCGGATCAGCATGCCGCGGTGAATACGT |
| | | TCCCGGGCCTTGTACACACCGCCCGTCACACCATGAGAGCCGGGGGGACCCGAAGTC |
| | | GATAGTCTAACCGCAAGGAGGACGTCGCCGAAGGTAAAACTGGTGATTGGGGTGAAG |
| | | TCGTAACAAGGTAGCCGTATCGGAAGGTGCGGCTGGATCACCTCCTTT (SEQ ID |
| | | NO: 102) |
| STS00005 | Ruminococcaceae | GCTTAGTGGCGGACTGGTGAGTAACGCGTGAGGAACCTGCCTTTCAGAGGGGGACAA |
| | unnamed sp 3 | CAGTTGGAAACGACTGCTAATACCGCATGATGCATATTGACCGCATGGTCGGTATGTC |
| | | AAAGATTTATCGCTGAAAGATGGCCTCGCGTCTGATTAGCTTGTTGGTGAGGTAACGG |
| | | CCCACCAAGGCGACGATCAGTAGCCGGACTGAGAGGTTGACCGGCCACATTGGGACT |
| | | GAGATACGGCCCAGACTCCTACGGGAGGCAGCAGTGGGGAATATTGGGCAATGGAC |
| | | GCAAGTCTGACCCAGCAACGCCGCGTGAAGGAAGAAGGCTTTCGGGTTGTAAACTTC |
| | | TTTGACAGGGGAAGAGTAGAAGACGGTACCCTGAAAACAAGCCACGGCTAACTACGT |
| | | GCCAGCAGCCGCGGTAATACGTAGGTGGCAAGCGTTGTCCGGATTTACTGGGTGTAA |
| | | AGGGCGTGTAGCCGGGAAGGCAAGTCAGATGTGAAATCTGGAGGCTCAACCTCCAAA |
| | | CTGCATTTGAAACTGTCTTTCTTGAGTATCGGAGAGGTAATCGGAATTCCTTGTGTAG |
| | | CGGTGAAATGCGTAGATATAAGGAGGAACACCAGTGGCGAAGGCGGATTACTGGAC |
| | | GACAACTGACGGTGAGGCGCGAAAGCGTGGGGAGCAAACAGGATTAGATACCCTGG |
| | | TAGTCCACGCTGTAAACGATCAATACTAGGTGTGCGGGGACTGACCCCCTGCGTGCC |
| | | GGAGTTAACACAATAAGTATTGCACCTGGGGAGTACGATCGCAAGGTTGAAACTCAA |
| | | AGGAATTGACGGGGGCCCGCACAAGCGGTGGATTATGTGGTTTAATTCGAAGCAACG |
| | | CGAAGAACCTTACCAGGGCTTGACATCCTACTAATGAAGCAGAGATGCATTAAGTGC |
| | | CCTTCGGGGAAAGTAGAGACAGGTGGTGCATGGTTGTCGTCAGCTCGTGTCGTGAGA |
| | | TGTTGGGTTAAGTCCCGCAACGAGCGCAACCCCTATTGTTAGTTGCTACGCAAGAGCA |
| | | CTCTAGCGAGACTGCCGTTGACAAAACGGAGGAAGGTGGGGACGACGTCAAATCATC |
| | | ATGCCCCTTATGTCCTGGGCCACACACGTAATACAATGGCGGTAAACAGAGGGATGC |
| | | AAAGCCGTGAGGTGGAGCGAACCCCTAAAAGCCGTCCCAGTTCGGATTGCAGGCTGC |
| | | AACCCGCCTGCATGAAGTCGGAATCGCTAGTAATCGCGGATCAGCATGCCGCGGTGA |
| | | ATACGTTCCCGGGCCTTGTACACACCGCCCGTCACACCATGAGAGTCGGGAACACCC |
| | | GAAGCCCGTAGCCTAACAGCAATGAGGGCGCGGTCGAAGGTGGGTTCGATAATTGGG |
| | | GTGAAGTCGTAACAAGGTAGCCGTATCGGAAGGTGCGGCTGGATCACCTCCTTT (SEQ |
| | | ID NO: 103) |
| STS00006 | Ruminococcaceae | TATAGAGAGTTTGATCCTGGCTCAGGACGAACGCTGGCGGCGTGCTTAACACATGCA |
| | unnamed sp 4 | AGTCGAACGGAGCACCCCTGAATGAGGTTTCGGCCAAAGGAAGGGAATGCTTAGTGG |
| | | CGGACTGGTGAGTAACGCGTGAGGAACCTGCCTTTCAGAGGGGGACAACAGTTGGAA |
| | | ACGACTGCTAATACCGCATGACACATGAATGGGGCATCCCATTGATGTCAAAGATTT |
| | | ATCGCTGAAAGATGGCCTCGCGTCCCATTAGCTAGTAGGCGGGGTAACGGCCCACCT |
| | | AGGCGACGATGGGTAGCCGGACTGAGAGGTTGACCGGCCACATTGGGACTGAGATAC |
| | | GGCCCAGACTCCTACGGGAGGCAGCAGTGGGGAATATTGGGCAATGGACGCAAGTCT |
| | | GACCCAGCAACGCCGCGTGAAGGAAGAAGGCTTTCGGGTTGTAAACTTCTTTTGTCA |
| | | GGGAACAGTAGAAGAGGGTACCTGACGAATAAGCCACGGCTAACTACGTGCCAGCA |
| | | GCCGCGGTAATACGTAGGTGGCAAGCGTTGTCCGGATTTACTGGGTGTAAAGGGCGT |
| | | GCAGCCGGGCTGGCAAGTCAGGCGTGAAATCCCAGGGCTCAACCCTGGAACTGCGTT |
| | | TGAAACTGCTGGTCTTGAGTACCGGAGAGGTCATCGGAATTCCTTGTGTAGCGGTGAA |
| | | ATGCGTAGATATAAGGAAGAACACCAGTGGCGAAGGCGGATGACTGGACGGCAACT |
| | | GACGGTGAGGCGCGAAAGCGTGGGGAGCAAACAGGATTAGATACCCTGGTAGTCCA |
| | | CGCTGTAAACGATCAATACTAGGTGTGCGGGGACTGACCCCCTGCGTGCCGCAGTTA |
| | | ACACAATAAGTATTGCACCTGGGGAGTACGATCGCAAGGTTGAAACTCAAAGGAATT |
| | | GACGGGGGCCCGCACAAGCGGTGGATTATGTGGTTTAATTCGAAGCAACGCGAAGAA |
| | | CCTTACCAGGGCTTGACATCCTACTAACGAAGTAGAGATACATTAGGTGCCCTTCGGG |
| | | GAAAGTAGAGACAGGTGGTGCATGGTTGTCGTCAGCT (SEQ ID NO: 104) |
| STS00007 | Ruminococcaceae | TTTAGAGAGTTTGATCCTGGCTCAGGACGAACGCTGGCGGCGTGCCTAACACATGCA |
| | unnamed sp 5 | AGTCGAACGGAGTTATTTAAATAGAACCCTTCGGGGTGACGTTTTAATAACTTAGTGG |
| | | CGGACGGGTGAGTAACGCGTGAGTAACCTGCCTTTCAGAGGGGGATAACGTCCTGAA |
| | | AAGGACGCTAATACCGCATGATATATTTGTGCCGCATGGTATGGATATCAAAGGAGC |
| | | AATCCGCTGGAAGATGGACTCGCGTCCGATTAGCTAGTTGGAGGGGTAACGGCCCAC |
| | | CAAGGCGACGATCGGTAGCCGGACTGAGAGGTTGAACGGCCACATTGGGACTGAGAC |
| | | ACGGCCCAGACTCCTACGGGAGGCAGCAGTGGGGGATATTGCGCAATGGGGGAAAC |
| | | CCTGACGCAGCAACGCCGCGTGAAGGAAGAAGGTTTTCGGATTGTAAACTTCTTTTCT |
| | | AAGGGACGAAGAAGTGACGGTACCTTAGGAATAAGCTCCGGCTAACTACGTGCCAGC |
| | | AGCCGCGGTAATACGTAGGGAGCAAGCGTTGTCCGGATTTACTGGGTGTAAAGGGTG |
| | | CGTAGGCGGCAATGCAAGTCAGATGTGAAATGCACGGGCTCAACCCGTGAGCTGCAT |
| | | TTGAAACTGTGTTGCTTGAGTGAGGTAGAGGCAGGCGGAATTCCCGGTGTAGCGGTG |
| | | AAATGCGTAGAGATCGGGAGGAACACCAGTGGCGAAGGCGGCCTGCTGGGCCTTAAC |
| | | TGACGCTGATGCACGAAAGCGTGGGTAGCAAACAGGATTAGATACCCTGGTAGTCCA |
| | | CGCTGTAAACGATGATTACTAGGTGTGGGGGGTCTGACCCCTTCCGTGCCGCAGTTAA |
| | | CACAATAAGTAATCCACCTGGGGAGTACGGCCGCAAGGTTGAAACTCAAAGGAATTG |
| | | ACGGGGGCCCGCACAAGCAGTGGAGTATGTGGTTTAATTCGAAGCAACGCGAAGAAC |
| | | CTTACCAGGTCTTGACATCCAGCTAACGAAGTAGAGATACATTAGGTGCCCTTCGGGG |
| | | AAAGCTGAGACAGGTGGTGCATGGTTGTCGTCAGCTCGTGTCGTGAGATGTTGGGTTA |
| | | AGTCCCGCAACGAGCGCAACCCTTGCTGTTAGTTGCTACGCAAGAGCACTCTAACAG |
| | | GACTGCCGTTGACAAAACGGAGGAAGGTGGGGACGACGTCAAATCATCATGCCCCTT |
| | | ATGACCTGGGCTACACACGTACTACAATGGCCGTCAACAGAGGGAAGCAAGACCGCG |
| | | AGGTGGAGCAAACCCCCAAAAACGGCCCCAGTTCGGATTGTAGGCTGCAACCCGCCT |
| | | ACATGAAGTCGGAATTGCTAGTAATCGCGGATCAGCATGCCGCGGTGAATACGTTCC |
| | | CGGGCCTTGTACACACCGCCCGTCACACCATGGGAGCCGGTAATACCCGAAGTCAGT |
| | | AGCCTAACCGCAAGGAGGGCGCTGCCGAAGGTAGGATTGGCGACTGGGGTGAAGTC |
| | | GTAACAAGGTAGCCGTATCGGAAGGTGCGGCTGGATCACCTCCTTT (SEQ ID NO: 105) |
| STS00008 | Ruminococcaceae | ACGAGAGTTTGATCCTGGCTCAGGACGAACGCTGGCGGCGTGCCTAACACATGCAAG |
| | unnamed sp 6 | TCGAACGAGAATCTTTGAACAGATCTTTTCGGAGTGACGTTCAAAGAGGAAAGTGGC |
| | | GGACGGGCGAGTAACGCGTGAGTAACCTGCCCATAAGAGGGGGATAATCCATGGAA |
| | | ACGTGGACTAATACCGCATATTGTAGTTAAGTTGCATGACTTGATTATGAAAGATTTA |
| | | TCGCTTATGGATGGACTCGCGTCAGATTAGATAGTTGGTGAGGTAACGGCTCACCAA |
| | | GTCAACGATCTGTAGCCGAACTGAGAGGTTGATCGGCCGCATTGGGACTGAGACACG |
| | | GCCCAGACTCCTACGGGAGGCAGCAGTGGGGAATATTGCGCAATGGGGGCAACCCTG |
| | | ACGCAGCAACGCCGCGTGCAGGAAGAAGGTCTTCGGATTGTAAACTGTTGTCGCAAG |
| | | GGAAGAAGACAGTGACGGTACCTTGTGAGAAAGTCACGGCTAACTACGTGCCAGCAG |
| | | CCGCGGTAATACGTAGGTGACAAGCGTTGTCCGGATTTACTGGGTGTAAAGGGCGCG |
| | | TAGGCGGACTGTCAAGTCAGTCGTGAAATACCGGGGCTTAACCCCGGGGCTGCGATT |
| | | GAAACTGACAGCCTTGAGTATCGGAGAGGAAAGCGGAATTCCTAGTGTAGCGGTGAA |
| | | ATGCGTAGATATTAGGAGGAACACCAGTGGCGAAGGCGGCTTTCTGGACGACAACTG |
| | | ACGCTGAGGCGCGAAAGTGTGGGGAGCAAACAGGATTAGATACCCTGGTAGTCCACA |
| | | CCGTAAACGATGGATACTAGGTGTAGGAGGTATCGACCCCTTCTGTGCCGCAGTTAAC |
| | | ACAATAAGTATCCCACCTGGGGAGTACGACCGCAAGGTTGAAACTCAAAGGAATTGA |
| | | CGGGGGCCCGCACAAGCAGTGGAGTATGTGGTTTAATTCGAAGCAACGCGAAGAACC |
| | | TTACCTGGGCTTGACATCCCTGGAATCGAGTAGAGATACTTGAGTGCCTTCGGGAATC |
| | | AGGTGACAGGTGGTGCATGGTTGTCGTCAGCTCGTGTCGTGAGATGTTGGGTTAAGTC |
| | | CCGCAACGAGCGCAACCCCTATTGTCAGTTGCCATCATTAAGTTGGGCACTCTGGCGA |
| | | GACTGCCGGTGACAAATCGGAGGAAGGTGGGGACGACGTCAAATCATCATGCCCCTT |
| | | ATGCCCAGGGCTACACACGTACTACAATGGCCGATAACAAAGTGCAGCGAAACCGTG |
| | | AGGTGGAGCGAATCACAAAACTCGGTCTCAGTTCAGATTGCAGGCTGCAACTCGCCT |
| | | GCATGAAGTTGGAATTGCTAGTAATCGCGGATCAGAATGCCGCGGTGAATACGTTCC |
| | | CGGGCCTTGTACACACCGCCCGTCACACCATGAGAGTCGATAACACCCGAAGCCTGT |
| | | GAGCTAACCTTTAGGAGGCAGCAGTCGAAGGTGGGGTTGATGATTGGGGTGAAGTCG |
| | | TAACAAGGTAGCCGTATCGGAAGGTGCGGCTGGATCACCTCCTTT (SEQ ID NO: 106) |
| STS00009 | Ruminococcaceae | ATTAAGAGTTTGATCCTGGCTCAGGACGAACGCTGGCGGCGCGCCTAACACATGCAA |
| | unnamed sp 7 | GTCGAACGAAGTTTCATAACGGAAGTTTTCGGATGGAAGATATGAAACTTAGTGGCG |
| | | GACGGGTGAGTAACACGTGAGCAACCTGCCTTTTAGAGGGGGATAACGTTTGGAAAC |
| | | GAACGCTAATACCGCATAACGTAGTCGATCGGCATCGATTGACTACCAAAGGAGCAA |
| | | TCCGCTGAAAGATGGGCTCGCGTCCGATTAGATAGTTGGCGGGGTAACGGCCCACCA |
| | | AGTCGACGATCGGTAGCCGGACTGAGAGGTTGATCGGCCACATTGGGACTGAGACAC |
| | | GGCCCAGACTCCTACGGGAGGCAGCAGTGGGGAATATTGCACAATGGGGGAAACCCT |
| | | GATGCAGCGACGCCGCGTGAGGGAAGAAGGTTTTCGGATTGTAAACCTCTGTCCTTG |
| | | GTGACGATAATGACGGTAGCCAAGGAGGAAGCCACGGCTAACTACGTGCCAGCAGCC |
| | | GCGGTAATACGTAGGTGGCAAGCGTTGTCCGGAATTACTGGGTGTAAAGGGAGCGTA |
| | | GGCGGGAAAGCAAGTTGAATGTTTAAACTATCGGCTCAACCGATAATCGCGTTCAAA |
| | | ACTGTTTTTCTTGAGTGAAGTAGAGGTAGGCGGAATTCCTAGTGTAGCGGTGAAATGC |
| | | GTAGATATTAGGAGGAACACCAGTGGCGAAGGCGGCCTACTGGGCTTTAACTGACGC |
| | | TGAGGCTCGAAAGCGTGGGTAGCAAACAGGATTAGATACCCTGGTAGTCCACGCCGT |
| | | AAACGATGATTACTAGGTGTGGGGGGATCAACCCTTCCGTGCCGCAGCAAACGCAAT |
| | | AAGTAATCCACCTGGGGAGTACGACCGCAAGGTTGAAACTCAAAGGAATTGACGGGG |
| | | ACCCGCACAAGCAGTGGAGTATGTGGTTTAATTCGAAGCAACGCGAAGAACCTTACC |
| | | AGGTCTTGACATCCAACGAACTCGCTAGAGATAGCAAGGTGCCCTTCGGGGAGCGTT |
| | | GAGACAGGTGGTGCATGGTTGTCGTCAGCTCGTGTCGTGAGATGTTGGGTTAAGTCCC |
| | | GCAACGAGCGCAACCCTTACTGATAGTTGCTACGCAAGAGCACTCTATCGGGACTGC |
| | | CGTTGACAAAACGGAGGAAGGTGGGGATGACGTCAAATCATCATGCCCCTTATGACC |
| | | TGGGCTACACACGTACTACAATGGCTATTAACAACGGGAAGCGAAGAGGTGACTCGG |
| | | AGCCAATCCAAAAAAATAGTCTCAGTTCGGATTGCAGGCTGCAACTCGCCTGCATGA |
| | | AGCCGGAATTGCTAGTAATCGCGGATCAGCATGCCGCGGTGAATACGTTCCCGGGTC |
| | | TTGTACACACCGCCCGTCACACCATGAGAGTTGGCAACACCCGAAGTCAGTAGTCTA |
| | | ACCGCAAGGAGGACGCTGCCGAAGGTGGGGTCGATGATTGGGGTGAAGTCGTAACA |
| | | AGGTAGCCGTATCGGAAGGTGCGGCTGGATCACCTCCTTT (SEQ ID NO: 107) |

### REFERENCES

The following references, to the extent that they provide exemplary procedural or other details supplementary to those set forth herein, are specifically incorporated herein by reference.
Callahan et al. 2016. DADA2: High-resolution sample inference from Illumina amplicon data. Nat. Methods 13, 581-583.
Edgar, R. C. 2010. Search and clustering orders of magnitude faster than BLAST. Bioinforma. Oxf. Engl. 26, 2460-2461.
Edgar, R. C. 2013. UPARSE: highly accurate OTU sequences from microbial amplicon reads. Nat. Methods 10, 996-998.
Frankel et al. 2017. Metagenomic shotgun sequencing and unbiased metabolomic profiling identify specific human gut microbiota and metabolites associated with immune checkpoint therapy efficacy in melanoma patients. Neoplasia. 2017 October; 19(10):848-855. doi: 10.1016/j.neo.2017.08.004.
Gopalakrishnan et al. 2018. Gut microbiome modulates response to anti-PD-1 immunotherapy in melanoma patients. Science January 5; 359(6371):97-103. doi: 10.1126/science.aan4236.
Routy et al 2018. Gut microbiome influences efficacy of PD-1-based immunotherapy against epithelial tumors. Science. 2018 Jan. 5; 359 (6371): 91-97. doi: 10.1126/science.aan3706.
Matson et al. 2018. The commensal microbiome is associated with anti-PD-1 efficacy in metastatic melanoma patients. Science. January 5; 359(6371):104-108. doi: 10.1126/science.aao3290.
Quast et al. 2013. The SILVA ribosomal RNA gene database project: improved data processing and web-based tools. Nucleic Acids Res. 41 (D1): D590-D596. doi: 10.1093/nar/gks1219.

1. A therapeutic composition comprising an effective amount of an isolated population of bacteria that are phylogenetic descendants of the most recent common ancestor (MRCA) of Faecalibacterium prausnitzii and Flavonifractor plautii.

2. A therapeutic composition comprising an effective amount of an isolated population of bacteria that have 16S rDNA sequence identity of at least 94.5% to 16S rDNA sequences of species belonging to the family Ruminococcaceae.

3. The therapeutic composition of claim 2, wherein the bacteria have 16S rDNA sequence identity of at least 98.7% to 16S rDNA sequences of species belonging to the family Ruminococcaceae.

4. A therapeutic composition comprising an effective amount of an isolated population of bacteria belonging to one or more genera within the family Ruminococcaceae, e.g., the genera Ruminococcus, Gemmiger, Faecalibacterium, Subdoligranulum or combinations thereof.

5. A therapeutic composition comprising an effective amount of an isolated population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

6. A therapeutic composition comprising an effective amount of an isolated population of bacteria belonging to one or more of the genera Alistipes, Bacteroides, Blautia, Clostridium, Eubacterium, Parabacteroides or combinations thereof.

7. A therapeutic composition comprising an effective amount of an isolated population of bacteria belonging to one or more of the genera Barnesiella, Bifidobacterium, Blautia, Erysipelotrichaceae, Odoribacter, Parabacteroides or combinations thereof.

8. A therapeutic composition comprising an effective amount of an isolated population of bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

9. A therapeutic composition comprising an effective amount of an isolated population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof.

10. A therapeutic composition comprising an effective amount of an isolated population of bacteria species selected from Alistipes senegalensis, Bacteroides dorei, Blautia_SC109, Clostridium_SC64, Eubacterium_biforme, Parabacteroides distasonis or combinations thereof.

11. A therapeutic composition comprising an effective amount of an isolated population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

12. The therapeutic composition of any of claims 4-7, wherein the therapeutic composition comprises bacteria belonging to two or more genera.

13. The therapeutic composition of any of claims 4-7, wherein the therapeutic composition comprises bacteria belonging to three or more genera.

14. The therapeutic composition of any of claims 4-7, wherein the therapeutic composition comprises bacteria belonging to four or more genera.

15. The therapeutic composition of any of claims 5-7, wherein the therapeutic composition comprises bacteria belonging to five or more genera.

16. The therapeutic composition of any of claims 8-11, wherein the therapeutic composition comprises bacteria belonging to two or more species.

17. The therapeutic composition of any of claims 8-11, wherein the therapeutic composition comprises bacteria belonging to three or more species.

18. The therapeutic composition of any of claims 8-11, wherein the therapeutic composition comprises bacteria belonging to four or more species.

19. The therapeutic composition of any of claims 8-11, wherein the therapeutic composition comprises bacteria belonging to five or more species.

20. A therapeutic composition comprising an effective amount of a purified population of bacteria that are phylogenetic descendants of the most recent common ancestor (MRCA) of Faecalibacterium prausnitzii and Flavonifractor plautii.

21. A therapeutic composition comprising an effective amount of a purified population of bacteria that have 16S rDNA sequence identity of at least 94.5% to 16S rDNA sequences of species belonging to the family Ruminococcaceae.

22. The therapeutic composition of claim 21, wherein the bacteria have 16S rDNA sequence identity of at least 98.7% to 16S rDNA sequences of species belonging to the family Ruminococcaceae.

23. A therapeutic composition comprising an effective amount of a purified population of bacteria belonging to one or more of the genera Ruminococcus, Gemmiger, Faecalibacterium, Subdoligranulum or combinations thereof.

24. A therapeutic composition comprising an effective amount of a purified population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

25. A therapeutic composition comprising an effective amount of a purified population of bacteria belonging to one or more of the genera Alistipes, Bacteroides, Blautia, Clostridium, Eubacterium, Parabacteroides or combinations thereof.

26. A therapeutic composition comprising an effective amount of a purified population of bacteria belonging to one or more of the genera Barnesiella, Bifidobacterium, Blautia, Erysipelotrichaceae, Odoribacter, Parabacteroides or combinations thereof.

27. A therapeutic composition comprising an effective amount of a purified population of bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

28. A therapeutic composition comprising an effective amount of a purified population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacteriumbiforme, Parabacteroides distasonis or combinations thereof.

29. A therapeutic composition comprising an effective amount of a purified population of bacteria species selected from Alistipes senegalensis, Bacteroides dorei, Blautia_SC109, Clostridium_SC64, Eubacteriumbiforme, Parabacteroides distasonis or combinations thereof.

30. A therapeutic composition comprising an effective amount of a purified population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

31. The therapeutic composition of any of claims 23-26, wherein the therapeutic composition comprises bacteria belonging to two or more genera.

32. The therapeutic composition of any of claims 23-26, wherein the therapeutic composition comprises bacteria belonging to three or more genera.

33. The therapeutic composition of any of claims 23-26, wherein the therapeutic composition comprises bacteria belonging to four or more genera.

34. The therapeutic composition of any of claims 24-26, wherein the therapeutic composition comprises bacteria belonging to five or more genera.

35. The therapeutic composition of any of claims 27-30, wherein the therapeutic composition comprises bacteria belonging to two or more species.

36. The therapeutic composition of any of claims 27-30, wherein the therapeutic composition comprises bacteria belonging to three or more species.

37. The therapeutic composition of any of claims 27-30, wherein the therapeutic composition comprises bacteria belonging to four or more species.

38. The therapeutic composition of any of claims 27-30, wherein the therapeutic composition comprises bacteria belonging to five or more species.

39. The therapeutic composition of any one of claims 1-38, further comprising an anticancer agent.

40. The therapeutic composition of claim 39, wherein the anticancer agent is a checkpoint inhibitor.

41. The therapeutic composition of claim 40, wherein the checkpoint inhibitor is selected from an anti-PD-1 antibody, an anti-CTLA-4 antibody, an anti-PD-L1 antibody or combinations thereof.

42. The therapeutic composition of claim 41, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors or STI-A1010.

43. The therapeutic composition of claim 39, wherein the anticancer agent is cyclophosphamide.

44. The therapeutic composition of any of claims 1-43, wherein each of the isolated populations of bacteria is present in the composition at a concentration of at least about 1×10² viable colony forming units.

45. The therapeutic composition of any of claims 1-44, wherein each isolated population of bacteria is present in the composition at a concentration of about 1×10² to 1×10⁹ viable colony forming units.

46. The therapeutic composition of any of claims 1-45, wherein a fraction of the isolated population of bacteria comprises a spore-forming bacteria.

47. The therapeutic composition of any of claims 1-45, wherein a fraction of the isolated population of bacteria is in spore form.

48. The therapeutic composition of any of claims 1-47, wherein the composition further comprises a pharmaceutically acceptable excipient.

49. The therapeutic composition of any of claims 1-47, wherein the composition is formulated for delivery to the intestine.

50. The therapeutic composition of any of claims 1-47, wherein the composition is enterically coated.

51. The therapeutic composition of any of claims 1-47, wherein the composition is formulated for oral administration.

52. The therapeutic composition of claim 51, wherein the composition is formulated into a food or beverage.

53. The therapeutic composition of any of claims 1-52, wherein the composition can reduce the rate of tumor growth in an animal model.

54. The composition of any one of claims 1-53, wherein the composition is formulated for multiple administrations.

55. The composition of any one of claims 1-54, wherein each of the populations of bacteria is present in the composition at a concentration of at least 1×10³ viable CFU.

56. A method of treating a cancer in a mammalian subject comprising administering to the subject a therapeutic composition comprising an effective amount of an isolated population of bacteria that are phylogenetic descendants of the most recent common ancestor (MRCA) of Faecalibacterium prausnitzii and Flavonifractor plautii.

57. A method of treating a cancer in a mammalian subject comprising administering to the subject a therapeutic composition comprising an effective amount of an isolated population of bacteria that have 16S rDNA sequence identity of at least 94.5% to 16S rDNA sequences of species belonging to the family Ruminococcaceae.

58. The method of claim 57, wherein the bacteria have 16S rDNA sequence identity of at least 98.7% to 16S rDNA sequences of species belonging to the family Ruminococcaceae.

59. A method of treating a cancer in a mammalian subject comprising administering to the subject a therapeutic composition comprising an effective amount of an isolated population of bacteria belonging to one or more of the genera Ruminococcus, Gemmiger, Faecalibacterium, Subdoligranulum or combinations thereof.

60. A method of treating a cancer in a mammalian subject comprising administering to the subject a therapeutic composition comprising an effective amount of an isolated population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

61. A method of treating a cancer in a mammalian subject comprising administering to the subject a therapeutic composition comprising an effective amount of an isolated population of bacteria belonging to one or more of the genera Alistipes, Bacteroides, Blautia, Clostridium, Eubacterium, Parabacteroides or combinations thereof.

62. A method of treating a cancer in a mammalian subject comprising administering to the subject a therapeutic composition comprising an effective amount of an isolated population of bacteria belonging to one or more of the genera Barnesiella, Bifidobacterium, Blautia, Erysipelotrichaceae, Odoribacter, Parabacteroides or combinations thereof.

63. A method of treating a cancer in a mammalian subject comprising administering to the subject a therapeutic composition comprising an effective amount of an isolated population of bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

64. A method of treating a cancer in a mammalian subject comprising administering to the subject a therapeutic composition comprising an effective amount of an isolated population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof.

65. A method of treating a cancer in a mammalian subject comprising administering to the subject a therapeutic composition comprising an effective amount of an isolated population of bacteria species selected from Alistipes senegalensis, Bacteroides dorei, Blautia_SC109, Clostridium_SC64, Eubacterium_biforme, Parabacteroides distasonis or combinations thereof.

66. A method of treating a cancer in a mammalian subject comprising administering to the subject a therapeutic composition comprising an effective amount of an isolated population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

67. The method of any of claims 59-62, wherein the therapeutic composition comprises bacteria belonging to two or more genera.

68. The method of any of claims 59-62, wherein the therapeutic composition comprises bacteria belonging to three or more genera.

69. The method of any of claims 59-62, wherein the therapeutic composition comprises bacteria belonging to four or more genera.

70. The method of any of claims 60-62, wherein the therapeutic composition comprises bacteria belonging to five or more genera.

71. The method of any of claims 63-66, wherein the therapeutic composition comprises bacteria belonging to two or more species.

72. The method of any of claims 63-66, wherein the therapeutic composition comprises bacteria belonging to three or more species.

73. The method of any of claims 63-66, wherein the therapeutic composition comprises bacteria belonging to four or more species.

74. The method of any of claims 63-66, wherein the therapeutic composition comprises bacteria belonging to five or more species.

75. A method of treating a cancer in a mammalian subject comprising administering to the subject a therapeutic composition comprising an effective amount of a purified population of bacteria that are phylogenetic descendants of the most recent common ancestor (MRCA) of Faecalibacterium prausnitzii and Flavonifractor plautii.

76. A method of treating a cancer in a mammalian subject comprising administering to the subject a therapeutic composition comprising an effective amount of a purified population of bacteria that have 16S rDNA sequence identity of at least 94.5% to 16S rDNA sequences of species belonging to the family Ruminococcaceae.

77. The method of claim 76, wherein the bacteria have 16S rDNA sequence identity of at least 98.7% to 16S rDNA sequences of species belonging to the family Ruminococcaceae.

78. A method of treating a cancer in a mammalian subject comprising administering to the subject a therapeutic composition comprising an effective amount of a purified population of bacteria belonging to one or more of the genera Ruminococcus, Gemmiger, Faecalibacterium, Subdoligranulum or combinations thereof.

79. A method of treating a cancer in a mammalian subject comprising administering to the subject a therapeutic composition comprising an effective amount of a purified population of bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

80. A method of treating a cancer in a mammalian subject comprising administering to the subject a therapeutic composition comprising an effective amount of a purified population of bacteria belonging to one or more of the genera Alistipes, Bacteroides, Blautia, Clostridium, Eubacterium, Parabacteroides or combinations thereof.

81. A method of treating a cancer in a mammalian subject comprising administering to the subject a therapeutic composition comprising an effective amount of a purified population of bacteria belonging to one or more of the genera Barnesiella, Bifidobacterium, Blautia, Erysipelotrichaceae, Odoribacter, Parabacteroides or combinations thereof.

82. A method of treating a cancer in a mammalian subject comprising administering to the subject a therapeutic composition comprising an effective amount of a purified population of bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

83. A method of treating a cancer in a mammalian subject comprising administering to the subject a therapeutic composition comprising an effective amount of a purified population of bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof.

84. A method of treating a cancer in a mammalian subject comprising administering to the subject a therapeutic composition comprising an effective amount of a purified population of bacteria species selected from Alistipes senegalensis, Bacteroides dorei, Blautia_SC109, Clostridium_SC64, Eubacterium_biforme, Parabacteroides distasonis or combinations thereof.

85. A method of treating a cancer in a mammalian subject comprising administering to the subject a therapeutic composition comprising an effective amount of a purified population of bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

86. The method of any of claims 78-81, wherein the therapeutic composition comprises bacteria belonging to two or more genera.

87. The method of any of claims 78-81, wherein the therapeutic composition comprises bacteria belonging to three or more genera.

88. The method of any of claims 78-81, wherein the therapeutic composition comprises bacteria belonging to four or more genera.

89. The method of any of claims 79-81, wherein the therapeutic composition comprises bacteria belonging to five or more genera.

90. The method of any of claims 82-85, wherein the therapeutic composition comprises bacteria belonging to two or more species.

91. The method of any of claims 82-85, wherein the therapeutic composition comprises bacteria belonging to three or more species.

92. The method of any of claims 82-85, wherein the therapeutic composition comprises bacteria belonging to four or more species.

93. The method of any of claims 82-85, wherein the therapeutic composition comprises bacteria belonging to five or more species.

94. The method of any one of claims 56-93, further comprising administering an anticancer agent to the subject.

95. The method of claim 94, wherein the anticancer agent is a checkpoint inhibitor.

96. The method of claim 95, wherein the checkpoint inhibitor is selected from an anti-PD-1 antibody, an anti-CTLA-4 antibody, an anti-PD-L1 antibody or combinations thereof.

97. The method of claim 95, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors, STI-A1010 or combinations thereof.

98. The method of claim 94, wherein the anticancer agent is cyclophosphamide.

99. The method of any of claims 56-98, wherein the isolated population of bacteria is administered at a concentration of at least about 1×10² viable colony forming units.

100. The method of any of claims 56-98, wherein the isolated population of bacteria is administered at a concentration of at a concentration of about 1×10² to 1×10⁹ viable colony forming units.

101. The method of any of claims 56-100, wherein a fraction of the isolated population of bacteria comprises a spore-forming bacteria.

102. The method of any of claims 56-100, where a fraction of the isolated population of bacteria is in spore form.

103. The method of any of claims 56-102, wherein the composition further comprises a pharmaceutically acceptable excipient.

104. The method of any of claims 56-103, wherein the composition is formulated for delivery to the intestine.

105. The method of method of any of claims 56-103, wherein the composition is enterically coated.

106. The method of any of claims 56-102, wherein the composition is formulated for oral administration.

107. The method of claim 106, wherein the composition is formulated into a food or beverage.

108. The method of any of claims 56-107, wherein the mammalian subject is a human.

109. The method of any of claims 56-107, wherein the cancer is selected from metastatic melanoma, melanoma of the skin, non-small cell lung cancer, kidney cancer, bladder cancer, head and neck cancers, Merkel cell skin cancer (Merkel cell carcinoma), or Hodgkin lymphoma.

110. The method of any of claims 56-109, wherein prior to administration of the isolated population of bacteria the subject is subjected to antibiotic treatment and/or a bowel cleanse.

111. The method of any one of claims 56-110, wherein the subject has previously been treated for the cancer.

112. The method of claim 111, wherein the subject has been determined to be a non-responder to the previous treatment.

113. The method of claim 111 or 112, wherein the subject has been determined to have a toxic response to the previous treatment.

114. The method of any one of claims 111-113, wherein the previous treatment comprises immune checkpoint blockade monotherapy or immune checkpoint blockade combination therapy.

115. The method of any one of claims 56-114, wherein the cancer is recurrent cancer.

116. A method of identifying a mammalian subject as a candidate for immune checkpoint therapy in combination with adjuvant microbiome therapy, the method comprising:
a) obtaining a microbiome sample from the subject,
b) determining the prevalence of the genera of bacteria in the microbiome sample, and
c) determining that the subject is a candidate for the therapy if the microbiome sample comprises bacteria that are phylogenetic descendants of the most recent common ancestor (MRCA) of Faecalibacterium prausnitzii and Flavonifractor plautii.

117. A method of identifying a mammalian subject as a candidate for immune checkpoint therapy in combination with adjuvant microbiome therapy, the method comprising:
a) obtaining a microbiome sample from the subject,
b) determining the prevalence of the genera of bacteria in the microbiome sample, and
c) determining that the subject is a candidate for the therapy if the microbiome sample comprises bacteria that have 16S rDNA sequence identity of at least 94.5% to 16S rDNA sequences of species belonging to the family Ruminococcaceae.

118. The method of claim 117, wherein the bacteria have 16S rDNA sequence identity of at least 98.7% to 16S rDNA sequences of species belonging to the family Ruminococcaceae.

119. A method of identifying a mammalian subject as a candidate for immune checkpoint therapy in combination with adjuvant microbiome therapy, the method comprising:
a) obtaining a microbiome sample from the subject,
b) determining the prevalence of the genera of bacteria in the microbiome sample, and
c) determining that the subject is a candidate for the therapy if the microbiome sample comprises bacteria belonging to one or more of the genera Ruminococcus, Gemmiger, Faecalibacterium, Subdoligranulum or combinations thereof.

120. A method of identifying a mammalian subject as a candidate for immune checkpoint therapy in combination with adjuvant microbiome therapy the method comprising:
a) obtaining a microbiome sample from the subject,
b) determining the prevalence of the genera of bacteria in the microbiome sample, and
c) determining that the subject is a candidate for the therapy if the microbiome sample comprises bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

121. A method of identifying a mammalian subject as a candidate for immune checkpoint therapy in combination with adjuvant microbiome therapy, the method comprising:
a) obtaining a microbiome sample from the subject,
b) determining the prevalence of the genera of bacteria in the microbiome sample, and
c) determining that the subject is a candidate for the therapy if the microbiome sample comprises bacteria belonging to one or more of the genera Alistipes, Bacteroides, Blautia, Clostridium, Eubacterium, Parabacteroides or combinations thereof.

122. A method of identifying a mammalian subject as a candidate for immune checkpoint therapy in combination with adjuvant microbiome therapy, the method comprising:
a) obtaining a microbiome sample from the subject,
b) determining the prevalence of the genera of bacteria in the microbiome sample, and
c) determining that the subject is a candidate for the therapy if the microbiome sample comprises one or more of the genera Barnesiella, Bifidobacterium, Blautia, Erysipelotrichaceae, Odoribacter, Parabacteroides or combinations thereof.

123. A method of identifying a mammalian subject as a candidate for immune checkpoint therapy in combination with adjuvant microbiome therapy, the method comprising:
a) obtaining a microbiome sample from the subject,
b) determining the prevalence of the species of bacteria in the microbiome sample, and
c) determining that the subject is a candidate for the therapy if the microbiome sample comprises bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

124. A method of identifying a mammalian subject as a candidate for immune checkpoint therapy in combination with adjuvant microbiome therapy, the method comprising:
a) obtaining a microbiome sample from the subject,
b) determining the prevalence of the species of bacteria in the microbiome sample, and
c) determining that the subject is a candidate for the therapy if the microbiome sample comprises bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof.

125. A method of identifying a mammalian subject as a candidate for immune checkpoint therapy in combination with adjuvant microbiome therapy, the method comprising:
a) obtaining a microbiome sample from the subject,
b) determining the prevalence of the species of bacteria in the microbiome sample, and
c) determining that the subject is a candidate for the therapy if the microbiome sample comprises bacteria species selected from Alistipes senegalensis, Bacteroides dorei, Blautia_SC109, Clostridium_SC64, Eubacterium_biforme, Parabacteroides distasonis or combinations thereof.

126. A method of identifying a mammalian subject as a candidate for immune checkpoint therapy in combination with adjuvant microbiome therapy, the method comprising:
a) obtaining a microbiome sample from the subject,
b) determining the prevalence of the species of bacteria in the microbiome sample, and
c) determining that the subject is a candidate for the therapy if the microbiome sample comprises bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof.

127. The method of any of claims 116-126, wherein the subject is determined to be a candidate for immune checkpoint inhibitor therapy.

128. The method of any one of claims 116-127, wherein the wherein the immune checkpoint therapy comprises immune checkpoint blockade monotherapy or immune checkpoint blockade combination therapy.

129. The method of any of claims 116-126, wherein the subject is determined to be a candidate for cyclophosphamide therapy.

130. The method of any of claims 116-129, wherein the mammalian subject is a human.

131. The method of any of claims 116-130, wherein the cancer is selected from metastatic melanoma, melanoma of the skin, non-small cell lung cancer, kidney cancer, bladder cancer, head and neck cancers, Merkel cell skin cancer (Merkel cell carcinoma), or Hodgkin lymphoma.

132. A therapeutic composition comprising an effective amount of an isolated population of bacteria belonging to the genera Ruminococcus, Gemmiger, Faecalibacterium and Subdoligranulum.

133. A therapeutic composition comprising an effective amount of an isolated population of bacteria belonging to the genera Alistipes, Bacteroides, Barnesiella, Bifidobacterium, Blautia, Clostridium, Eubacterium, Erysipelotrichaceae, Odoribacter and Parabacteroides.

134. A therapeutic composition comprising an effective amount of an isolated population of bacteria belonging to the genera Barnesiella, Bifidobacterium, Blautia, Erysipelotrichaceae, Odoribacter and Parabacteroides.

135. A therapeutic composition comprising an effective amount of an isolated population of bacteria species Alistipes senegalensis, Bacteroides dorei, Blautia_SC109, Clostridium_SC64, Eubacteriumbiforme and Parabacteroides distasonis.

136. A therapeutic composition comprising an effective amount of an isolated population of bacteria species Barnesiella intestinihominis, Bifidobacterium bifidum, Bifidobacterium longum, Blautia_SC102, Blautia_SC109, Clostridium innocuum, Odoribacter splanchnicus and Parabacteroides distasonis.

137. A therapeutic composition comprising an effective amount of a purified population of bacteria belonging to one or more of the species listed in Tables 1A, 1B, 2A, 2B, 3A, 3B, 4A, 4B, 5A, 5B, 6A, 6B, 7A, 7B, 8A, 8B, 10 or 11.

138. A therapeutic composition comprising an effective amount of a purified population of bacteria belonging to two or more of the species listed in Tables 1A, 1B, 2A, 2B, 3A, 3B, 4A, 4B, 5A, 5B, 6A, 6B, 7A, 7B, 8A, 8B, 10 or 11.

139. A therapeutic composition comprising an effective amount of a purified population of bacteria belonging to three or more of the species listed in Tables 1A, 1B, 2A, 2B, 3A, 3B, 4A, 4B, 5A, 5B, 6A, 6B, 7A, 7B, 8A, 8B, 10 or 11.

140. A therapeutic composition comprising an effective amount of a purified population of bacteria belonging to four or more of the species listed in Tables 1A, 1B, 2A, 2B, 3A, 3B, 4A, 4B, 5A, 5B, 6A, 6B, 7A, 7B, 8A, 8B, 10 or 11.

141. A therapeutic composition comprising an effective amount of a purified population of bacteria belonging to one or more of the species listed in Table 1A.

142. A therapeutic composition comprising an effective amount of a purified population of bacteria belonging to one or more of the species listed in Table 1B.

143. A therapeutic composition comprising an effective amount of a purified population of bacteria belonging to one or more of the species listed in Table 10.

144. A therapeutic composition comprising an effective amount of a purified population of bacteria belonging to one or more of the species listed in Table 11.

145. A therapeutic composition comprising an effective amount of a purified population of bacteria belonging to two or more of the species listed in Table 1A.

146. A therapeutic composition comprising an effective amount of a purified population of bacteria belonging to two or more of the species listed in Table 1B.

147. A therapeutic composition comprising an effective amount of a purified population of bacteria belonging to two or more of the species listed in Table 10.

148. A therapeutic composition comprising an effective amount of a purified population of bacteria belonging to two or more of the species listed in Table 11.

149. A therapeutic composition comprising an effective amount of an isolated population of bacteria belonging to one or more of the species in a clade selected from clade 101, clade 14, clade 126, clade 61, clade 125, clade 135, or combinations thereof.

150. A therapeutic composition comprising an effective amount of purified population of bacteria belonging to one or more of the species in a clade selected from clade 101, clade 14, clade 126, clade 61, clade 125, clade 135, or combinations thereof.

151. A therapeutic composition comprising an effective amount of an isolated population of bacteria belonging to one or more of the species in the phylogenetic tree of FIG. 6.

152. A therapeutic composition comprising an effective amount of a purified population of bacteria belonging to one or more of the species in the phylogenetic tree of FIG. 6.

153. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacterial species that are phylogenetic descendants of the most recent common ancestor (MRCA) of Faecalibacterium prausnitzii and Flavonifractor plautii.

154. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises bacterial species that have 16S rDNA sequence identity of at least 94.5% to 16S rDNA sequences of species belonging to the family Ruminococcaceae.

155. The method of claim 154, wherein the bacteria have 16S rDNA sequence identity of at least 98.7% to 16S rDNA sequences of species belonging to the family Ruminococcaceae.

156. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

157. A method of identifying a mammalian subject as a donor whose feces are useful for fecal matter transfer, the method comprising:
a) obtaining a microbiome sample from the potential donor,
b) determining the prevalence and/or abundance of the species of bacteria in the microbiome sample, and
c) determining that the donor's feces is useful for fecal matter transfer if the microbiome sample comprises one or more of the bacteria species in one or more of clade 101, clade 14, clade 126, clade 61, clade 125 or clade 135.

158. A method of identifying a mammalian subject as a donor whose feces are useful for fecal matter transfer, the method comprising:
a) obtaining a microbiome sample from the potential donor,
b) determining the abundance of the species of bacteria in the microbiome sample, and
c) determining that the donor's feces is useful for fecal matter transfer if the microbiome sample comprises bacterial species that are phylogenetic descendants of the most recent common ancestor (MRCA) of Faecalibacterium prausnitzii and Flavonifractor plautii.

159. A method of identifying a mammalian subject as a donor whose feces are useful for fecal matter transfer, the method comprising:
a) obtaining a microbiome sample from the potential donor,
b) determining the abundance of the species of bacteria in the microbiome sample, and
c) determining that the donor's feces is useful for fecal matter transfer if the microbiome sample comprises bacterial species that have 16S rDNA sequence identity of at least 94.5% to 16S rDNA sequences of species belonging to the family Ruminococcaceae.

160. The method of claim 159, wherein the bacteria have 16S rDNA sequence identity of at least 98.7% to 16S rDNA sequences of species belonging to the family Ruminococcaceae.

161. A method of identifying a mammalian subject as a donor whose feces are useful for fecal matter transfer, the method comprising:
a) obtaining a microbiome sample from the potential donor,
b) determining the abundance of the species of bacteria in the microbiome sample, and
c) determining that the donor's feces is useful for fecal matter transfer if the microbiome sample comprises one or more bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

162. A method of identifying a mammalian subject as a donor whose feces are useful for fecal matter transfer, the method comprising:
a) obtaining a microbiome sample from the potential donor,
b) determining the abundance of the species of bacteria in the microbiome sample, and
c) determining that the donor's feces is useful for fecal matter transfer if the microbiome sample comprises one or more of the bacteria species in one or more of clade 101, clade 14, clade 126, clade 61, clade 125 or clade 135.

163. A therapeutic composition derived from fecal matter from a donor identified using the method of any one of claims 153-162.

164. The therapeutic composition of claim 163, further comprising a pharmaceutically acceptable excipient.

165. The therapeutic composition of claim 163, wherein the therapeutic composition comprises bacteria that are in vegetative and/or spore form.

166. The therapeutic composition of claim 163, wherein the therapeutic composition further comprises a checkpoint inhibitor.

167. The therapeutic composition of claim 166, wherein the checkpoint inhibitor is selected from anti-PD-1 antibody, an anti-CTLA-4 antibody, an anti-PD-L1 antibody or combinations thereof.

168. The therapeutic composition of claim 166, wherein the checkpoint inhibitor is selected from pembrolizumab, nivolumab, atezolizumab, avelumab, durvalumab, ipilimumab, pidilizumab, AMP-224, AMP-514, STI-A1110, TSR-042, RG-7446, BMS-936559, BMS-936558, MK-3475, CT 011, MPDL3280A, MEDI-4736, MSB-0020718C, AUR-012, LAG-3, OX40 inhibitors, OX40L inhibitors, TIGIT inhibitors, STI-A1010 or combinations thereof.

169. A of treating a cancer in a mammalian subject comprising administering to the subject a therapeutic composition of any one of claims 163-168.

170. A method of treating cancer comprising administering an anticancer treatment to a subject determined to have a microbiome sample comprising bacteria that are phylogenetic descendants of the most recent common ancestor (MRCA) of Faecalibacterium prausnitzii and Flavonifractor plautii.

171. A method of treating cancer comprising administering an anticancer treatment to a subject determined to have a microbiome sample comprising bacteria that have 16S rDNA sequence identity of at least 94.5% to 16S rDNA sequences of species belonging to the family Ruminococcaceae.

172. The method of claim 171, wherein the bacteria have 16S rDNA sequence identity of at least 98.7% to 16S rDNA sequences of species belonging to the family Ruminococcaceae.

173. A method of treating cancer comprising administering an anticancer treatment to a subject determined to have a microbiome sample comprising bacteria belonging to one or more of the genera Ruminococcus, Gemmiger, Faecalibacterium, Subdoligranulum or combinations thereof.

174. A method of treating cancer comprising administering an anticancer treatment to a subject determined to have a microbiome sample comprising bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof.

175. A method of treating cancer comprising administering an anticancer treatment to a subject determined to have a microbiome sample comprising bacteria belonging to one or more of the genera Alistipes, Bacteroides, Blautia, Clostridium, Eubacterium, Parabacteroides or combinations thereof.

176. A method of treating cancer comprising administering an anticancer treatment to a subject determined to have a microbiome sample comprising one or more of the genera Barnesiella, Bifidobacterium, Blautia, Erysipelotrichaceae, Odoribacter, Parabacteroides or combinations thereof.

177. A method of treating cancer comprising administering an anticancer treatment to a subject determined to have a microbiome sample comprising bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof.

178. A method of treating cancer comprising administering an anticancer treatment to a subject determined to have a microbiome sample comprising bacteria species selected from Alistipes senegalensis, Barnesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof.

179. A method of treating cancer comprising administering an anticancer treatment to a subject determined to have a microbiome sample comprising bacteria species selected from Alistipes senegalensis, Bacteroides dorei, Blautia_SC109, Clostridium_SC64, Eubacterium_biforme, Parabacteroides distasonis or combinations thereof.

180. A method of treating cancer comprising administering an anticancer treatment to a subject determined to have a microbiome sample comprising bacteria species selected from Barnesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof178. A method comprising evaluating a microbiome profile for bacteria that are phylogenetic descendants of the most recent common ancestor (MRCA) of Faecalibacterium prausnitzii and Flavonifractor plautii in a sample from a subject.

181. A method comprising evaluating a microbiome profile for bacteria that have 16S rDNA sequence identity of at least 94.5% to 16S rDNA sequences of species belonging to the family Ruminococcaceae in a sample from a subject.

182. The method of claim 181, wherein the bacteria have 16S rDNA sequence identity of at least 98.7% to 16S rDNA sequences of species belonging to the family Ruminococcaceae.

183. A method comprising evaluating a microbiome profile for bacteria belonging to one or more of the genera Ruminococcus, Gemmiger, Faecalibacterium, Subdoligranulum or combinations thereof in a sample from the subject.

184. A method comprising evaluating a microbiome profile for bacteria belonging to one or more of the genera Alistipes, Bacteroides, Barnesiella, Bifidobacterium, Blautia, Clostridium, Eubacterium, Erysipelotrichaceae, Odoribacter, Parabacteroides or combinations thereof in a sample from a subject.

185. A method comprising evaluating a microbiome profile for bacteria belonging to one or more of the genera Alistipes, Bacteroides, Blautia, Clostridium, Eubacterium, Parabacteroides or combinations thereof in a sample from a subject.

186. A method comprising evaluating a microbiome profile for one or more of the genera Barnesiella, Bifidobacterium, Blautia, Erysipelotrichaceae, Odoribacter, Parabacteroides or combinations thereof in a sample from a subject.

187. A method comprising evaluating a microbiome profile for bacteria species selected from Eubacterium siraeum, Clostridium leptum (GCF_000154345), Anaerotruncus colihominis, Subdoligranulum variabile, Clostridium methylpentosum, Pseudoflavonifractor capillosus, Ethanoligenens harbinense (GCF_000178115), Ruminococcus albus (GCF_000179635), Ruminococcus champanellensis (GCF_000210095), Flavonifractor plautii, Oscillibacter valericigenes, Oscillibacter ruminantium, Clostridium sporosphaeroides, Ruminococcus callidus, Ruminococcus flavefaciens (GCF_000518765), Clostridium jeddahense, Clostridium viride, Ruminococcus albus (GCF_000621285), Agathobaculum desmolans, Ruminococcus bicirculans, Ruthenibacterium lactatiformans, Clostridium phoceensis, Intestinimonas massiliensis, Anaeromassilibacillus senegalensis, Ruminococcus champanellensis (GCF_001312825), Bittarella massiliensis, Butyricicoccus porcorum, Acutalibacter muris, Clostridium leptum (GCF_002556665), Ruminococcus bromii (GCF_002834225, Monoglobus pectinilyticus, Ethanoligenens harbinense (GCF_003020045), Neglecta timonensis, Anaerotruncus rubiinfantis, Massilioclostridium coli, Angelakisella massiliensis, Sporobacter termitidis, Negativibacillus massiliensis, Massilimaliae massiliensis, Intestinibacillus massiliensis, Eubacterium coprostanoligenes, Provencibacterium massiliense, Papillibacter cinnamivorans, Clostridium merdae, Marasmitruncus massiliensis, Massilimaliae timonensis, Pygmaiobacter massiliensis, Clostridium minihomine, Neobitarella massiliensis, Faecalibacterium prausnitzii, Ruminococcus flavefaciens (GCF_000174895), Ruminococcaceae bacterium D16, Ruminococcus albus (GCF_000178155), Anaerotruncus sp_G3 2012, Oscillibacter sp 1 3, Clostridiales bacterium NK3B98, Oscillibacter sp KLE 1728, Firmicutes bacterium ASF500, Ruminococcus sp FC2018, Ruminococcus sp NK3A76, Ruminococcus flavefaciens (GCF_000701945), Ruminococcus sp HUN007, Bacterium MS4, Intestinimonas butyriciproducens, Oscillibacter sp ER4, Candidatus Soleaferrea massiliensis, Clostridium cellulosi, Clostridia bacterium UC5 1 2F7, Clostridia bacterium UC5 1 1E11, Clostridia bacterium UC5 1 1D1, Fournierella massiliensis, Clostridium sp W14A, Ruminococcaceae bacterium CPB6, Flavonifractor sp An92, Flavonifractor sp An91, Flavonifractor sp An306, Anaerofilum sp An201, Anaeromassilibacillus sp An200, Pseudoflavonifractor sp An187, Pseudoflavonifractor sp An184, Anaeromassilibacillus sp An172, Gemmiger sp An120, Flavonifractor sp An100, Flavonifractor sp An10, Eubacteriaceae bacterium CHKCI005, Ruminococcaceae bacterium P7, Ruminococcus bromii (GCF_900101355), Ruminococcus sp YE78, Ruminococcaceae bacterium FB2012, Ruminococcaceae bacterium Marseille P2935, Hydrogenoanaerobacterium saccharovorans, Ruminococcaceae bacterium D5, Oscillibacter sp PC13, Pseudoflavonifractor sp Marseille P3106, Neglecta sp Marseille P3890, Clostridium sp SN20, Anaerotruncus sp AT3, Anaeromassilibacillus sp Marseille P3876, Gemmiger formicilis (STS00001), Ruminococcaceae unnamed sp 1 (STS00002), Ruminococcaceae unnamed sp 2 (STS00003), Gemmiger formicilis (STS00004), Ruminococcaceae unnamed sp 3 (STS00005), Ruminococcaceae unnamed sp 4 (STS00006), Ruminococcaceae unnamed sp 5 (STS00007), Ruminococcaceae unnamed sp 6 (STS00008), Ruminococcaceae unnamed sp 7 (STS00009) or combinations thereof in a sample from a subject.

188. A method comprising evaluating a microbiome profile for bacteria species selected from Alistipes senegalensis, Bamesiella intestinihominis, Bacteroides dorei, Bifidobacterium bifidum, Bifidobacterium longum, Blautia_SC102, Blautia_SC109, Clostridium_SC64, Clostridium innocuum, Odoribacter splanchnicus, Eubacterium_biforme, Parabacteroides distasonis or combinations thereof in a sample from a subject.

189. A method comprising evaluating a microbiome profile for bacteria species selected from Alistipes senegalensis, Bacteroides dorei, Blautia_SC109, Clostridium_SC64, Eubacterium_biforme, Parabacteroides distasonis or combinations thereof in a sample from a subject.

190. A method comprising evaluating a microbiome profile for bacteria species selected from Bamesiella intestinihominis, Bifidobacterium bifidum, Bifidobacterium longum, Blautia_SC102, Blautia_SC109, Clostridium innocuum, Odoribacter splanchnicus, Parabacteroides distasonis or combinations thereof in a sample from a subject.

191. The method of any one of claims 181-190, wherein the method further comprises comparing the microbiome profile to a control microbiome.

192. The method of claim 191, wherein the control microbiome comprises a microbiome sample from a subject determined to be a responder to an anticancer treatment.

193. The method of claim 191, wherein the control microbiome comprises a microbiome sample from a subject determined to be a non-responder to an anticancer treatment.
